(19) United States
(12) Patent Application Publication
(10) Pub. No.: US 2022/0088183 A1
(43) Pub. Date: Mar. 24, 2022

(54) CHIMERIC ONCOLYTIC HERPESVIRUS THAT STIMULATES AN ANTITUMOR IMMUNE RESPONSE

(71) Applicant: THE RESEARCH INSTITUTE AT NATIONWIDE CHILDREN'S HOSPITAL, Columbus, OH (US)

(72) Inventors: Kevin A. Cassady, Columbus, OH (US); Justin C. Roth, Birmingham, AL (US)

(21) Appl. No.: 17/271,403

(22) PCT Filed: Aug. 30, 2019
(86) PCT No.: PCT/US19/49047

§ 371 Date: Feb. 25, 2021

Related U.S. Application Data
(60) Provisional application No. 62/725,809, filed on Aug. 31, 2018.
(60) Provisional application No. 62/731,365, filed on Sep. 14, 2018.

(51) Int. Cl. A61K 39/245; A61K 39/00

(52) U.S. Cl. CPC A61K 39/245; A61K 2039/80; A61K 2039/5254; A61K 39/0011

(57) ABSTRACT

A chimeric oncolytic virus is described that includes a herpesvirus having a modified nucleic acid sequence, including a modification of the herpesvirus gamma (1)34.5 gene (γ₁34.5) or a nucleic acid with at least about 70% homology to the γ₁34.5 gene that reduces its expression; a second viral nucleic acid sequence encoding a PKR evasion protein that does not cause virulence; and a third nucleic acid sequence encoding a tumor-associated antigen. Methods of using the chimeric oncolytic virus to treat subjects having cancer, or to vaccinate subjects at risk of developing cancer, are also described.

### CROSS-REFERENCE TO RELATED APPLICATIONS

This application claims the benefit of U.S. Provisional Application No. 62/725,809, filed Aug. 31, 2018, and U.S. Provisional Application No. 62/731,365, filed Sep. 14, 2018, both of which are incorporated herein by reference.

### GOVERNMENT FUNDING

The present invention was made with government support under Grant No. CA071933 awarded by the National Institutes of Health. The US government has certain rights in this invention.

### SEQUENCE LISTING

The instant application contains a Sequence Listing which has been submitted electronically in ASCII format and is hereby incorporated by reference in its entirety. Said ASCII copy, created on Aug. 24, 2018, is named NCH-027861US PRO SEQUENCE LISTING_ST25 and is 8,621 bytes in size.

### BACKGROUND

Cancer immunotherapy is a novel treatment option that involves priming the immune system for tumor cell eradication. Immunotherapies have been used to overcome the limited efficacy of the classic therapeutic options like surgery, radio-, chemo-, or antibody-therapy for patients with advanced stage solid tumor. While some tumors have been successfully treated with immunotherapeutic approaches others have been more resistant. Solid tumors and tumors with lower mutational loads (e.g. Pediatric Cancers) are harder to target. These lower mutational burden however, often express membrane associated fetal antigens (e.g. EphA2, GD2, IL13Ra2, EGFR VIII) that contribute their transformation and provide potential immunotherapeutic targets. Gros et al., Nature medicine, 22:433-8 (2016).

Two promising areas in immunotherapy involve pulsing dendritic cells with a cocktail of antigen peptides to vaccinate against the tumor (Phuphanich et al., Cancer immunology, immunotherapy: CII, 62:125-35 (2013)) and oncolytic viruses (OV). Markert et al., Mol Ther., 17:199-207 (2009). Although they are distinct approaches, both strategies likely achieve efficacy through induction of anti-tumor immunity. Brown M C, Gromeier M., Curr Opin Virol., 13:81-5 (2015). Viro-immunotherapy is based on biologic anticancer agents that preferentially target tumor cells while sparing normal cells. Oncolytic HSVs have been safely used in clinical trials in a wide range of cancer types including brain tumors. With improved mechanistic understanding of how viral replication and host immune mediated responses contribute to the anti-tumor response, newer next gene have been engineered to improve their efficacy and/or safety profile. Ghonime M G, Cassady K A., Cancer immunology research 2018; 6:1499-510

Gliomas are the most frequently occurring primary malignant brain tumors, with glioblastoma multiforme (GBM) being one of the most fatal and treatment-refractory cancers. Since median time to progression and median survival of these patients have changed minimally in the past fifty years, new multimodal treatment strategies are needed. Genetically-modified HSV are attractive as replication-competent, oncolytic vectors, and their genome facilitates high level transgene expression for multimodal treatment approaches. Although their safety has been demonstrated in clinical trials, first generation oHSVs are limited by poor replication in tumors. Markert et al., Rev Med Virol, 10(1): p. 17-30 (2000). Poor replication arises from safety precautions that dictate removal of the γ₁34.5 gene, which prevents neurovirulence, but also abrogates viral resistance to host interferon (IFN) signaling pathways that trigger translational arrest. Wakimoto et al., Gene Ther, 10(11): p. 983-90 (2003).

A number of oHSV genes required for efficient replication also actively suppress host gene expression and antigen processing pathways. HSV-1 encoded ICP47, US3 kinase, and gB proteins suppress peptide loading on—or surface expression of—MHC class I and II molecules. Fruh et al., Nature, 375(6530): p. 415-8 (1995); Imai et al., PLoS One, 8(8): p. e72050 (2013). Similarly, the virion host shutoff (VHS) protein in oHSV reduces host gene expression, including potential tumor antigens, by degrading host cell transcripts in infected cells. Taddeo et al., J Virol, 87(8): p. 4516-22 (2013) The concerted action of these viral proteins allow for selective viral gene expression and cloaking of infected cells from immune surveillance mechanisms. Therefore, the use of oHSV for eliciting effective anti-GBM responses will likely require viruses that effectively reverse the immunosuppressive state in GBMs and specifically enhance acquired responses to TAAs.

oHSV infection itself is thought to transiently reverse the immunosuppressive tumor microenvironment and stimulate adaptive responses. Iizuka et al., Int J Cancer, 118(4): p. 942-9 (2006). Host cell anti-viral responses induce IFN and cytokine signaling that culminate in recruitment and activation of innate immune cells (neutrophils, NK cells, dendritic cells (DCs) and macrophages) and subsequent stimulation of adaptive (CD4+, CD8+) responses. Miller et al., Cancer Res, 60(20): p. 5714-22 (2000). Although this contributes to viral clearance, it is also believed to indirectly stimulate antitumor responses. Parker et al., Cancer Gene Ther, 12(4): p. 359-68 (2005). A number of studies support this hypothesis. First, oHSV engineered to express proinflammatory genes (IL-12, IL-18, IL-4, TNF-α) have enhanced anti-tumor effects. For example, treatment with an IL-12- and CCL2-expressing oHSV increased recruitment of activated macrophages and T cells and improved survival without decreasing viral replication. Second, preexisting HSV immunity improves oHSV efficacy and survival and this survival advantage is lost in immune-suppressed mice. Miller et al., Mol Ther, 7(6): p. 741-7 (2003). Finally, transcriptional array analyses from a Phase Ib clinical trial of the Δγ₁34.5 oHSV (G207) suggest that anti-viral immune responses contributed to anti-GBM activity, as long-term survivors (>6 ms) exhibited greater inflammatory and interferon-stimulated gene expression, compared to non-responders (survival <3 ms).

Virotherapy is a mature experimental therapy and in some cases has FDA approval. Talimogene laherparepvec (T-VEC), an attenuated herpes simplex virus incorporating a granulocyte-macrophage colony-stimulating factor (GM-CSF) transgene, is the first in class FDA approved oncolytic HSV for treatment of advanced, unresectable stage 3 and 4 melanoma by intralesional injection. Pol et al., Oncoimmunology, 5:e1115641 (2016). As an immunotherapeutic, the repeated dosing of virus and combining virus with other immunomodulators improved clinical response in patients with therapy resistant melanoma. C134 is another next-generation oHSV with improved protein translation and replication over first generation oHSVs in cells with defective PAMP sensing and IFN signaling. In non-malignant cells, C134 induces IRF3 mediated IFN and Cytokine signaling thus restricting efficient viral replication in the tumor cells. Cassady et al., Journal of Virology, 86:610-4 C134 (2012), maintains late viral protein synthesis and replicates better than 1st gen viruses and this leads to increased cytopathic effect (CPE) and antigen load but remains as safe as the parent Δγ134.5 HSV (18-20). Not only the oncolytic HSV has direct anti-tumor activity caused by viral replication and lysis in infected cells but also it elicits an immune response that contributes to the overall anti-tumor activity. Introducing an OV does cause cellular damage and lead to the release of tumor antigens from virally-infected cells, pro-inflammatory pathogen-associated molecular pattern (PAMP) and damage-associated molecular pattern (DAMP), cytokines and chemokines produced during viral infection stimulate the immune response and reverse tumor associated immunosuppression. Russell S J, Barber G N., Cancer cell, 33:599-605 (2018).

Because of their intrinsic antigenicity, tumors have evolved to evade immune surveillance, and have sluggish pathogen-associated molecular pattern (PAMP) and damage-associated molecular pattern (DAMP) responses and defective antigen presentation by down-regulating antigen processing machinery such as the major histocompatibility complex (MHC) I pathway, proteosome subunits latent membrane protein (LMP)2 and LMP7, tapasin, and transporter associated with antigen processing (TAP) protein. Johnsen et al., J. of immunology, 163:4224-31 (1999) Thus, the expression of tumor antigens is downregulated and the ability of cytotoxic T lymphocytes to recognize apparent tumor cells and promote tolerance is impeded. Maeurer et al., J Clin Invest., 98(7):1633-41 (1996). Replication of the oncolytic virus causes cell death, ultimately inducing pro-inflammatory DAMP and PAMP responses, and promoting phagocytosis of dead or damaged virus-infected tumor cells. Viral infections are well-described at breaking immune tolerance and inducing auto-immunity to self-antigen. Steed A L, Stappenbeck T S., Curr Opin Immunol., 31:102-7 (2014) HSV is no exception and can induce a systemic auto-immune reaction (erythema multiforme), (Lucchese A., Autoimmun Rev, 17:576-81 (2018)) a T cell mediated keratitis (Buela K A, Hendricks R L, J Immunol, 194:379-87 (2015)), and a persistent auto-immune encephalitis (Nosadini et al., Dev Med Child Neurol 59:796-805 (2017)). The virus induces a robust inflammatory and immune mediated response during lytic infection. Like all viruses, it has evolved to survive the antiviral response and confines the peak of the immune response during its lytic phase. Cassady et al., Viruses, 4; 8 pii E43 (2016). Over time this inflammatory and immune mediated response restricts viral lytic infection: eliciting innate and adaptive immune responses that result in long-term immune responses mediated by T cells. Melzer et al., Biomedicines, 5(1). pii: E8 (2017) While oHSV-infection recruits immune effectors, it also reduces host gene expression in the infected cell enabling selective viral gene expression and suppressing immediate host immune detection. C134's ability to maintain protein translation in infected cells while stimulating the IFN response make it particularly well suited as multimodal therapeutic platform.

### SUMMARY

Gliomas are the most frequently occurring primary malignant brain tumors, with glioblastoma multiforme (GBM) being one of the most fatal and treatment-refractory cancers. Based on the mortality rates and the morbidity associated with current regimens, it is clear that new therapeutic options are needed. Oncolytic herpes viruses (oHSV) represent a novel therapeutic for treatment-refractory cancers and have demonstrated efficacy in early phase clinical trials. oHSV mediate direct anti-tumor effects through lytic replication in tumors cells, but more recent data suggest that the indirect immune-stimulating effects of oHSV may have a greater impact on their efficacy. In this regard, oHSV infections trigger host cell anti-viral signaling pathways that prime both anti-viral and anti-tumor immune responses. The inventors have created a novel oHSV (C134) that synthesizes proteins and replicates better in the tumor (direct oncolytic properties) and has enhanced immune stimulation potential (indirect properties). They have also shown that expression of tumor proteins (tumor associated antigens: TAAs) will stimulate an immune response against the tumor and that by engineering the TAAs such that they are secreted and bind to specialized antigen presenting immune cells will “vaccinate” and induce a long-term anti-tumor immune response even after the virus is gone. The inventors have also demonstrated that they can enhance tumor-specific immune responses, rather than those directed at viral antigens, by engineering C134 to express tumor antigens that are secreted from the infected cells and targeted to professional antigen presenting cells.

### BRIEF DESCRIPTION OF THE SEQUENCES

SEQ ID NO: 1: is a γ₁34.5 gene derived from a herpes simplex virus 1: atggcccgcc
gccgccgcca tcgcggcccc cgccgccccc ggccgcccgg gcccacgggcgccgtcccaa ccgcacagtc ccaggtaacc
tccacgccca actcggaacc cgcggtcagg agcgcgcccg cggccgcccc gccgccgccc cccgccggtg ggcccccgcc
ttcttgttcg ctgctgctgc gccagtggct ccacgttccc gagtccgcgt ccgacgacga cgatgacgac gactggccgg
acagcccccc gcccgagccg gcgccagagg cccggcccac cgccgccgcc ccccggcccc ggcccccacc
gcccggcgtg ggcccggggg gcggggctga cccctcccac cccccctcgc gccccttccg ccttccgccg cgcctcgccc
tccgcctgcg cgtcaccgcg gagcacctgg cgcgcctgcg cctgcgacgc gcgggcgggg agggggcgcc
ggagcccccc gcgacccccg cgacccccgc gacccccgcg acccccgcga cccccgcgcg ggtgcgcttc tcgccccacg
tccgggtgcg ccacctggtg gtctgggcct cggccgcccg cctggcgcgc cgcggctcgt gggcccgcga gcgggccgac
cgggctcggt tccggcgccg ggtggcggag gccgaggcgg tcatcgggcc gtgcctgggg cccgaggccc gtgcccgggc
cctggcccgc ggagccggcc cggcgaactc ggtctaa
SEQ ID NO: 2 is an IRS-1 sequence derived from a human cytomegalovirus: atggcccagc
gcaacggcat gtcgccgcgc cccccgcccc ttggtcgcgg ccgcggggcc ggagggcctt cgggggttgg ttcctctcct
ccttcttctt gtgtgccgat gggagcgccg tcaacagcgg gcactggtgc gagtgctgcg gctacgacga cgccgggcca
cggcgtccac cgggtagaac cccgcgggcc gccgggcgcc cctccgagta gcggcaacaa tagcaacttt tggcacggcc
cggagcgcct gttgctgtct cagattccgg tggagcgcca ggcgctgacg gagctggaat accaggccat gggcgccgtg
tggcgcgcgg cglittlggc caacagcacg ggccgcgcca tgcgcaagtg gtcgcagcgc gacgcgggca cgctgctgcc
gctcggacgg ccgtacggat tctacgcgcg ggtgacgccg cgcagccaga tgaacggcgt gggcgcgacg gacctgcgtc
aactgtcgcc gcgggacgcg tggatcgtac tggtggctac cgtggtgcac gaggtggacc ccgcagccga cccgacggtg
ggcgacaagg ccggccatcc cgagggtctg tgcgcgcagg acggactgta cctggcgctg ggcgccgggt tccgcgtgtt
cgtgtacgac ctggcaaaca acacgctgat cctagcggcg cgcgacgcgg acgagtggtt tcggcacggc gcgggcgagg
tggtgcggct gtaccgctgc aaccggctgg gcgtgggcac cccgcgcgcg acgctgctgc ctcagccggc gctccgacag
acgttgctgc gcgccgagga ggcgacggcg ctcggacggg agctgcgccg gcggtgggcc ggcacgacgg
tggcgctgca gacgccgggc aggcgactgc agccgatggt actgctgggc gcgtggcagg agctggcgca gtacgagccg
ttcgcgtcgg cgccgcaccc cgcgtcgctg ctgacggccg tgcgtcggca cctgaaccag cgtctgtgct gcggctggct
ggcgctgggc gcggtgctgc ccgcgcggtg gctgggctgc gcggcggggc cggcgacggg gacggcggcg
gggacgacgt cgccgccagc ggcgagcggc acggagacgg aggccgccgg cggggacgcg ccgtgcgcga
tagcgggagc cgtggggtcc gctgtacctg tgcctccgca gccgtacggc gccgccggcg ggggcgcgat ttgcgtgcct
aacgcggacg cgcacgcggt ggtcggggcg gacgcggcag cagcagcggc gccgacggtg atggtgggtt cgacagcgat
ggcgggtccg gcggcgtcgg ggaccgtgcc gcgcgccatg ctggtggtgc tgctggacga gctgggcgcc gtgttcgggt
actgcccgct ggacgggcac gtgtacccgc tggcggcgga gctgtcgcac tttctgcgcg cgggcgtgct gggcgcgctg
gcgctgggac gcgagtcggc gcccgccgcc gaggccgcgc ggcggctgct gcccgagctg gaccgcgagc
agtgggagcg gccgcgctgg gacgcgctgc acctgcaccc gcgcgccgcg ctgtgggcgc gcgagccgca
cgggcagtgg gagttcatgt ttcgcgaaca acgcggtgac cccataaatg atcccctcgc atttcgtctt tcggacgctc
gaactctcgg tctcgacctc accaccgtca tgacagagcg tcaaagtcaa ttgcccgaaa agtatatcgg tttctatcag
attaggaaac ctccttggct catggaacaa cctccacccc catctcgcca aaccaaaccg gacgctgcaa cgatgccccc
accgctcagt gctcaggcaa gcgtcagcta cgcgctccga tacgatgacg agtcctggcg cccgctcagc acagttgacg
accacaaagc ctggttggat ctcgacgaat cacattgggt cctcggggac agccgacccg acgatataaa acaacgcaga
ctgctgaagg ccactcaacg acgaggcgcc gaaatcgaca gacccatgcc tgtcgtgcct gaagaatgtt acgaccaacg
cttcactacc gaaggccacc aggtcatccc gttgtgcgcg tccgaacccg aggatgacga cgaagatcct acctacgacg
aattgccgtc gcgcccaccc cagaaacata agccgccaga caaacctccg cgcttatgca aaacgggccc cggcccacct
ccgctgccgc caaagcaacg gcacggttcc accgacggaa aagtttctgc gccccgacag tcggagcatc ataaaagaca
gacccgaccg ccaaggccgc caccgcccaa attcggggat agaaccgcgg cccatctctc gcaaaatatg cgggacatgt
acctcgatat gtgtacatct tcgggccaca ggccacggcc gccagcacct ccgcggccga aaaaatgtca aacacacgcc
cctcaccacg ttcatcattg a.
SEQ ID NO: 3 is a TRS-1 sequence derived from a human cytomegalovirus: ttattgagca
ttgtaatggt agtgtgtggc tatattagaa aacgtgacgc gtcgcatgtc gcggcacaat ctggcagcgg ggtcggggta
gggtacggtg ggaggcatgt acacagatgg aacaaaagca gaagtaacgt gagaaggagc atacagtcca gtatccagcg
gttcctgagt agcaccaccc atcaactgaa tgccctcatg agtaaaagtc tgcgggcgac agcccttggg gaccgttggc
atgggacgat caatctccaa accacagcgt aacaccgttt tcttccaacg tcgttgatag acgtcgtttt tacggttact
cccaagaacc cagaaagtct cgtccaagtc gtaccaggaa tcttctccgg ggagacgcga cggtttccaa tcctcgtcgt
ctcgtctcaa agcacgtccc aaactggctt gaggagtcaa cggtggttct gtgggtcggg tgtagcgcga gtgttttccc
ttcatgagcg attcatcctc cttgccttta ggctttttgg tctttttgtg tatcatctgg ccgccggcct ccataaccac cgtggccaag
tccagtccca gagcttgagc gtcggcgcgg cgtcgggcgt cttgcaggta gtcttccaca tttgcacaga tggccgggtg
tttggtggct agggtgagga cctcagcctc gccgcgaccc ggacgtagca aaaaagccaa ctgcccgtgc ggctcgcgcg
cccacagcgc ggcgcgcggg tgcaggtgca gcgcgtccca gcgcggccgc tcccactgct cgcggtccag ctcgggcagc
agccgccgcg cggcctcggc ggcgggcgcc gactcgcgtc ccagcgccag cgcgcccagc acgcccgcgc
gcagaaagtg cgacagctcc gccgccagcg ggtacacgtg cccgtccagc gggcagtacc cgaacacggc gcccagctcg
tccagcagca ccaccagcat ggcgcgcggc acggtccccg acgccgccgg acccgccatc gctgtcgaac ccaccatcac
cgtcggcgcc gctgctgctg ccgcgtccgc cccgaccacc gcgtgcgcgt ccgcgttagg cacgcaaatc gcgcccccgc
cggcggcgcc gtacggctgc ggaggcacag gtacagcgga ccccacggct cccgctatcg cgcacggcgc gtccccgccg
gcggcctccg tctccgtgcc gctcgccgct ggcggcgacg tcgtccccgc cgccgtcccc gtcgccggcc ccgccgcgca
gcccagccac cgcgcgggca gcaccgcgcc cagcgccagc cagccgcagc acagacgctg gttcaggtgc
cgacgcacgg ccgtcagcag cgacgcgggg tgcggcgccg acgcgaacgg ctcgtactgc gccagctcct gccacgcgcc
cagcagtacc atcggctgca gtcgcctgcc cggcgtctgc agcgccaccg tcgtgccggc ccaccgccgg cgcagctccc
gtccgagcgc cgtcgcctcc tcggcgcgca gcaacgtctg tcggagcgcc ggctgaggca gcagcgtcgc gcgcggggtg
cccacgccca gccggttgca gcggtacagc cgcaccacct cgcccgcgcc gtgccgaaac cactcgtccg cgtcgcgcgc
cgctaggatc agcgtgttgt ttgccaggtc gtacacgaac acgcggaacc cggcgcccag cgccaggtac agtccgtcct
gcgcgcacag accctcggga tggccggcct tgtcgcccaa cgtcgggtcg gctgcggggt ccacctcgtg caccacggta
gccaccagta cgatccacgc gtcccgcggc gacagttgac gcaggtccgt cgcgcccacg ccgttcatct ggctgcgcgg
cgtcacccgc gcgtagaatc cgtacggccg tccgagcggc agcagcgtgc ccgcgtcgcg ctgcgaccac ttgcgcatgg
cgcggcccgt gctgttggcc aaaaacgccg cgcgccacac ggcgcccatg gcctggtatt ccagctccgt cagcgcctgg
cgctccaccg gaatctgaga cagcaacagg cgctccgggc cgtgccaaaa gttgctattg ttgccgctac tcggaggggc
gcccggcggc ccgcggggtt ctacccggtg gacgccgtgg cccggcgtcg tcgtagccgc agcactcgca ccagtgcccg
ctgtggacgg cgctcccatc ggcacacaag aagaaggagg agaggaacca acccccgaag gccctccggc cccgcggccg
cgaccaaggg gcggggggcg cggcgacatg ccgttgcgct gggccat.
SEQ ID NO: 4 is a shared 130 amino acid region of IRS1 and TRS1 sequence derived from a
human cytomegalovirus: atggcccagc gcaacggcat gtcgccgcgc cccccgcccc ttggtcgcgg
ccgcggggcc ggagggcctt cgggggttgg ttcctctcct ccttcttctt gtgtgccgat gggagcgccg tccacagcgg
gcactggtgc gagtgctgcg gctacgacga cgccgggcca cggcgtccac cgggtagaac cccgcgggcc gccgggcgcc
cctccgagta gcggcaacaa tagcaacttt tggcacggcc cggagcgcct gttgctgtct cagattccgg tggagcgcca
ggcgctgacg gagctggaat accaggccat gggcgccgtg tggcgcgcgg cgtattggc caacagcacg ggccgcgcca
tgcgcaagtg gtcgcagcgc.

SEQ ID NO: 5 is “C170” from—HSV-C134, the Complete Viral Genome v1 Chimeric HSV expressing EphA2 Full-ML (C134+pCK1201), see FIG. 19D.

SEQ ID NO: 6 is >C171_from_—_HSV-C134_Complete_Viral_Genome_v1 Chimeric HSV expressing EphA2-Full-MLM (C154+pCK1200), see FIG. 19E.

SEQ ID NO: 7 is >C172_from_—_HSV-C134_Complete_Viral_Genome_v1 Chimeric HSV expressing EphA2-Ecto-ML (C154+pCK1205), see FIG. 19F.

SEQ ID NO: 8 is >C173_from_—_HSV-C134_Complete_Viral_Genome_v1 Chimeric HSV expressing EphA2-Ecto-MLM (C154+pCK1207), see FIG. 19G.

SEQ ID NO: 9 is >C174_from_—_HSV-C134_Complete_Viral_Genome_v1 Chimeric HSV expressing EphA2-Endo-ML (C154+pCK1210), see FIG. 19H.

SEQ ID NO: 10 is >C175_from_—_HSV-C134_Complete_Viral_Genome_v1 Chimeric HSV expressing EphA2 Endo-MLM (C154+pCK1212), see FIG. 19I.

SEQ ID NO: 11 is hEphA2 ML extracted sequence derived from a human sequence:
gcctatggga atgaaagacc ccacctgtag gtttggcaag ctaggatcaa ggtcaggaac agagaaacag gagaatatgg
gccaaacagg atatctgtgg taagcagttc ctgccccgct cagggccaag aacagttgga acaggagaat atgggccaaa
caggatatct gtggtaagca gttcctgccc cgctcagggc caagaacaga tggtccccag atgcggtccc gccctcagca
gtttctagag aaccatcaga tgtttccagg gtgccccaag gacctgaaat gaccctgtgccttatttgaa ctaaccaatc
agttcgcttc tcgcttctgt tcgcgcgctt ctgctccccg agctctatat aagcagagct ggtttagtga accgtcagat
cgcctggaga cgccatcca gctgattga cctccataga agacaccgac tctagctaga ggatctccta ggaagctggc
cgcacaaagt ggtaccggat cccgggtcga ccatggagct ccaggcagcc cgcgcctgct tcgccctgct gtggggctgt
gcgctggccg cggccgcggc ggcgcagggc aaggaagtgg tactgctgga ctttgctgca gctggagggg agctcggctg
gctcacacac ccgtatggca aagggtggga cctgatgcag aacatcatga atgacatgcc gatctacatg tactccgtgt
gcaacgtgat gtctggcgac caggacaact ggctccgcac caactgggtg taccgaggag aggctgagcg tatcttcatt
gagctcaagt ttactgtacg tgactgcaac agcttccctg gtggcgccag ctcctgcaag gagactttca acctctacta
tgccgagtcg gacctggact acggcaccaa cttccagaag cgcctgttca ccaagattga caccattgcg cccgatgaga
tcaccgtcag cagcgacttc gaggcacgcc acgtgaagct gaacgtggag gagcgctccg tggggccgct cacccgcaaa
ggcttctacc tggccttcca ggatatcggt gcctgtgtgg cgctgctctc cgtccgtgtc tactacaaga agtgccccga
gctgctgcag ggcctggccc acttccctga gaccatcgcc ggctctgatg caccttccct ggccactgtg gccggcacct
gtgtggacca tgccgtggtg ccaccggggg gtgaagagcc ccgtatgcac tgtgcagtgg atggcgagtg gctggtgccc
attgggcagt gcctgtgcca ggcaggctac gagaaggtgg aggatgcctg ccaggcctgc tcgcctggat tattaagtt
tgaggcatct gagagcccct gcttggagtg ccctgagcac acgctgccat cccctgaggg tgccacctcc tgcgagtgtg
aggaaggctt cttccgggca cctcaggacc cagcgtcgat gccttgcaca cgacccccct ccgccccaca ctacctcaca
gccgtgggca tgggtgccaa ggtggagctg cgctggacgc cccctcagga cagcgggggc cgcgaggaca ttgtctacag
cgtcacctgc gaacagtgct ggcccgagtc tggggaatgc gggccgtgtg aggccagtgt gcgctactcg gagcctcctc
acggactgac ccgcaccagt gtgacagtga gcgacctgga gccccacatg aactacacct tcaccgtgga ggcccgcaat
ggcgtctcag gcctggtaac cagccgcagc ttccgtactg ccagtgtcag catcaaccag acagagcccc ccaaggtgag
gctggagggc cgcagcacca cctcgcttag cgtctcctgg agcatccccc cgccgcagca gagccgagtg tggaagtacg
aggtcactta ccgcaagaag ggagactcca acagctacaa tgtgcgccgc accgagggtt tctccgtgac cctggacgac
ctggccccag acaccaccta cctggtccag gtgcaggcac tgacgcagga gggccagggg gccggcagca aggtgcacga
attccagacg ctgtccccgg agggatctgg caacttggcg gtgattggcg gcgtggctgt cggtgtggtc ctgcttctgg
tgctggcagg agttggcttc tttatccacc gcaggaggaa gaaccagcgt gcccgccagt ccccggagga cgtttacttc
tccaagtcag aacaactgaa gcccctgaag acatacgtgg acccccacac atatgaggac cccaaccagg ctgtgttgaa
gttcactacc gagatccatc catcctgtgt cactcggcag aaggtgatcg gagcaggaga gtttggggag gtgtacaagg
gcatgctgaa gacatcctcg gggaagaagg aggtgccggt ggccatcaag acgctgaaag ccggctacac agagaagcag
cgagtggact tcctcggcga ggccggcatc atgggccagt tcagccacca caacatcatc cgcctagagg gcgtcatctc
caaatacaag cccatgatga tcatcactga gtacatggag aatggggccc tggacaagtt ccttcgggag aaggatggcg
agttcagcgt gctgcagctg gtgggcatgc tgcggggcat cgcagctggc atgaagtacc tggccaacat gaactatgtg
caccgtgacc tggctgcccg caacatcctc gtcaacagca acctggtctg caaggtgtct gactttggcc tgtcccgcgt
gctggaggac gaccccgagg ccacctacac caccagtggc ggcaagatcc ccatccgctg gaccgccccg gaggccattt
cctaccggaa gttcacctct gccagcgacg tgtggagctt tggcattgtc atgtgggagg tgatgaccta tggcgagcgg
ccctactggg agttgtccaa ccacgaggtg atgaaagcca tcaatgatgg cttccggctc cccacaccca tggactgccc
ctccgccatc taccagctca tgatgcagtg ctggcagcag gagcgtgccc gccgccccaa gttcgctgac atcgtcagca
tcctggacaa gctcattcgt gcccctgact ccctcaagac cctggctgac tttgaccccc gcgtgtctat ccggctcccc
agcacgagcg gctcggaggg ggtgcccttc cgcacggtgt ccgagtggct ggagtccatc aagatgcagc agtatacgga
gcacttcatg gcggccggct acactgccat cgagaaggtg gtgcagatga ccaacgacga catcaagagg attggggtgc
ggctgcccgg ccaccagaag cgcatcgcct acagcctgct gggactcaag gaccaggtga acactgtggg gatccccatc
gggaattcag aacaaaaact catctcagaa gaagatctag gaggcggcgg gtcaggtgga ggtggctctg gcggtggcgg
ttaaaattcc gactcactat agggcgaatt aattccggag atctctagat ccggagagac gatggcagga gccgcgcata
tatacgcttg gagccagccc gccctcacag ggcgggccgc ctcgggggcg ggactggcca atcggcggcc gccagcgcgg
cggggcccgg ccaaccagcg tccgccgagt cttcggggcc cggcccattg ggcgggagtt accgcc
SEQ ID NO: 12 hEphA2 MLM extracted sequence derived from a human sequence:
gcctatggga atgaaagacc ccacctgtag gtttggcaag ctaggatcaa ggtcaggaac agagaaacag gagaatatgg
gccaaacagg atatctgtgg taagcagttc ctgccccgct cagggccaag aacagttgga acaggagaat atgggccaaa
caggatatct gtggtaagca gttcctgccc cgctcagggc caagaacaga tggtccccag atgcggtccc gccctcagca
gtttctagag aaccatcaga tgtttccagg gtgccccaag gacctgaaat gaccctgtgc cttatttgaa ctaaccaatc
agttcgcttc tcgcttctgt tcgcgcgctt ctgctccccg agctctatat aagcagagct ggtttagtga accgtcagat
cgcctggaga cgccatccac gctgattga cctccataga agacaccgac tctagctaga ggatctccta ggaagctggc
cgcacaaagt ggtaccggat cccgggtcga ccatggagct ccaggcagcc cgcgcctgct tcgccctgct gtggggctgt
gcgctggccg cggccgcggc ggcgcagggc aaggaagtgg tactgctgga ctttgctgca gctggagggg agctcggctg
gctcacacac ccgtatggca aagggtggga cctgatgcag aacatcatga atgacatgcc gatctacatg tactccgtgt
gcaacgtgat gtctggcgac caggacaact ggctccgcac caactgggtg taccgaggag aggctgagcg tatcttcatt
gagctcaagt ttactgtacg tgactgcaac agcttccctg gtggcgccag ctcctgcaag gagactttca acctctacta
tgccgagtcg gacctggact acggcaccaa cttccagaag cgcctgttca ccaagattga caccattgcg cccgatgaga
tcaccgtcag cagcgacttc gaggcacgcc acgtgaagct gaacgtggag gagcgctccg tggggccgct cacccgcaaa
ggcttctacc tggccttcca ggatatcggt gcctgtgtgg cgctgctctc cgtccgtgtc tactacaaga agtgccccga
gctgctgcag ggcctggccc acttccctga gaccatcgcc ggctctgatg caccttccct ggccactgtg gccggcacct
gtgtggacca tgccgtggtg ccaccggggg gtgaagagcc ccgtatgcac tgtgcagtgg atggcgagtg gctggtgccc
attgggcagt gcctgtgcca ggcaggctac gagaaggtgg aggatgcctg ccaggcctgc tcgcctggat tattaagtt
tgaggcatct gagagcccct gcttggagtg ccctgagcac acgctgccat cccctgaggg tgccacctcc tgcgagtgtg
aggaaggctt cttccgggca cctcaggacc cagcgtcgat gccttgcaca cgacccccct ccgccccaca ctacctcaca
gccgtgggca tgggtgccaa ggtggagctg cgctggacgc cccctcagga cagcgggggc cgcgaggaca ttgtctacag
cgtcacctgc gaacagtgct ggcccgagtc tggggaatgc gggccgtgtg aggccagtgt gcgctactcg gagcctcctc
acggactgac ccgcaccagt gtgacagtga gcgacctgga gccccacatg aactacacct tcaccgtgga ggcccgcaat
ggcgtctcag gcctggtaac cagccgcagc ttccgtactg ccagtgtcag catcaaccag acagagcccc ccaaggtgag
gctggagggc cgcagcacca cctcgcttag cgtctcctgg agcatccccc cgccgcagca gagccgagtg tggaagtacg
aggtcactta ccgcaagaag ggagactcca acagctacaa tgtgcgccgc accgagggtt tctccgtgac cctggacgac
ctggccccag acaccaccta cctggtccag gtgcaggcac tgacgcagga gggccagggg gccggcagca aggtgcacga
attccagacg ctgtccccgg agggatctgg caacttggcg gtgattggcg gcgtggctgt cggtgtggtc ctgcttctgg
tgctggcagg agttggcttc tttatccacc gcaggaggaa gaaccagcgt gcccgccagt ccccggagga cgtttacttc
tccaagtcag aacaactgaa gcccctgaag acatacgtgg acccccacac atatgaggac cccaaccagg ctgtgttgaa
gttcactacc gagatccatc catcctgtgt cactcggcag aaggtgatcg gagcaggaga gtttggggag gtgtacaagg
gcatgctgaa gacatcctcg gggaagaagg aggtgccggt ggccatcaag acgctgaaag ccggctacac agagaagcag
cgagtggact tcctcggcga ggccggcatc atgggccagt tcagccacca caacatcatc cgcctagagg gcgtcatctc
caaatacaag cccatgatga tcatcactga gtacatggag aatggggccc tggacaagtt ccttcgggag aaggatggcg
agttcagcgt gctgcagctg gtgggcatgc tgcggggcat cgcagctggc atgaagtacc tggccaacat gaactatgtg
caccgtgacc tggctgcccg caacatcctc gtcaacagca acctggtctg caaggtgtct gactttggcc tgtcccgcgt
gctggaggac gaccccgagg ccacctacac caccagtggc ggcaagatcc ccatccgctg gaccgccccg gaggccattt
cctaccggaa gttcacctct gccagcgacg tgtggagctt tggcattgtc atgtgggagg tgatgaccta tggcgagcgg
ccctactggg agttgtccaa ccacgaggtg atgaaagcca tcaatgatgg cttccggctc cccacaccca tggactgccc
ctccgccatc taccagctca tgatgcagtg ctggcagcag gagcgtgccc gccgccccaa gttcgctgac atcgtcagca
tcctggacaa gctcattcgt gcccctgact ccctcaagac cctggctgac tttgaccccc gcgtgtctat ccggctcccc
agcacgagcg gctcggaggg ggtgcccttc cgcacggtgt ccgagtggct ggagtccatc aagatgcagc agtatacgga
gcacttcatg gcggccggct acactgccat cgagaaggtg gtgcagatga ccaacgacga catcaagagg attggggtgc
ggctgcccgg ccaccagaag cgcatcgcct acagcctgct gggactcaag gaccaggtga acactgtggg gatccccatc
gggaattcag aacaaaaact catctcagaa gaagatggag gcggcgggtc aggtggaggt ggctctggcg gtggcggttc
ttgttggacg ctggatcggg ggtattgctc ctaattccga ctcactatag ggcgaattaa ttccggagat ctctagatcc
ggagagacga tggcaggagc cgcgcatata tacgcttgga gccagcccgc cctcacaggg cgggccgcct cgggggcggg
actggccaat cggcggccgc cagcgcggcg gggcccggcc aaccagcgtc cgccgagtct tcggggcccg gcccattggg
cgggagttac cgcc.
SEQ ID NO: 13 hEphA2 GPNMB target derived from a human sequence: 1 gcctatggga
atgaaagacc ccacctgtag gtttggcaag ctaggatcaa ggtcaggaac agagaaacag gagaatatgg gccaaacagg
atatctgtgg taagcagttc ctgccccgct cagggccaag aacagttgga acaggagaat atgggccaaa caggatatct
gtggtaagca gttcctgccc cgctcagggc caagaacaga tggtccccag atgcggtccc gccctcagca gtttctagag
aaccatcaga tgtttccagg gtgccccaag gacctgaaat gaccctgtgc cttatttgaa ctaaccaatc agttcgcttc
tcgcttctgt tcgcgcgctt ctgctccccg agctctatat aagcagagct ggtttagtga accgtcagat cgcctggaga
cgccatccac gctgttttga cctccataga agacaccgac tctagctaga ggatctccta ggaagctggc cgcacaaagt
ggtaccggat cccgggtcga ccatggagct ccaggcagcc cgcgcctgct tcgccctgct gtggggctgt gcgctggccg
cggccgcggc ggcggatact cttgtgacgc gttcgaattt ttcttttaag cagggcaagg aagtggtact gctggacttt
gctgcagctg gaggggagct cggctggctc acacacccgt atggcaaagg gtgggacctg atgcagaaca tcatgaatga
catgccgatc tacatgtact ccgtgtgcaa cgtgatgtct ggcgaccagg acaactggct ccgcaccaac tgggtgtacc
gaggagaggc tgagcgtatc ttcattgagc tcaagtttac tgtacgtgac tgcaacagct tccctggtgg cgccagctcc
tgcaaggaga ctttcaacct ctactatgcc gagtcggacc tggactacgg caccaacttc cagaagcgcc tgttcaccaa
gattgacacc attgcgcccg atgagatcac cgtcagcagc gacttcgagg cacgccacgt gaagctgaac gtggaggagc
gctccgtggg gccgctcacc cgcaaaggct tctacctggc cttccaggat atcggtgcct gtgtggcgct gctctccgtc
cgtgtctact acaagaagtg ccccgagctg ctgcagggcc tggcccactt ccctgagacc atcgccggct ctgatgcacc
ttccctggcc actgtggccg gcacctgtgt ggaccatgcc gtggtgccac cggggggtga agagccccgt atgcactgtg
cagtggatgg cgagtggctg gtgcccattg ggcagtgcct gtgccaggca ggctacgaga aggtggagga tgcctgccag
gcctgctcgc ctggattat taagtttgag gcatctgaga gcccctgctt ggagtgccct gagcacacgc tgccatcccc
tgagggtgcc acctcctgcg agtgtgagga aggcttcttc cgggcacctc aggacccagc gtcgatgcct tgcacacgac
ccccctccgc cccacactac ctcacagccg tgggcatggg tgccaaggtg gagctgcgct ggacgccccc tcaggacagc
gggggccgcg aggacattgt ctacagcgtc acctgcgaac agtgctggcc cgagtctggg gaatgcgggc cgtgtgaggc
cagtgtgcgc tactcggagc ctcctcacgg actgacccgc accagtgtga cagtgagcga cctggagccc cacatgaact
acaccttcac cgtggaggcc cgcaatggcg tctcaggcct ggtaaccagc cgcagcttcc gtactgccag tgtcagcatc
aaccagacag agccccccaa ggtgaggctg gagggccgca gcaccacctc gcttagcgtc tcctggagca tccccccgcc
gcagcagagc cgagtgtgga agtacgaggt cacttaccgc aagaagggag actccaacag ctacaatgtg cgccgcaccg
agggtttctc cgtgaccctg gacgacctgg ccccagacac cacctacctg gtccaggtgc aggcactgac gcaggagggc
cagggggccg gcagcaaggt gcacgaattc cagacgctgt ccccggaggg atctggcaac ttggcggtga ttggcggcgt
ggctgtcggt gtggtcctgc ttctggtgct ggcaggagtt ggcttcttta tccaccgcag gaggaagaac cagcgtgccc
gccagtcccc ggaggacgtt tacttctcca agtcagaaca actgaagccc ctgaagacat acgtggaccc ccacacatat
gaggacccca accaggctgt gttgaagttc actaccgaga tccatccatc ctgtgtcact cggcagaagg tgatcggagc
aggagagttt ggggaggtgt acaagggcat gctgaagaca tcctcgggga agaaggaggt gccggtggcc atcaagacgc
tgaaagccgg ctacacagag aagcagcgag tggacttcct cggcgaggcc ggcatcatgg gccagttcag ccaccacaac
atcatccgcc tagagggcgt catctccaaa tacaagccca tgatgatcat cactgagtac atggagaatg gggccctgga
caagttcctt cgggagaagg atggcgagtt cagcgtgctg cagctggtgg gcatgctgcg gggcatcgca gctggcatga
agtacctggc caacatgaac tatgtgcacc gtgacctggc tgcccgcaac atcctcgtca acagcaacct ggtctgcaag
gtgtctgact ttggcctgtc ccgcgtgctg gaggacgacc ccgaggccac ctacaccacc agtggcggca agatccccat
ccgctggacc gccccggagg ccatttccta ccggaagttc acctctgcca gcgacgtgtg gagctttggc attgtcatgt
gggaggtgat gacctatggc gagcggccct actgggagtt gtccaaccac gaggtgatga aagccatcaa tgatggcttc
cggctcccca cacccatgga ctgcccctcc gccatctacc agctcatgat gcagtgctgg cagcaggagc gtgcccgccg
ccccaagttc gctgacatcg tcagcatcct ggacaagctc attcgtgccc ctgactccct caagaccctg gctgactttg
acccccgcgt gtctatccgg ctccccagca cgagcggctc ggagggggtg cccttccgca cggtgtccga gtggctggag
tccatcaaga tgcagcagta tacggagcac ttcatggcgg ccggctacac tgccatcgag aaggtggtgc agatgaccaa
cgacgacatc aagaggattg gggtgcggct gcccggccac cagaagcgca tcgcctacag cctgctggga ctcaaggacc
aggtgaacac tgtggggatc cccatcggga attcagaaca aaaactcatc tcagaagaag atctaggagg cggcgggtca
ggtggaggtg gctctggcgg tggcggttaa aattccgact cactataggg cgaattaatt ccggagatct ctagatccgg
agagacgatg gcaggagccg cgcatatata cgcttggagc cagcccgccc tcacagggcg ggccgcctcg ggggcgggac
tggccaatcg gcggccgcca gcgcggcggg gcccggccaa ccagcgtccg ccgagtcttc ggggcccggc ccattgggcg
ggagttacc cc.

### BRIEF DESCRIPTION OF THE FIGURES

FIG. 1 provides a schematic representation showing EphA2 derivatives expressed from C134. (A) Configuration of the C134 oHSV, including IRS1 expression from the UL3/UL4 intergenic region, and deletion of γ₁34.5. The pCK1136 shuttle vector was used to insert an Egr1-driven GFP expression cassette into C134, designated C154 (B). (C-F) Murine EphA2 derivatives will be cloned into pCK1136 for recombination and replacement of GFP in C154. The wild type secretory leader sequence will be maintained and a C-terminal Myc tag will be utilized to distinguish endogenous from C134-expressed proteins. (C) Full-length EphA2. (D-F) Secreted truncation variants of EphA2, encompassing the extracellular (Ecto, D, E) or intracellular (Endo, F) EphA2 domains will be generated using the native leader sequence and deletion of the transmembrane domain. (E, F) Secreted EphA2 variants with a C-terminal DC-targeting domain.

FIGS. 2A and 2B provide graphs showing a composite of oHSV recovery and survival studies in A) U87 IC tumor model where C134 replicates 1000× greater than a Δγ₁34.5 virus (C101) and in a Neuro2A model B), where it has no replication advantage (Inserts: viral recovery at 48 hpi)

FIGS. 3A and 3B provide images showing C134 induced humoral and cell-mediated immunity. (A) Serum-based detection of humoral reactivity to DBT and Neuro2A cell lysates. Serum was collected from naive Balb/c mice, or Balb/c mice challenged with DBT tumors and treated with Saline, R3616, or C134. The serum was diluted 1:2048 and used as a primary for Western analysis. An excess of splenocytes isolated from the same animals were added to a confluent monolayer of DBT cells (10:1). After three days, cell expansion was compared to cells plated in the absence of DBT stimulation, and cell mediated cytotoxicity was visualized by May-Grunwald staining of the remaining DBT cells (B).

FIGS. 4A-4C provide graphs showing GPNMB-specific phage and fusion protein binding. (A) A GPNMB-specific consensus sequence was identified after 4 rounds of phage panning to GPNMB-Fc. GPNMB binding specificity of a phage clone (B), or a FcE engineered with a secretory leader and genetically fused to the GPNMB-binding consensus sequence (C) were added to a mixture of 293T and 293T-GPNMB cells. GPNMB expression and phage/secreted protein binding were assessed by flow cytometry.

FIGS. 5A and 5B provide graphs showing malignant glioma tumor lines can be divided based upon oHSV replication into sensitive and resistant cell lines. DBT tumors are highly resistant MG tumor cells. Analysis of tumor cell susceptibility to HSV infection (% HSV positive) and the absolute numbers of cells remaining (% relative cell count). MGs can be stratified into oHSV-sensitive and -resistant tumors based upon their susceptibility to viral cytolysis and shows that DBT tumor cells are highly oHSV resistant. B) Viral recovery studies from DBT CNS tumors show that C134 and R3616 replicate equivalently in these CNS tumors. Limiting plaque dilutions and qPCR of HSV DNA were performed from the oHSV treated mouse CNS tumors and show no difference in R3616 (first generation oHSV) and C134 (second generation oHSV) replication.

FIG. 6A and FIG. 6B provide graphs showing in vivo DBT tumor studies showing that mechanisms other than direct viral mediated cytolysis contribute to oHSV anti-tumor activity. Our previous studies show that C134 has improved direct oncolytic activity on oHSV sensitive tumors. However, some tumors (e.g., DBT, CT2A, GL261) are resistant to HSV replication and viral cytolytic activity. Orthotopic DBT tumor-bearing mice were treated with oHSV (1×10⁷ pfu) or saline. C134 significantly improved survival (p=0.003). R3616 a ICP34.5 (−) virus did not improve survival over saline treatment; nonetheless, a fraction (˜⅓) of the R3616 or C134 mice were long term survivors. (>37 d). Survivors were re-challenged with DBT tumors in another location (flank). Survivors had significantly slower tumor growth than naïve (tumor inexperienced) control mice suggesting that the anti-tumor immune response contributed to the long-term survival in these survivors.

FIG. 7 provides a graph showing that repeated C134 treatment extends survival. To identify if repeated dosing of C134 can extend survival, a Winn type assay was performed. In brief, Balb/C mice were implanted with oHSV-infected tumor cells (MOI 1) and then re-treated 8 days later (1×10⁷ pfu). The results show that similar to past studies the DBT tumors were rapidly fatal to mice treated with saline (median survival 14 d). In contrast, the C134 repeated dosing extended survival of the mice with DBT brain tumors (median survival 55 d compared to 33 d in the single dose study). A cohort was also treated with a C134-foreign antigen expressing virus (EGFP) to identify if foreign antigen expression from the virus improved the anti-tumor immune response. A control cohort was inoculated with mitomycin C treated tumor cells. Mito-C treated tumor cells do not replicate but remain viable exposing mice to tumor antigens (independent of oHSV treatment) for a subsequent study shown in FIG. 9.

FIGS. 8A and 8B provide a schematic and graph showing pro-inflammatory cytokine expression improves survival. A) Schematic of C002 a C134 based mIL-12 expressing oHSV. Treatment of Balb/C mice bearing orthotopic DBT tumors with 1×10⁷ pfu of C134 or C002 (C134+mIL-12) significantly improves survival over saline therapy. Expression of the T cell activating pro-inflammatory cytokine (IL-12) from C134 further extends C134 anti-tumor activity in the DBT syngeneic tumor model.

FIG. 9 provides a graph showing oHSV treatment stimulates an anti-tumor immune response superior to that seen in naïve mice or those who had prior tumor antigen exposure (Mito-C) and significantly reduced tumor growth in re-challenged mice.

FIG. 10A-C provides graphs showing HSV-immunity and its effect upon oHSV anti-tumor efficacy. We hypothesized that similar to pro-inflammatory cytokine expression, mice with pre-existing HSV immunity would exhibit enhanced T cell activity that would translate into an improved anti-tumor effect. To test this hypothesis, we immunized Balb/C cohorts with HSV (optiprep R3616 3.33×10⁶ pfu IM d21 and d7 prior to tumor implantation). We then implanted DBT tumors (1×10⁵ cells), and treated the HSV immune and HSV naïve cohorts with 1×10⁷ pfu of oHSV (C134 (FIG. 10B) or C002 (FIG. 10C)) or Saline 1 (FIG. 10A) week after tumor implantation (consistent with our previous studies). The results show that prior HSV immunity had no effect upon saline or C134 survival.

FIGS. 11A-11C provide composite images and schematics of C134 based tumor antigen expressing oHSVs. A) confocal microscopy shows cellular distribution of expressed EphA2 in cells (Myc tagged-EphA2, Trans-Golgi Network, DAPI-Nucleus). Full length EphA2 is membrane-associated, whereas the intracellular and extracellular domains distribute within the cell. B) Schematic of C170-C175, the C134 EphA2 tumor antigen expressing constructs including epitope domains at the 3′ end that enable us to identify the viral expressed antigen and direct it to antigen expressing cells. C) Immunostaining studies show that the oHSV-expressed full length and intracellular EphA2 domains remain cell associated (C170, C171, C174, and C175) whereas the oHSV-expressed EphA2 extracellular domain is secreted into the media (C172, C173).

FIGS. 12A-12D provide schematic representations and images showing EphA2 expressing virus construction and validation. (A) Schematic overview of antigen expressing virus—C170 and C172 were constructed by homologous recombination using a C134 based virus. This introduced the sequence encoding the full length C57bl6 EphA2 gene (C170) or Extracellular domain (C172) driven by the strong MND promoter into the g134.5 gene domain. (B) DNA hybridization studies confirmed the anticipated DNA fragment sizes distinct from the parent virus. (C). Immunofluorescence showed distinct cellular distribution characteristics (C170 membrane associated staining, C172 IC staining): Green color EphA2, Red staining of the Trans-golgi network, Blue color Nuclear staining with DAPI (D). Western blot of infected mouse glioma cells and supernatants shows that C170 expresses the anticipated 125 kd cell associated protein and C172 produces a 60 kd cell associated and secreted protein.

FIGS. 13A-D provide graphs showing viral Replication (FIGS. 13A and 13B) and Cytotoxicity (FIGS. 13C and 13D) in B6 murine glioma (CT2A) (FIGS. 13A and C) and MPNST (FIGS. 13B and D) (67C4) cells. (A). Viral Recovery studies show that C170 and C172 replicate similar to the parent virus (C134) in the murine tumor lines of interest. (B) Viral Cytotoxicity assay using incucyte and infection at an multiplicity of infection (PFU/Cell) of 1 shows that C134 and the C170 induce similar cytotoxicity and that the tumor lines exhibit differences in direct cellular cytotoxicity.

FIGS. 14A and 14B provide graphs showing in vivo testing of C170 in 2 different syngeneic tumor models. (A). C170 is the only virus tested that significantly improves survival in the CT2A orthotopic model. Top Panel shows a schematic of experimental design and virus treatment. Lower Panel shows Kaplan Meier curve showing improved median and overall survival following C170-treatment (B). C170 also significantly reduced tumor growth in the highly resistant murine 67C4 murine MPNST tumor model (Mann-Whitney analysis of unpaired samples, *p value<0.05, **p value<0.005 two-tailed analysis).

FIGS. 15A-15H provide graphs and images showing CT2A Brain tumor TIL Immunophenotyping: Analysis of CT2A Tumor infiltrating Leukocytes (TIL) after saline-perfusion —(A) Proportional Pie Chart Summary of overall TILs isolated from Saline (41,948), C134 (275,594), and C170 (273.174) treated mice at D6 post-injection and the relative lymphocyte (pink) and Myeloid (Black, Orange and Blue) composition. Numbers below pie chart represent absolute numbers of leukocytes/brain sample. (B) C170 significantly increases overall T cell infiltrates. (C) Both oHSVs (C134 and C170) increase CD4 populations, (D). C170 significantly increases CD8 T cytotoxic cells and (E) activated (CD25+) CD8 T cytotoxic cells. (F). Example of gating and representative flow plots for further CD8 phenotypic analysis for CD8-effector-like (CD44+, CD62L−) and central memory-like (CD44+, CD62L+) population analysis. (G). Both C134 and C170 increase CD8 T effector-like populations (CD44+, CD62L−), but only C170 increases the (H) CD8 central memory-like population at D6 post-treatment.

FIGS. 16A-16I provide graphs and images showing the immunophenotypic analysis of Saline and oHSV treated 67C4 flank tumors shows that (A) C170 significantly decreases the relative proportion of CD11b myeloid population in the tumor and (B) significantly decreases the immunosuppressive MDSC-like (CD11b+, GR1+) population when compared to saline or C134 treated samples. C170 treatment significantly increases the (CD8+, CD44+, CD62L+) central memory-like population within the tumor similar to our brain tumor model results. In addition to TIL changes, C170 treatment also has effects on the peripheral populations (D) Representative example of initial T cell (CD90+) and Myeloid (CD11b+) population gating. C170 treatment reduces (E) CD11b (+) cells and (F) CD11b+, GR1+ MDSC-like populations in the periphery. C170 does not significantly increase the (G) T cell or (H) CD4(+) T cell proportions but it does increases (I) the CD8 T cell population in the periphery.

FIGS. 17A and 17B provides a scheme and graph showing abscopal effects and immune memory against the tumor. C170-treated brain tumor survivors suppress CT2A tumor re-growth better than naïve (mice never exposed to tumor) or C134-treated survivors upon CT2A flank tumor re-challenge. (A) Experimental design schematic shown (B) tumor growth curves show significant decrease in tumor growth after CT2A implantation in the flanks of naïve or oHSV treated long-term survivors.

FIGS. 18A-18F provide a summary of T cell function studies from Saline and oHSV treated mice shows that C170 treatment induces an antigen specific T cell response in the periphery of long term survivors. Splenocytes from Saline (Blue columns) or oHSV-treated mice (red column C134, Green Column C170) were analyzed. At the start there was no difference in the populations used peptide pulsing; however, after pulsing with 10 um EphA2 or 10 uM OVA peptide (negative control), C170-treated mice significantly increase their activated (B) CD25(+), (C) GZMB (+), and (D) CD25+, GZMB+ dual staining CD8+ populations indicative of an EphA2 specific population response. (E) Representative flow plot shown of the CD8(+) GZMB (+) population and gating and the (F) GZMB CD25 dual positive gating populations.

FIGS. 19A-19I provide schematic representations of exemplary chimeric oncolytic viruses described herein (FIGS. 19A-19C) and the entire viral genomic sequence for C170 (FIG. 19D), C171 (FIG. 19E), C172 (FIG. 19F), C173 (FIG. 19G). C174 (FIG. 19H), and C175 (FIG. 19I).

### DETAILED DESCRIPTION

The present invention provides a chimeric oncolytic virus that includes a herpesvirus having a modified nucleic acid sequence, including a modification of the herpesvirus gamma (1)34.5 gene (γ₁34.5) or a nucleic acid with at least about 70% homology to the γ134.5 gene that reduces its expression; a second viral nucleic acid sequence encoding a PKR evasion protein that does not cause virulence; and a third nucleic acid sequence encoding a tumor-associated antigen. Methods of using the chimeric oncolytic virus to treat subjects having cancer, or to vaccinate subjects at risk of developing cancer, are also described.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein and in the appended claims, the singular forms “a”, “and”, and “the” include plural referents unless the context clearly dictates otherwise. Thus, for example, reference to “a sample” also includes a plurality of such samples and reference to “the splicing regulator protein” includes reference to one or more protein molecules, and so forth.

As used herein, the term “about” refers to +/−10% deviation from the basic value.

As used herein the term “nucleic acid” or “oligonucleotide” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Other such modifications are well known to those of skill in the art. Thus, the term nucleic acid also encompasses nucleic acids with substitutions or modifications, such as in the bases and/or sugars.

The term “base” encompasses any of the known base analogs of DNA and RNA. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

As used herein, the term “tumor” refers to any neoplastic growth, proliferation or cell mass whether benign or malignant (cancerous), whether a primary site lesion or metastases.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

An “oncolytic virus” refers to a virus that preferentially infects and kills cancer cells. The infected cancer cells are destroyed by oncolysis, leading to the release of new infectious virus particles that go on to infect other cancer cells.

A “chimeric virus,” refers to a virus comprising nucleic acid sequences from different viruses. For example, a chimeric virus can be a virus including nucleic acid material from a herpesvirus and a cytomegalovirus.

Unless defined otherwise, all technical and scientific terms used herein have the same meaning as commonly understood by one of ordinary skill in the art to which this invention belongs.

### Chimeric Oncolytic Viruses

In one aspect, the invention provides chimeric oncolytic virus, comprising a herpesvirus having a modified nucleic acid sequence, comprising a modification of the herpesvirus gamma (1)34.5 gene (γ₁34.5) or a nucleic acid with at least about 70% homology to the γ₁34.5 gene that reduces its expression; a second viral nucleic acid sequence encoding a PKR evasion protein that does not cause virulence; and a third nucleic acid sequence encoding a tumor-associated antigen.

Selective replication of these HSV recombinants in tumors can be achieved by deletion of the viral neurovirulence gene, γ₁34.5. Deletion of the HSV-1 neurovirulence gene allows the safe administration of these oncolytic viruses. Although Δγ₁34.5 viruses are capable of entry into non-dividing normal cells, these viruses cannot replicate efficiently except in actively dividing cells such as tumor cells. Chou et al., Science 250(4985): 1262-6 (1990). Therefore such viruses are tumor-selective viruses. Δγ₁34.5 viruses have shown significant efficacy for therapy of brain malignancies in preclinical animal models, and have been demonstrated to be safe in Phase I and II trails in both the U.S. (Markert et al., Gene Ther. 7(10): 867-74 (2000)) and Great Britain (Rampling et al., Gene Ther. 7(10): 859-66 (2000)). However, attenuated HSV-1 (Δγ₁34.5) recombinants are unable to efficiently synthesize viral proteins and this limits viral replication. Shah et al., J. Neurooncol. 65(3): 203-26 (2003). However, inclusion of a PKR evasion gene that inhibits PKR-mediated protein shutoff without neurovirulence.

The chimeric oncolytic virus includes a modification of the herpesvirus gamma (1)34.5 gene (γ₁34.5) or a nucleic acid with at least about 70% homology to the γ₁34.5 gene that reduces its expression. Modifications that can be made to the γ₁34.5 gene include one or more mutations, deletions, insertions and substitutions. Thus, the modification to the herpesvirus nucleic acid sequence can comprise the complete or partial deletion of the γ₁34.5 gene (SEQ ID NO: 1) from HSV-1. The modification can comprise an inserted exogenous stop codon or other nucleotide or nucleotides. The modification can comprise the mutation or deletion of the promoter or the insertion of an exogenous promoter that alters expression of the γ₁34.5 gene. The modification can comprise one or more inserted nucleotides that results in a codon frame-shift. Furthermore, the second viral nucleic acid sequence of the chimera could be substituted for the γ₁34.5 gene. The modification to the γ₁34.5 gene can also be a modification of a nucleic acid with at least about 70-99% homology, including 70%, 75%, 80%, 85%, 90%, or 95% homology, to the γ₁34.5 gene. In some embodiments, modification of the herpesvirus γ₁34.5 gene comprises a deletion or mutation of the γ₁34.5 gene. Methods for making the modifications described herein are well known to those skilled in the art.

The chimeric oncolytic viruses of the present invention are based on the Herpesvirus. Genetically modified herpesvirus are attractive as oncolytic vectors for a number of reasons: 1) procedures for constructing recombinant herpesvirus are well established; 2) multiple genes can be deleted and/or replaced with therapeutic foreign genes without affecting the replication capacity of the virus; 3) considerable experience with the biology of herpesvirus and its behavior in humans and nonhuman primates exists in the literature; and 4) modified herpesviruses can be engineered to retain sensitivity to standard antiviral drug therapy as a “built-in” safety feature. Furthermore, the genome size of the Herpes Simplex Virus, 152 kb, allows transfer of genes 30 kb or more in size.

There are more than 120 animal herpesviruses. All herpesviruses are divided into three subsets: the alpha (a), beta (P) and gamma (γ) herpesviruses. There are 8 human herpesviruses, which are split between the three subsets. Alpha Herpesviruses include Herpes Simplex Virus 1 (HSV-1), HSV-2, and Varicella Zoster Virus (VZV). Beta Herpesviruses include Human Cytomegalovirus (HCMV), Human Herpesvirus 6 (HHV-6), and Human Herpesvirus 7 (HHV-7). Gamma Herpesvirus include Epstein Barr Virus (EBV) and Gamma Kaposi's Sarcoma Herpesvirus. Accordingly, in some embodiments the herpesvirus included in the chimeric oncolytic virus is an α herpesvirus, while in further embodiments the herpesvirus included in the chimeric oncolytic virus is an HSV-1 herpesvirus.

The chimeric oncolytic virus comprises a modification of the herpesvirus gamma (1)34.5 gene (γ₁34.5) or a nucleic acid with at least about 70% homology to the γ₁34.5 gene that reduces its expression and a second viral nucleic acid sequence encoding a PKR evasion protein that does not cause virulence. The herpesvirus nucleic acid modification causes reduced expression of a protein kinase R (PKR) evasion gene as compared to expression of the evasion gene in the absence of the modification. The second viral sequence encodes a protein that comprises the protein synthesis function of the PKR evasion gene without the neurovirulence function of the gene. Therefore, the chimeric virus has a reduced neurovirulence as compared to a wild-type herpesvirus. Also as disclosed herein, the provided chimeric virus has enhanced protein synthesis and/or replication as compared to existing attenuated herpesviruses, such as, for example, Δγ₁34.5 HSV. The second nucleic acid sequence of the provided chimeric virus enhances protein synthesis or replication as compared to the protein synthesis or replication of the chimeric virus in the absence of the second viral nucleic acid sequence. The second nucleic acid sequence of the provided chimeric virus can enhance protein synthesis and replication by inhibiting the activation of PKR, inhibiting the phosphorylation of eIF-2α, or enhancing the dephosphorylation of eIF-2α.

The second viral nucleic acid sequence of the chimeric oncolytic virus comprises one phenotype of the PKR evasion gene, protein synthesis and replication in infected tumor cells, but not the other phenotype of the PKR evasion gene, PKR-mediated virulence, e.g., neurovirulence. In other words, the second viral nucleic acid sequence inhibits PKR-mediated protein shutoff without neurovirulence. Thus, the second viral nucleic acid sequence can be any PKR evasion gene or comparable gene that does not cause virulence. The second viral nucleic acid sequence can be derived from homologous viruses. Thus, the second viral nucleic acid sequence of the provided chimeric virus can be an α herpesvirus nucleic acid sequence, P herpesvirus nucleic acid sequence, or γ herpesvirus nucleic acid sequence. Thus, the viral nucleic acid sequence of the provided chimeric virus can be a cytomegalovirus (CMV) nucleic acid sequence.

Examples of suitable nucleic acid sequences that can be used in the provided chimeric virus include, but are not limited to, IRS-1 (SEQ ID NO: 2) and TRS-1 (SEQ ID NO: 3), or homologous genes thereof. The provided chimeric virus can comprise an IRS-1 gene. The provided chimeric virus can also comprise a nucleic acid having at least about 70-99% homology, including about 70%, 75%, 80%, 85%, 90%, 95% homology to the IRS-1 gene. The provided chimeric virus can comprise a TRS-1 gene, or homologous genes thereof. The provided chimeric virus can also comprises a nucleic acid having at least about 70-99% homology, including about 70%, 75%, 80%, 85%, 90%, 95% homology, to the TRS-1 gene.

Human cytomegalovirus (HCMV) IRS1 and TRS1 proteins have a shared 130 amino acid (aa) region that independently interacts with two eukaryotic genes, Nedd4 and TSG101, involved in vesicular transport and lysosomal sorting in the cell. As described in the examples below a chimeric virus comprising either TRS1 or IRS1 have a similar protein synthesis phenotype. Thus, the provided chimeric virus can comprise the nucleic acid sequence that corresponds to the shared 130 aa region of IRS1 and TRS1 (SEQ ID NO: 4). The provided chimeric virus can also comprise a nucleic acid having at least about 70-99% homology, including about 70%, 75%, 80%, 85%, 90%, 95% homology to SEQ ID NO:4.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least the material related to nucleic acid alignment. It is understood that any of the methods typically can be used and that, in certain instances, the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method, even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of the calculation methods, although, in practice, the different calculation methods will often result in different calculated homology percentages.

The disclosed nucleic acids may contain, for example, nucleotide analogs or nucleotide substitutes. Non-limiting examples of these and other molecules are discussed herein. It is understood that, for example, when a vector is expressed in a cell, the expressed mRNA will typically be made up of A, C, G, and U.

A nucleotide analog is a nucleotide which contains some type of modification of either the base, sugar, or phosphate moieties. Modifications to nucleotides are well known in the art and include for example, 5-methylcytosine (5-me-C), 5-hydroxymethyl cytosine, xanthine, hypoxanthine, and 2-aminoadenine.

Also provided herein is a viral vector comprising the herein disclosed chimeric oncolytic virus, wherein the chimeric oncolytic virus further comprises a tumor-associated antigen. Thus, a method of delivering a tumor-associated antigen to a cell is provided, comprising contacting the target cell with the herein provided viral vector. The delivery can be in vivo or ex vivo. The chimeric oncolytic virus of a viral vector can include a gene encoding a modified HSV glycoprotein required for virus entry. Recombinant HSV have been constructed that exclusively enter tumor cells through tumor-specific receptors. Zhou and Roizman, J. Virol. 79(9): 5272-7 (2005).

Nucleic acids, such as the ones described herein, that are delivered to cells typically contain expression controlling systems. For example, the inserted genes in viral and retroviral systems usually contain promoters and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Preferred promoters controlling transcription from vectors in mammalian host cells may be obtained from various sources, for example, the genomes of viruses such as polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, ax-fetoprotein and insulin), typically one will use an enhancer from a eukaryotic cell virus for general expression. Preferred examples include, but are not limited to, the SV40 enhancer, the cytomegalovirus early promoter enhancer, the polyoma enhancer, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating drugs.

The promoter region can act as a constitutive promoter to maximize expression of the region of the transcription unit to be transcribed. In certain constructs, the promoter region can be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR. It has been shown that all specific regulatory elements can be cloned and used to construct expression vectors that are selectively expressed in specific cell types such as melanoma cells. For example, the glial fibrillary acidic protein (GFAP) promoter has been used to selectively express genes in cells of glial origin. Such tumor specific promoters can also be incorporated into the chimeric viruses as well as the viral vectors described herein.

Expression vectors used in eukaryotic host cells may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs are well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences, alone or in combination with the above sequences, to improve expression from, or stability of, the construct.

Viral vectors can include a nucleic acid sequence encoding a marker product. This marker product is used to determine if the gene has been delivered to the cell and once delivered is being expressed. Marker genes include, for example, the E. Coli lacZ gene, which encodes β-galactosidase, and green fluorescent protein (GFP). Markers can also be used in imaging techniques. Thus, a chimeric vector that encodes a marker could be used to visualize a cancer cell or tumor. The size of the marked region or the intensity of the marker can be used to evaluate the progression, regression, or cure of cancer, for example.

As used herein a “marker” means any detectable tag that can be attached directly (e.g., a fluorescent molecule integrated into a polypeptide or nucleic acid) or indirectly (e.g., by way of activation or binding to an expressed genetic reporter, including activatable substrates, peptides, receptor fusion proteins, primary antibody, or a secondary antibody with an integrated tag) to the molecule of interest. A “marker” is any tag that can be visualized with imaging methods. The detectable tag can be a radio-opaque substance, radiolabel, a fluorescent label, a light emitting protein, a magnetic label, or microbubbles (air filled bubbles of uniform size that remain in the circulatory system and are detectable by ultrasonography, as described in Ellega et al. Circulation, 108:336-341, 2003, which is herein incorporated in its entirety). The detectable tag can be selected from the group consisting of gamma-emitters, beta-emitters, and alpha-emitters, positron-emitters, X-ray emitters, ultrasound reflectors (microbubbles), and fluorescence-emitters suitable for localization. Suitable fluorescent compounds include fluorescein sodium, fluorescein isothiocyanate, phycoerythrin, Green Fluorescent Protein (GFP), Red Fluorescent Protein (RFP), Texas Red sulfonyl chloride (de Belder & Wik, Carbohydr. Res. 44(2):251-57 (1975)), as well as compounds that are fluorescent in the near infrared such as Cy5.5, Cy7, and others. Also included are genetic reporters detectable following administration of radiotracers such as hSSTr2, thymidine kinase (from herpes virus, human mitochondria, or other) and NIS (sodium/iodide symporter). Light emitting proteins include various types of luciferase. Those skilled in the art will know, or will be able to ascertain with no more than routine experimentation, other fluorescent compounds that are suitable for labeling the molecule.

In vivo monitoring can be carried out using, for example, bioluminescence imaging, planar gamma camera imaging, SPECT imaging, light-based imaging, magnetic resonance imaging and spectroscopy, fluorescence imaging (especially in the near infrared), diffuse optical tomography, ultrasonography (including untargeted microbubble contrast, and targeted microbubble contrast), PET imaging, fluorescence correlation spectroscopy, in vivo two-photon microscopy, optical coherence tomography, speckle microscopy, small molecule reporters, nanocrystal labeling and second harmonic imaging. Using the aforementioned imaging technologies, reporter genes under control of various inflammation specific promoters are detected following specific induction.

These technologies can be applied in combination with other imaging technologies. For example, tumor mass monitoring can be accomplished using tumor cells positive for CMV-luciferase. In addition, two luciferase enzymes can be imaged at the same time, for example, using CMV-luciferase (from firefly) and cox2L-luciferase (from Renilla). Other reporters and promoters can be used in conjunction with these examples, some examples of which are disclosed above.

### Tumor-Associated Antigens

The chimeric oncolytic virus includes a third nucleic acid sequence encoding a tumor-associated antigen. As used herein, a “tumor-associated antigen” comprises any antigen produced by a tumor cell. A “tumor-associated antigen” can be an antigen present only in a tumor cell and not on any other cell, or it may be an antigen present in some tumor cells and also in some normal cells. Tumor-associated antigens can include, for example, products of mutated oncogenes and tumor suppressor genes, overexpressed or aberrantly expressed cellular proteins, tumor antigens produced by oncogenic viruses, oncofetal antigens, altered cell surface glycolipids and glycoproteins or cell-type specific differentiation antigens.

In some embodiments, the chimeric oncolytic virus is capable of expressing a plurality of different tumor-associated antigens. For example, in some embodiments, the herpesvirus includes a fourth nucleic acid sequence encoding a different tumor-associated antigen from that encoded by the third nucleic acid sequence.

Various antigens (e.g., tumor-associated antigens, microbial antigens) or antigenic portions thereof can be selected for use as antigens of interest from among those antigens known in the art or determined by immunoassay to be able to bind to antibody or MHC molecules (antigenicity) or generate an immune response (immunogenicity) as described above. Additional, useful antigens or derivatives thereof can also be identified by various criteria, such as the antigen's involvement in cancer, (Norrby (1985) Vaccines 85, Lerner, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen.

While any antigen of interest can be employed in the methods and compositions provided herein, non-limiting examples include tumor-associated antigens or antigenic portions thereof that are associated with, derived from, or predicted to be associated with a cancer. In such instances the tumor-associated antigen of interest can be from any type of cancer, including, but not limited to, adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer, or any antigenic portion thereof. In some embodiments, the tumor-associated antigen is a glioblastoma-associated antigen. In some embodiments, the tumor-associated antigen is one found on the cancer being treated.

Many types of tumor cells express antigens that are not found in normal cells. These antigens, known as tumor-associated antigens, have been intensively studied as targets for therapeutic anti-cancer vaccines. Exemplary tumor-associated antigens are lymphocyte antigen 6 complex, locus K (LY6K), cell division cycle associated 1 (CDCA1), insulin-like growth factor-II mRNA-binding protein 3 (IMP-3), kinesin family member 20A (KIF20A), glypican-3 (GPC3), forkhead box M1 (FOXM1), cadherin 3 (CDH3), secreted protein acidic and rich in cysteine (SPARC), cell division cycle 45 ligand (CDC45L), DEP domain containing 1 (DEPDC1), M-phase phosphoprotein 1 (MPHOSPH1), prostate-specific antigen (PSA), prostate-specific membrane antigen (PSMA), human epidermal growth factor receptor2/neuroblastoma (HER2/neu), carcinoembryonic antigen (CEA), mutated epidermal growth factor receptor (EGFR), melanoma antigen (MAGE), mucin-1 (MUC-1), and New York esophageal squamous cell carcinoma 1 (NY-ESO-1), BAGE, GAGE, MAGE, NY-ESO-1, SSX, gp100, Melan-A/Mart-1, Tyrosinase, Mammaglobin-A, p53, livin, survivin, β-Actin/4/m, Myosin/m, HSP70-2/m, HLA-A2-R170J, GM2, GD2, GD3, MUC-1, sTn, globo-H, WT1, PRI, E75, ras, AFP, URLC10, VEGFR1 and 2, mutant p53, NY-ESO-1, HPV16 E7, β-catenin, CDK4, CDC27, α-actinin-4, TRP1/gp75, TRP2, gangliosides, WT1, EphA2, EphA3, CD20, telomerase, MART-1, or an antigenic portion thereof. See Hirayama et al. 2016, Int. Immunol. Advance Access May 28 pp 1-26. In some embodiments, the tumor-associated antigen is EphA2.

Moreover, also encompassed are variants of the aforementioned tumor-associated antigens. Such variants have at least the same essential antigenic activity as the specific tumor-associated antigens. Moreover, it is to be understood that a variant as referred to in accordance with the present invention shall have an amino acid sequence which differs due to at least one amino acid substitution, deletion and/or addition, wherein the amino acid sequence of the variant is still, preferably, at least 50%, 60%, 70%, 80%, 85%, 90%, 92%, 95%, 97%, 98%, or 99% identical with the amino sequence of the specific tumor-associated antigen. The degree of identity between two amino acid sequences can be determined by algorithms well known in the art.

The third nucleic acid sequence can be inserted into the nucleotide sequence expressing the chimeric oncolytic virus using the methods known to those skilled in the art and described herein. For example, the pCK1166 vector (Cassady et al., J. Virol 86(a), p. 610-4 (2012)) can be used for recombination-based insertion of the transgene expression cassette including the sequence for expressing the tumor-associated antigen. In some embodiments, the third nucleic acid sequence is inserted into the chimeric oncolytic virus at the γ₁34.5 locus.

In some embodiments, the tumor-associated antigen is modified to include a binding protein or to increase its secretion by cells infected by the chimeric oncolytic virus. For example, in some embodiments the tumor-associated antigen includes a dendritic cell-binding peptide. In other embodiments, the tumor-associated antigen is a secreted protein. Suitable dendritic cell-binding peptides include those that bind to the dendritic cell-associated heparan sulfate proteoglycan-integrin ligand.

### Methods of Making

The chimeric oncolytic viruses disclosed herein and the compositions necessary to perform the disclosed methods can be made using any method known to those of skill in the art for that particular reagent or compound unless otherwise specifically noted. For example, the nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

The chimeric oncolytic viruses and viral vectors can be made recombinantly as set forth in the examples or by other methods of making recombinant viruses as described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Similar methods are used to introduce a gene of interest in methods of making the viral vector described herein. For example, recombinant viruses can be constructed using homologous recombination after DNA co-transfection. In this example, cells can be co-transfected with at least two different viruses containing the genes of interest and progeny virus plaque can be purified based upon loss of marker expression. Final verification of the correct genetic organization of candidate viruses can be verified by DNA hybridization studies using probes to the nucleic acids as described herein.

The nucleic acid sequences described herein may be obtained using standard cloning and screening techniques, from natural sources such as genomic DNA libraries or can be synthesized using well known and commercially available techniques.

When the nucleic acid sequences are used recombinantly, the nucleic acid sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. The nucleic acid sequence may also contain non-coding 5′ and 3′ sequences, such as transcribed, non-translated sequences, splicing and polyadenylation signals, ribosome binding sites and sequences that stabilize mRNA.

The nucleic acids may be used as hybridization probes for cDNA and genomic DNA or as primers for a nucleic acid amplification (PCR) reaction, to isolate full-length cDNAs and genomic clones encoding polypeptides and to isolate cDNA and genomic clones of other genes (including genes encoding homologs and orthologs from different species) that have a high sequence similarity.

The nucleic acids described herein, including homologs and orthologs from species, may be obtained by a process which comprises the steps of screening an appropriate library (as understood by one of ordinary skill in the art) under stringent hybridization conditions with a labeled probe or a fragment thereof; and isolating full-length cDNA and genomic clones containing said polynucleotide sequence. Such hybridization techniques are well known to the skilled artisan.

### Cancer Treatment

In one aspect, the present invention provides a method of treating cancer by in a subject by contacting a cancer cell of the subject with a chimeric oncolytic virus that includes a herpesvirus having a modified nucleic acid sequence. The modified nucleic acid sequence includes a modification of the herpesvirus gamma (1)34.5 gene (γ₁34.5) or a nucleic acid with at least about 70% homology to the γ₁34.5 gene that reduces its expression; a second viral nucleic acid sequence encoding a PKR evasion protein that does not cause virulence; and a third nucleic acid sequence encoding a tumor-associated antigen. The chimeric oncolytic virus can be any of the variants and embodiments described herein. For example, in some embodiments, the herpesvirus is an HSV-1 herpesvirus, while in further embodiments the second viral nucleic acid sequence is a cytomegalovirus (CMV) nucleic acid.

The invention provides a method of treating cancer in a subject in need thereof using the chimeric oncolytic herpesvirus described herein. The term “cancer” refers to a proliferative disorder caused or characterized by a proliferation of cells which have lost susceptibility to normal growth control. Cancers of the same tissue type usually originate in the same tissue, and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived).

Methods of treating cancer in a subject by contacting a cancer cell of the subject with a chimeric oncolytic virus are described. The contracting step can be performed in vivo or ex vivo. The target cell can be a solid tumor cell. The disclosed chimeric virus can also be used to treat a precancer condition such as cervical and anal dysplasia, other dysplasia, severe dysplasia, hyperplasia, atypical hyperplasia, or neoplasia. Thus, the target cell can be a adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, or pancreatic cancer. The target cells can be ectodermally-derived cancer cells. The target cells can be brain cancer cells. Thus, the target cell can be a neuroblastoma cell, glioma cell, or glioblastoma cell. The target cell can be a breast cancer cell. The target cell can be a hepatoblastoma cell or liver cancer cell. The method of killing a targeted cell can further comprise additional steps known in the art for promoting cell death.

Also provided herein is a method of treating cancer in a subject comprising contacting a cancer cell with the herein provided chimeric oncolytic virus. The cancer can be selected from the group consisting of adenocarcinoma, sarcoma, glioma, glioblastoma, neuroblastoma, plasmacytoma, histiocytoma, melanoma, adenoma, myeloma, hepatoblastoma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer. Thus, the cancer can be a glioma. In further embodiments, the cancer can be a glioblastoma. The cancer can be a neuroblastoma. The cancer can be a breast cancer. The cancer can also be pancreatic cancer or hepatoblastoma.

Glioblastomas (GBMs) suppress infiltrating and peripheral immune cell function. The tumors secrete TGF-β, IL-10 and prostaglandin E2, which downregulate T lymphocyte immune recognition and cytokine production. Regulatory T cells (T_{Regs}) and tumor associated macrophages within the tumor contribute to elevated IL-10 production, which functionally impairs infiltrating T effector cells. Several tumor antigens have been identified that are specifically expressed or upregulated in GBM, but immunosuppression in the tumor microenvironment and dysfunctional antigen processing pathways in malignant cells attenuate acquired immune responses. Mohme et al., Cancer Treat Rev, 40(2):248-58 (2014)

As described herein, the chimeric oncolytic virus used for cancer treatment includes a third nucleic acid sequence encoding a tumor-associated antigen. The tumor-associated antigen can be an antigen present only in a tumor cell and not on any other cell, or it may be an antigen present in some tumor cells and also in some normal cells. In some embodiments, tumor-associated antigen is one found on the cancer being treated. For example, EphA2 is a tumor-associated antigen commonly expressed by glioblastoma. Accordingly, when treating glioblastoma, the chimeric oncolytic virus can be modified to express the tumor-associated antigen EphA2.

An anti-viral immune response contributes to the effect of the chimeric oncolytic virus. The chimeric oncolytic virus induces interferon signaling, which recruits both the innate (e.g., neutrophils, NK cells, and macrophages) and adaptive (CD4+, CD8+) immune responses, as well as improved antigen recognition. The chimeric oncolytic virus also reverses the immunosuppressive tumor environment, and stimulates anti-tumor immune recognition. Including tumor-associated antigens in the chimeric oncolytic virus enhances the vaccine approach, resulting in a chimeric oncolytic virus provides a persistent antitumor effect based on a vaccination effect.

Methods in accordance with the invention include administration of the chimeric oncolytic virus alone, or combination therapies wherein the animal is also undergoing one or more cancer therapies selected from the group consisting of surgery, chemotherapy, radiation therapy, thermotherapy, immunotherapy, hormone therapy and laser therapy.

In general any combination therapy will include one or more of chemotherapeutics, targeting agents like antibodies; kinase inhibitors; hormonal agents and the like. Combination therapies can also include conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, anti-cancer agents that are well known in the art and can be used as a treatment in combination with the compositions described herein include, but are not limited to As used herein, a first line “chemotherapeutic agent” or first line chemotherapy is a medicament that may be used to treat cancer, and generally has the ability to kill cancerous cells directly.

Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinizing releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

### Immunization Against Cancer

Another aspect of the invention provides a method of immunizing a subject against cancer. The method includes administering to the subject a chimeric oncolytic virus, comprising a herpesvirus having a modified nucleic acid sequence, comprising: a modification of the herpesvirus gamma (1)34.5 gene (γ₁34.5) or a nucleic acid with at least about 70% homology to the γ₁34.5 gene that reduces its expression; a second viral nucleic acid sequence encoding a PKR evasion protein that does not cause virulence; and a third nucleic acid sequence encoding a tumor-associated antigen, wherein the chimeric oncolytic virus is administered under conditions effective to immunize the subject against cancer. In some embodiments, the chimeric oncolytic virus is administered together with a pharmaceutically acceptable carrier.

The chimeric oncolytic virus can be any of the variants and embodiments described herein. For example, in some embodiments, the herpesvirus is an HSV-1 herpesvirus, while in further embodiments the modification of the herpesvirus γ₁34.5 gene comprises a deletion or mutation of the γ₁34.5 gene.

The chimeric oncolytic virus can be used to immunize a subject against any type of cancer described herein. For example, in some embodiments, the cancer is selected from the group consisting of adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer cell. In a further embodiment, the cancer is glioblastoma.

Immunization can be used to decrease the likelihood that cancer will develop in a subject. In some embodiments, the chimeric oncolytic virus is administered to a subject who has been identified as being at risk of developing cancer (e.g., glioblastoma). A subject can be at risk of developing cancer as a result of a family history of developing cancer, the identification of genes associated with increased cancer risk, or the exposure to radiation or other carcinogenic material.

### Formulations and Methods of Administration

The chimeric oncolytic viruses and viral vectors described herein can be administered in vitro or in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

Pharmaceutical compositions may include carriers, thickeners, diluents, buffers, preservatives, surface active agents and the like in addition to the molecule, in this case virus or viral vector, of choice. Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of a pharmaceutically-acceptable carriers include, but are not limited to, saline, Ringer's solution and dextrose solution. The pH of the solution is preferably from about 5 to about 8, and more preferably from about 7 to about 7.5. Further carriers may include sustained release preparations such as semipermeable matrices of solid hydrophobic polymers containing the antibody, which matrices are in the form of shaped articles, e.g., films, liposomes or microparticles. It will be apparent to those skilled in the art that certain carriers may be more preferable depending upon, for instance, the route of administration and concentration of composition being administered.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as; for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The viruses and vectors can be administered in a number of ways depending on whether local or systemic treatment is desired, and on the area to be treated. Administration may be topical, oral, by inhalation, or parenterally, for example by intravenous drip, subcutaneous, intraperitoneal or intramuscular injection. The disclosed viruses and vectors can be administered intravenously, intraperitoneally, intramuscularly, subcutaneously, intracavity, or transdermally. Thus, administration of the provided viruses and vectors to the brain can be intracranial, subdural, epidural, or intra-cisternal. For example, the provided viruses and vectors can be administered directly into the tumors by stereotactic delivery. It is also understood that delivery to tumors of the CNS can be by intravascular delivery if the virus or vector is combined with a moiety that allows for crossing of the blood brain barrier and survival in the blood. Thus, agents can be combined that increase the permeability of the blood brain barrier. Agents include, for example, elastase and lipopolysaccharides. The provided viruses and vectors are administered via the carotid artery. In another aspect, the provided viruses and vectors are administered in liposomes, such as those known in the art or described herein. The provided viruses and vectors can be administered to cancers not in the brain intravascularly or by direct injection into the tumor.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein for the methods taught therein.

It is also possible to link molecules (conjugates) to viruses or viral vectors to enhance, for example, cellular uptake. Conjugates can be chemically linked to the virus or viral vector. Such conjugates include but are not limited to lipid moieties such as a cholesterol moiety. (Letsinger et al., Proc. Natl. Acad. Sci. USA, 1989, 86:6553-6556).

The viruses and viral vectors described herein may be administered, for example, by convection enhanced delivery, which has been used with adenovirus and AAV to increase the distribution of the virus thorough bulk flow in the tumor interstitium. Chen et al., J. Neurosurg. 103(2):311-319 (2005) Genetic modifications have also been used to enhance viral spread. For example, insertion of the fusogenic glycoprotein gene produced an oncolytic virus with enhanced antiglioma effect. Fu et al., Mol. Ther. 7(6): 748-54 (2003). Therefore, the viral vectors described herein may comprise such a gene.

### Dosages

The exact amount of the compositions required will vary from subject to subject, depending on the species, age, weight and general condition of the subject, the severity of the disease being treated, the particular virus or vector used, its mode of administration and the like. Thus, it is not possible to specify an exact amount for every composition. However, an appropriate amount can be determined by one of ordinary skill in the art using only routine experimentation given the teachings herein.

Effective dosages and schedules for administering the compositions may be determined empirically, and making such determinations is within the skill in the art. For example, there are several brain tumor models that provide a mechanism for rapid screening and evaluation of potential toxicities and efficacies of experimental therapies. There are six separate human glioma xenograft models used for critical studies. Pandita et al., Genes Chromosomes Cancer 39(1): 29-36 (2004). There is also available a spontaneously derived syngeneic glioma model that does not express foreign antigens commonly associated with chemically or virally induced experimental tumors. Hellums et al., Neuro-oncol. 7(3): 213-24 (2005). Other animals models for a variety of cancers can be obtained, for example, from The Jackson Laboratory, 600 Main Street Bar Harbor, Me. 04609 USA, which provides hundreds of cancer mouse models. Both direct (histology) and functional measurements (survival) of tumor volume can be used to monitor response to oncolytic therapy. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy.

The dosage ranges for the administration of the compositions are those large enough to produce the desired effect in which the symptoms of the disease are affected. The dosage should not be so large as to cause adverse side effects, such as unwanted cross-reactions, anaphylactic reactions, and the like. The dosage can be adjusted by the individual physician in the event of any counterindications. Dosage can vary, and can be administered in one or more dose administrations daily, for one or several days.

Viral recovery and immunohistochemistry have been used successfully to monitor viral replication and spread in vivo. Bioluminescent and fluorescent protein expression by the virus can also be used to indirectly monitor viral replication and spread in the tumor. Genes encoding fluorescent reporter proteins (d2EGFP and dsRED monomer) or bioluminescent markers (firefly luciferase) are commonly used in recombinant viruses. Not only do these facilitate the screening and selection of recombinant viruses in vitro. The reporter genes also allow indirect monitoring of viral activity in in vivo studies.

The provided chimeric viruses require lower dosing as compared to existing attenuated herpesviruses. The provided chimeric virus significantly improves survival as compared to conventional attenuated herpesviruses, such as, for example, Δγ₁34.5 HSV, and is effective at lower doses. For example, the disclosed chimeric oncolytic virus is effective at from about 10³ pfu, including 10⁴, 10⁵, 10⁶, 10⁷, 10⁸, and 10⁹ pfu, or any amount in between. Thus, the dose of chimeric virus can be from 5×10³ to 5×10⁶ pfu, more preferably from 5×10⁴ to 5×10⁵.

The following examples are included for purposes of illustration and are not intended to limit the scope of the invention.

### EXAMPLES

### Example 1: oHSV Construction and Dendritic Cell Targeting

GBMs are one of the most fatal and treatment-refractory cancers leading to interest in experimental therapies. Two types of experimental therapies (dendritic cell immunotherapy and oHSV therapy) have demonstrated efficacy in recent early-phase clinical trials for adult GBM. Phuphanich et al., Cancer Immunol Immunother, 62(1): p. 125-35 (2013). Although they are distinct approaches, both strategies likely achieve efficacy through induction of anti-tumor immunity. Oncolytic HSV efficacy was previously attributed to direct replication-based lysis of tumor cells; however, viral activation of host cell antiviral responses is increasingly recognized as another significant effect, as these responses recruit potent anti-tumor effectors and stimulate acquired immune responses. Tumor cell selectivity of many oHSV are based on deletion of the γ₁34.5 neurovirulence gene (Δγ₁34.5 oHSV), which renders them safe for direct inoculation into the CNS, but also debilitates their replication in tumors. To overcome this, a chimeric Δγ₁34.5 oHSV (C134) was created that expresses the IRS1 gene from a distantly-related herpes virus, Human Cytomegalovirus (HCMV). Shah et al., Gene Ther, 14(13): p. 1045-54 (2007). C134 maintains protein translation unlike first generation oHSVs and this leads to improved viral replication in tumor cells without making the virus more virulent. C134 elicits a robust IFN response, culminating in improved recruitment and activation of innate and acquired effector cell populations. Cassady et al., J Virol, 86(1): p. 610-4 (2012).

The inventors have harnessed C134's protein translation and immunostimulatory properties to increase cross sensitization to tumor antigens by overexpressing glioma antigens directly from the viral genome. They previously demonstrated that virus-based transgene expression is efficient and may circumvent HSV pathways that suppress host cell gene expression, including potential tumor associated antigens (TAAs), upon infection. Shu et al., Proc Natl Acad Sci USA, 110(18): p. E1669-75 (2013). In addition, they have engineered C134-expressed TAA to be secreted from infected cells for targeted binding to dendritic cells (DCs). Antigen processing pathways in GBM and HSV-infected cells are attenuated, whereas HSV infection enhances MHC expression in neighboring cells, and recruits activated DCs into the surrounding tissue. Therefore, secreted TAA have a better chance of uptake and cross-presentation in APCs, and fusion of DC-targeting elements to TAAs should enhance this activity

Compared to Δγ₁34.5 oHSV, improved animal survival following C134 treatment appears to be mediated, in part, through stimulation of anti-tumor immunity. Previous studies have demonstrated that repeated oHSV administration in flank GBM models can induce acquired anti-GBM immunity. Iizuka et al., Int J Cancer, 118(4): p. 942-9 (2006). The inventors compared the level and specificity of immune responses induced following C134 and Δγ134.5 oHSV administration. The extent to which each virus induces anti-tumor responses over those directed towards viral antigens was also assessed. The inventors believe that C134 enhances acquired anti-tumor immune responses and these responses are the primary mechanism for its efficacy.

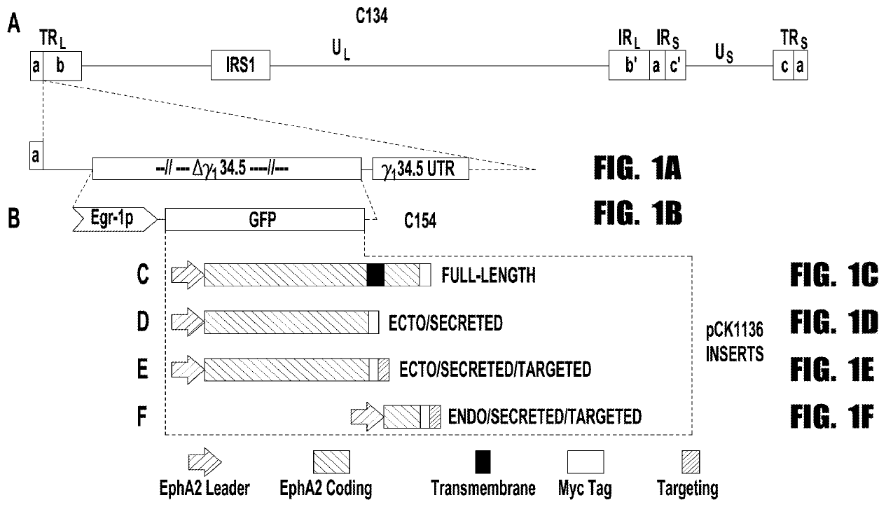

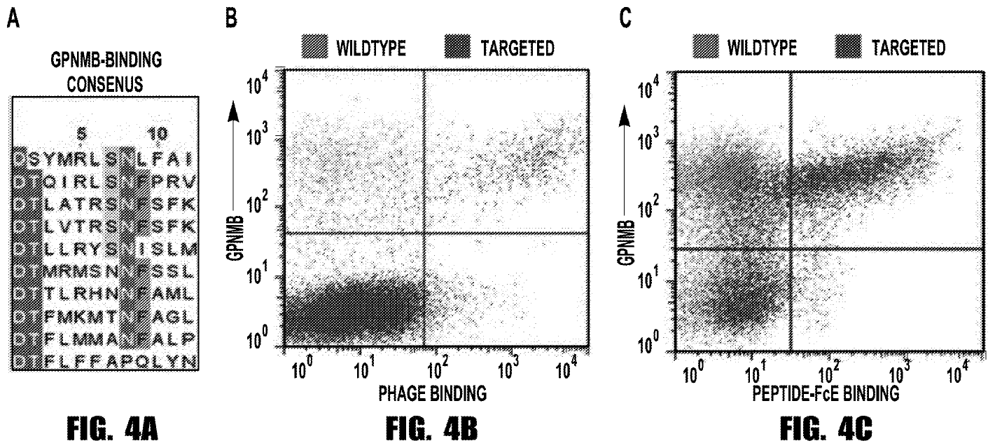

Construction of EphA2 shuttle vectors: The pCK1136 vector was used for recombination-based insertion of transgene expression cassettes into the γ₁34.5 locus of C134 (FIG. 1A, B). Cassady et al., J Virol, 86(1): p. 610-4 (2012). This vector utilizes the murine Egr-1 promoter to drive transgene expression. The inventors previously used this strategy to make a GFP-expressing derivative of C134 (C154). Four separate EphA2 derivatives were inserted into pCK1134, as depicted in FIG. 1C-F: The first will encode the full-length EphA2 reading frame with a C-terminal Myc tag (FIG. 1C). For secreted variants, the EphA2 coding sequence will be truncated to encompass either the extracellular domain (FIG. 1D-E) or the cytosolic domain. Fusion of the first 28 N-terminal residues of EphA2 to the cytosolic domain (aa 575-977) restores the cleavage potential of the EphA2 leader sequence, which should reroute the intracellular domain for secretion (FIG. 1F). All constructs will contain a C-terminal Myc tag to distinguish C134-expressed EphA2 from that of infected tumor cells. The inventors have identified a number of peptides that target DC cells (FIG. 4). For DC-targeting, the murine DC-binding peptide coding sequence can be engineered into the C-terminus of secreted EphA2 variants (FIG. 1E-F).

oHSV construction and validation: Each pCK1136-EphA2 variant was used for recombination with C154 by co-transfection into rabbit skin cells. Individual GFP-negative plaques were serially passaged and purified for further characterization. The EphA2 expression cassettes within viral clones were sequenced. To verify virus-based EphA2 expression results in elevated EphA2 levels, GL261 cells were infected with C134 and C134-EphA2 derivatives. EphA2 levels (total and Myc-tagged) were measured by Western analysis of infected cell lysates. Expression of secreted variants was evaluated by ELISA, and DC targeting was assessed by flow cytometric detection of binding to dendritic cells. Viral recovery assays was used to ensure all oHSV have equivalent replication rates.

The inventors evaluated whether C134-based antigen expression enhances GBM-specific CTL responses and survival in murine orthotopic GBM models. Orthotopic GL261 tumors will be established in syngeneic C57BL/6 mice. The first set of studies compared survival in mice implanted i.c. with cells that are infected with C134 or C134 expressing the full-length EphA2 variant ex vivo. The mice were re-treated on Days 5 and 15 post-transplant, and EphA2-specific responses were evaluated.

The inventors also determined whether secretion and targeting improves EphA2-specific responses. Animal studies were carried out to assess whether secretion of DC-targeted EphA2 variants (FIG. 1E, F) improves the survival. The inventors also evaluated a non-targeted EphA2 variant (FIG. 1D) to ascertain whether secretion and/or secretion with targeting is responsible for improved results. The experiments showed that EphA2-expressing C134 derivatives, particularly variants that are secreted and targeted to DCs, will improve survival of mice with GL261 tumors.

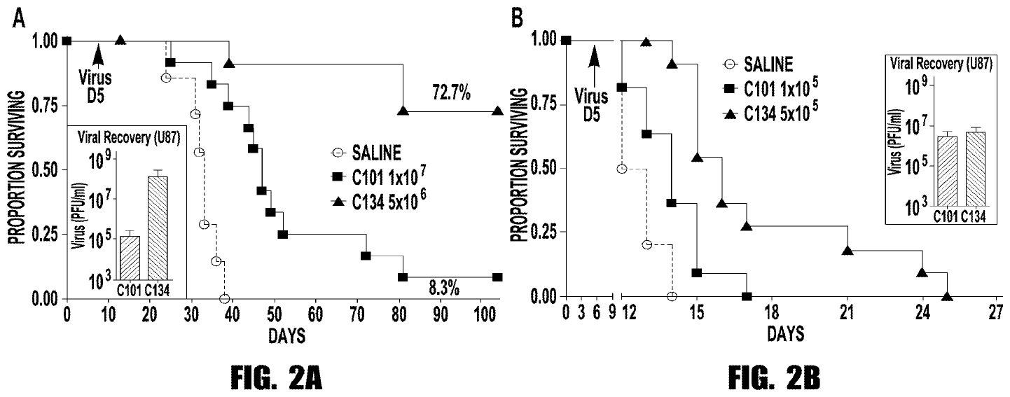

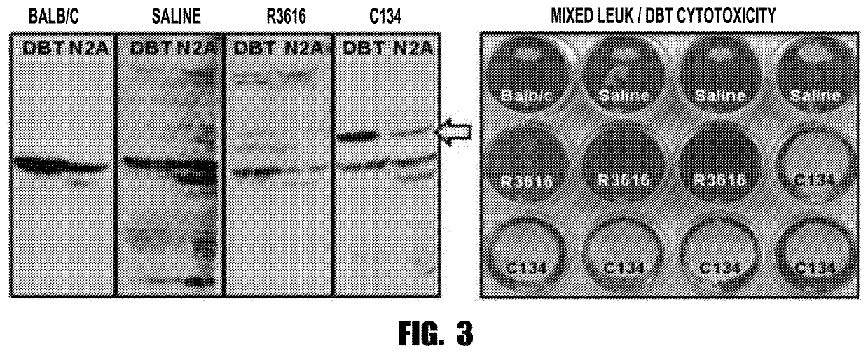

C134 mediates replication-dependent and independent survival benefits in murine brain tumor models. Preliminary studies in the human U87 GBM cell line demonstrated that C134 has a 1000-fold higher replication rate, compared to a Δγ₁34.5 virus, which translates into improved survival in immunodeficient animals with U87 tumors (FIG. 2A). However, C134 is also protective in immunocompetent mice bearing syngeneic Neuro2A tumors, in which there is no replication advantage (FIG. 2B). These data suggest that C134 may also have enhanced immunostimulatory potential. To test this phenomenon in a syngeneic GBM model, mice were implanted with i.c. DBT cells and treated with saline, Δγ₁34.5 (R3616), or C134 oHSV. Surviving animals in each cohort were subsequently rechallenged with DBT flank tumors. Serum and splenocytes obtained from surviving animals were then assessed for immunoreactivity. As shown in FIG. 3A, serum collected from naive and all treatment groups detected a common band in both DBT and Neuro2A (N2A) cell lysates. However, C134-treated mouse sera recognized a unique ˜60 KDa band in extracts of both cell lines (arrow), even after a 4000-fold dilution. Similarly, DBT cell cytotoxicity was achieved with a 10-fold excess of mixed splenocytes collected from C134-treated mice (FIG. 3B). These studies demonstrate C134-based stimulation of anti-tumor immunity, and suggest these effects can be enhanced by directing immunity to specific TAAs.

In addition to C134-based expression of EphA2 variants for induction of anti-tumor immunity, the inventors evaluated incorporate DC-targeting domains to enhance these responses. Using Phage Display, they previously identified a peptide that binds to myeloid derived DCs, and the peptide maintains binding function when incorporated as N- and C-terminal protein fusions. Alberti et al., Gene Ther, 20(7): p. 733-41 (2013). They also recently identified 12-mer peptides that bind to the dendritic cell-associated heparan sulfate proteoglycan-integrin ligand (DCHIL/GPNMB) (FIG. 4A). GPNMB expression on dendritic cells prevents memory and effector T cell activation through co-inhibitor interactions with syndecan-4. These peptides may allow for both targeting of antigens to GPNMB-expressing APC and inhibition of GPNMB-mediated T-cell suppression.

### Example 2: Evaluation of a DBT Malignant Glioma Cell Line for Modeling the oHSV Anti-Tumor Response

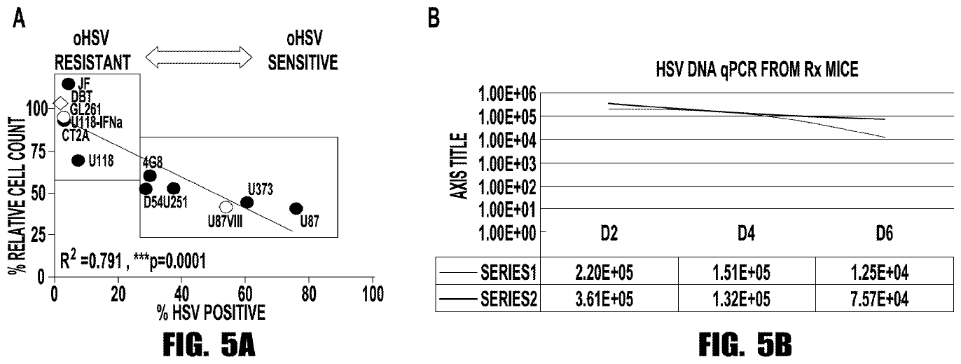

Increasingly, the immune response is recognized as an important component for long term anti-tumor effect contributing to anti-tumor therapies (biologics, radiation therapy, chemotherapy). In the past, researchers have questioned the importance of the immune response and its role in oHSV mediated anti-tumor activity. These studies have allowed us to develop an in vivo model that is primarily dependent upon the immune mediated anti-tumor response. As shown in FIG. 5A, the DBT malignant glioma cell line in contrast to other glioma cell lines is highly resistant to oHSV infection and cytopathic effect.

Using this model, the inventors were able to identify differences in the oncolytic activity between our first and second generation recombinant viruses and how cytokine expression, dosing schedule, and prior immunity influence the indirect antitumor response. In contrast to past CNS tumor models, viral yields are equivalent between 1st and 2nd generation oHSVs in vivo and direct viral cytolysis does not significantly contribute to the anti-tumor activity in this syngeneic glioma (FIG. 5); thus allowing us to assess immune mediated anti-tumor effect in isolation.

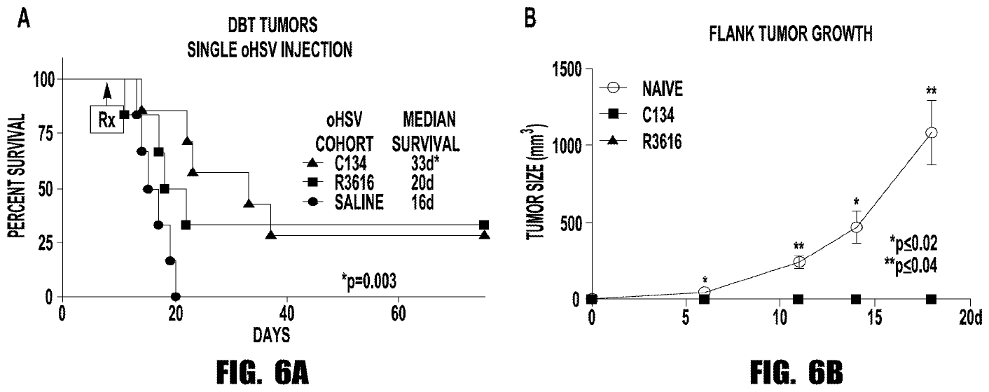

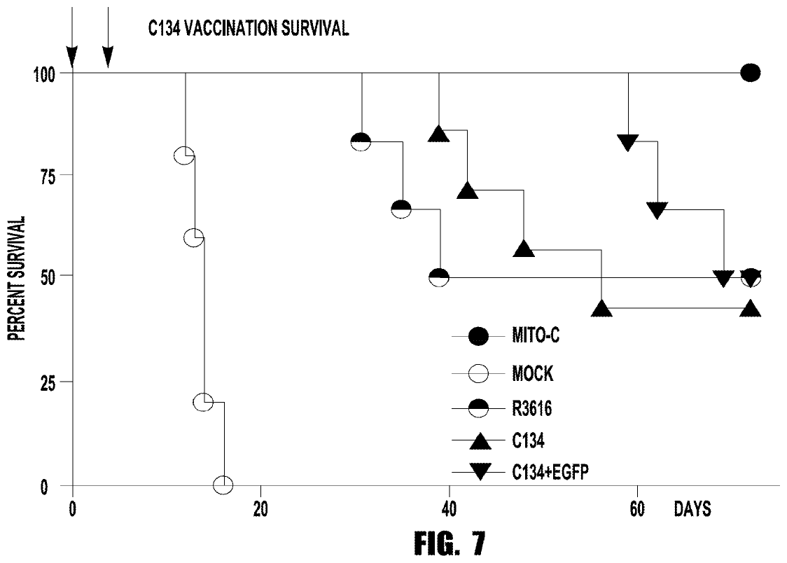

In the DBT model, C134 is again superior to first generation Δγ₁34.5 oHSV (FIG. 6A) despite equivalent viral replication in the CNS tumors (FIG. 5B). This suggests that there are differences in either the cytopathic activity of the viruses (independent of replication) or that the immune-mediated response differs. In past neuroblastoma-based studies, the inventors identified that the immune response contributes to this difference. Implantation of N2A tumors in the CNS of athymic nude mice eliminated any survival advantage for the oHSV anti-tumor therapy. To test if the immune response contributed to this anti-tumor response, DBT tumor cells were implanted in the flanks of survivors and naïve mice. The previously oHSV treated survivor mice suppressed tumor growth during re-challenge, suggesting that a circulating anti-tumor immunity had developed in these survivors. In contrast the implanted tumors grew uncontrolled in the oHSV and Tumor inexperienced naïve mice. These studies reinforce that the anti-tumor immune response contributes to oHSV therapy and that this is not unique to a single animal or tumor type.

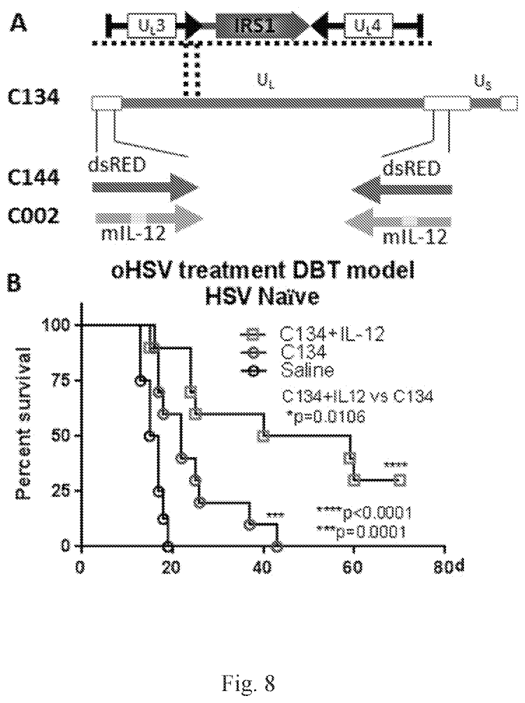

A winn-type assay was performed to determine if the anti-tumor immune response could be primed. These studies identified that repeated dosing of all of the viruses tested improves oHSV anti-tumor activity for both our 1st generation and our 2nd generation C134 oHSVs (FIG. 7). These studies also suggest that foreign antigen expression from the oHSV may extend survival (FIG. 7: C154: C134+EGFP). Studies also verified that priming the immune response by pro-inflammatory mIL-12 cytokine expression from C134 also extends survival in a glioma model (FIG. 8, C002 vs C134). Previous studies showed a similar benefit in a neuroblastoma CNS model; these studies confirm this for glioma as well.

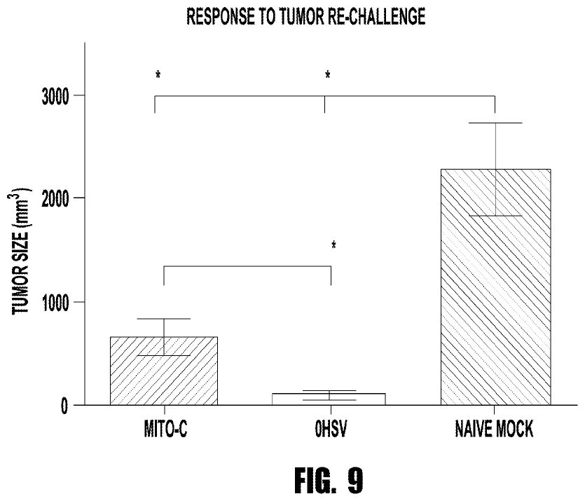

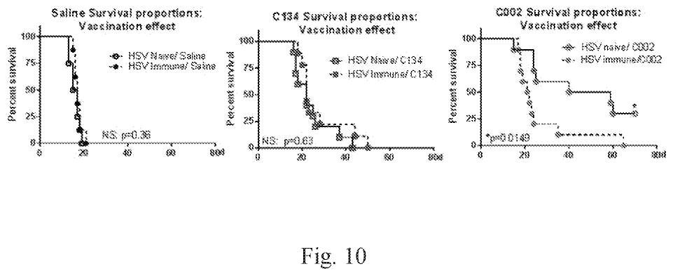

Studies also identified long term anti-tumor benefits from oHSV therapy. oHSV treatment improved durable anti-tumor immunity over mice exposed to tumor antigens alone (FIG. 9). The oHSV-mediated anti-tumor immunity appears to be a circulating immune response and did not require resident memory cells in that the surviving mice were able to reject tumors implanted in a distant site.

The inventors had hypothesized that prior immunity may improve the immune response and increase bystander damage to the tumor; however, their findings suggested that pre-existing HSV immunity does not improve the oHSV anti-tumor effect and did not improve survival (FIGS. 10A&B). For the “unarmed” oHSVs (R3616 and C134), prior immunity was of no benefit but it was also not detrimental.

Unexpectedly, it was found that prior immunity was detrimental to the C134-based mIL-12 cytokine expressing virus C002 (FIG. 10C). As discussed previously, IL-12 expression (which enhances T cell activation) significantly improved C134 anti-tumor activity (FIG. 8). The inventors had anticipated that C002 would improve the anti-tumor activity of these primed T cells. However, it was discovered that HSV immunity adversely affected this recombinant and eliminated any advantage provided by the IL-12 expression, rendering it only as effective as a C134 in HSV-immune mice. The inventors have determined that C002 does not replicate as well during the initial days post-inoculation in mice with prior immunity. Quantitative PCR studies are also examining viral IL-12 transgene expression. The loss of IL-12 expression could explain why C002 (C134+mIL12) behaved similar to a C134 oHSV in this study.

The findings were unanticipated and suggest that there are different contributors to oHSV activity in the HSV naïve and HSV experienced individuals and raise several new questions. Is this loss of oHSV activity specific to “armed” transgene expressing viruses or are 11-12 expressing viruses unique in their ability to enhance the anti-HSV immune response? Does prior immunity limit the replication of all of our oHSVs (1st generation Δγ134.5 and C134) shifting the direct and indirect anti-tumor contribution in immunized animals? Our past studies have used survival as our measure for overall oHSV anti-tumor activity. This readout combines direct and indirect anti-tumor effect. While past studies showed no change in “net anti-tumor effect”, prior immunity may shift the relative contribution direct (viral cytolysis) and indirect immune mediated anti-tumor effect. This is an especially important issue with our newer generation armed oHSVs (like C002 or M002) that rely on robust viral gene expression.

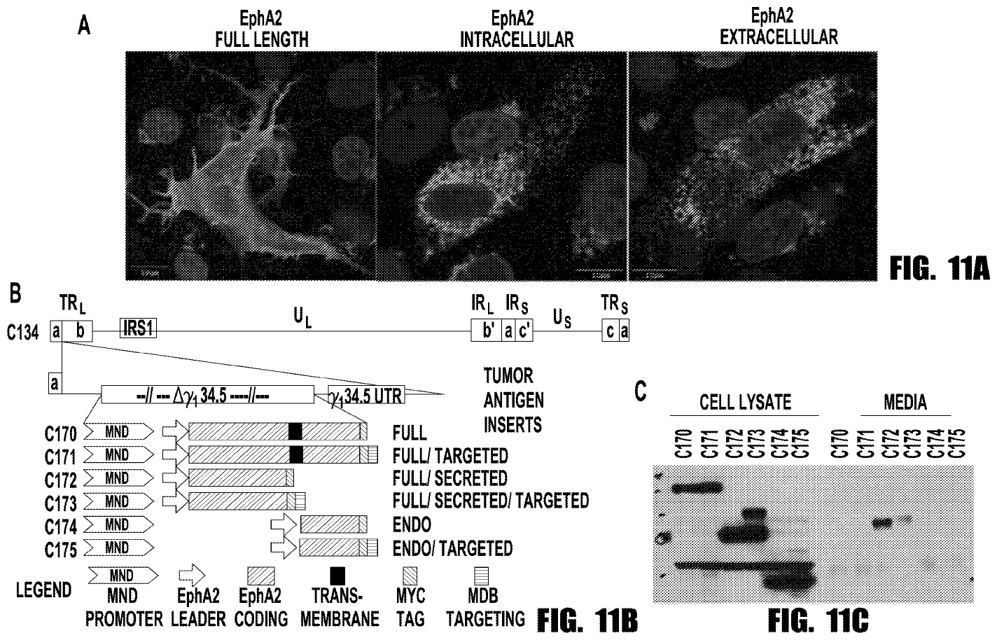

The inventors created and validated 6 different C134-based tumor antigen expressing viruses (summarized in FIG. 11). Two isolates for each of the 6 viruses (12 viruses) were collected, named (C170-181) and validated by DNA hybridization, transgene expression, intracellular antigen localization, and cellular secretion during infection (FIG. 5). Remaining to be accomplished: Show evidence of MBP tag function (binding to antigen presenting cells [APCs]) in vitro and in vivo.

In conclusion, the inventors were able to validate a tumor model that unequivocally shows that the immune response contributes to oHSV therapy and that this response can be modulated to improve durable anti-tumor effects. They also found that it was not enough to produce an immune response against the virus. In fact, while focusing on the anti-viral immune response did not hinder the first or second generation oHSVs, it did affect survival of mice treated with the armed cytokine-expressing virus. Development of tumor antigen expressing oHSVs (C170-C181) allows for further dissect the role of the anti-tumor and anti-viral immune response.

### Example 3: Antigen-Expressing Oncolytic Virus—A Multimodal Anti-Tumor Vaccine Targeting Shared Antigens

To determine if virus based tumor associated fetal antigens (TAFAs) expression improves anti-tumor immunity in tumors resistant to OV immunotherapy, the inventors created a C134 based virus (C170) that encodes the murine (B6) TAFA Ephrin A2. Using both in vitro and in vivo B6 syngeneic models, they investigated how viral TAFA expression influences OV anti-tumor activity and immune response against the EphA2. The results show that C134 based EphA2 expression improves antitumor effect, changing the tumor associated immune infiltrates and improving anti-tumor memory response, abscopal effect and eliciting specific anti-EphA2 T cell response when compared to the C134 treated cohort. These exciting findings confirm that OVs can be modified to enhance immune recognition of “self” TAFAs and that this strategy can be harnessed as an approach to use viruses for in vivo anti-tumor vaccination.

### Materials and Methods

### Cell Lines and Viruses

CT2A cells were kindly provided by Dr. Seyfried Boston College and were propagated in Dulbecco's modified eagle medium (DMEM) supplemented with 10% fetal bovine serum (FBS). 67C-4 was kindly provided by Dr. Tim Cripe and was developed and provided to him by his collaborator Dr. Nancy Ratner and maintained in DMEM supplemented with 10% FBS.

Tumor lines were tested negative for mycoplasma contamination using the ATCC universal Mycoplasma detection kit. Tumor cells with relative low passage numbers (<12 passages) were used in the study before returning for a “low” passage form of the cell line to minimize genetic drift in our studies. Viruses have been previously described (Ghonime et al., Translational oncology, 11:86-93 (2017)), but in brief, HSV-1 (F) strain and R3616, the Δγ₁134.5 recombinant, were kindly provided by Dr. Bernard Roizman (University of Chicago, Chicago, Ill.). Chou et al., Science, 250:1262-6 (1990). C134 has been described previously. Shah et al., Gene Ther, 14:1045-54 (2007). Briefly, C134 is a Δγ₁134.5 virus that contains the HCMV IRS1 gene under control of the CMV IE promoter in the UL3/UL4 intergenic region. Cassady K A., J Virol, 79:8707-15 (2005). C154 is an EGFP-expressing version of C134.

### Viral Spread Assay (In Vitro)

B76, B96, 67C-4 and 5NPCIS cells were plated into clear, 48-well flat-bottom polystyrene tissue culture-treated microplates (Corning, N.Y., USA) and allowed to adhere overnight at 37° C. Cells were infected the following day with an EGFP-expressing second-generation oHSV-1 (C154) at the indicated multiplicity of infection (MOI), and the plates monitored using the IncuCyte ZOOM platform, which was housed inside a cell incubator at 37° C. with 5% CO₂ until the end of the assay. Nine images per well from three replicates were taken every 3 hours for 3 days using a 10× objective lens and then analyzed using the IncuCyte™ Basic Software. Green channel acquisition time was 400 ms in addition to phase contrast.

### Animal Studies

Animal studies were approved by the Nationwide Children's Hospital Institutional Animal Care and Use Committee (IACUC; protocol number AR16-00088) and performed in accordance with guidelines established by NIH Guide for the Care and Use of Laboratory Animals. Two syngeneic C57/B16 tumor models were used in these studies: an intracerebral CT2A glioma tumor model and a flank 67C4 malignant peripheral nerve sheath tumor (MPNST) model.

For the flank tumor model, 2×10⁶ 67C-4 MPNST cells were injected subcutaneously into the flanks of 6- to 8-week-old C57BL/6 mice (Envigo, Frederick, Md.). Tumor sizes were measured biweekly by caliper after implantation, and tumor volume was calculated by length×width×depth. When tumors reached 25-150 mm³ in size, animals were pooled and randomly divided into the specified groups, discussed below, with comparable average tumor size. Mice were treated with vehicle, C134 or C170 (3.5×10⁷ in 100 μL 10% glycerol in PBS) intratumorally (IT) on day 4 (1 day after the last RUX dose) and again a week later. Studies were repeated 3 times to ensure biological validity.

For survival studies, animals were monitored for tumor volumes three times per week after the initial treatment, until total tumor volume/mouse exceeded 2000 mm³ or an individual tumor was >1500 mm³. Once overall tumor size exceeded these criteria, mice were sacrificed based upon IACUC requirements. For cell recruitment analysis and in vivo gene expression, tumors were harvested, as described below, 6 days after the initial C134 or C170 injection. Tumors were washed in PBS and finely minced into small pieces. Then tissues were digested in RPMI 1640 containing collagenase D (2 mg/mL; Roche) and DNase I (0.01 mg/mL; Roche) for 30 min at 37° C. on a shaking platform. After collagenase digestion, the medium containing the mononuclear cells was strained and centrifuged at 400×g for 10 minutes at 4° C., and the resulting cells were resuspended in RPMI 1640 supplemented with 1% FBS and penicillin/streptomycin, and then used for flow cytometry analysis and RNA extraction. For the CD8 depletion studies, mice were treated with RUX similar to that described above, but upon initiation of the RUX therapy, mice were randomized into anti-CD8 depletion or isotype treatment cohorts. Mice were treated with 100 μg of anti-CD8 (Clone 2.43, Bio X Cell, West Lebanon, N.H.) or the isotype control (Clone LTF-2, rat IgG2a. Bio X Cell, West Lebanon, N.H.) intraperitoneally (IP) twice weekly throughout the experiment. Mice were then treated with IT C134 as described above. To quantify CD8 depletion, mice underwent a tail vein bleed (1 week after initiating CD8 depletion) and the CD8+ T-cell populations were analyzed using FITC-conjugated anti-CD8b (Clone: H35-17.2, eBioscience).

### IC Tumor Implantation and Treatment

6- to 8-week-old C57BL/6 mice were obtained from (Envigo, Frederick, Md.). For survival studies, 1×10⁵ CT2A in 5% methylcellulose were intracerebrally injected into syngeneic C57BL/6 mice and treated 5 days later with vehicle or virus (1×10⁷ PFU/10 μl) as described previously. Russell S J, Barber G N., Cancer cell, 33:599-605 (2018) Mice were assessed daily and moribund mice killed sacrificed and date recorded. Survival curves were determined using Kaplan-Meier analysis and median survivals and 95% confidence intervals calculated. Log-rank test was applied to compare survival between groups. Studies were repeated twice to ensure biologic validity.

Viral Replication (in vivo)

67C-4 tumors were established in 6- to 8-week-old female C57BL/6 as described above. When tumors reached 25-150 mm³ in size, mice were randomized and treated with C134 or C170 intratumoraly (3.5×10⁷ pfu in 100 μL 10% glycerol and PBS). On days 1, 3, and 5 post-virus treatment, tumor samples were harvested and homogenized. DNA was extracted by DNeasy blood and tissue kit (Qiagen, Germantown, Md.) per the manufacturer's instructions. Virus recovery was measured by Tagman quantitative PCR. Ghonime et al., Translational oncology, 11:86-93 (2017). Briefly, extracted DNA samples were incubated with the following HSV-specific primers and probes for HSV polymerase. HSV genome equivalents of the amplified product were measured from triplicate samples using an StepOne Plus real-time PCR system (Applied Biosystems, Foster City, Calif.) and compared against logarithmic dilutions of a positive control DNA sequence (10⁶-10¹ copies). Descriptive statistical analyses (mean and SD) were used to compare differences in DNA copy numbers between samples using Prism 78.0 statistical software (GraphPad).

### RNA Isolation

Total RNA was isolated from tumor samples using the Direct-zol RNA Miniprep Plus kit (Zymo Research, Irvine, Calif.) according to the manufacturer's instructions. RNA quantity and purity was determined using a NanoDrop 2000 Spectrophotometer (Thermo Fisher Scientific, Charlotte, N.C.). 2 μg of total RNA was used to synthesize cDNA using SuperScript III Reverse Transcriptase (Life Technologies, Carlsbad, Calif.) according to the manufacturer's instructions.

### Flow Cytometry

Single-cell suspensions from tumors were obtained as described previously. Leddon et al., Molecular therapy oncolytics, 1:14010 (2015) Briefly, tumors were washed in PBS and finely minced into small pieces. Then tissues were digested in RPMI 1640 containing collagenase D (2 mg/mL; Roche) and DNase I (0.01 mg/mL; Roche) for 30 minutes at 37° C. on a shaking platform. After collagenase digestion, the medium containing the mononuclear cells was filtered and centrifuged at 400×g for 10 minutes at 4° C., and the resulting cells were resuspended in PBS supplemented with 1% FBS and then used for flow cytometry analysis. Single-cell suspensions from tumors were lysed with RBC lysis buffer (Sigma) and blocked with 5% mouse Fc blocking reagent (2.4G2, BD Biosciences, San Jose, Calif.) in FACS buffer (1% FBS and 1 mM EDTA in PBS). Cells were labeled with the following antibody staining panels for analysis of the adaptive immune cells: (1) CD11b-Violet 421 (M1/70), CD4-BV785 (GK1.5), CD25-PE (7D4), CD8a-BV510 (53-6.7), CD3ε-BV 711 (145-2C11), CD44-APC, CD45-BV605, NKp46-PE-Cy7 and B220-AF488 (RA3-6B2) from BioLegend (San Diego, Calif., USA). Dead cells were excluded by staining with Live/Dead Near/IR staining (APC-Cy7) (Thermo Fisher Scientific, Charlotte, N.C.). Single samples were stained with the above staining panels for 30 minutes on ice and washed one time with FACS buffer. After labeling, cells were fixed in 10% paraformaldehyde and analyzed on a BD FACS LSR II (BD Biosciences). Analysis was carried out using the FlowJo software, version 10.0.3 (Tree Star Inc., Ashland, Oreg.).

### IncuCyte ZOOM Viral Spread Assay

Cells were plated into 96-well flat clear bottom polystyrene tissue-culture treated microplates (Corning, N.Y., USA) and allowed to adhere for overnight. C134 or C170 were added at indicated MOI and the plates were transferred into the IncuCyte ZOOM platform which was housed inside a cell incubator at 37° C. with 5% CO₂, until the end of the assay. Four images per well from three technical replicates were taken every 3 hours for 3 days using a 10× objective lens and then analyzed using the IncuCyte™ Basic Software. Green channel acquisition time was 400 ms in addition to phase contrast.

### Western Blotting

Cellular lysates form tumor samples were collected on ice in disruption buffer (10 mM Tris-Cl pH 8.0, 1 mM EDTA, 1% Triton X100, 0.1% sodium deoxycholate, 0.1% SDS, 140 mM NaCl, 20% β-mercaptoethanol, 0.04% bromophenol blue) with complete, mini protease inhibitor cocktail (Roche, Indianapolis, Ind.). The protein concentrations were determined using Pierce™ BCA Protein Assay Kit (Thermo Scientific, Rockford, Ill.). Samples were denatured at 98° C. for 5 minutes, chilled on ice, separated by PAGE, transferred to a nitrocellulose membrane (Thermo Scientific, Rockford Ill.) and blocked for 1 hour at room temperature with 5% dry milk (S.T. Jerrell Co.) or bovine serum albumin (Fisher Scientific, Rockford, Ill.). Membranes were incubated overnight at 4° C. with primary antibody diluted in Tris-buffered saline with 0.1% Tween-20 (TBST). Primary antibodies against RIG-I (clone D14G6), MDA-5 (clone D74E4), and p-STAT-1 (clone 58D6) were purchased from Cell Signaling Technology and against actin (clone C4) from Chemicon. Membranes were repeatedly washed with TBST, incubated for 1 hour with HRP-conjugated goat anti-rabbit (Pierce) for RIG-I, MDA-5, and p-STAT-1 or goat anti-mouse (Pierce) for actin diluted in TBST (1:20,000 dilution) at room temperature, and subsequently washed with TBST. Membranes were developed using SuperSignal West Pico Chemiluminescent Substrate (Thermo Scientific, Rockford, Ill.) and exposed to x-ray film (Research Products International).

Peptide-Pulsing of Splenocytes with Class I(Kb)-Restricted Peptide Epitope of EphA2

Splenocytes (5×10⁵) from the treated tumor bearing mice were plated in round-bottom 96-well plates and stimulated or not with 10 μM EphA2 peptide (671-FSHHNIIRL-679) for 6 hours. Samples were incubated with protein transport inhibitor containing 1 μl/mL Brefeldin A (Golgi-plug™, BD Biosciences, San Jose, Calif.) for 6 h hours prior to flow cytometry staining and CD8 T lymphocytes were analyzed by flow cytometry for granzyme B intracellular staining and activation (CD25).

### Statistical Analysis

Statistical analysis was performed using Prism 8 (GraphPad Software). One-way ANOVA with correction for multiple comparisons (Holk-Sidham or Kruskal Wallace as specified) was used for analysis involving multiple cell lines or otherwise specified. For comparing tumor growth over time between two treatment groups, two-way ANOVA with Sidak's multiple comparisons test was used. Survival was assessed using log-rank assay and data shown using Kaplan-Meier curves. For all analyses, the cutoff for statistical significance was set at P<0.05. The following notation was used: (ns) P>0.05, (*) P≤0.05, (**) P≤0.01, (***) P≤0.001.

### Results

Construction and Characterization of an EphA2-Expressing oHSV

The ability of the immune system to distinguish between normal and cancer cells is essential for cancer immunotherapy and based on retaining sufficient antigenicity by the malignant cells. Coulie et al., Nature reviews Cancer, 14:135-46 (2014). However, tumor cells may lose their antigenicity to evade immune-mediated elimination. Loss of antigenicity can occur by several mechanisms such as the immune selection of cancer cells that lack or mutate immunogenic tumor antigens, dysregulation of antigen processing machinery, or impediment of tumor antigen presentation (e.g., downregulation or loss of major histocompatibility expression). Schreiber et al., Science, 331:1565-70 (2011). The inventors sought an approach that would enhance the immune recognition and antigenicity of therapeutically resistant. They hypothesized that by engineering an oncolytic virus that expresses shared fetal antigens (expressed in many tumors), we could improve the immunotherapeutic response against these fetal antigens capitalizing on the virus's natural tendency to break immune tolerance. Ehl et al., J Exp Med., 187:763-74 (1998).

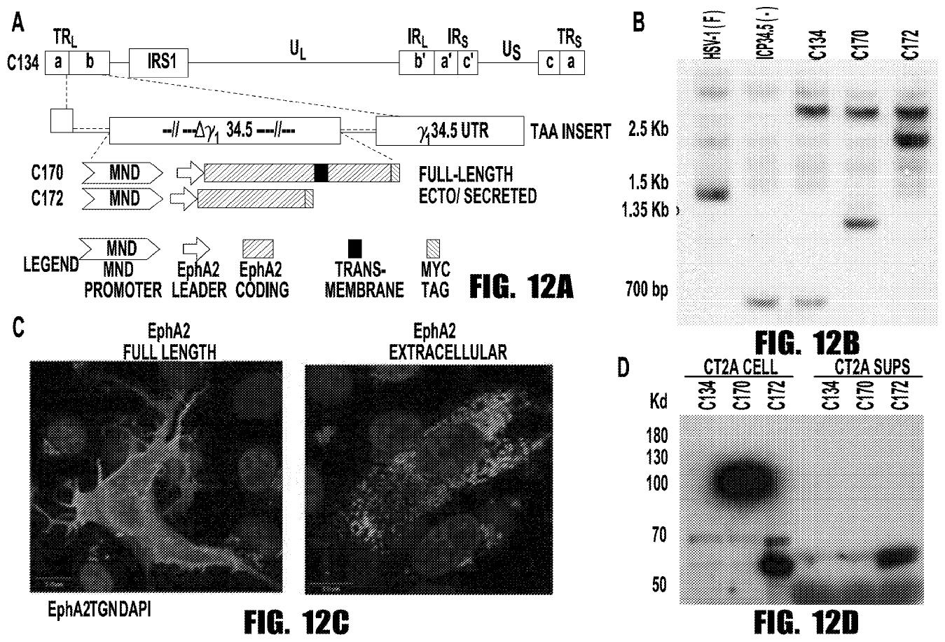

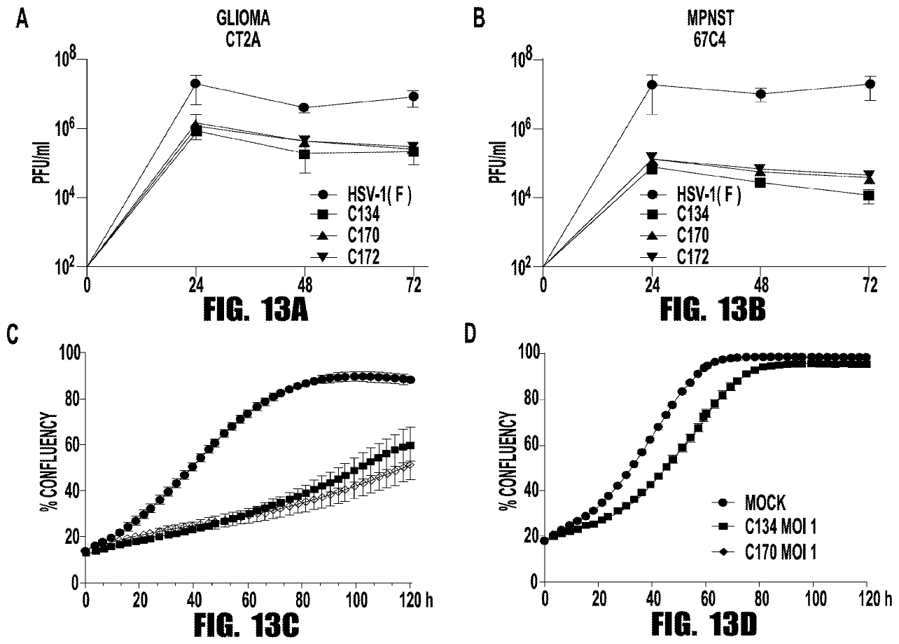

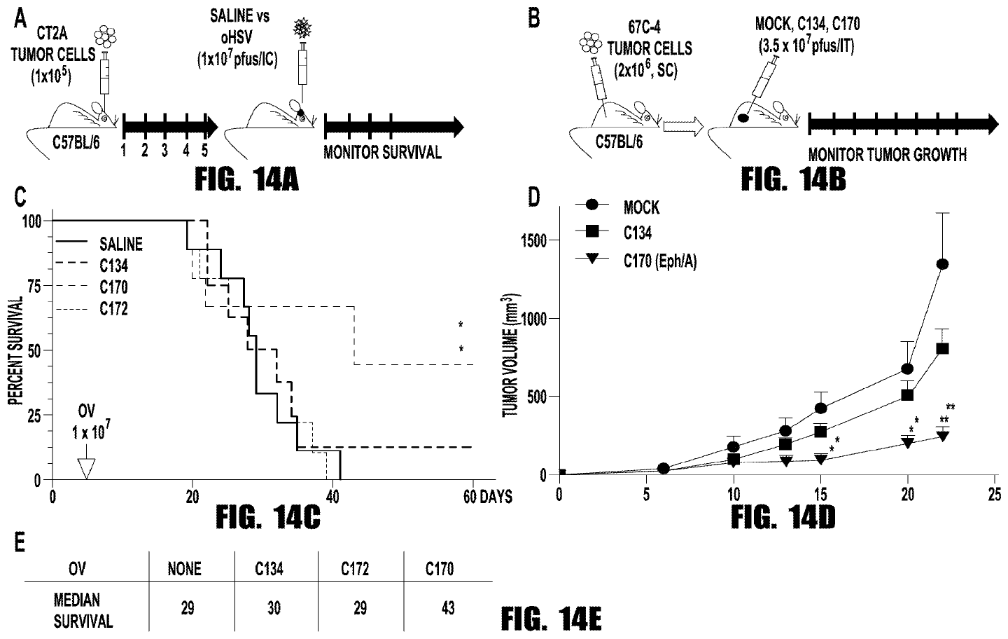

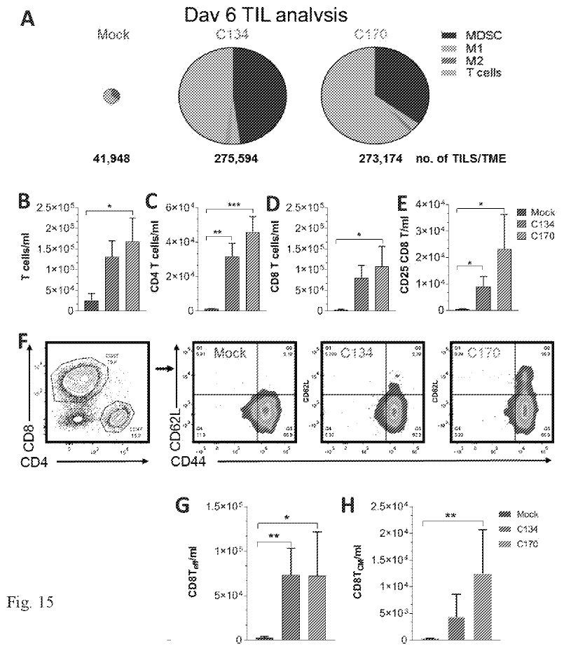

To test this hypothesis, an oncolytic HSV was created that expresses a tumor associated “shared” fetal antigen (EphrinA2) widely expressed in malignant gliomas, sarcomas, and many carcinomas (breast, prostrate, ovarian, pancreatic and colorectal cancer). Tandon et al., Expert Opin Ther Targets. 15:31-51 (2011) Because the studies were focused on using the virus as a flexible platform to present these “self” tumor associated embryonic antigens, oHSV recombinants (C170 and C172) were constructed that express the full length and the secreted extracellular domain of the C57BL/6 Ephrin type-A receptor 2 (EphA2) as outlined in FIG. 12A. HSV recombinants containing the EphA2 gene were verified genetically by DNA hybridization studies and EphA2 protein expression was verified by western blot for right protein size (FIG. 12B), immunofluorescence for localization (FIG. 12C), and by flow cytometry (FIG. 12D) in cell culture based studies. The results show that C170 expresses cell associated EphA2 of the predicted MW 97 kd (FIG. 12B) and with a membrane distribution in infected cells (FIG. 12C). Flow cytometry studies also show that C170 infection increases the EphA2 surface expression above that naturally occurring in either CT2A (C57BL/6-based MG) and 67C-4 (C57BL/6-based MPNST) tumor lines. As indicated above, the goal of this new virus was to improve the immunotherapeutic response to OV treated tumors. The inventors therefore first evaluated how EphA2 insertion and expression influenced viral replication, spread and direct oncolytic activity. To test this, C170 and C134 (the parent virus) replication and cytopathic activity were compared in human and murine tumor lines. As anticipated both viruses have comparable viral replication in CT2A (murine MG line), U87 (Human MG line), 8814 (human MPNST) and 67C-4 (murine MPNST line). This suggests that the new transgene insert did not change virus replication in the tumor lines. Real time evaluation of cell proliferation using Incuyte zoom showed that both C134 and C170 reduce CT2A tumor cell growth equivalently and that in the highly resistant 67C-4 model, neither virus (C134 nor C170) significantly suppress cell growth directly even at a high multiplicity of infection (FIG. 13A-D). In contrast, both viruses inhibited CT2A tumor cell growth in vitro at an MOI 1 and 10. The results show that C170 and C134 replicate similarly, spread and cytopathic activity in vero cells and in human and murine tumor lines in vitro and highlight differences in oHSV oncolytic activity in two different C57BL/6 tumor lines in vitro.

### C170 Improves Reduces Tumor Growth and Improves Survival and in Two Different Highly-Resistant Syngeneic Models

The inventor next sought to evaluate the antitumor activity of the new EphA2-expressing oHSV-1 (C170 and C172) in a therapeutically resistant CT2A syngeneic brain tumor model. CT-2A tumors are an C57BL/6-based anaplastic astrocytoma established by Siefried et al. using chemical induction. Seyfried et al., Molecular and chemical neuropathology, 17:147-67 (1992). The CT2A tumor model recapitulates many of the characteristics of human GBMs that make them so difficult to treat. Like human tumors the CT-2A tumors are radio and chemo resistant, infiltrative with high intra-tumoral cellular heterogeneity, and share similarities with neural stem cells (forming neurospheres when cultured in serum-free media and express stem cell markers such as CD133, Oct, and nestin. Oh et al., J Transl Med., 12:107 (2014). Mice bearing orthotopic CT2A tumors were oHSV-(1×10⁷ PFU) or saline-treated and survival was monitored. Consistent with the in vitro studies, C134 and C170 replicate equally in this tumor model in vivo. Although of equivalent replication capacity, only C170 was able to improve overall animal survival. The median survival for C170 mice was 43 days compared to 29 and 30 days for mock, C172 and C134-treated mice, respectively (FIG. 14A). Furthermore, some of the C170 and a limited number of C134-treated mice cleared their tumors suggesting that the immune response may contribute to oHSV anti-tumor activity. The initial studies also showed that while C170 (C134 based virus expressing the full length EphA2 including both EC and IC domains) improved survival, the C172 virus expressing the secreted EC domain of EphA2 was ineffective and was no different than Saline treatment. This suggests that certain EphA2 domains expressed by the virus were instrumental in OV antitumor effect.

To determine if this difference in oHSV anti-tumor activity was limited to the CT2A tumor model, full-length EphA2 expressing oHSV and C134 was examined in a resistant syngeneic tumor model (67C4 MPNSTs) that shares the same tumor antigen; EphA2. Ghonime et al., Cancer immunology research, 6:1499-510 (2018) Also, C172 was excluded from these studies as it didn't show any survival benefit in the CT2A model. The inventors confirmed the expression of EphA2 on 67C-4 tumor cells and found upregulation of EphA2 expression upon C170 infection, as shown in FIG. 14B. They found that a single treatment of C170 significantly attenuated tumor growth compared to C134 and mock treated groups suggesting that C170 has a better antitumor activity and might be effective in any tumor model that shares EphA2 antigen.

### C170 Treatment Alters Leukocyte Infiltrates in Brain Tumors

As indicated above, both C134 and C170 replicated equivalently in the tumor models and therefore, based upon other studies by the inventors, were anticipated to have equivalent direct anti-tumor activity. They next focused on immune response related to OV-treatment. To evaluate this, the immune cell infiltrates from treated animals were examined to identify differences between C170, C134 and saline treated mice, treated animals were sacrificed, saline perfused them and harvested brains from mock, C134 and C170-treated tumor-bearing mice and compared the immune cell infiltrates. The results show that both the parent oncolytic HSV-1 (C134) and the EphA2-expressing virus (C170) increased leukocyte migration to the tumor as shown in FIG. 15. Of note as shown the pie chart, C170 exhibited a slight increase in the T cell to Myeloid balance with T cells predominating in the C170 treatment cohort. C134 also induced a sizeable T cell infiltrate but along with this also enhanced the myeloid recruitment to the brain. Next, the T cell population's composition was examined and identified that both C134 and C170 induced T cell migration, but only C170 induced a statistically significant increase in the absolute number of T cells in TME (FIG. 15). Both viruses caused significant increase in the number of CD4T, but only C170 caused a significant increase in the number of CD8T cells which might suggest a key role of these cells in the observed antitumor effect. To elucidate the underlying mechanism of the observed antitumor effect, the different subsets of CD8T cells were examined. It was found that both viruses induced CD8T activation as shown in Figure by the CD25 activation marker and increased the number of the effectors CD8 T (CD8 TEFF) cells in TME with a trend of higher number in C170 treated mice. However, only C170 significantly increased the central memory CD8T cells (CD8 TCM) population numbers as shown in FIG. 15. Hu G, Wang S., Scientific reports, 7:10376 (2017).

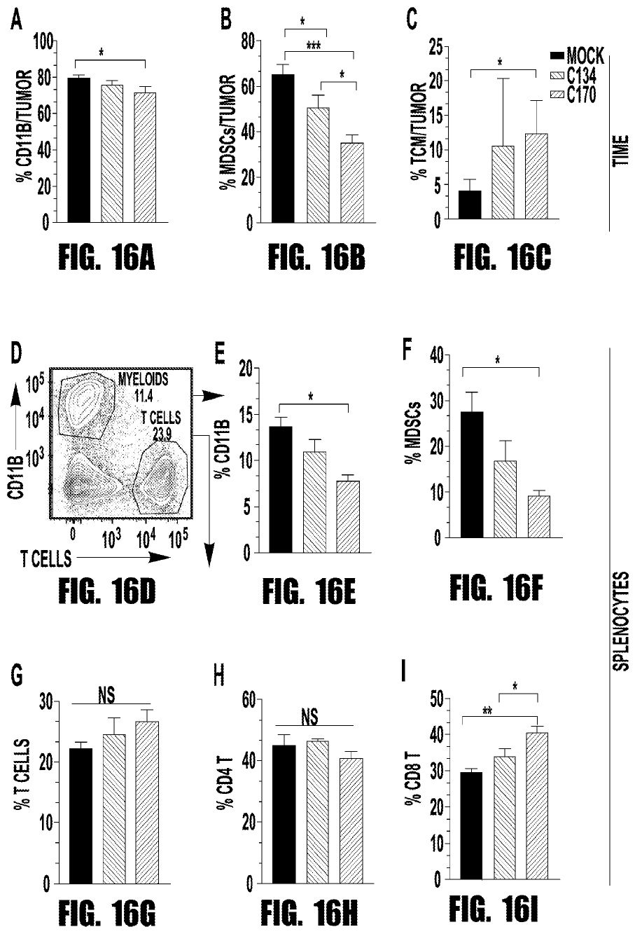

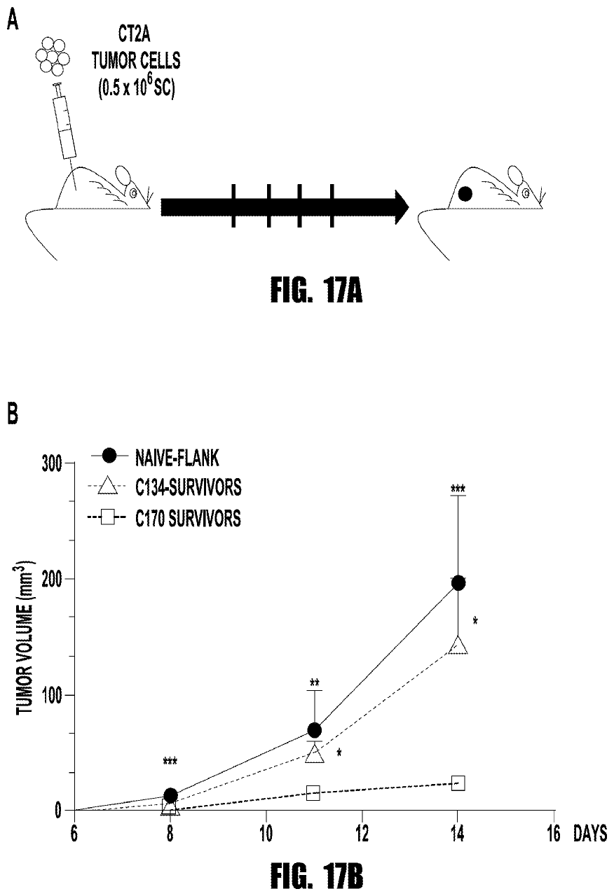

The inventors also examined the oHSV treated 67C-4 MPNST tumors and the periphery for immune cells profile. C170 treated tumors again demonstrated a reduction in the in myeloid population and myeloid-derived suppressors cells (MDSCs) and a significant increase in the TCM population consistent with results from the brain tumor model and (FIG. 16). Furthermore, splenocytes from the saline and oHSVs treated animals showed a significant decrease in the myeloid population and myeloid-derived suppressors cells (MDSCs) and a significant increase in the CD8T cells in C170-treated 67C-4 tumor-bearing mice (FIG. 16). Prior studies have shown that this shift in myeloid and MDSCs population (FIG. 16) and increases in the CD8 T cell population is associated with consistent with results from the brain tumor model. Katoh H, Watanabe M., Mediators of inflammation 2015, 159269 (2015).

### Tumor-Antigen Expressing Oncolytic Virus Develop Systemic Memory and a Durable Anti-Tumor Antigen Response

Based upon the results showing that C170 treated mice induces a central memory population in the tumor, the inventors next examined if the treated mice who responded to oHSV therapy developed systemic memory against the virus expressed tumor antigen.

C170 and C134 produced long term survivors in the CT2A model providing a unique opportunity to test this hypothesis using a functional study. The long-term survivors from the CT2A from the C134 and C170-treated cohorts and age-matched naïve mice were therefore re-challenged to check the development of durable antitumor response. As shown in FIG. 17A-17B, when long term survivors and naïve mice were challenged with CT2A flank tumors, only the C170 long term survivors reduced tumor growth. Consistent with the earlier phenotype data, this suggests that C170 uniquely induces a durable memory population that can circulate and identify the CT2A tumors leading to an abscopal effect (i.e., shrinking of tumors outside the scope of the localized treatment).

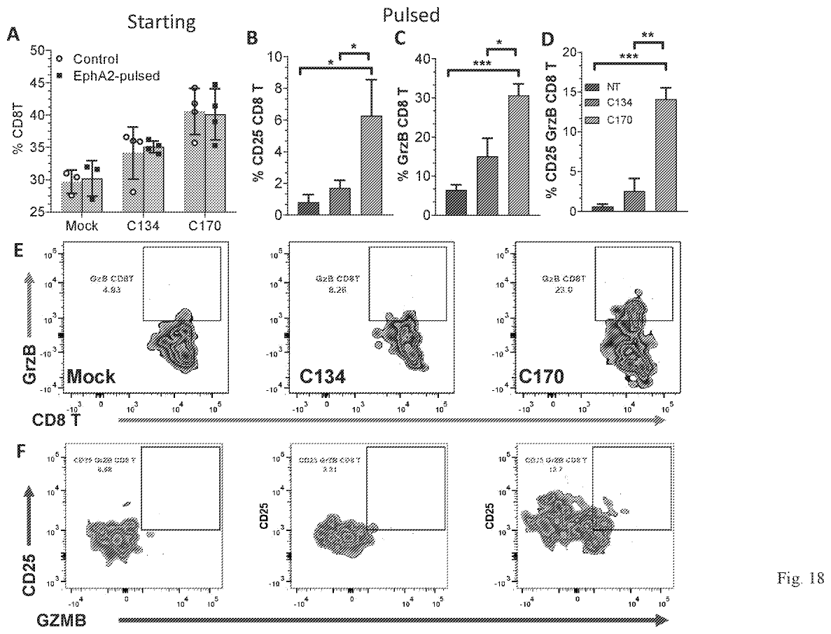

To further investigate this memory response, 67C4 tumor model samples were then examined. In contrast to the brain tumor model, the mice treated in the 67-C4 tumor studies were all sacrificed at the same time (when naïve mice tumors were >1500 mm³) as well as at day 6 post treatment in another study. This allowed the T cell functional response differences between the different treatment cohorts and the development of systemic memory in the periphery against oncolytic virus-expressed tumor antigen EphA2 to be examined. Peptide-pulsing functional assay with the MHC-1 restricted immunodominant peptide of the EphA2 (H-2Db; 671-CFSHHNIIRL-679) for 6 hours. While there was no change in the percentage of the CD8T after 6 hours of stimulation, it was found that only CD8T from mice treated with C170 exhibited a robust activation (CD25 expression) and expression of the effector cytokines (GzmB) as shown in FIG. 18 suggesting that these mice have circulating antigen-specific CD8T cells that could respond to tumor antigens upon re-exposure.

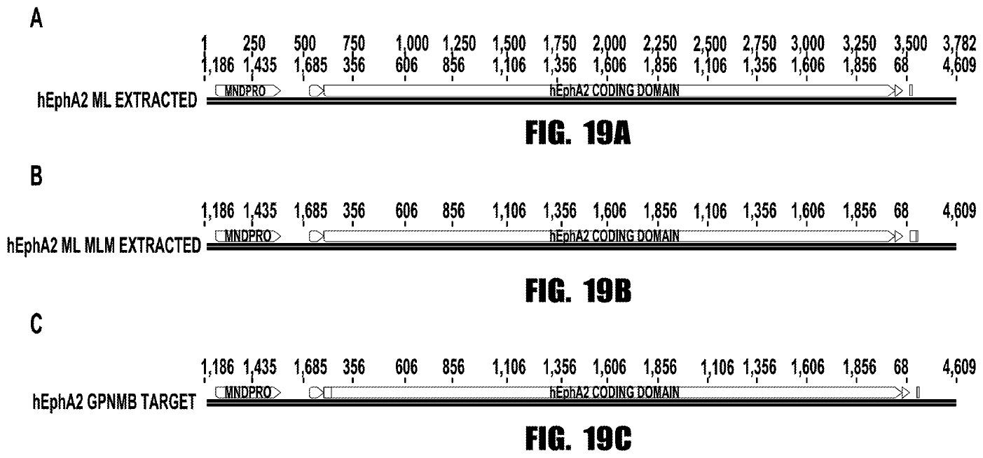

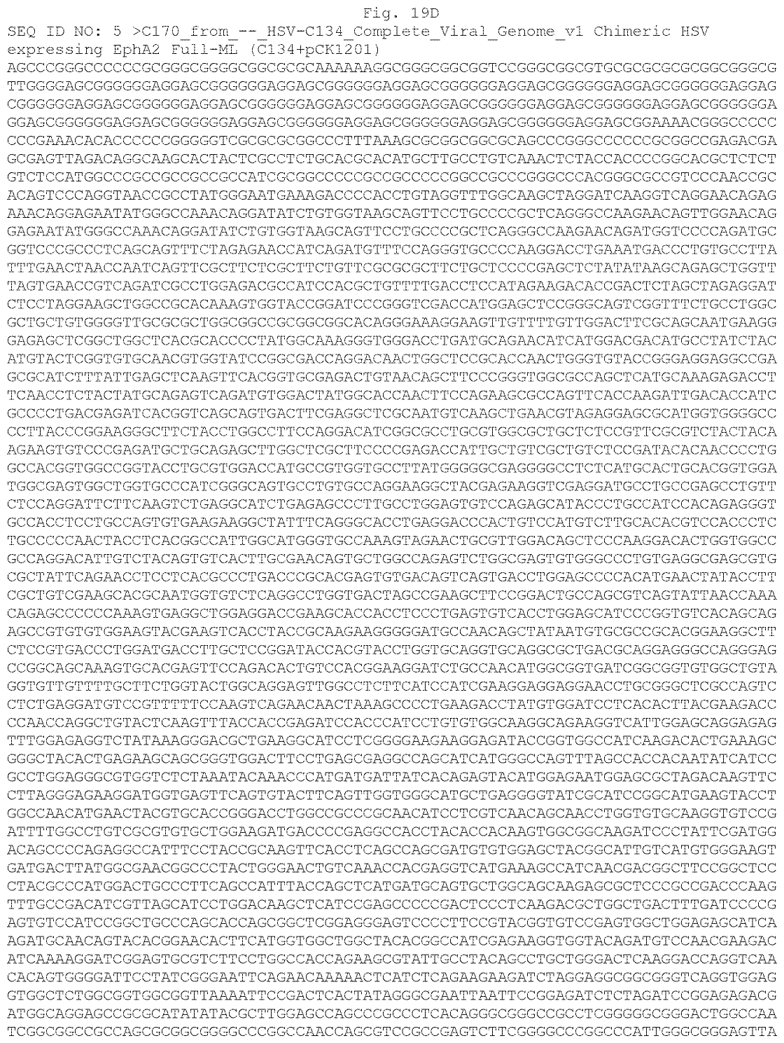

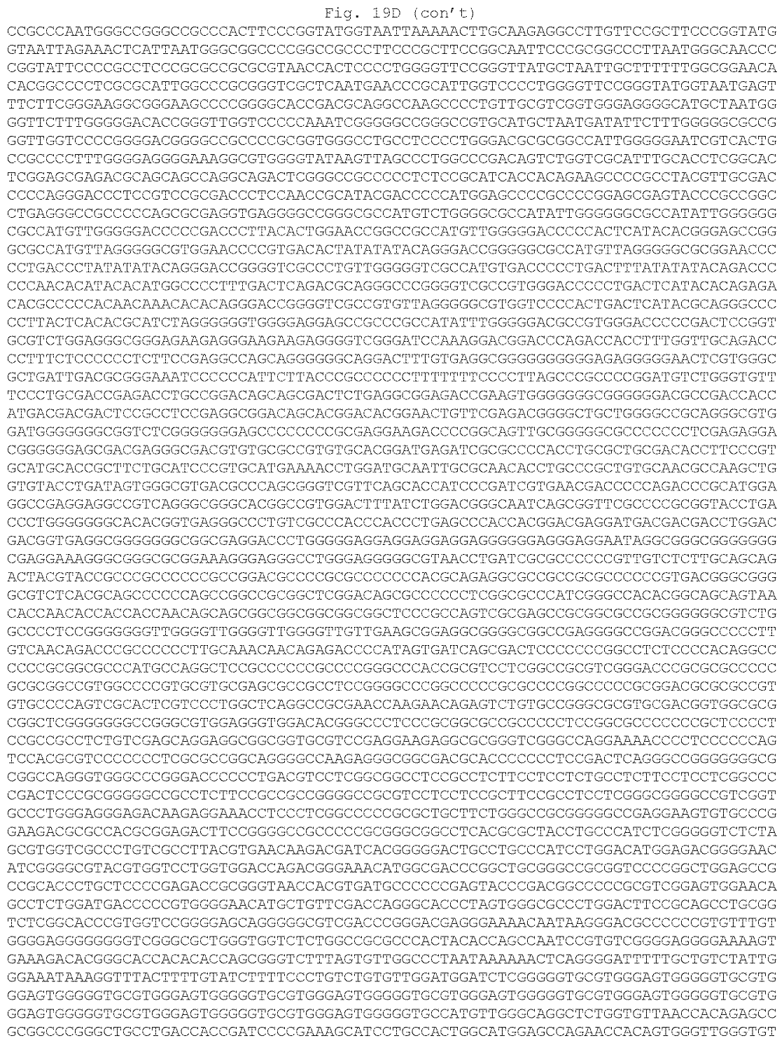

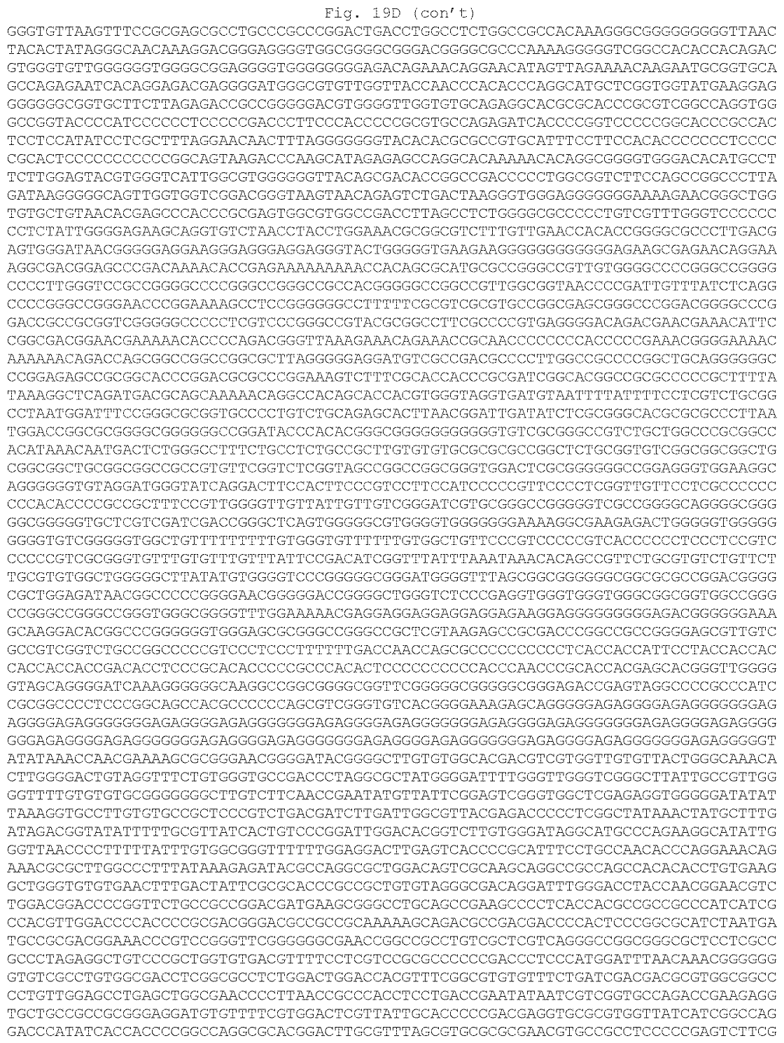

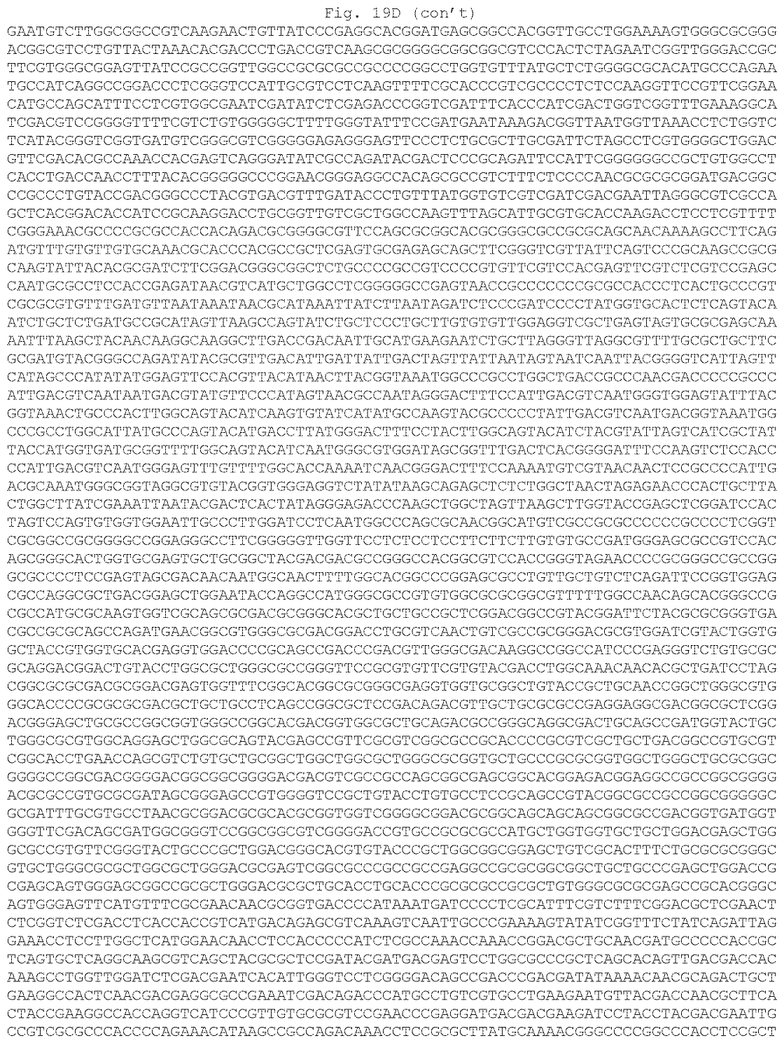

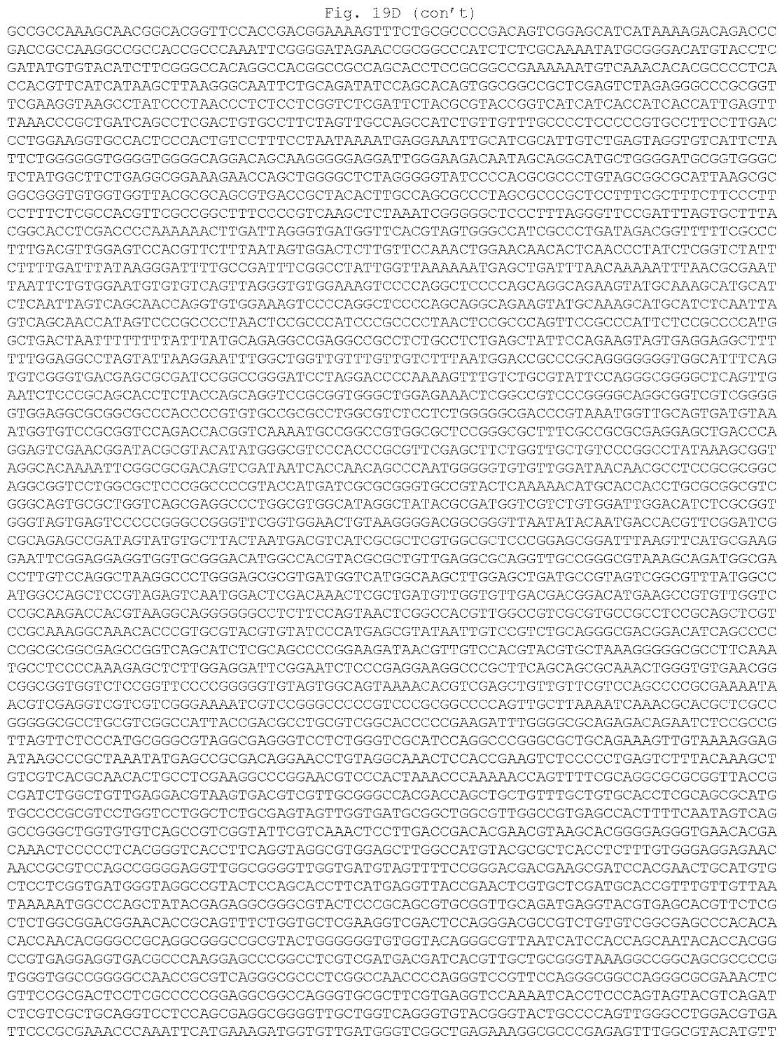

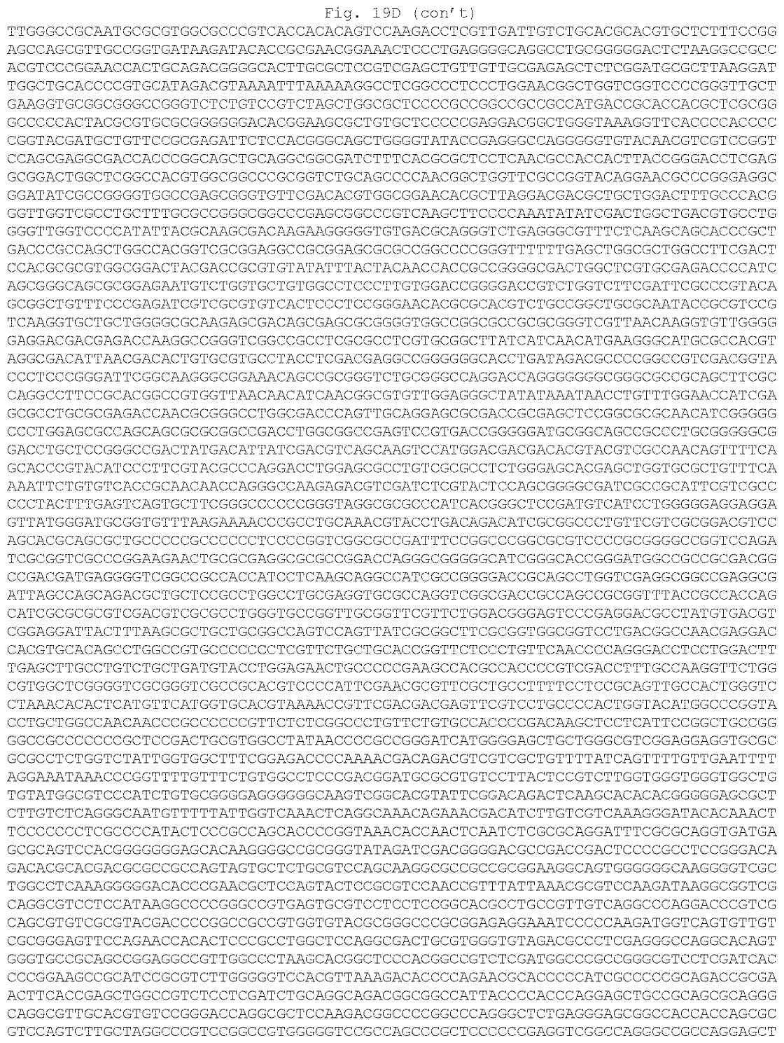

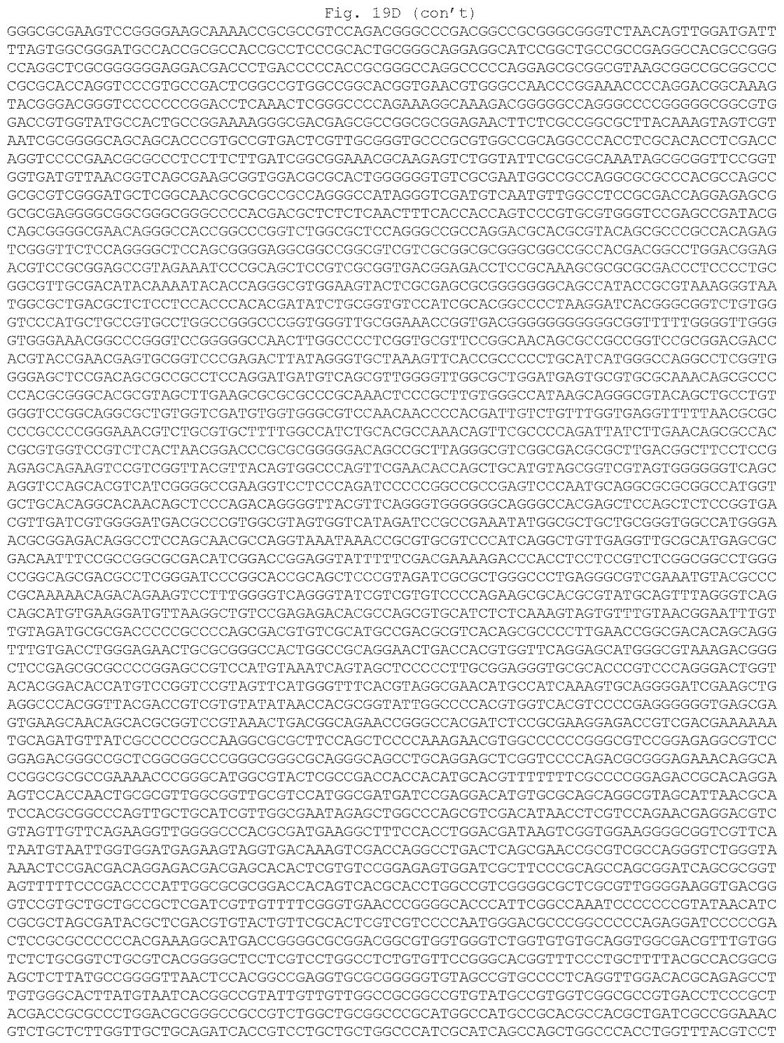

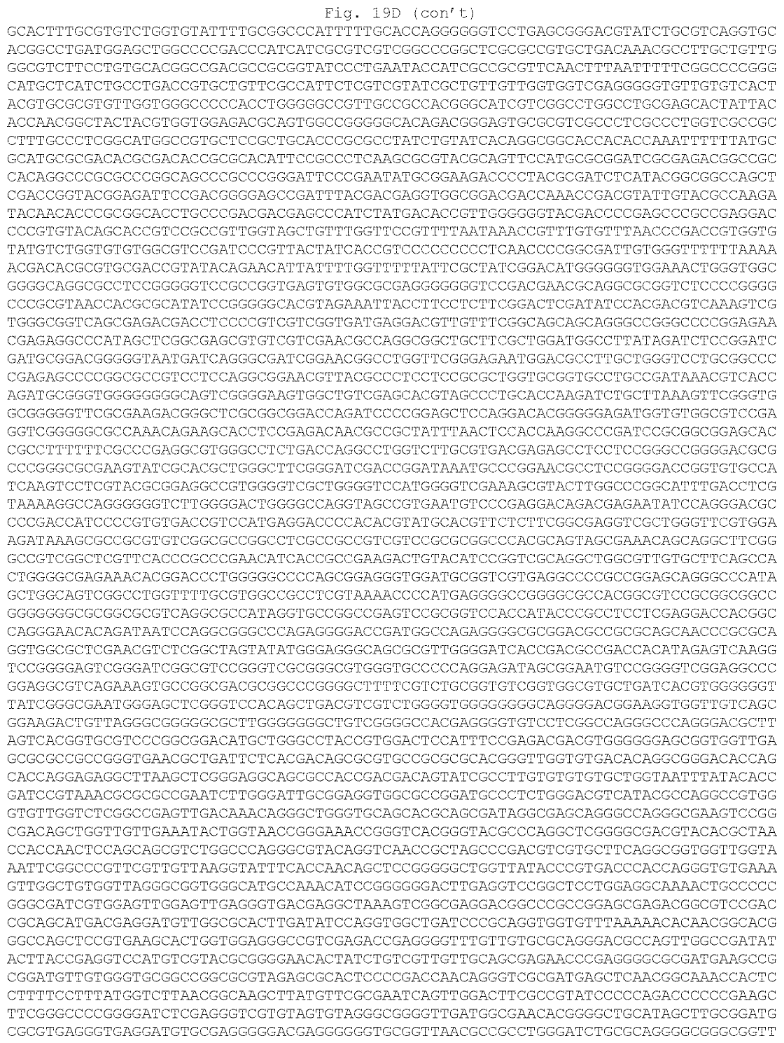

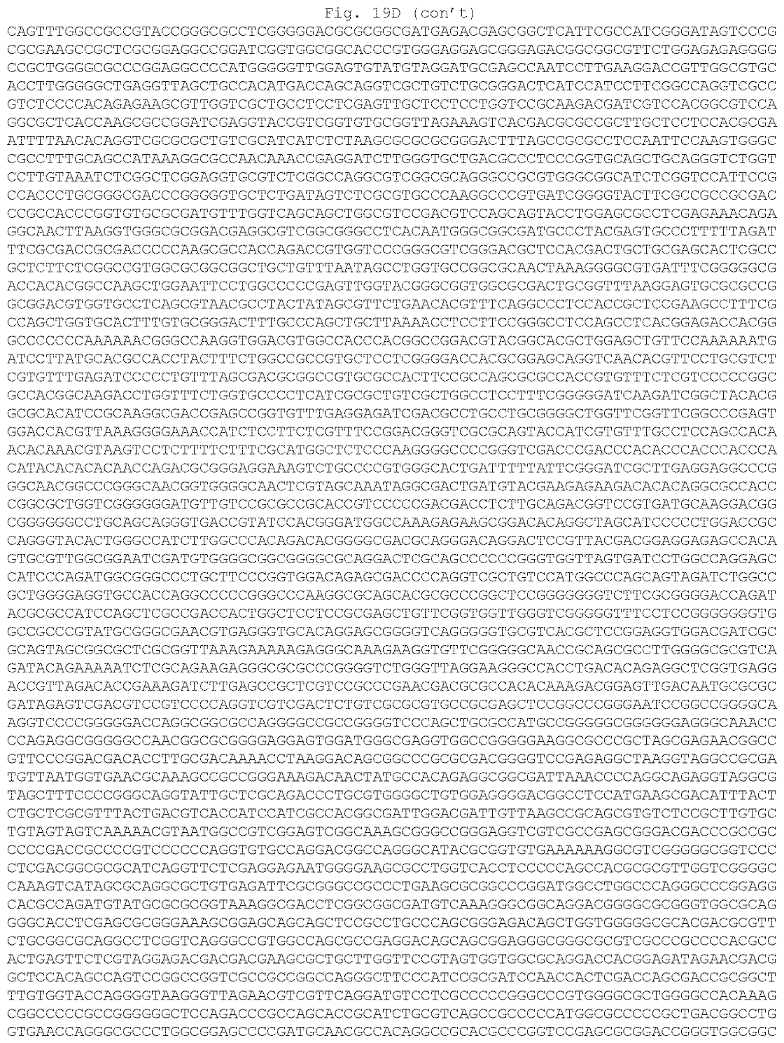

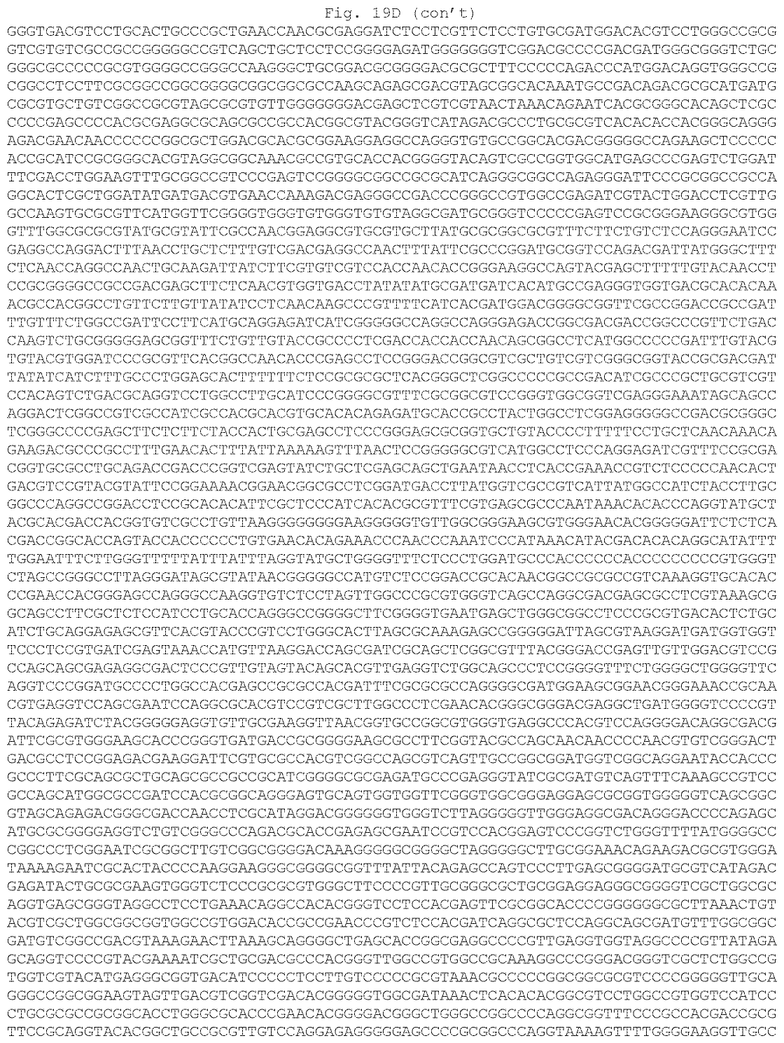

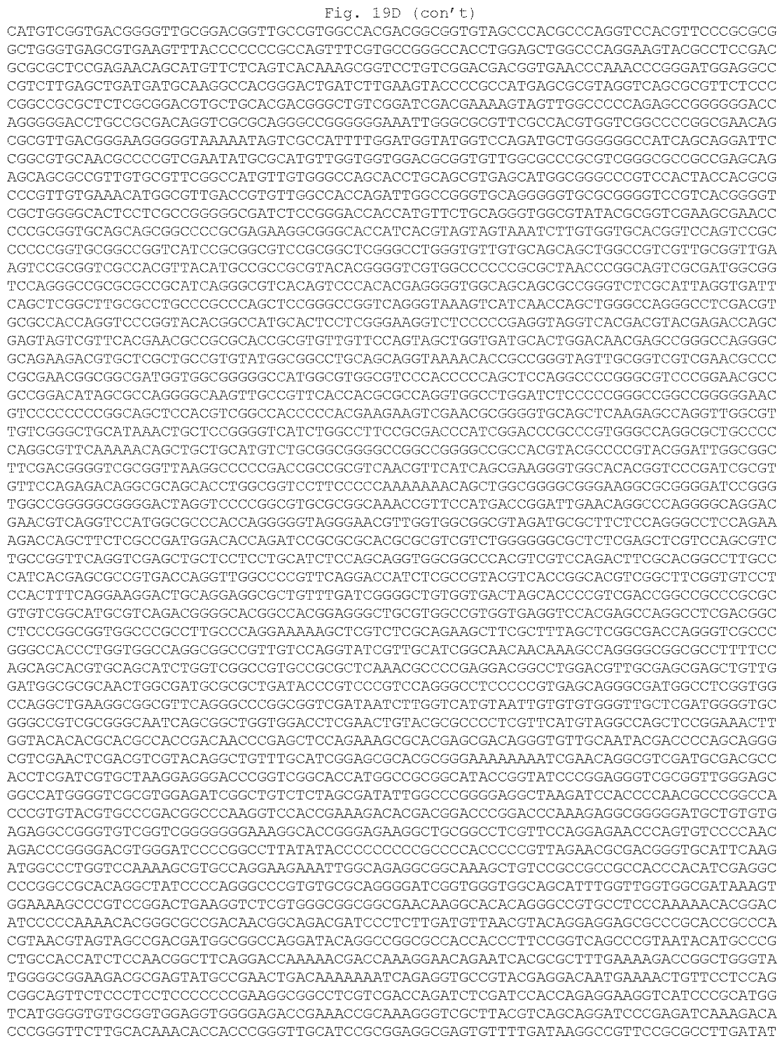

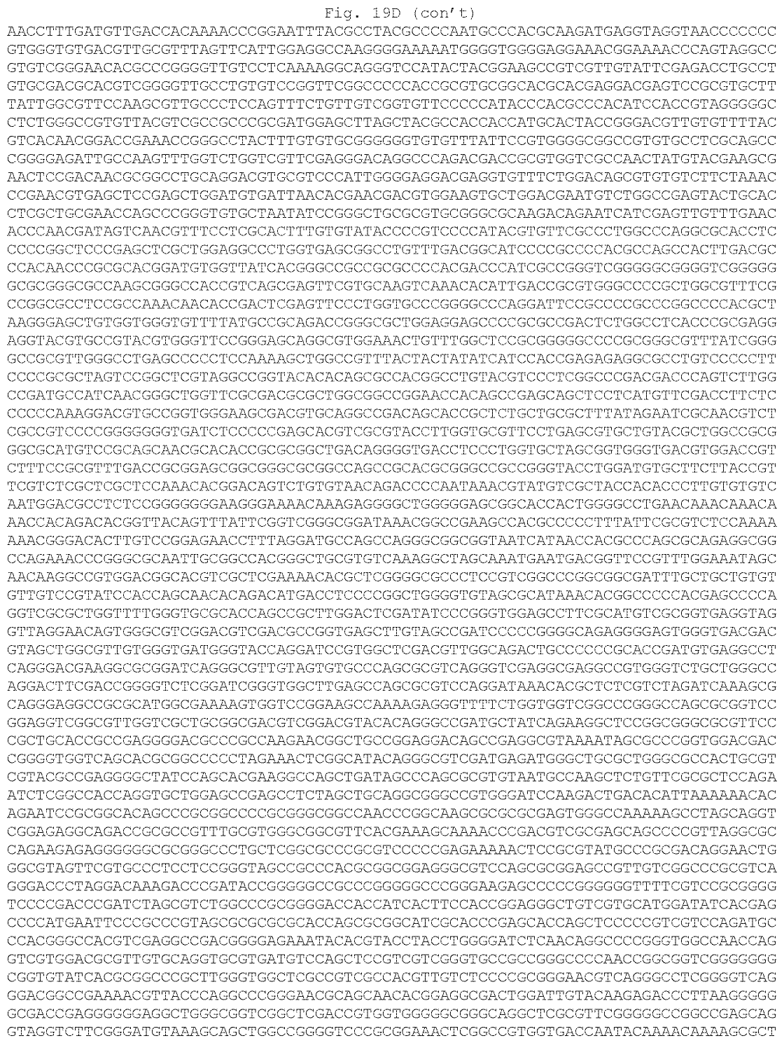

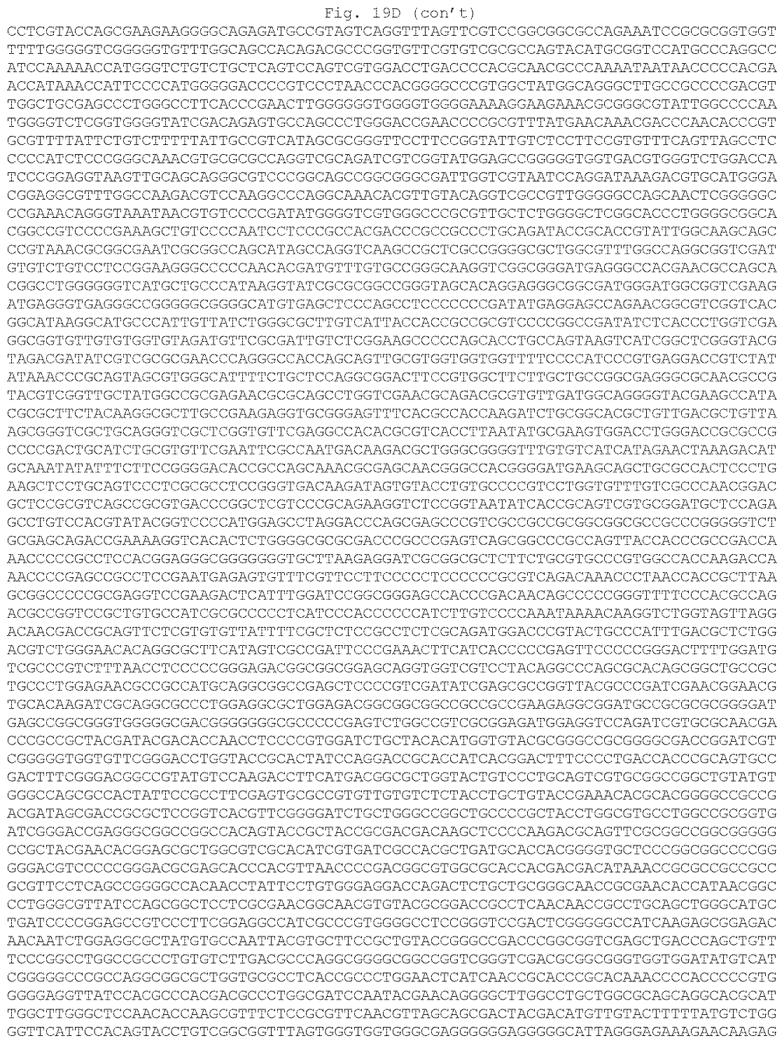

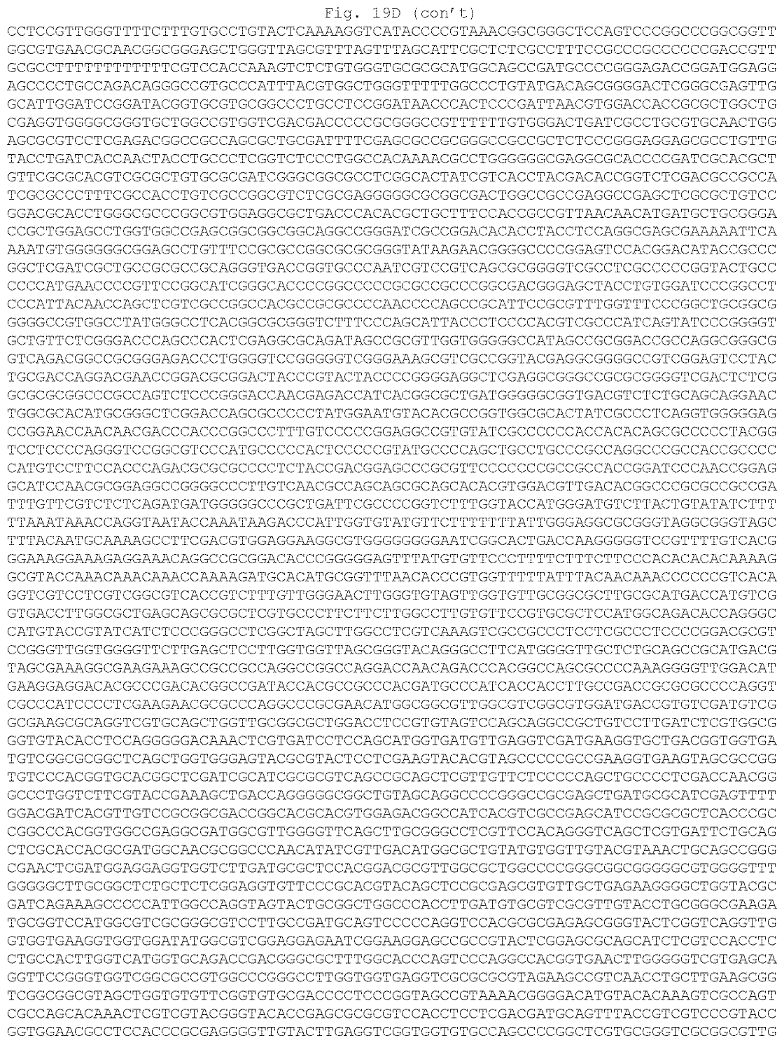

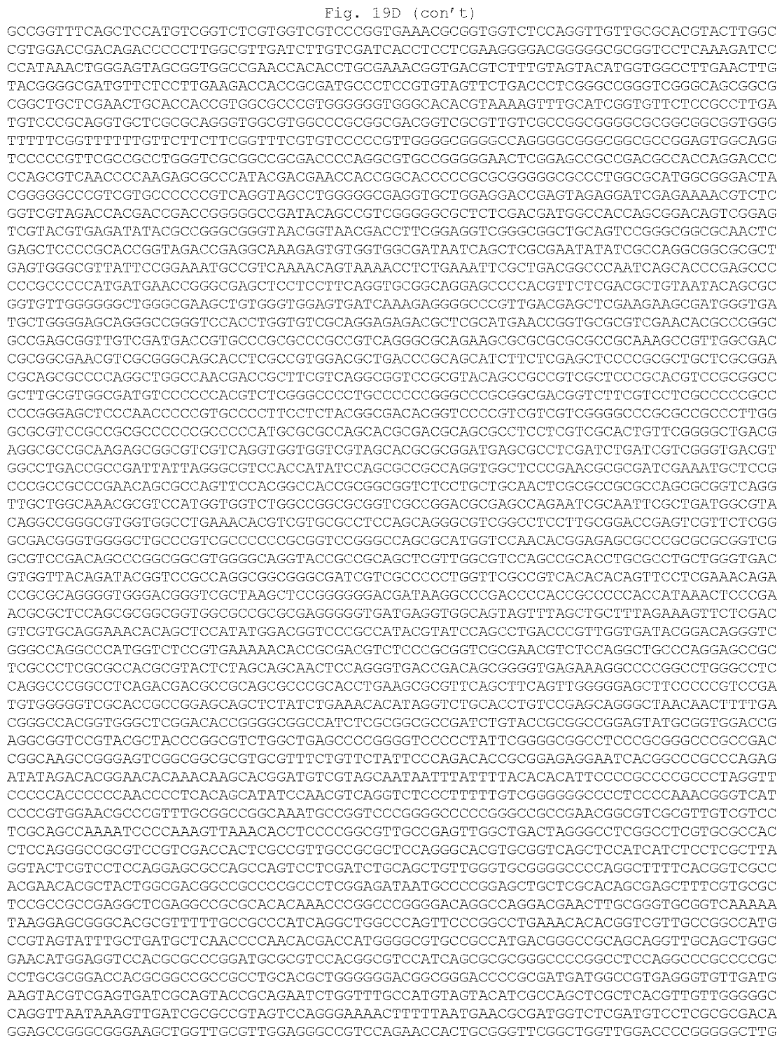

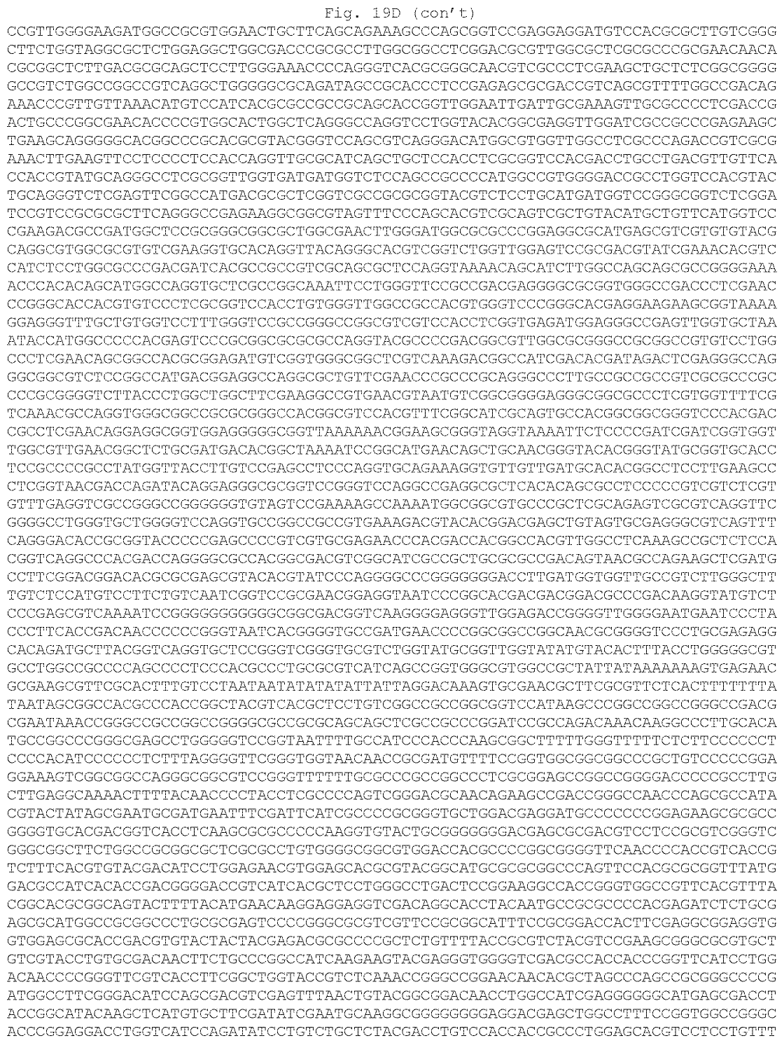

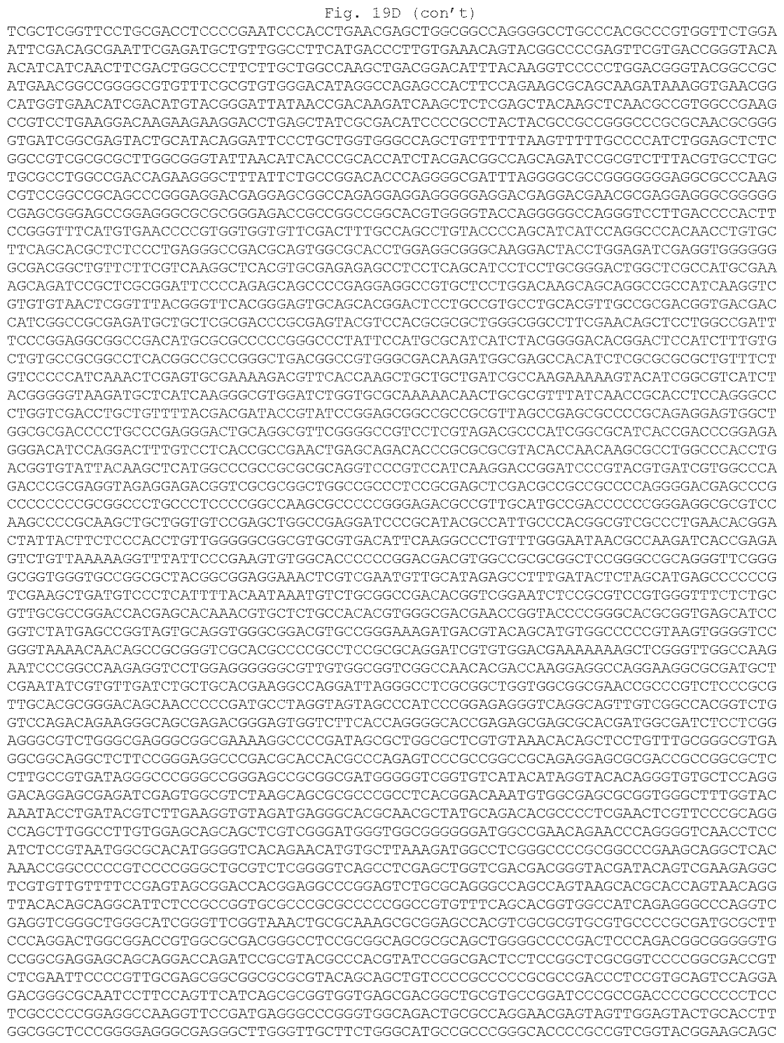

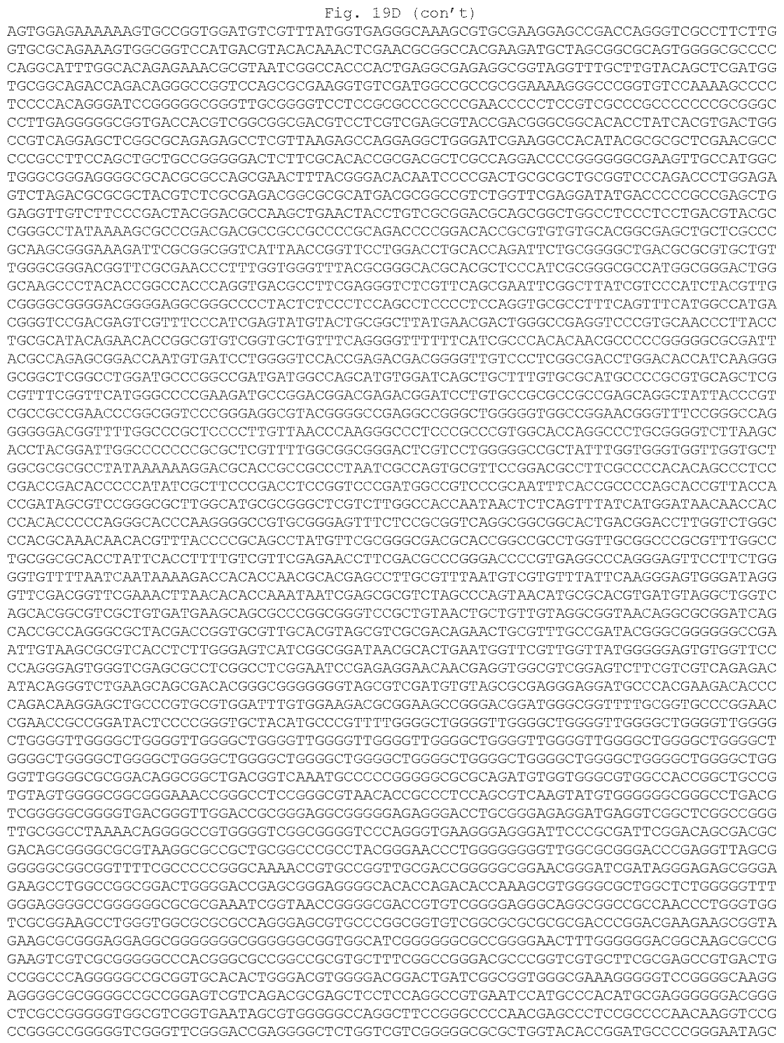

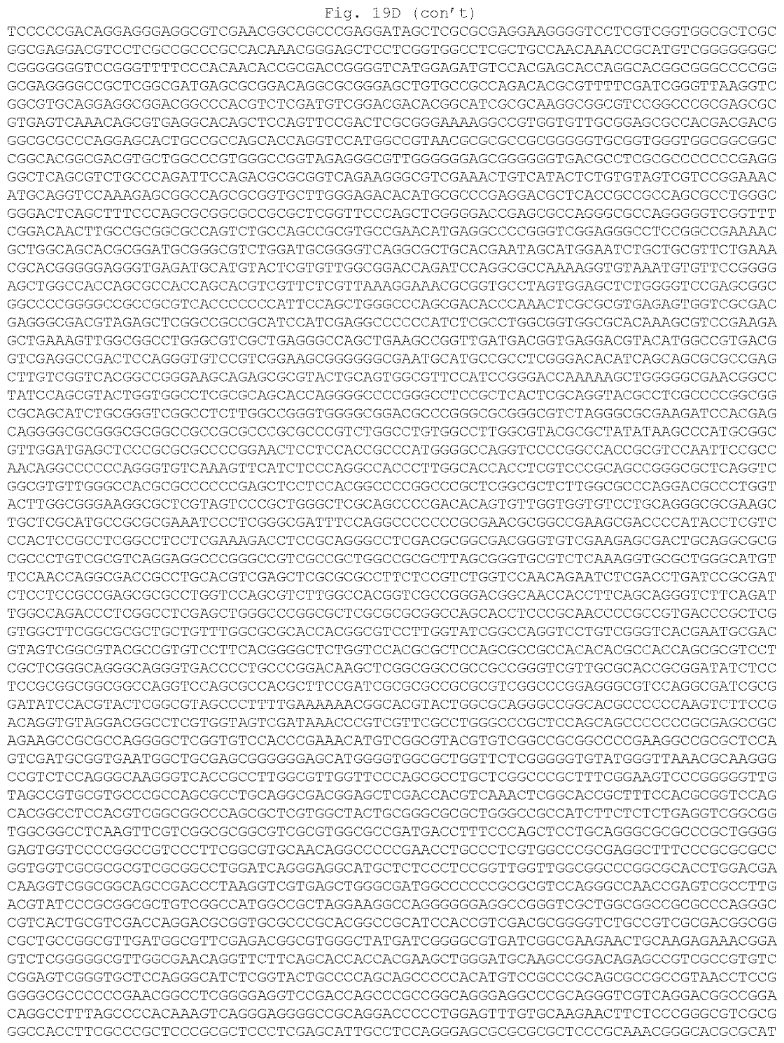

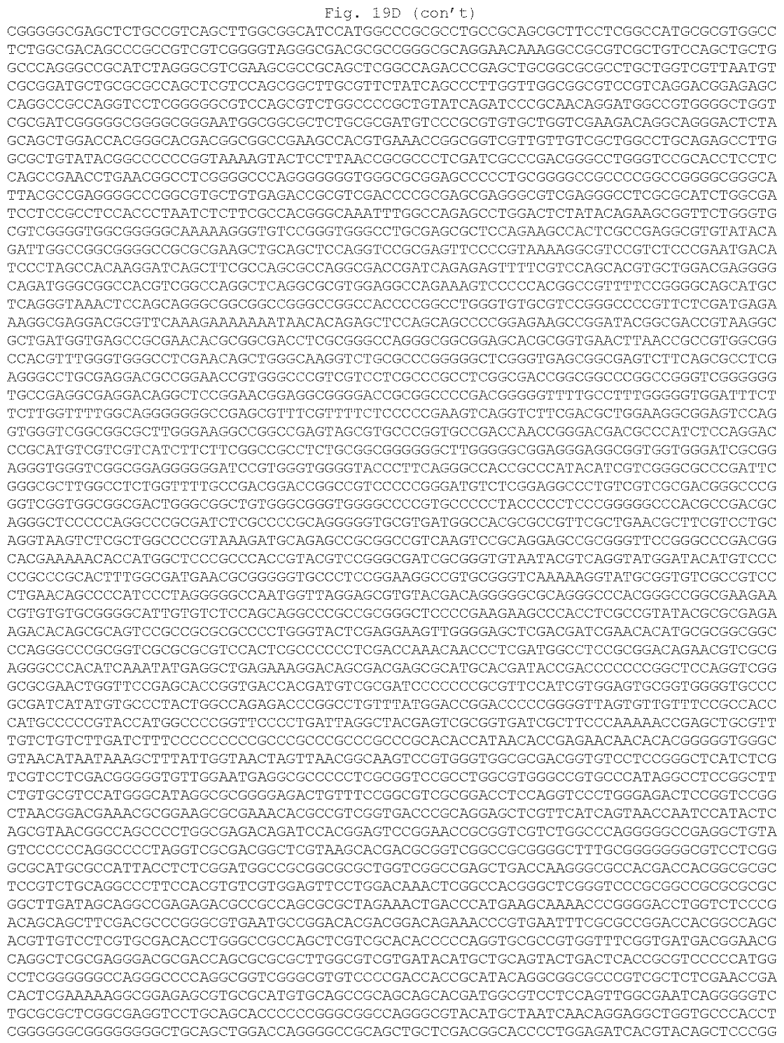

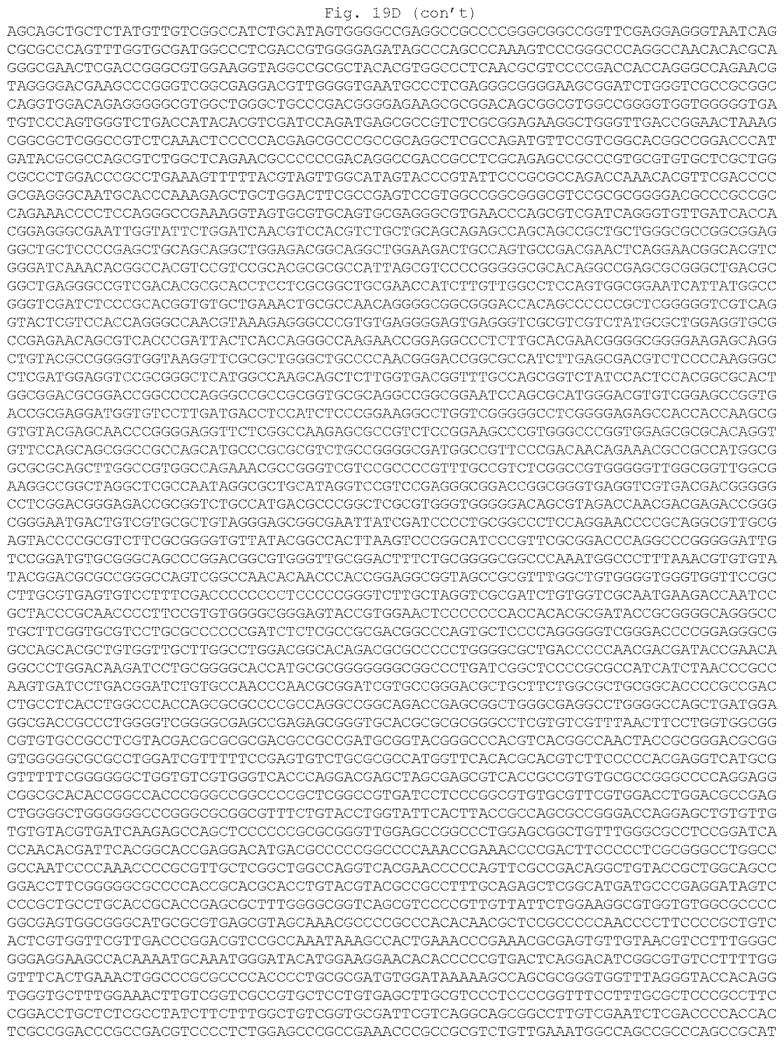

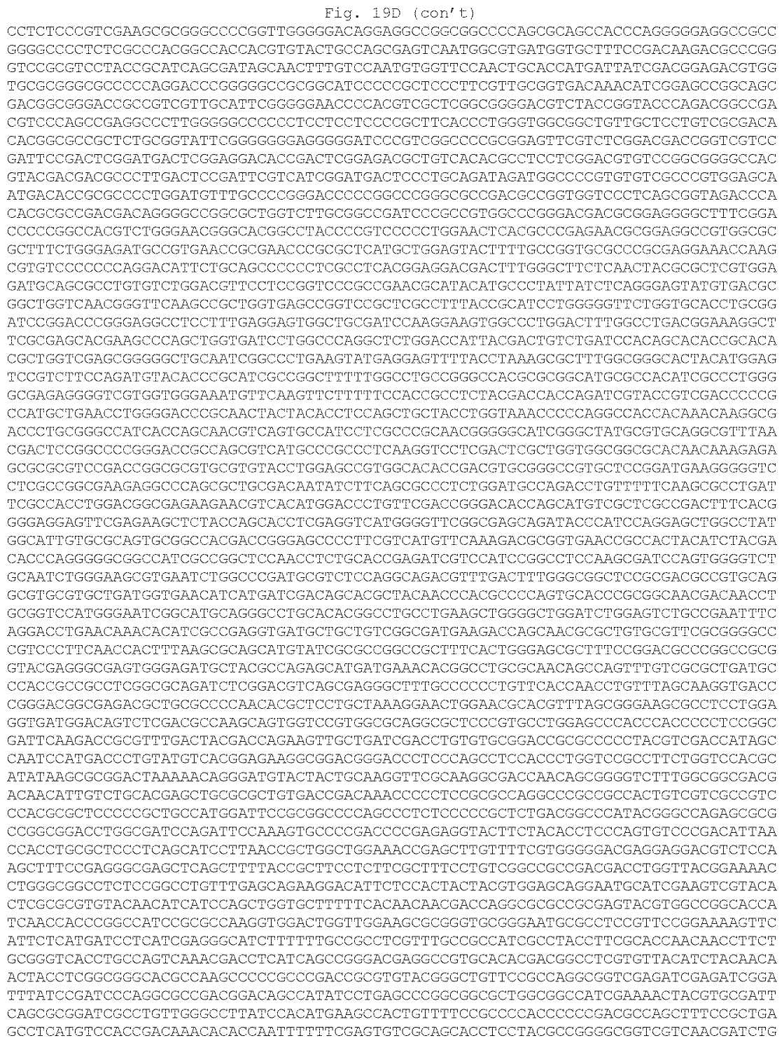

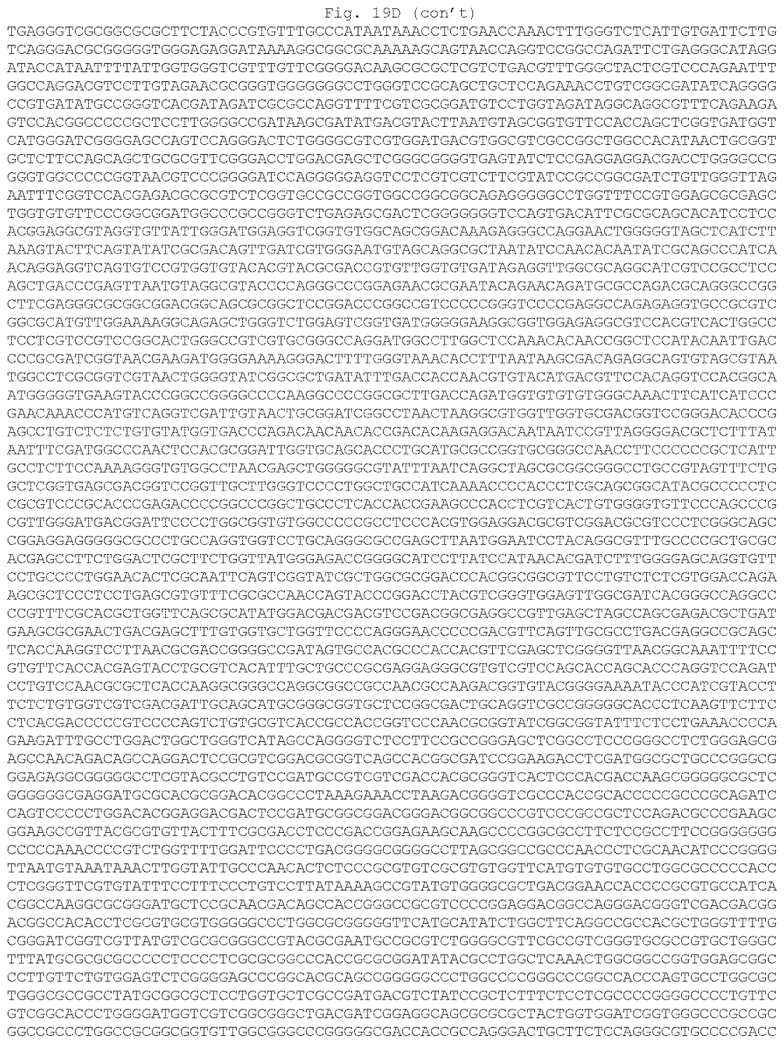

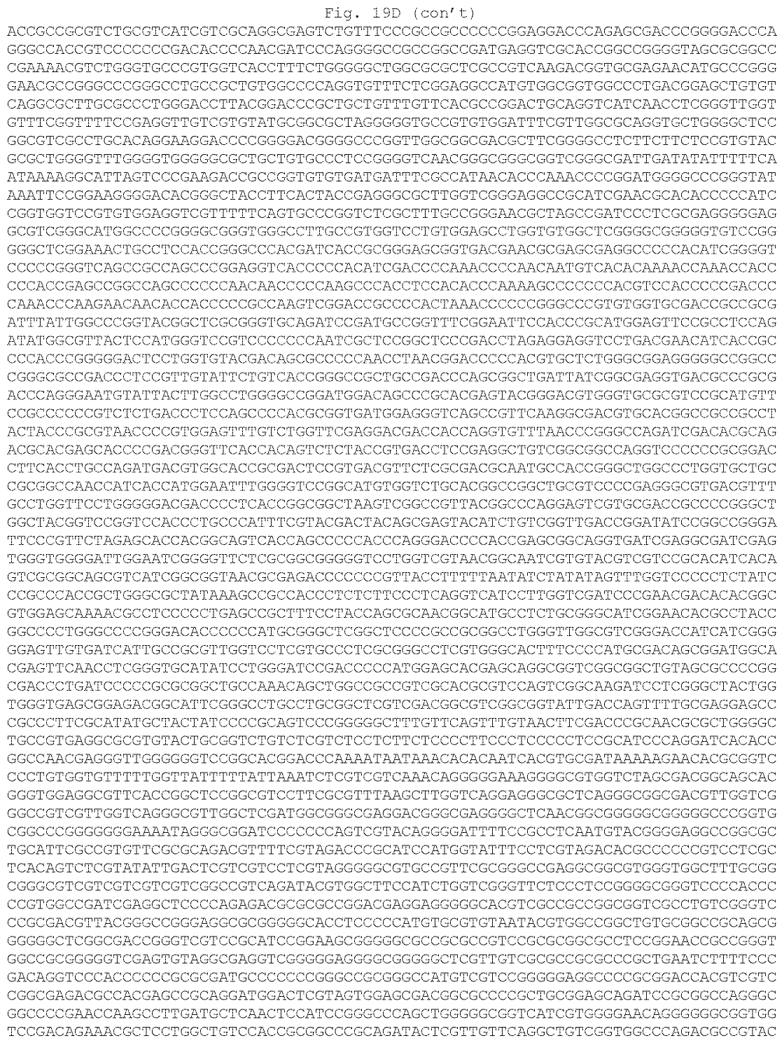

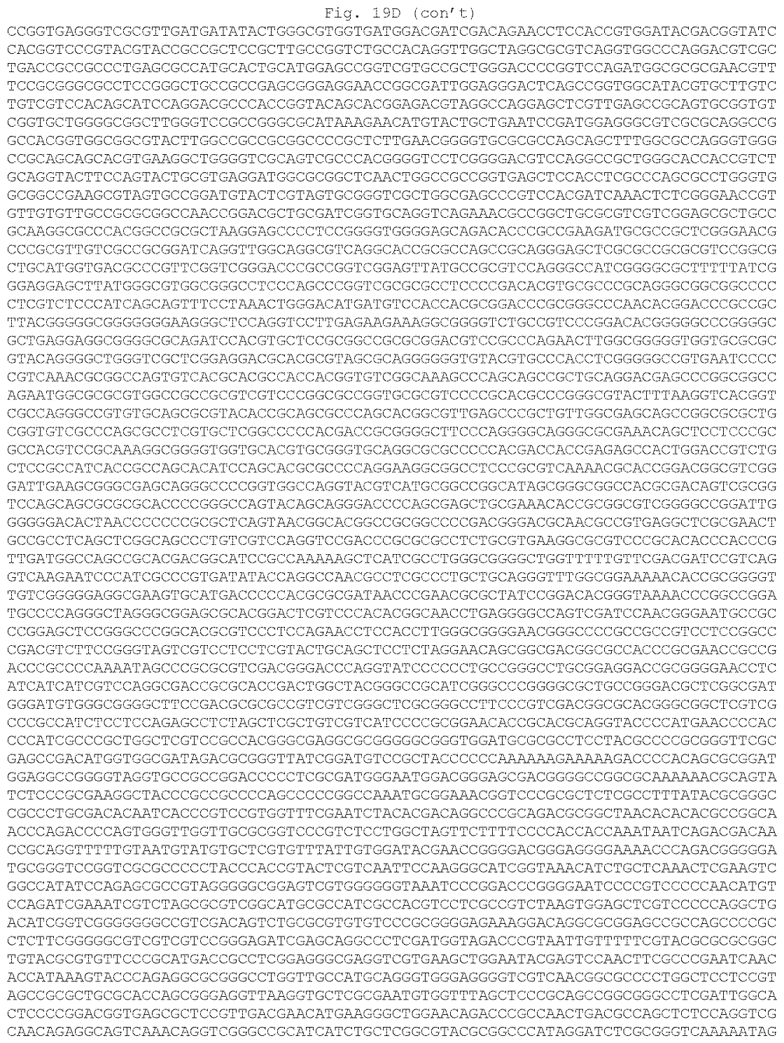

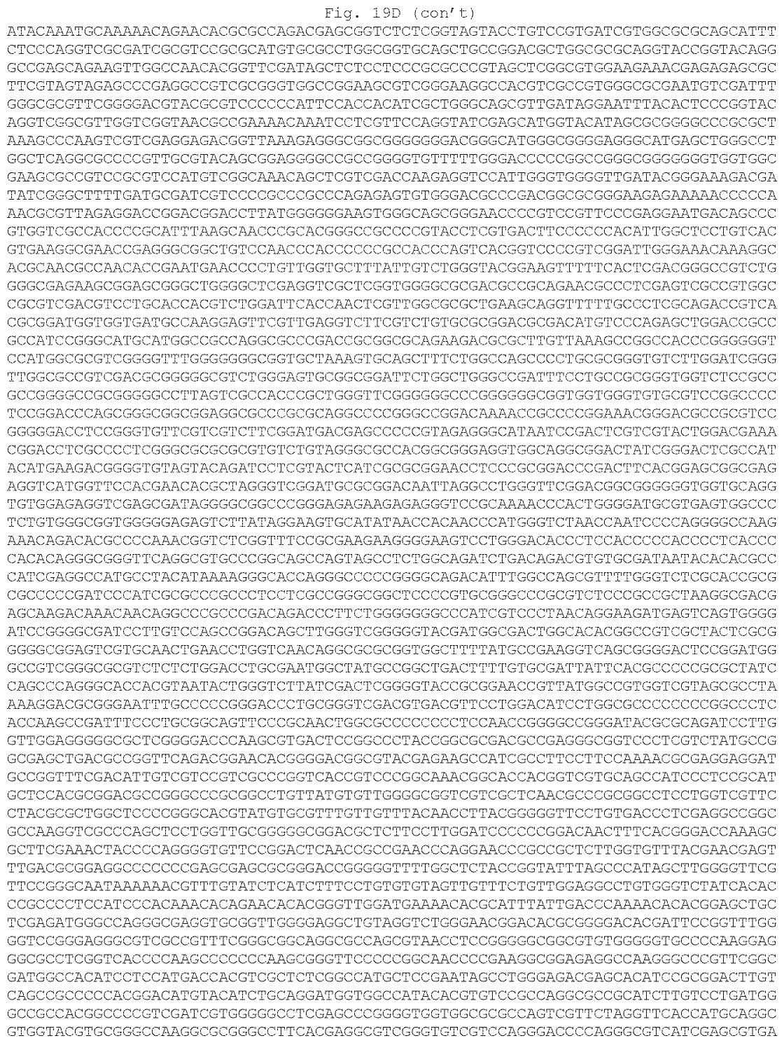

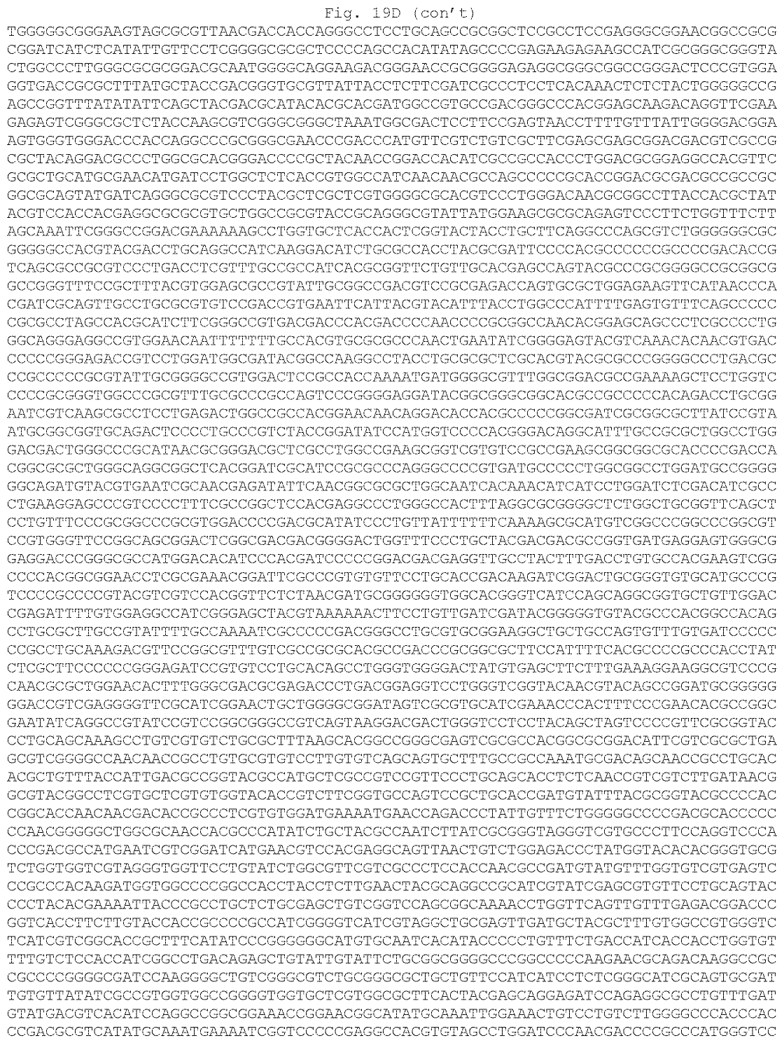

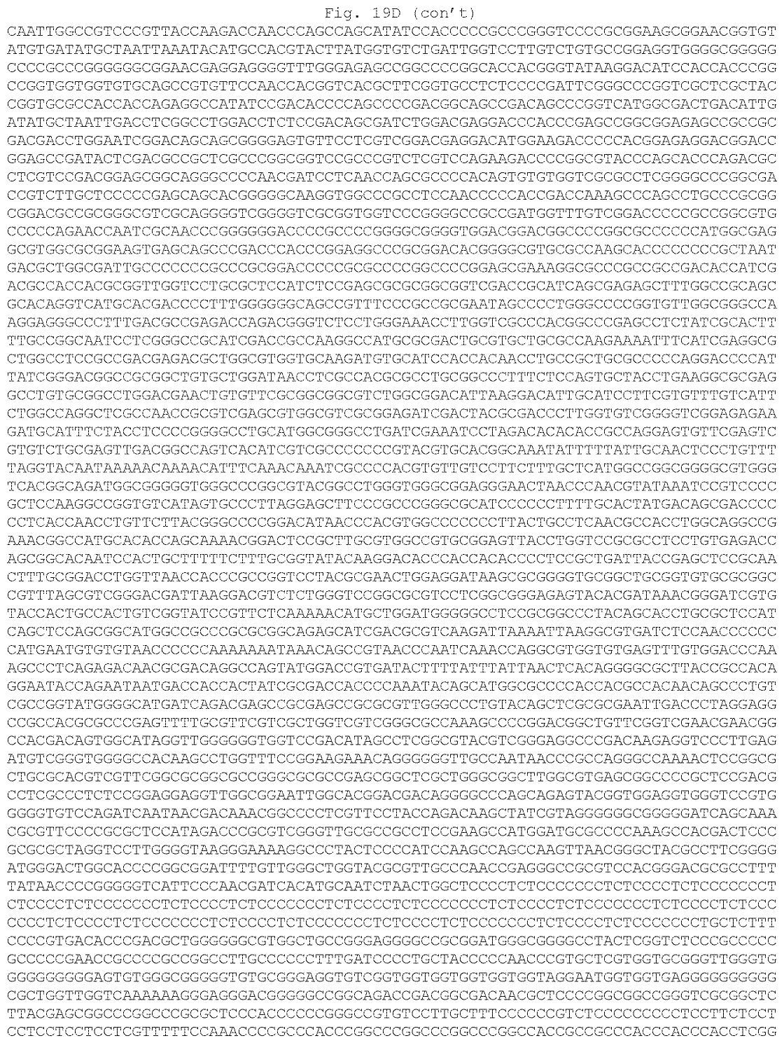

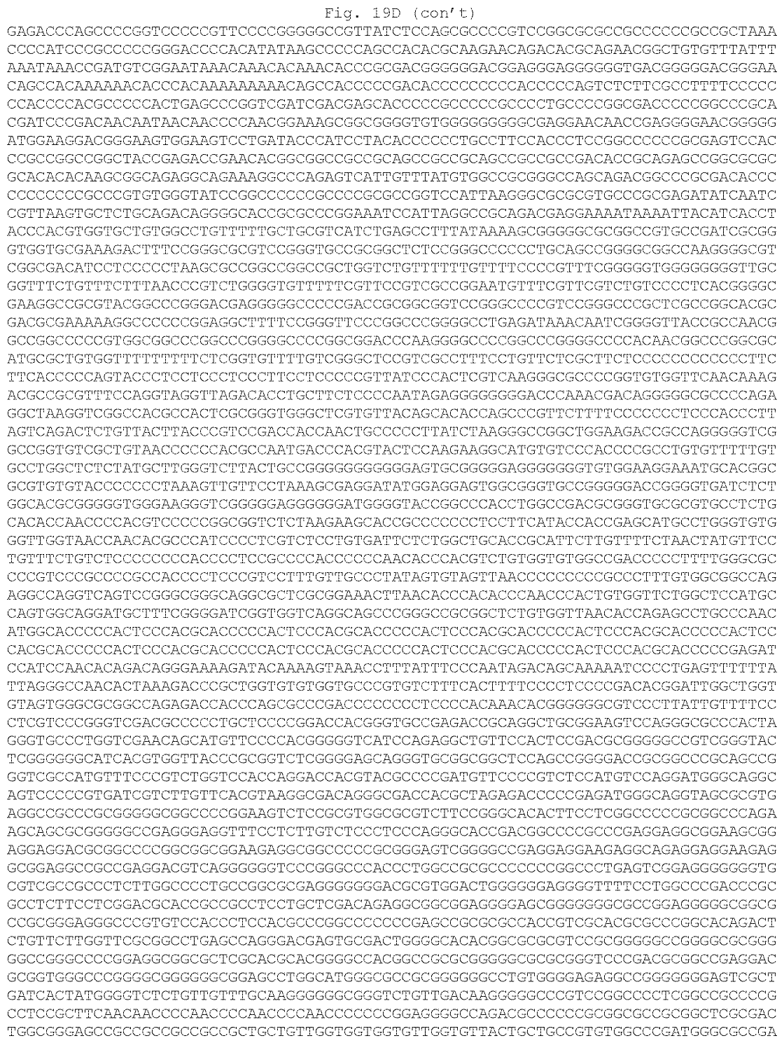

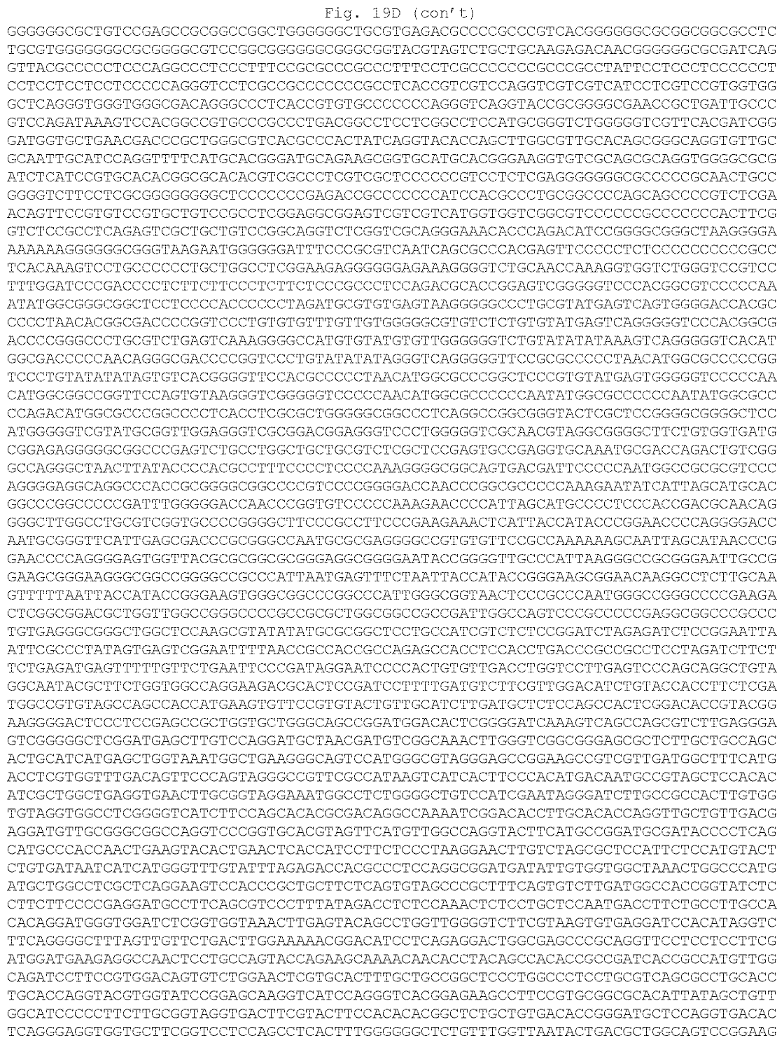

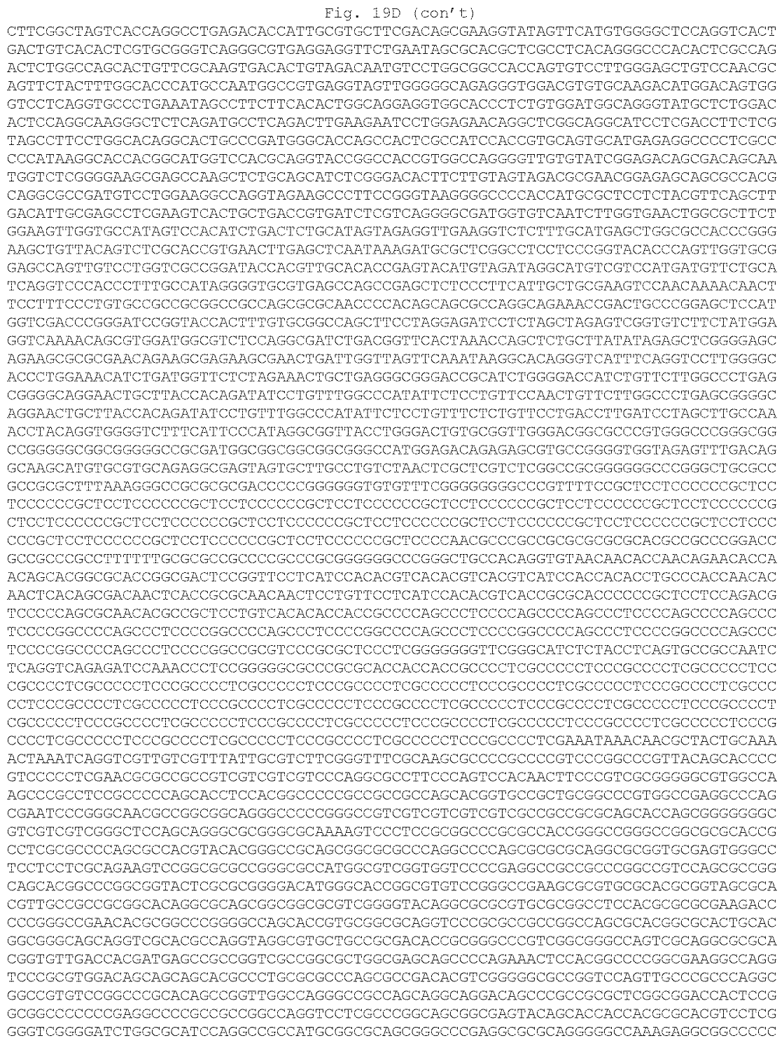

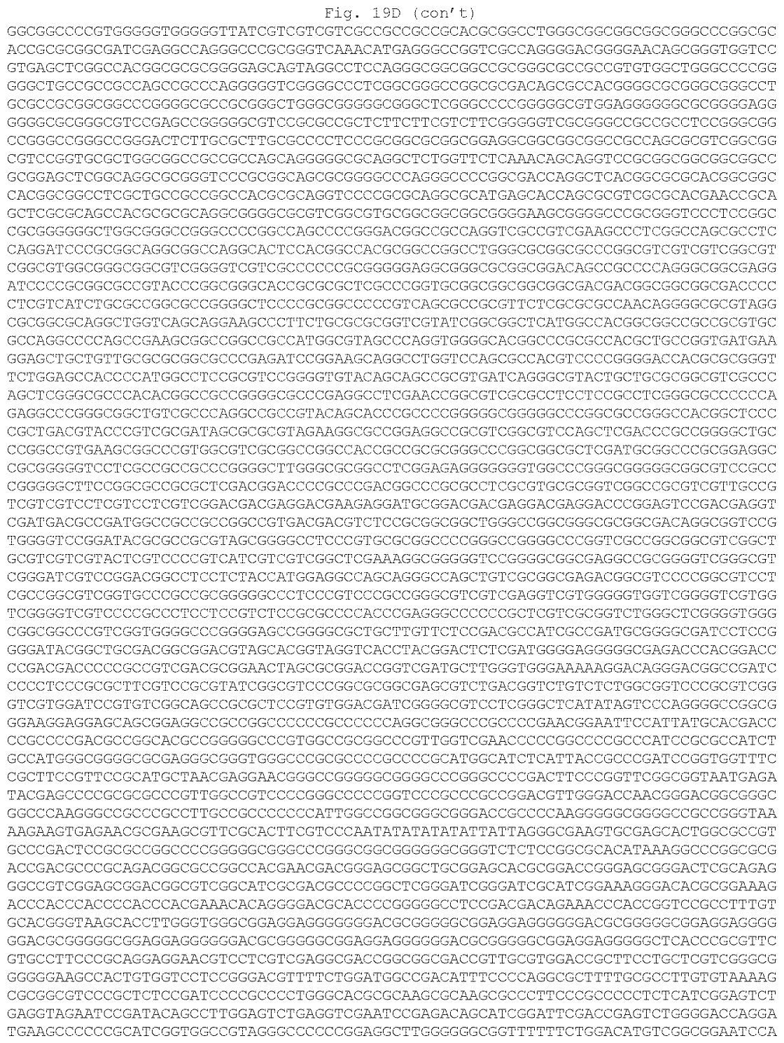

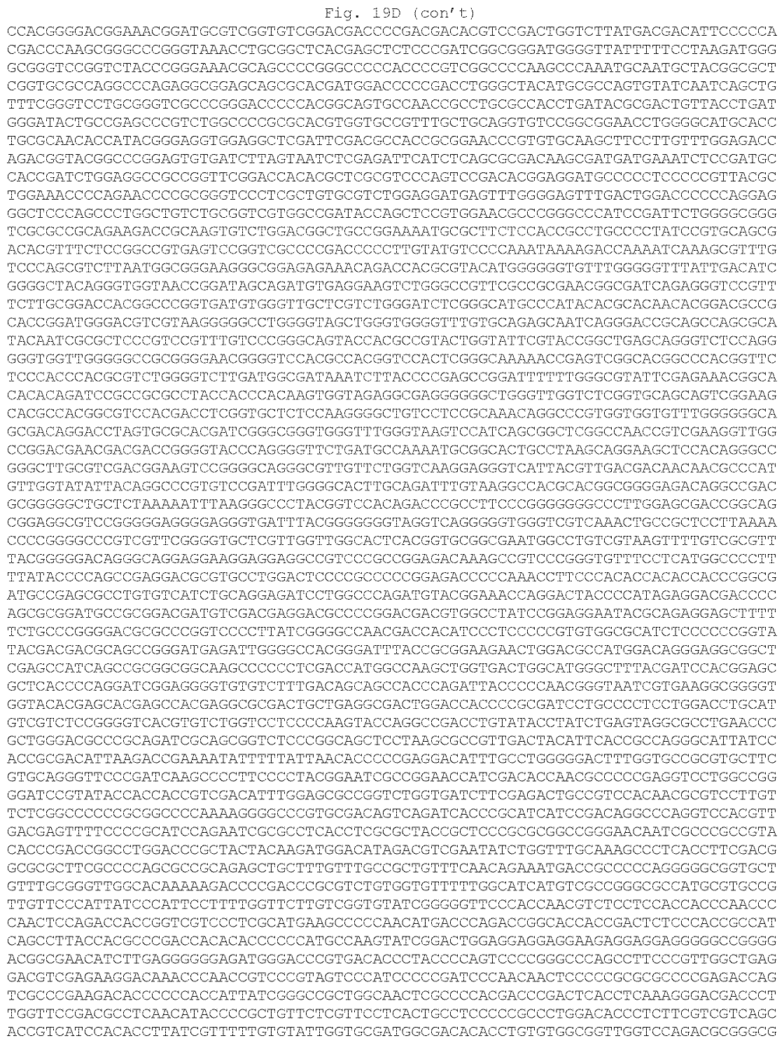

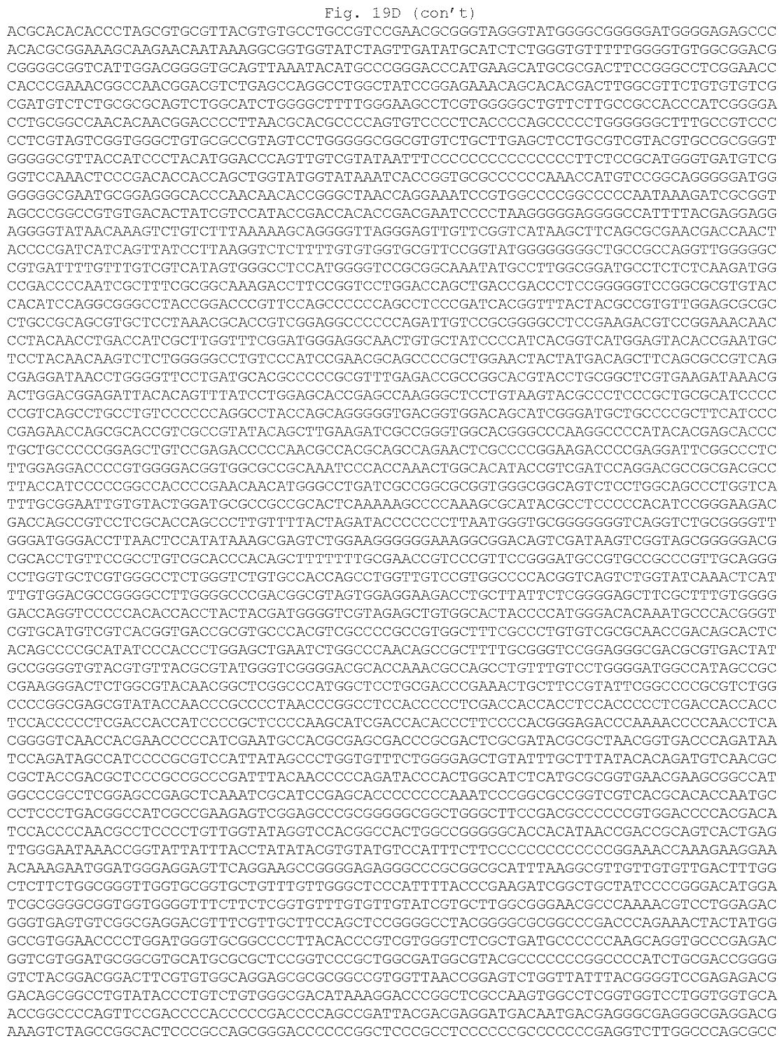

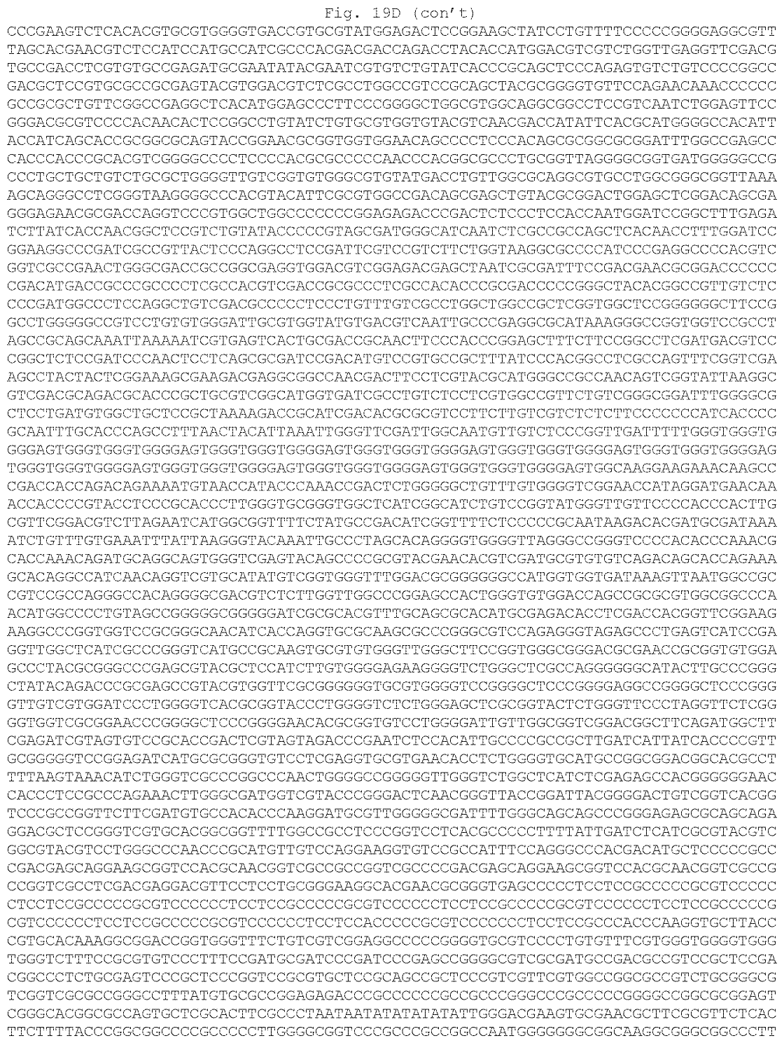

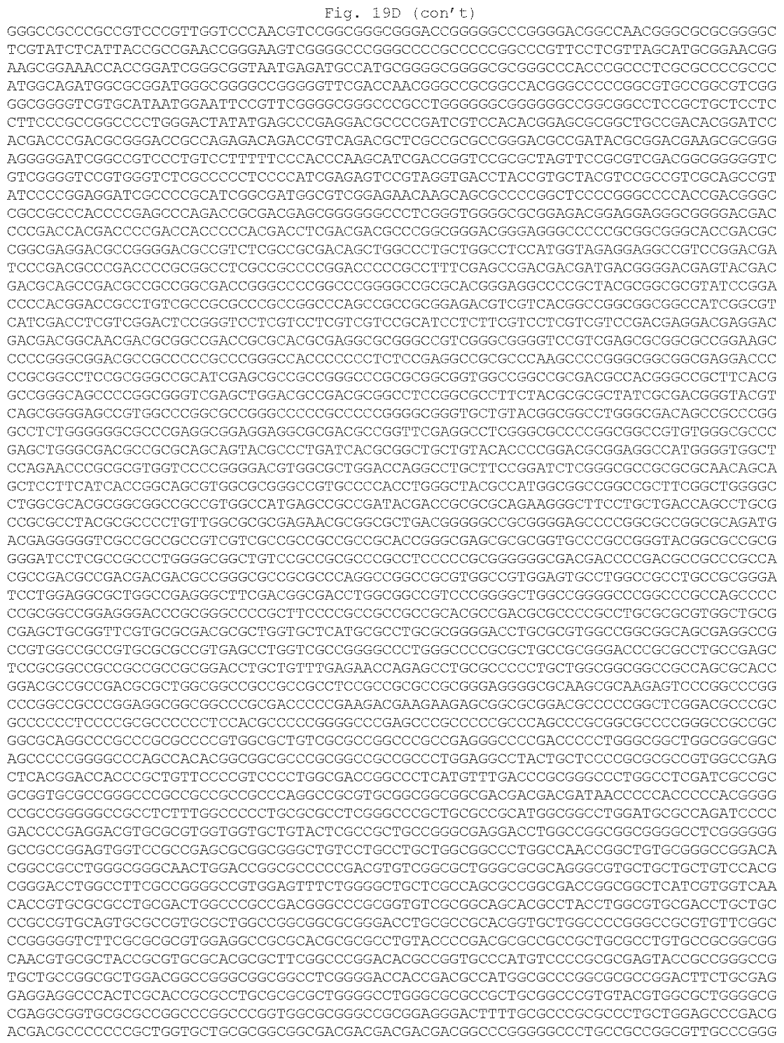

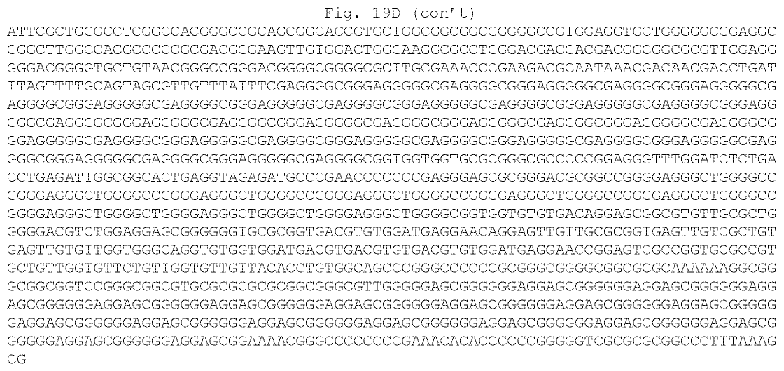

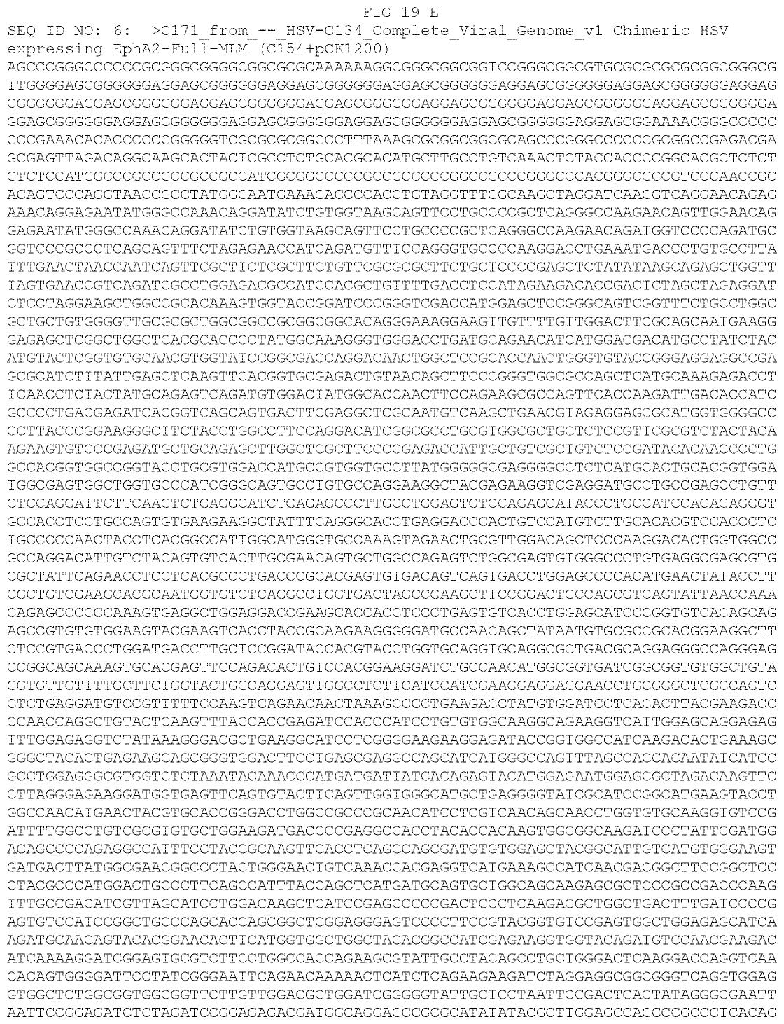

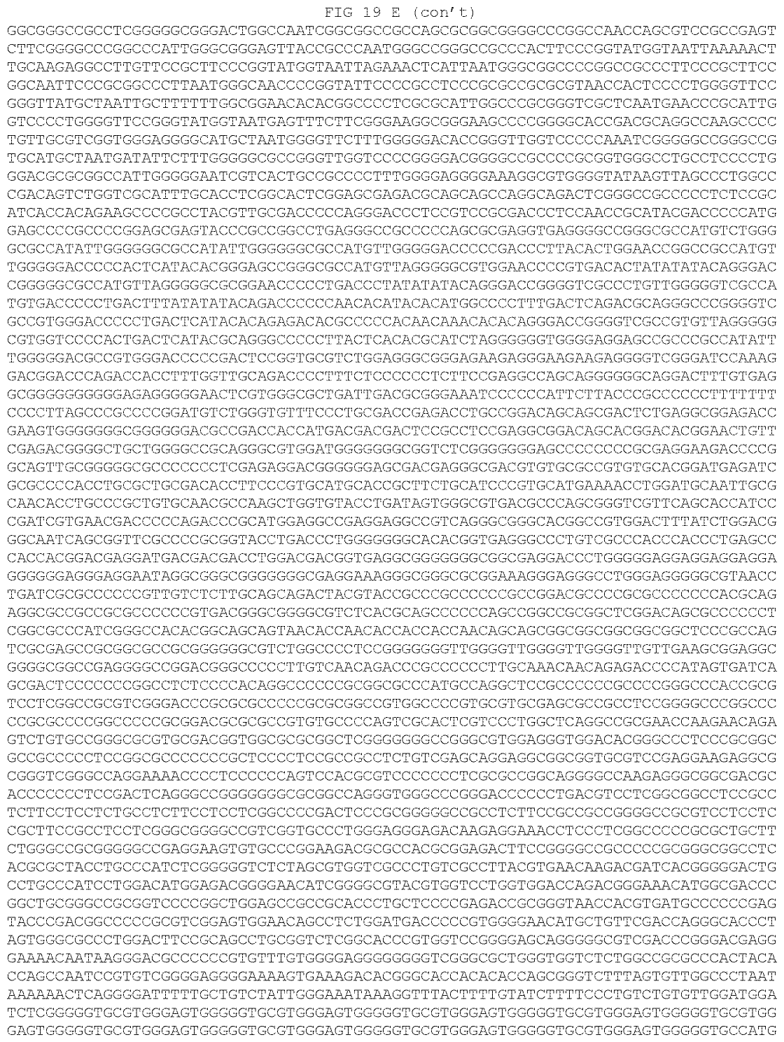

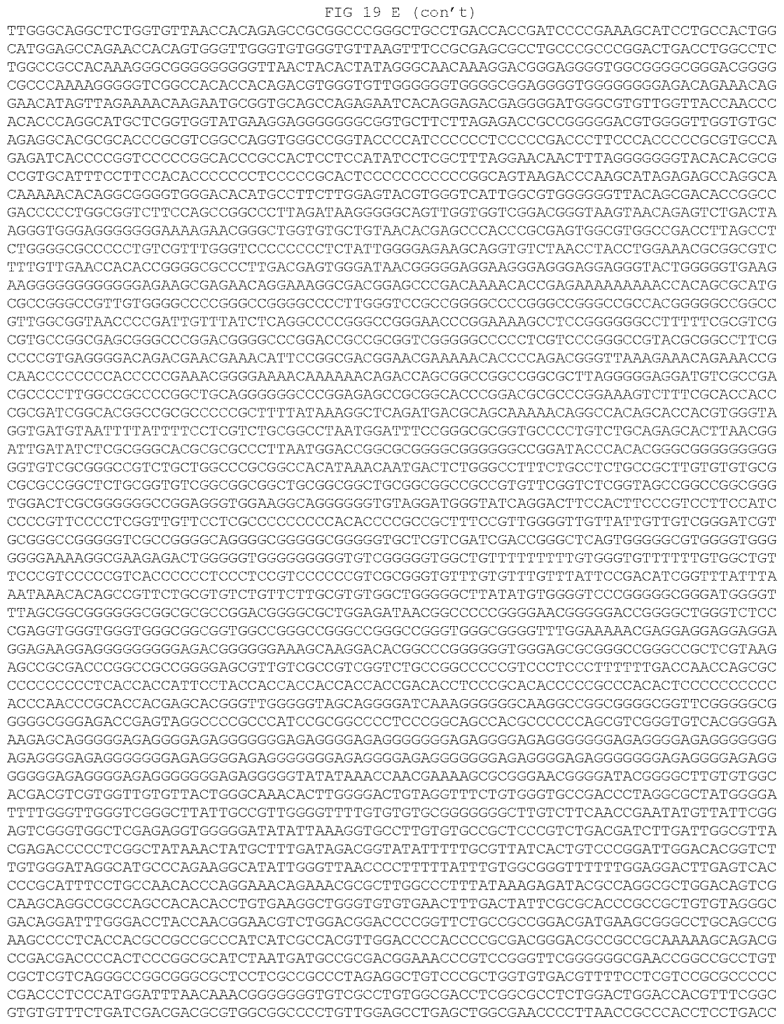

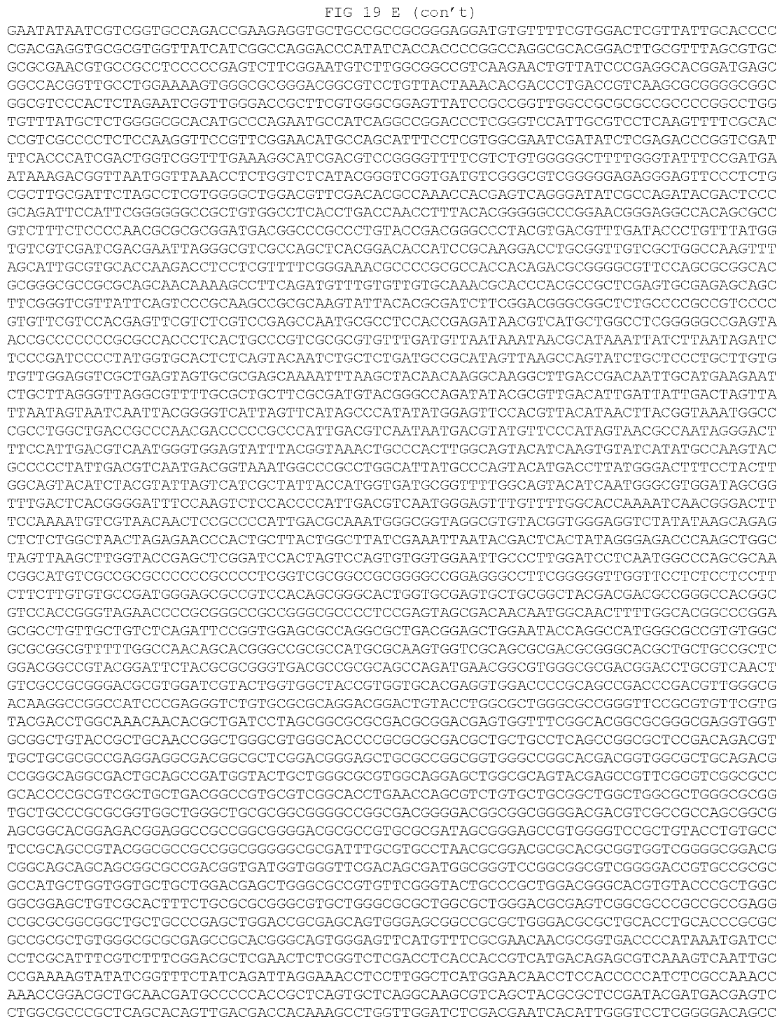

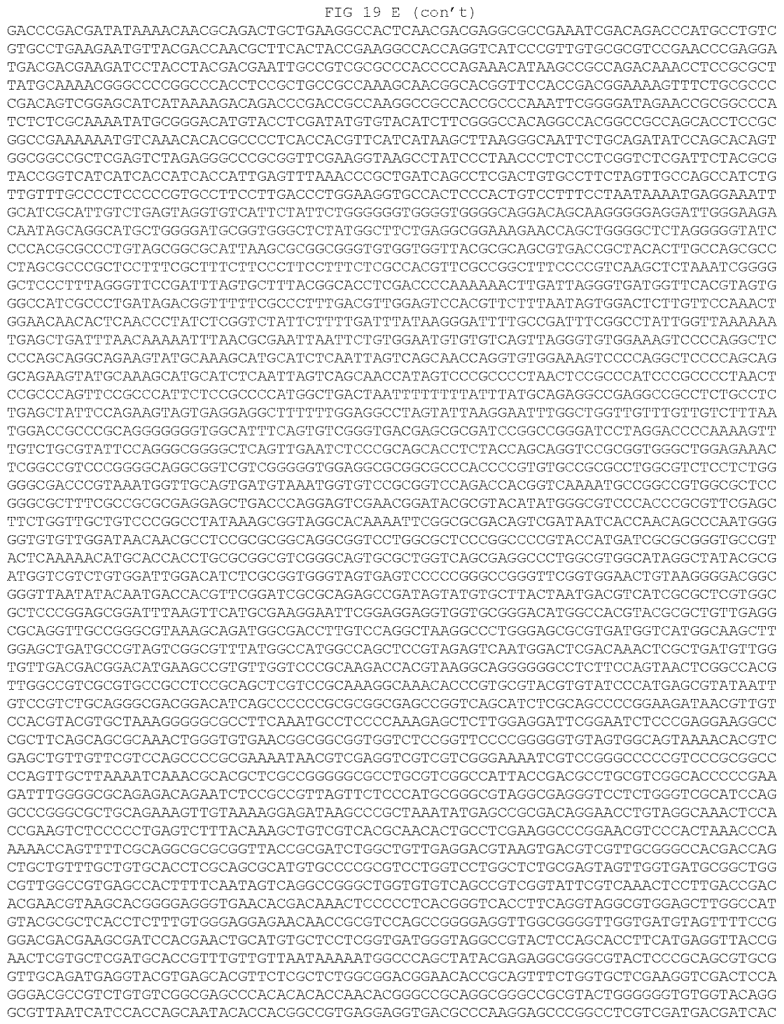

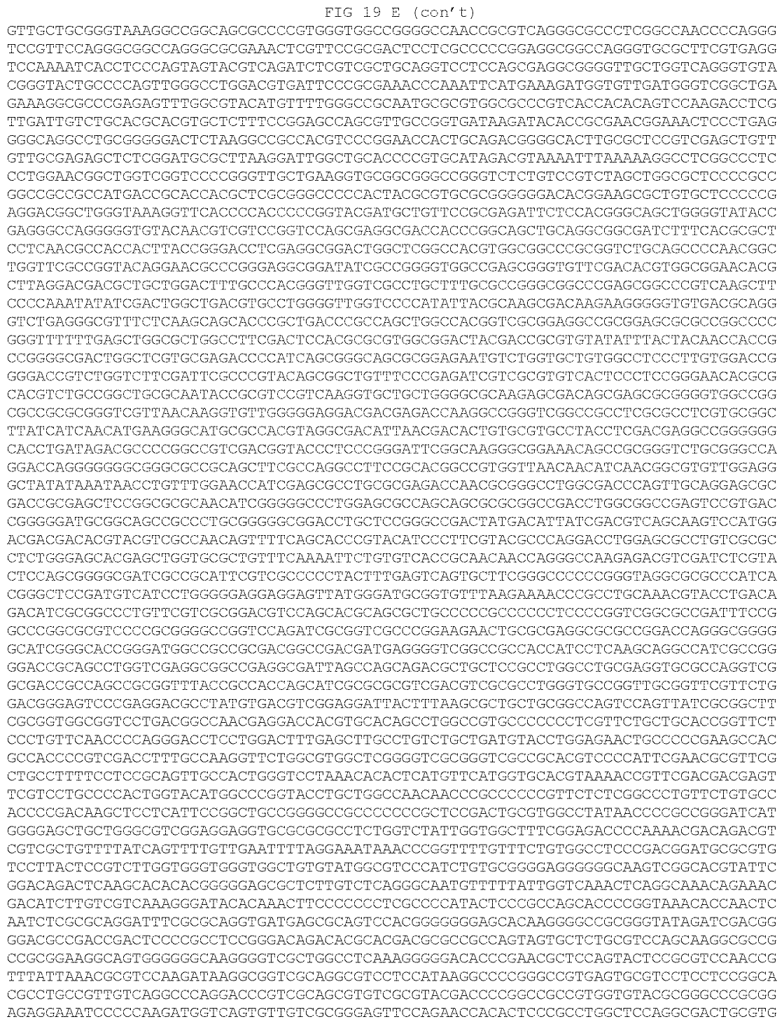

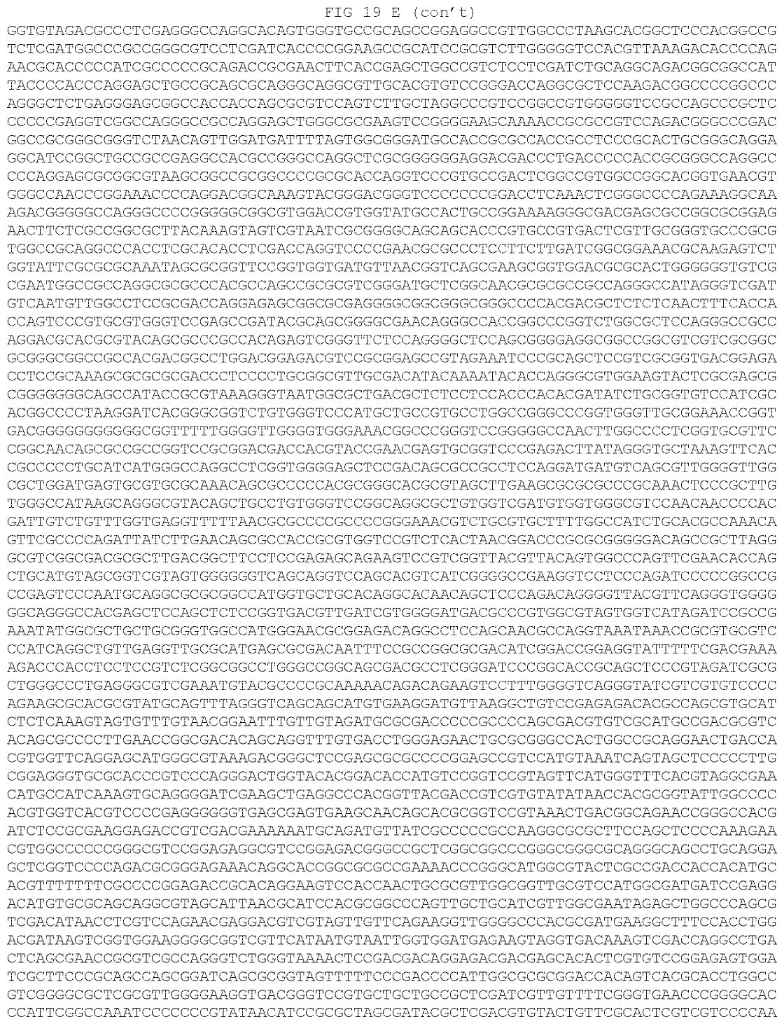

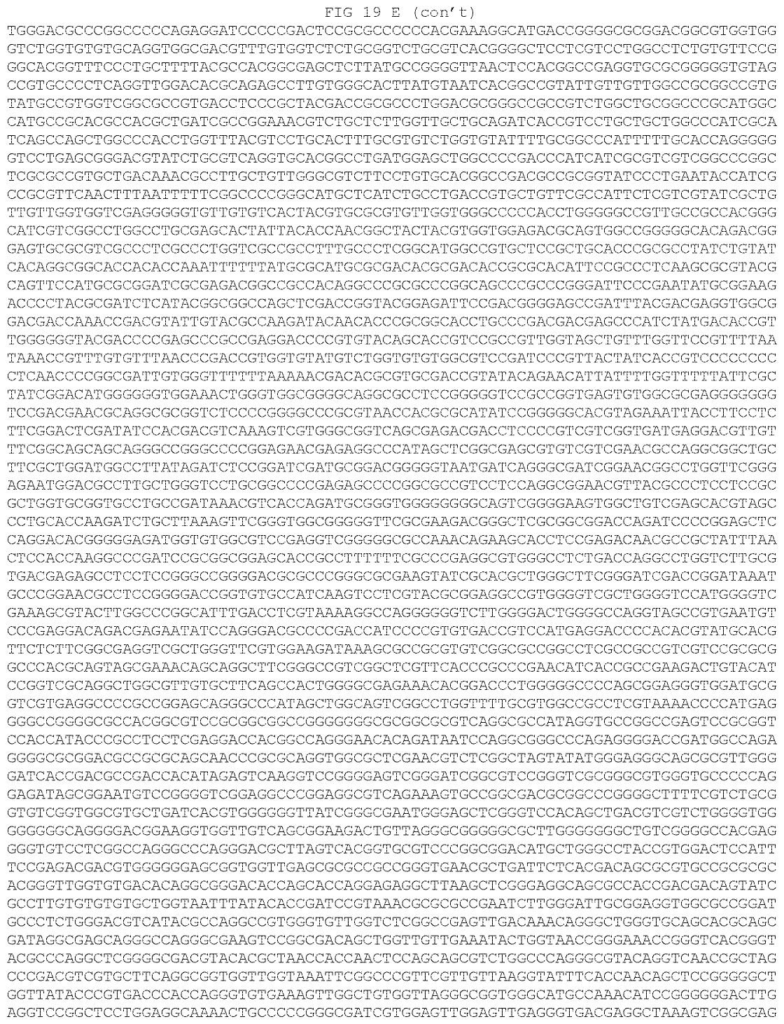

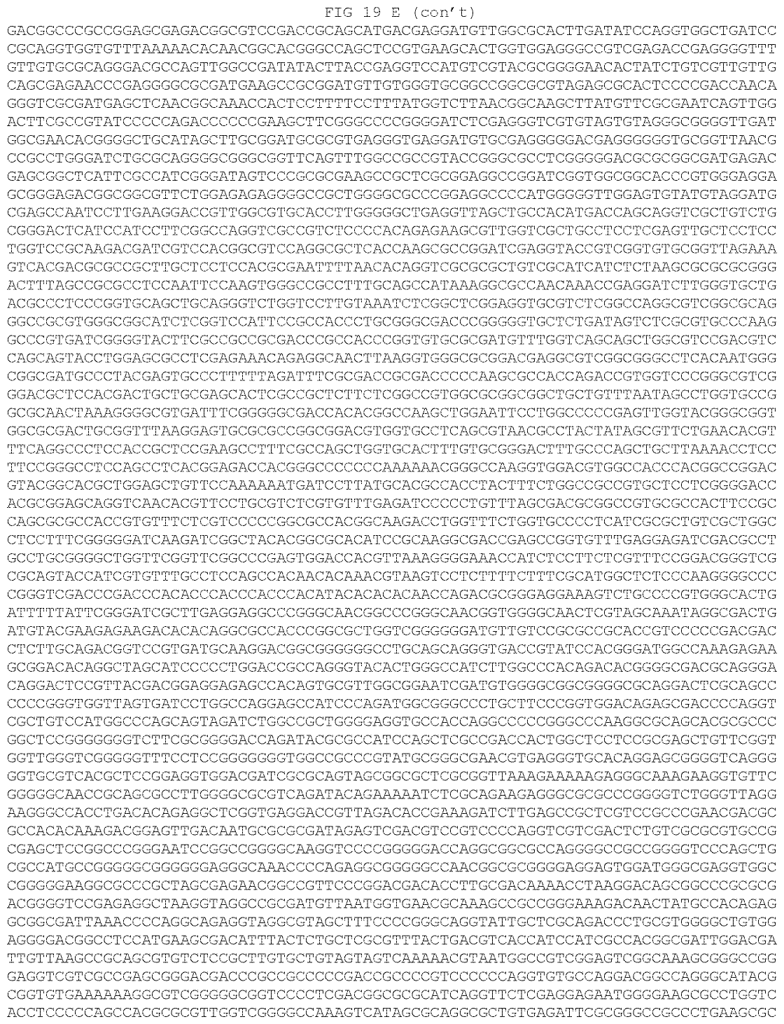

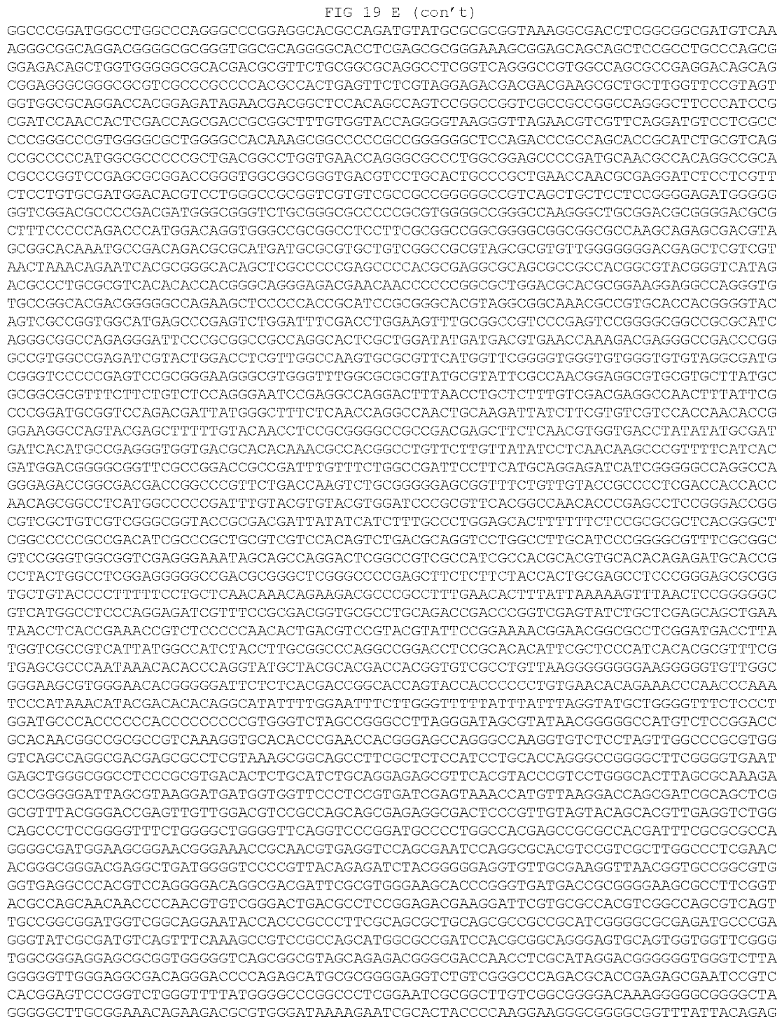

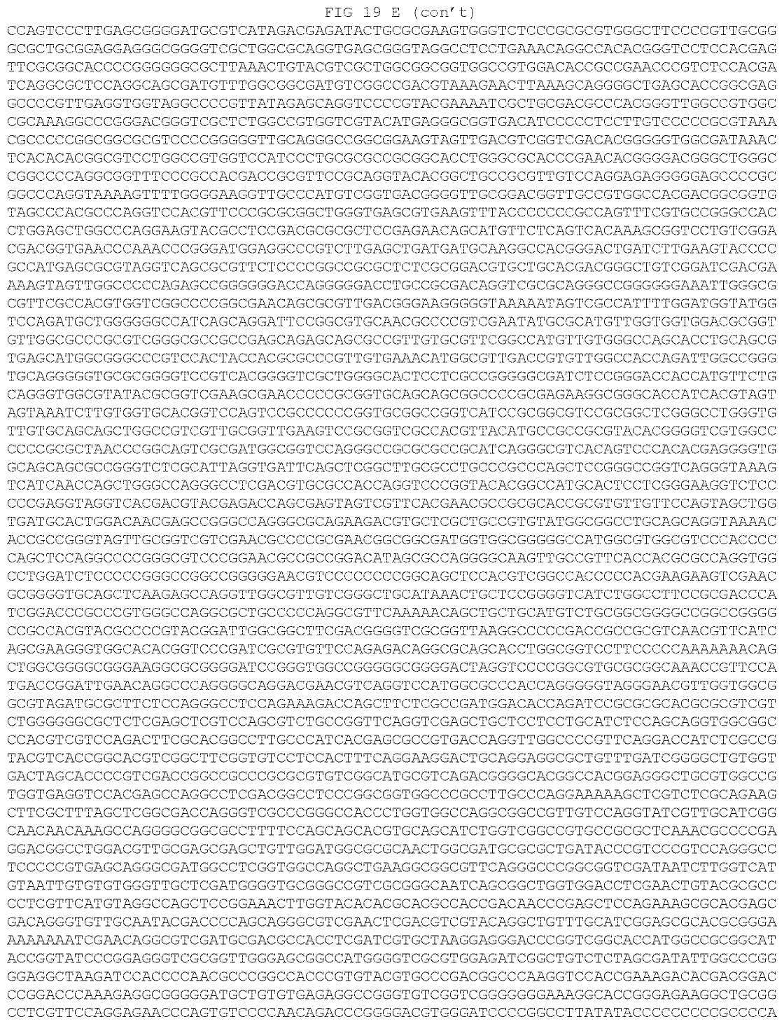

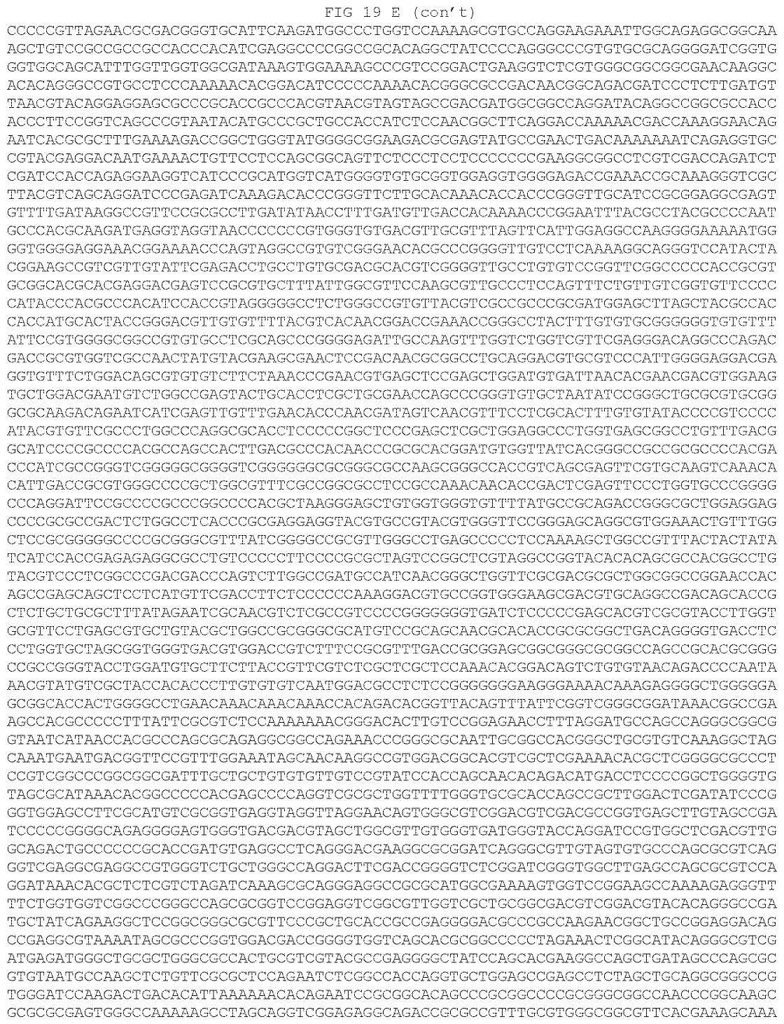

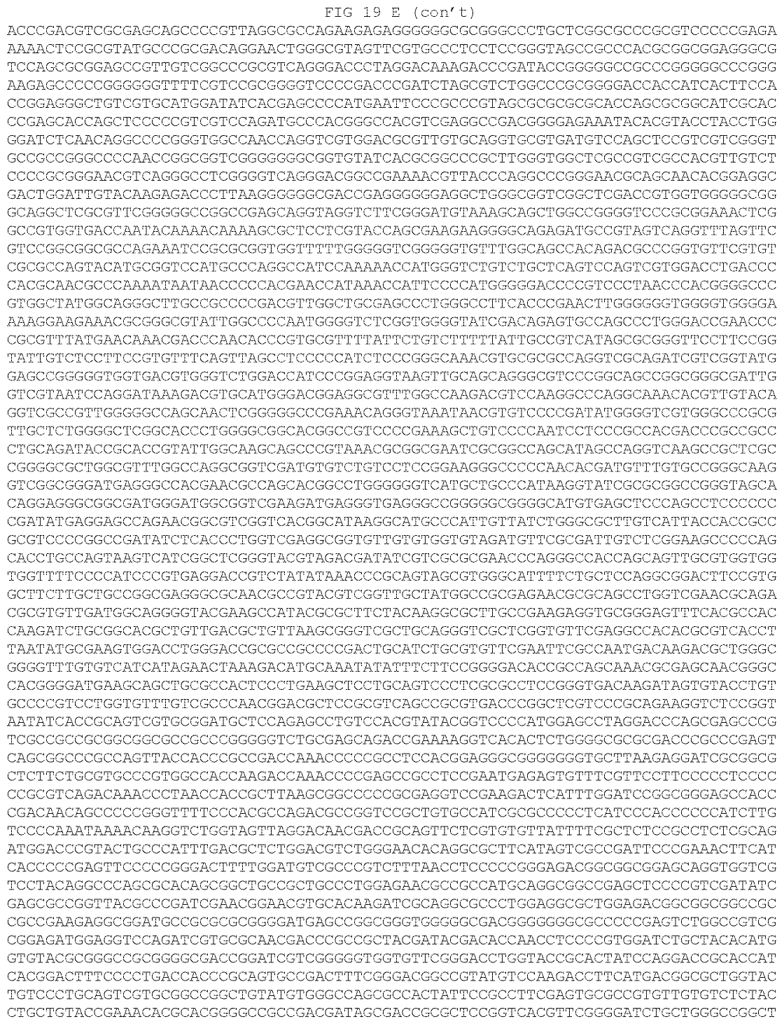

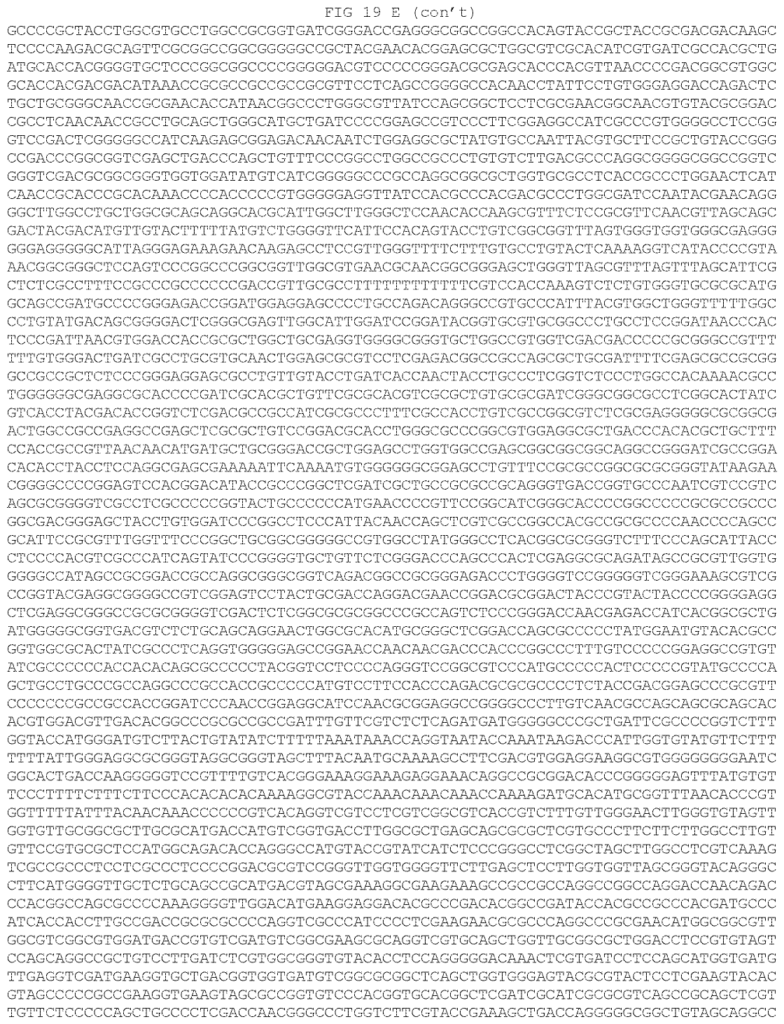

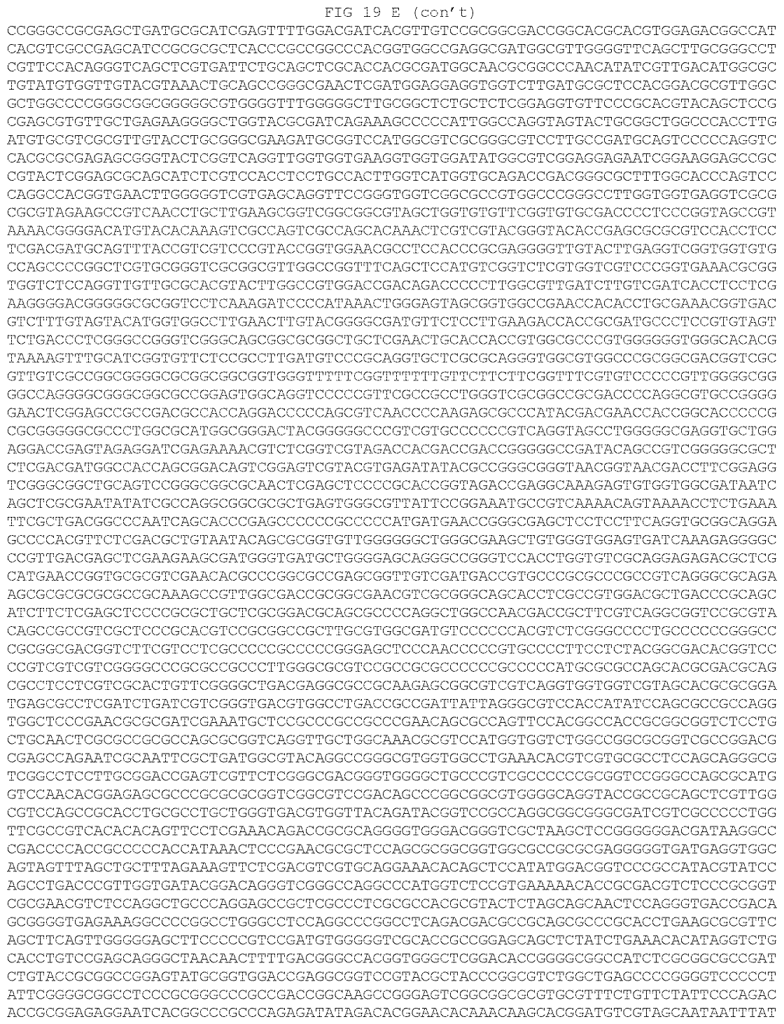

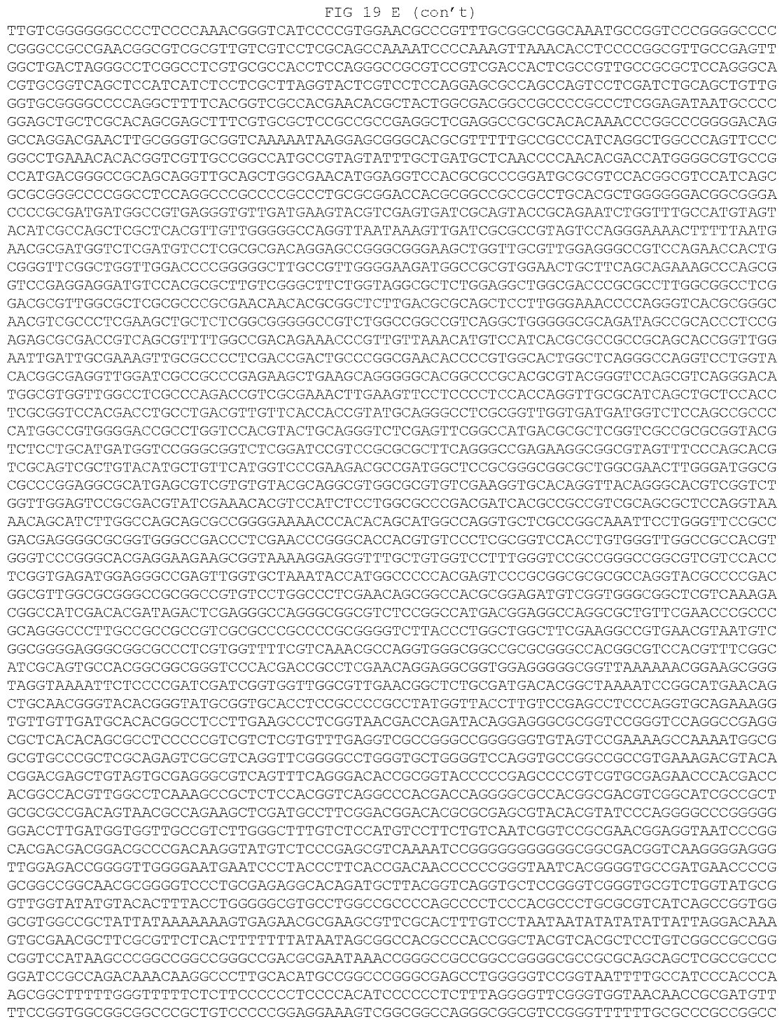

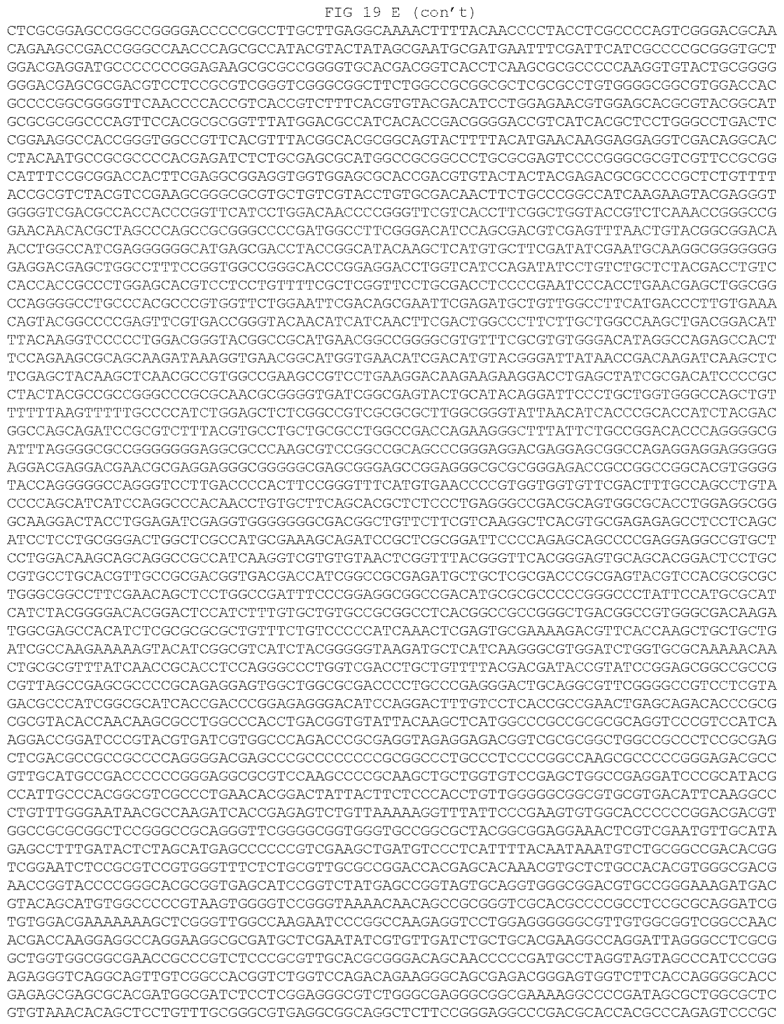

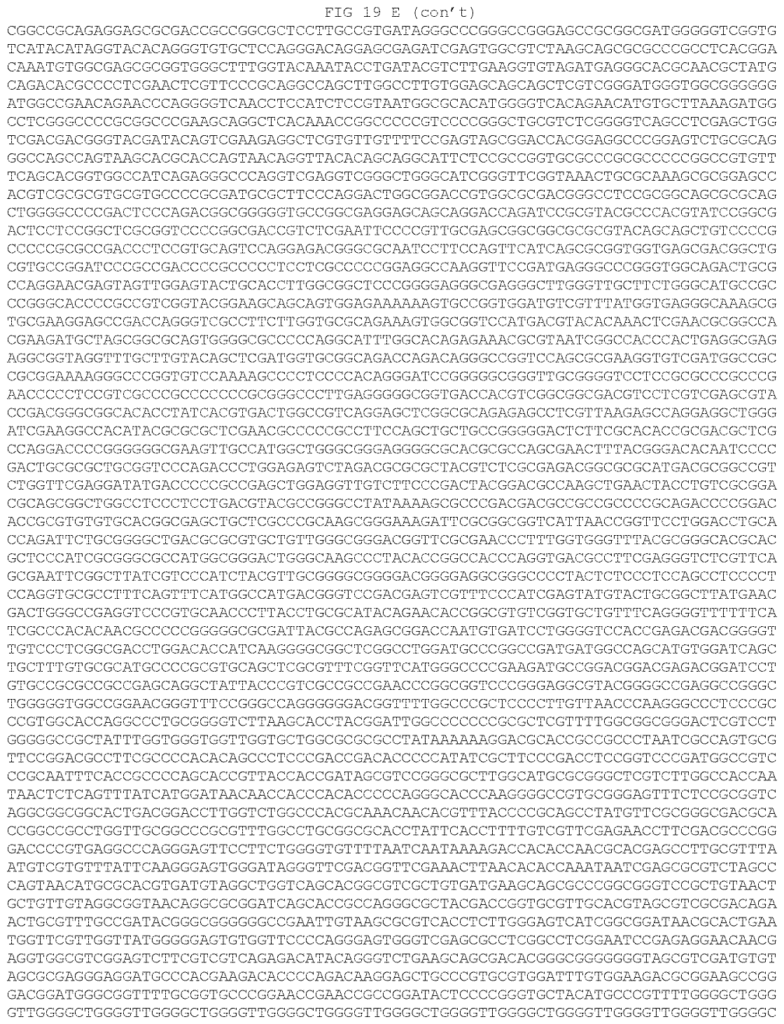

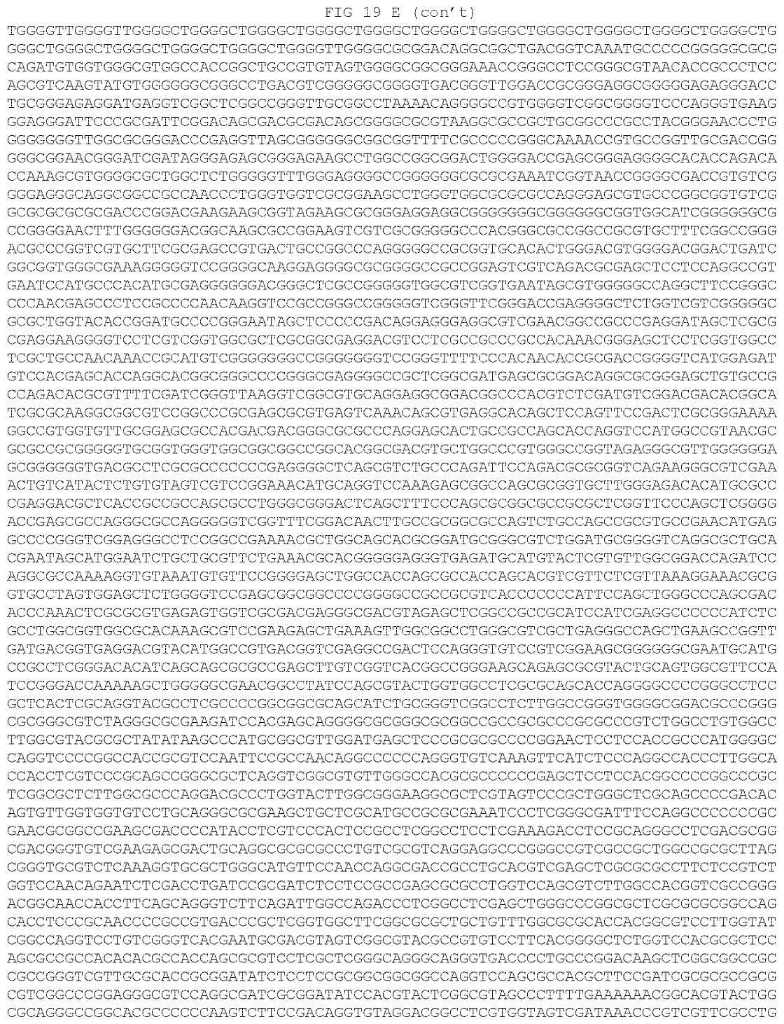

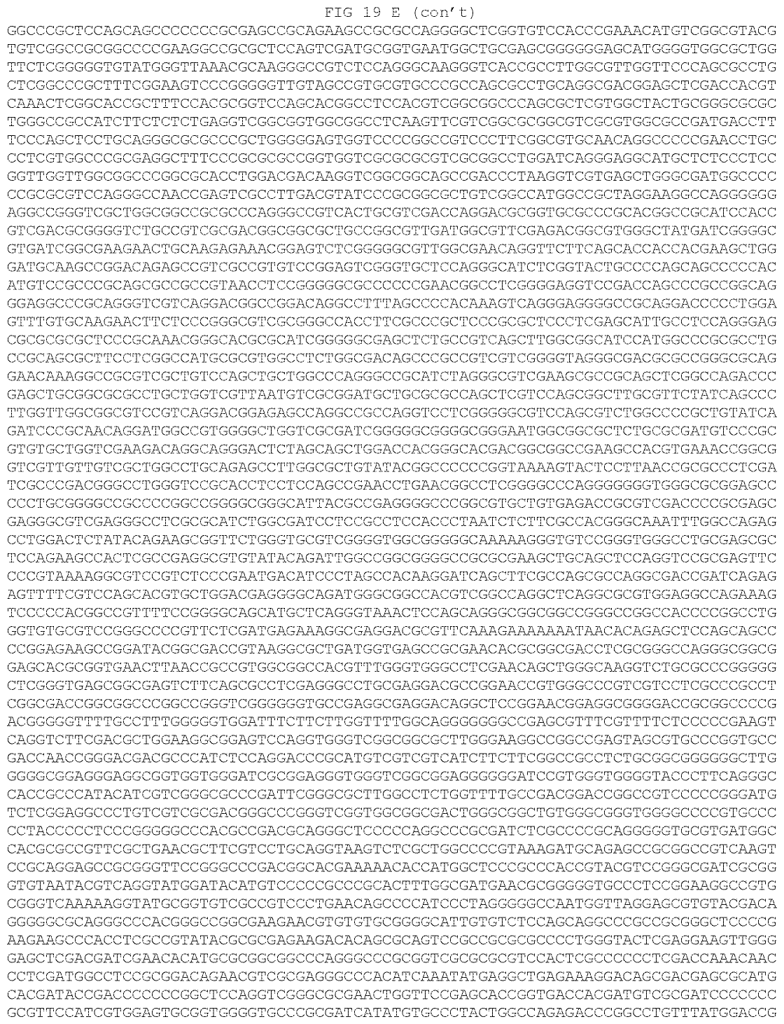

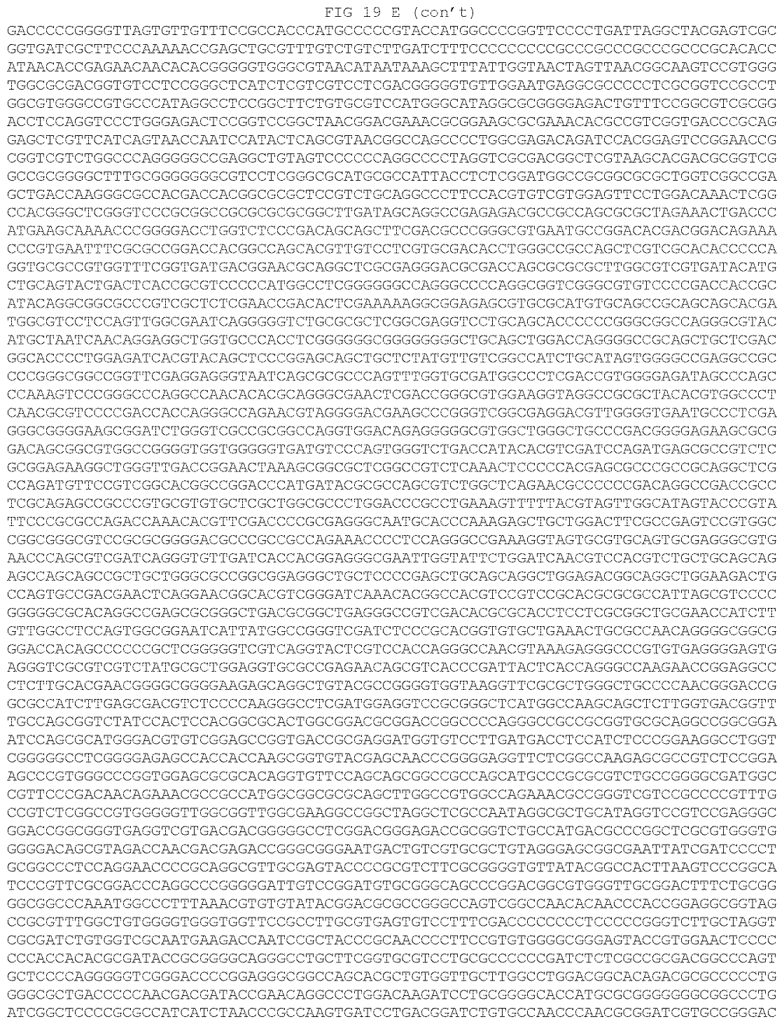

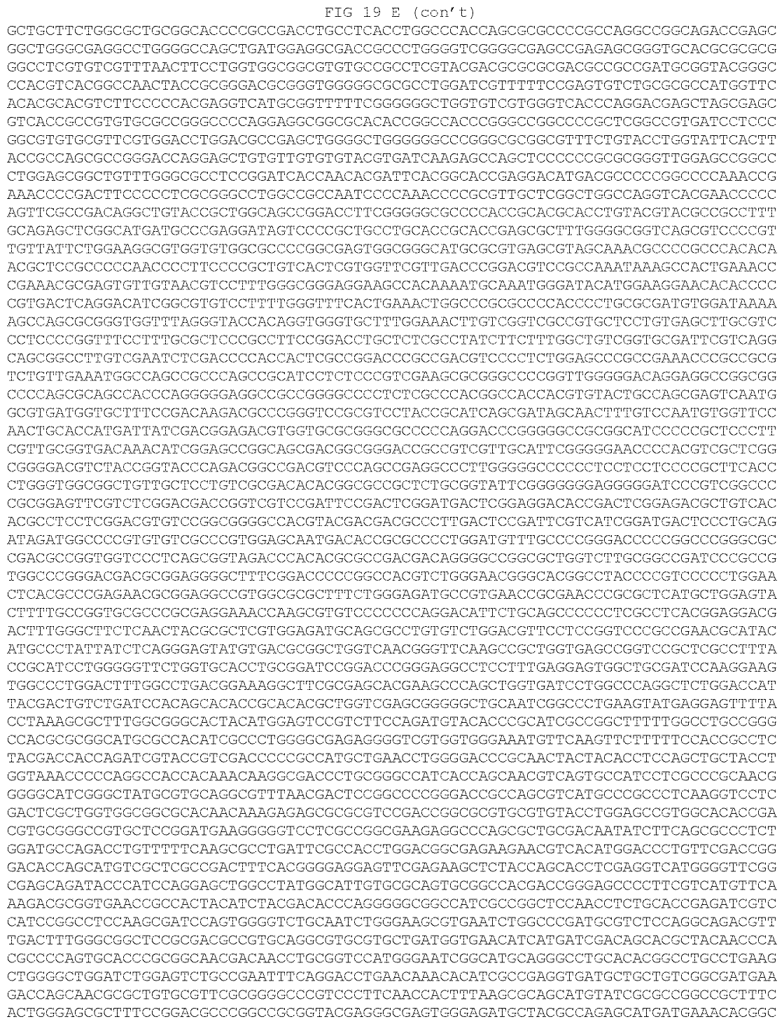

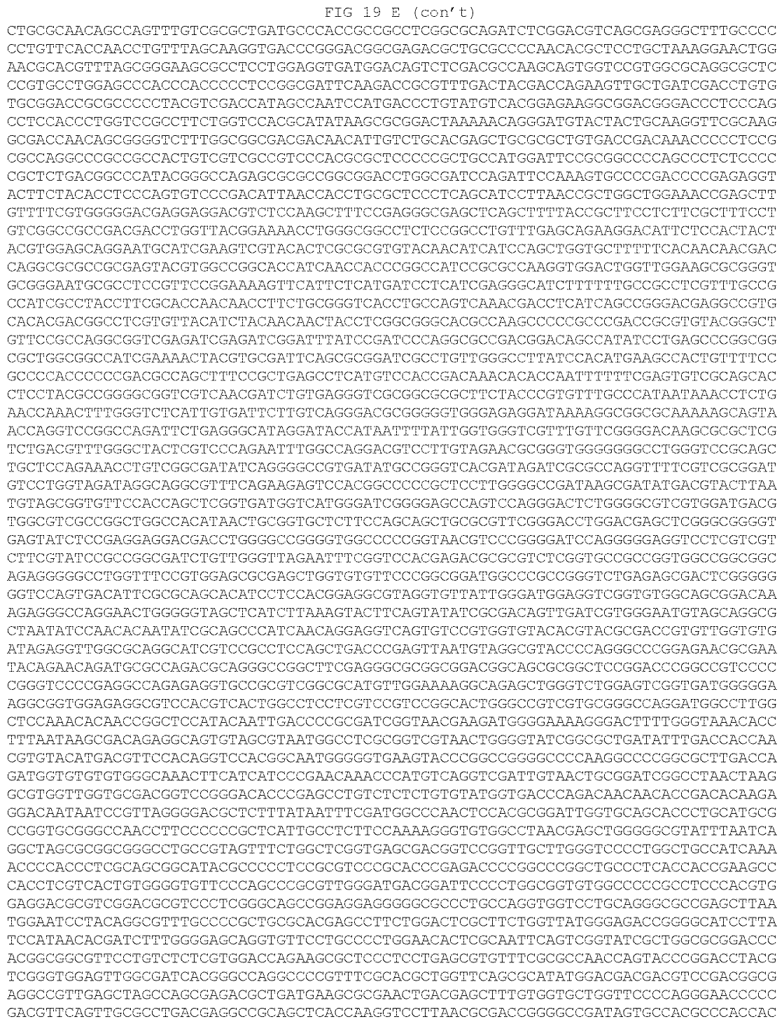

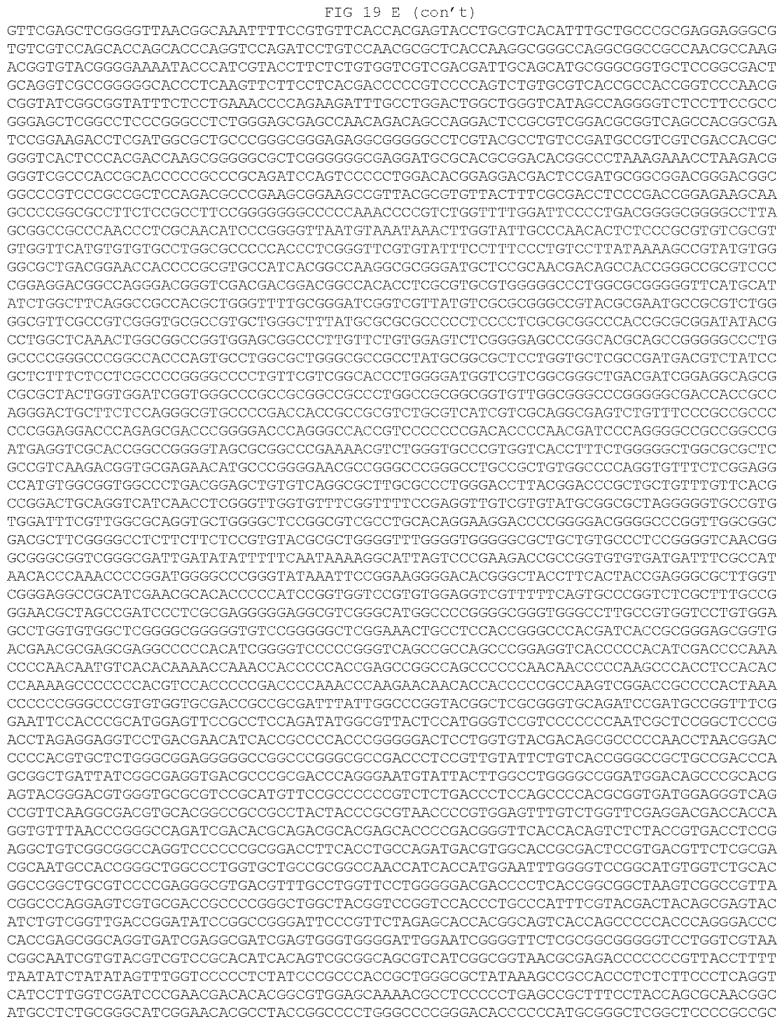

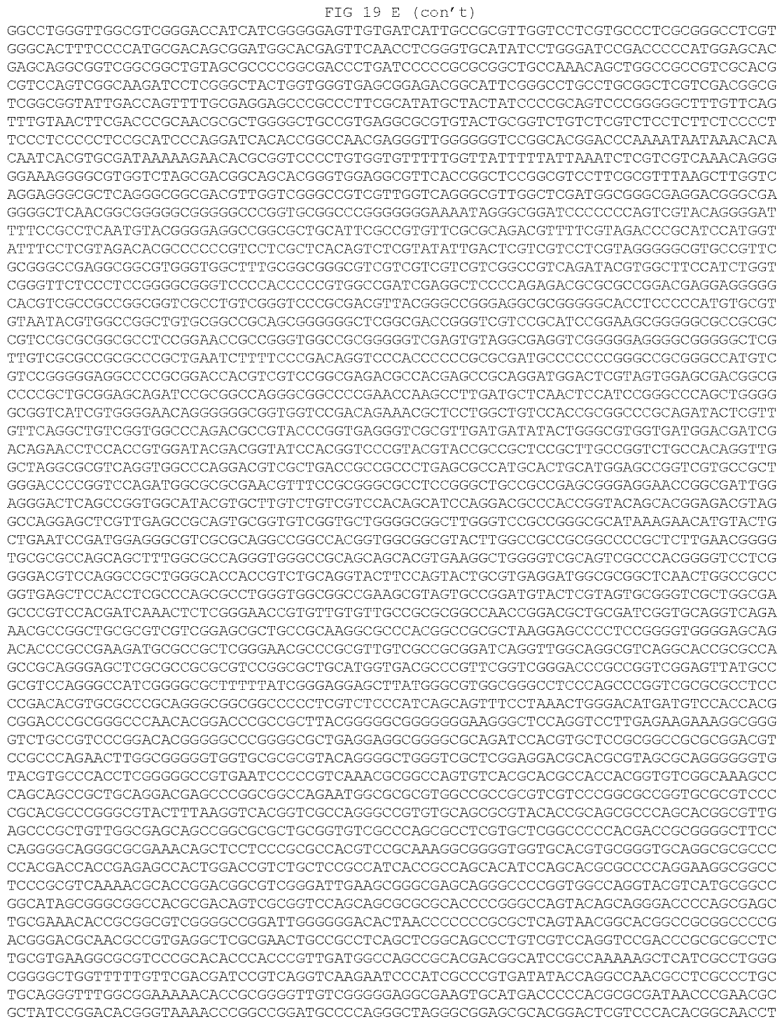

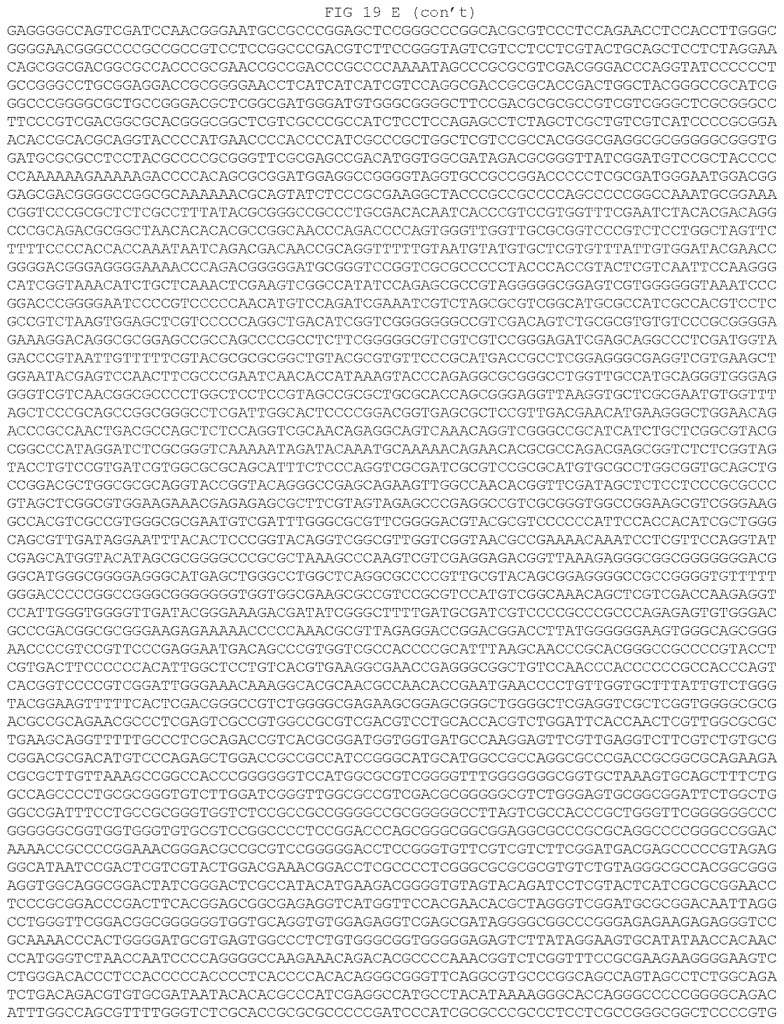

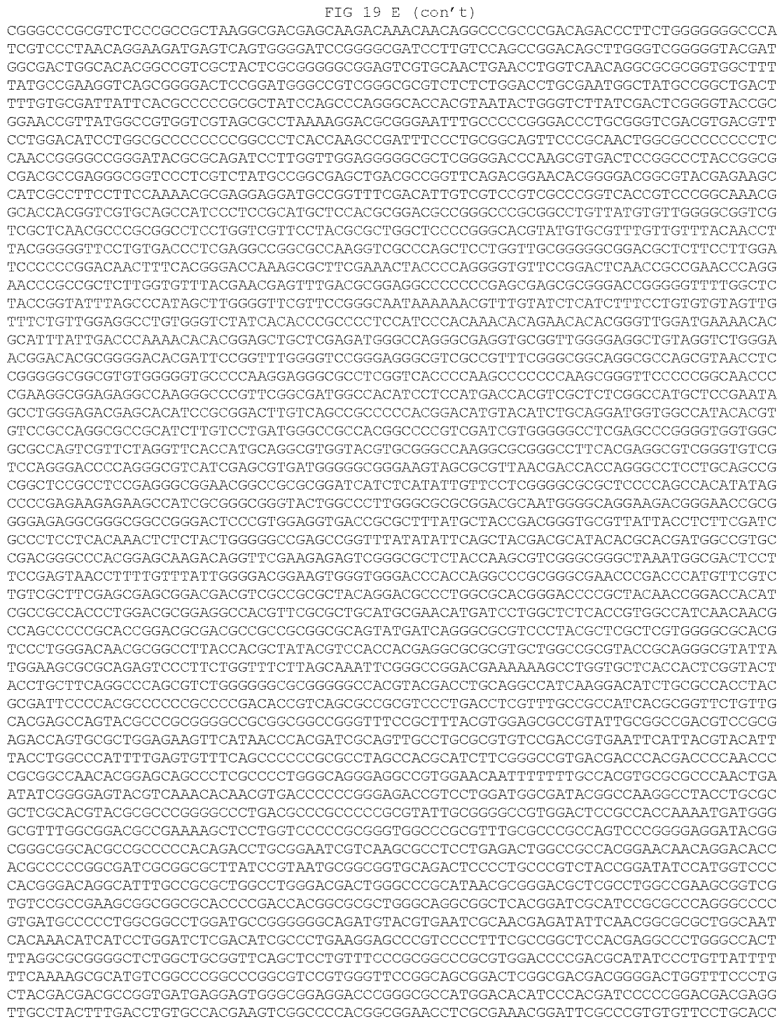

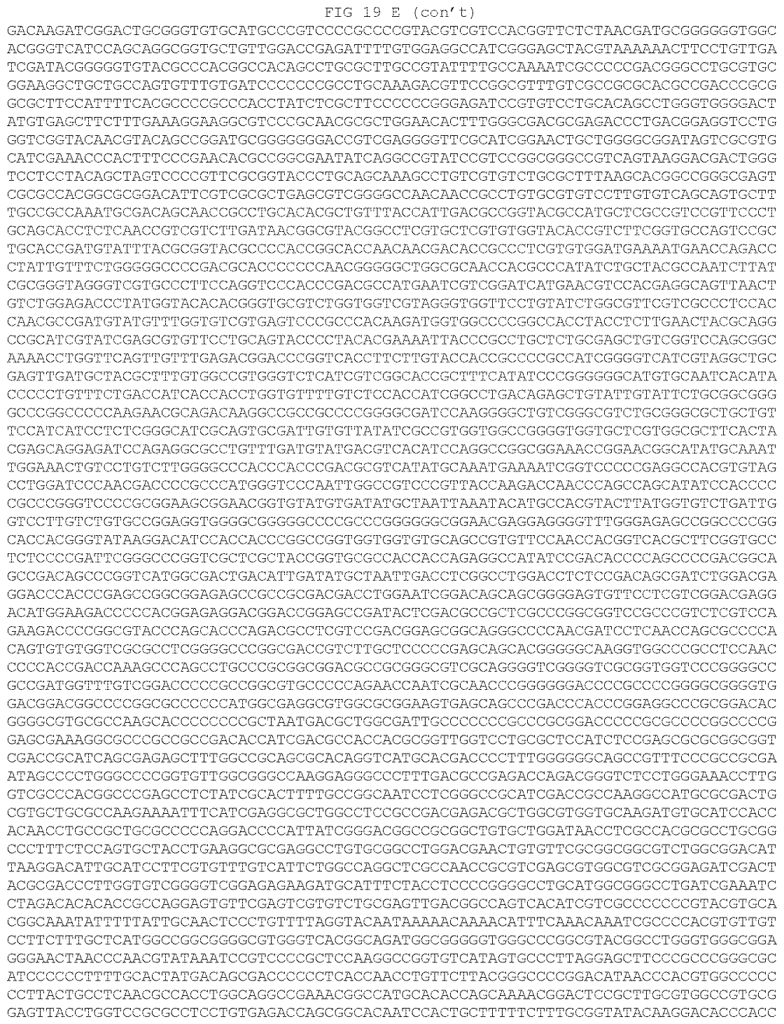

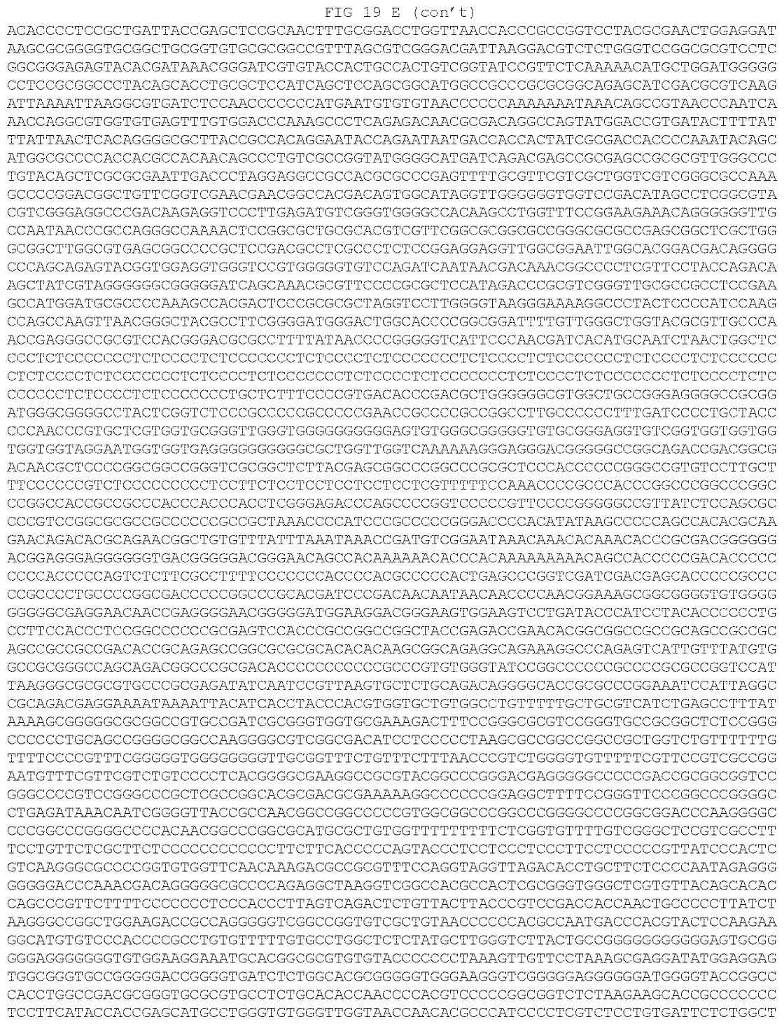

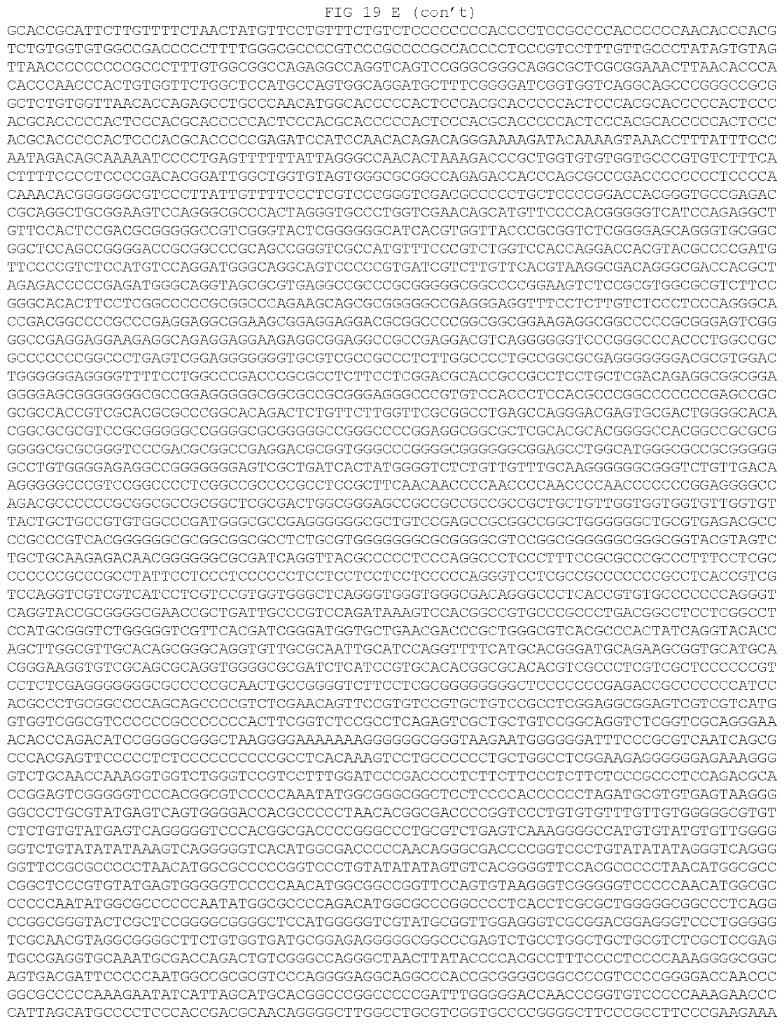

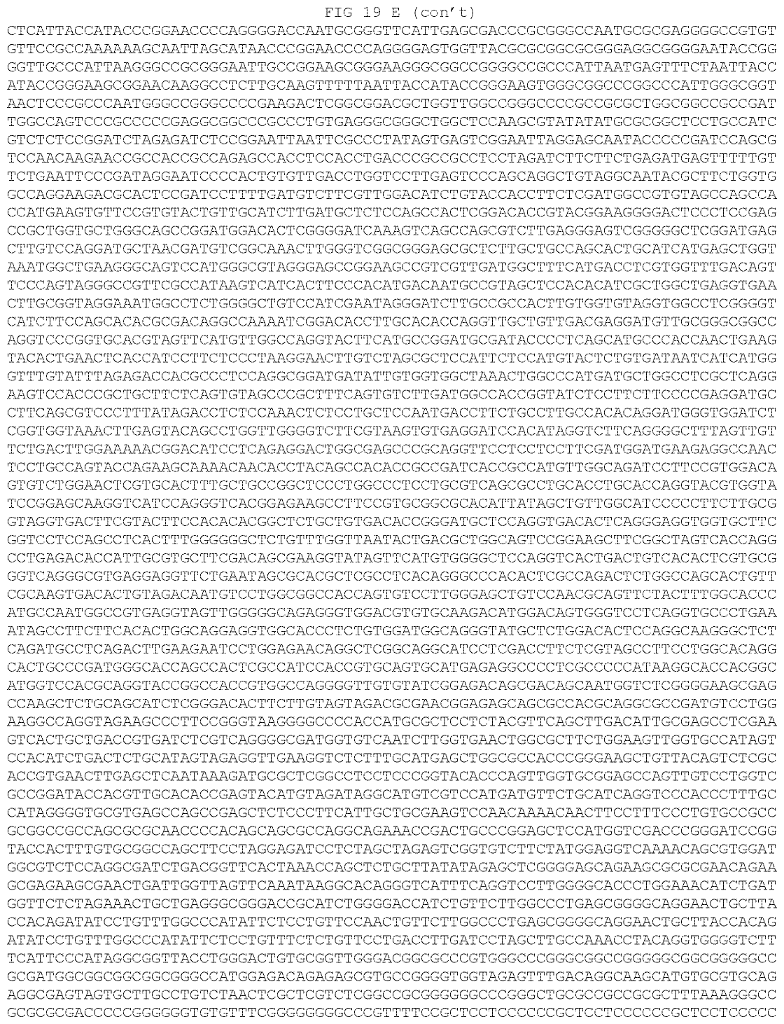

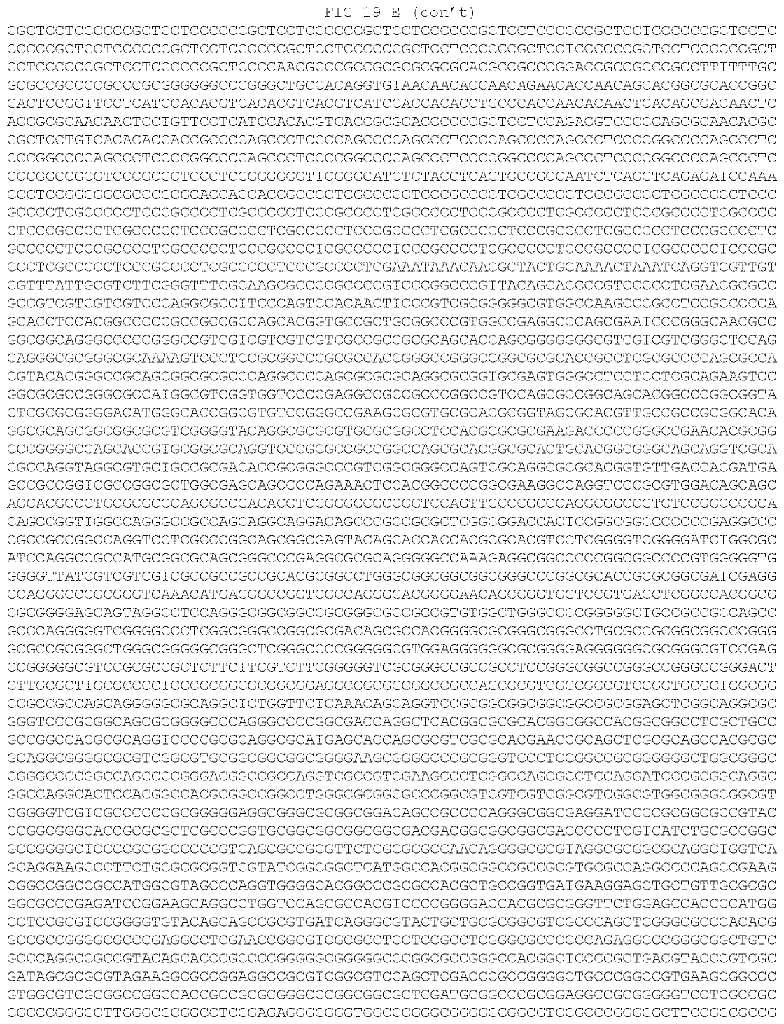

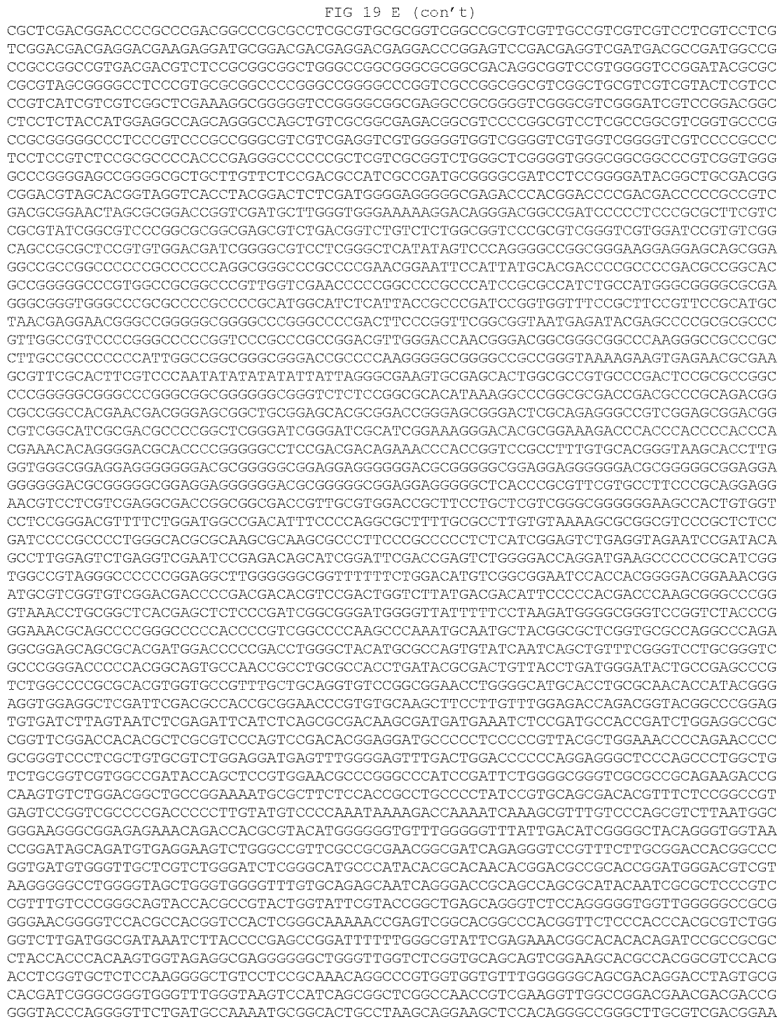

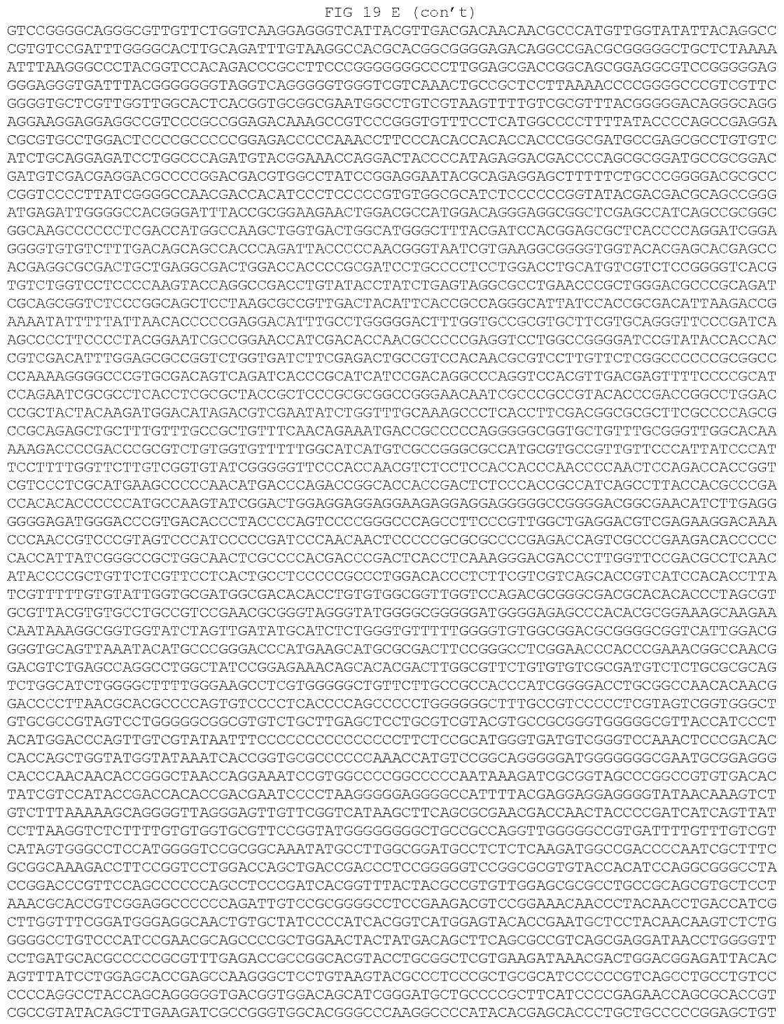

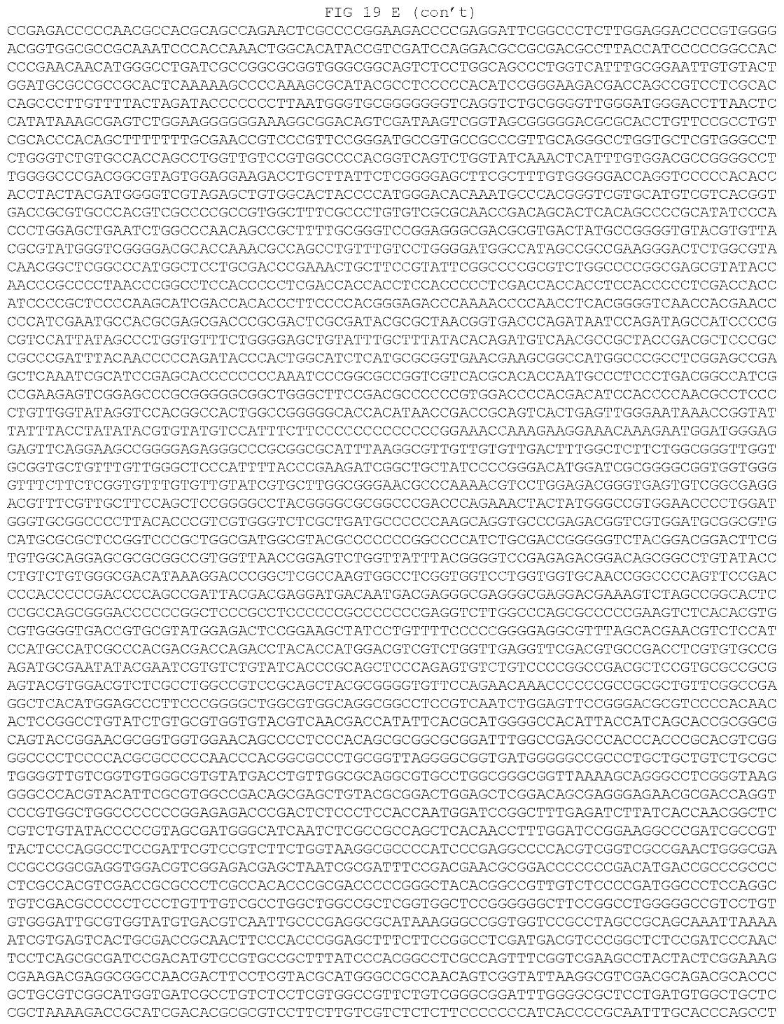

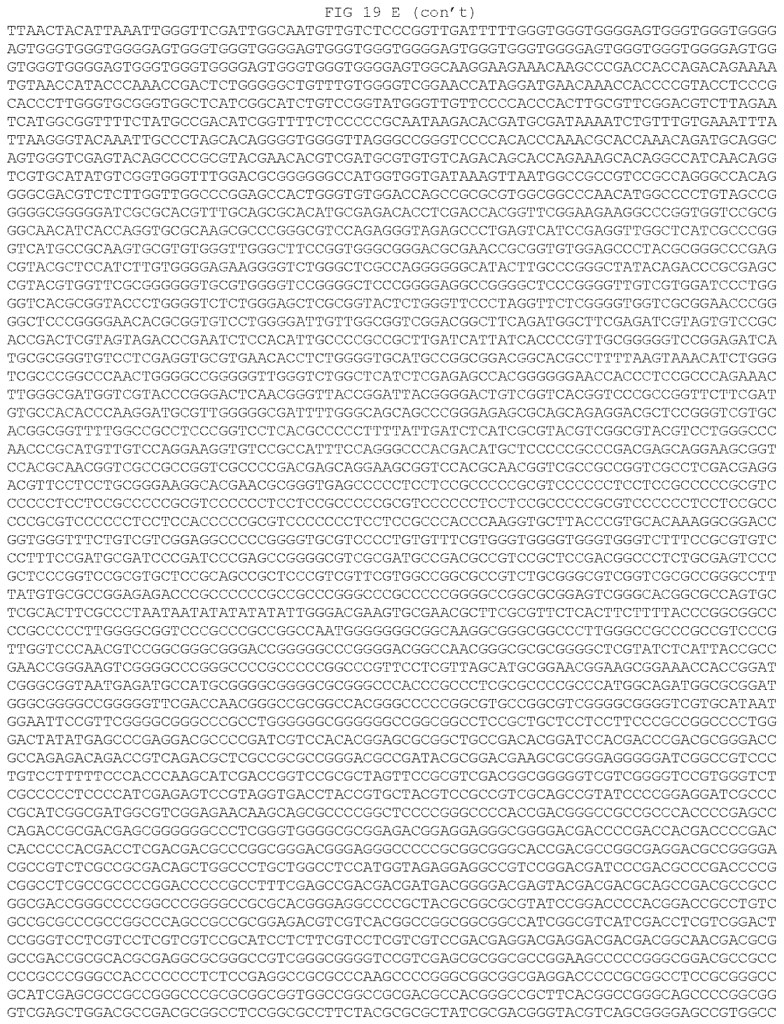

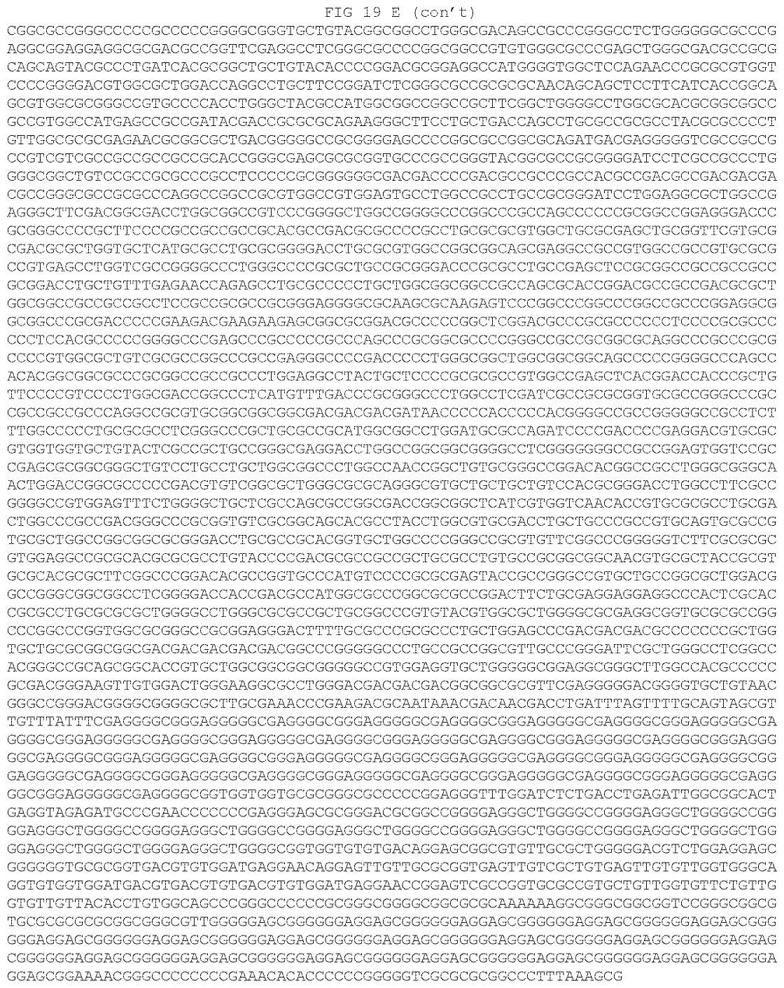

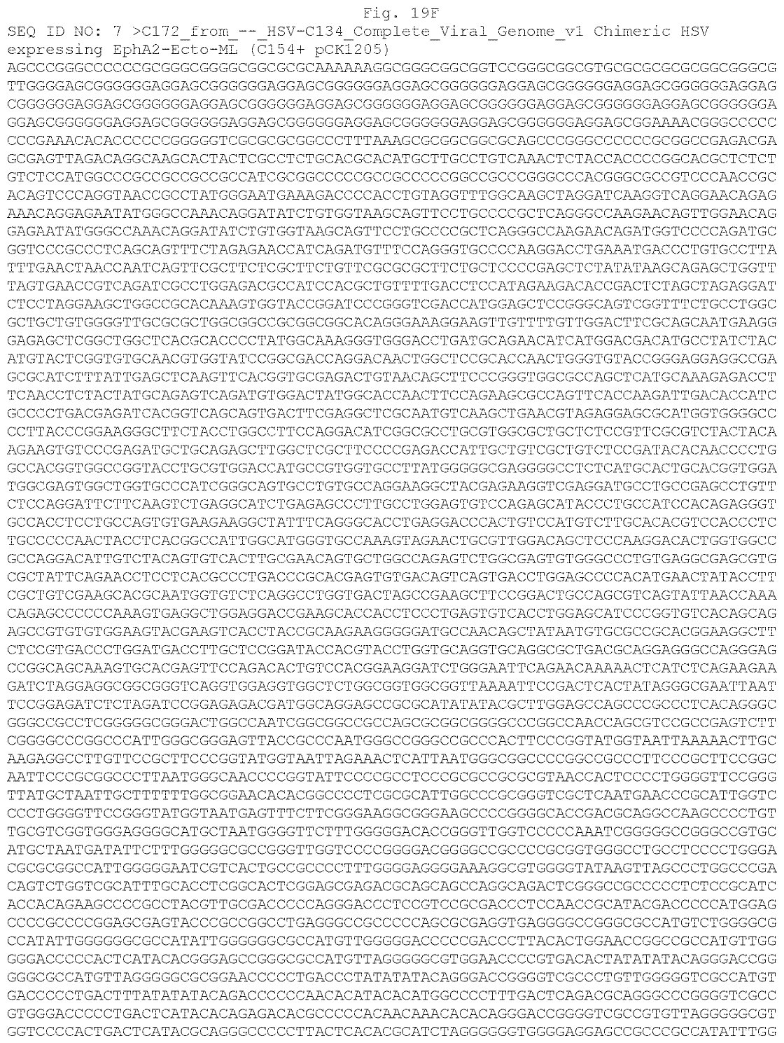

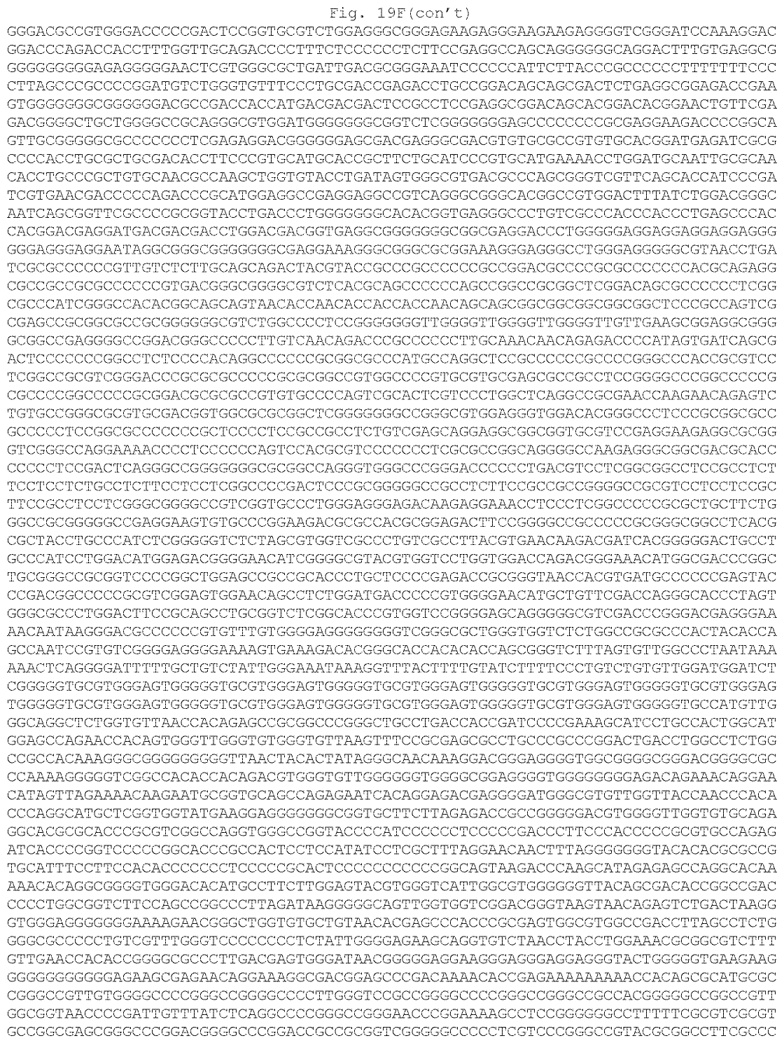

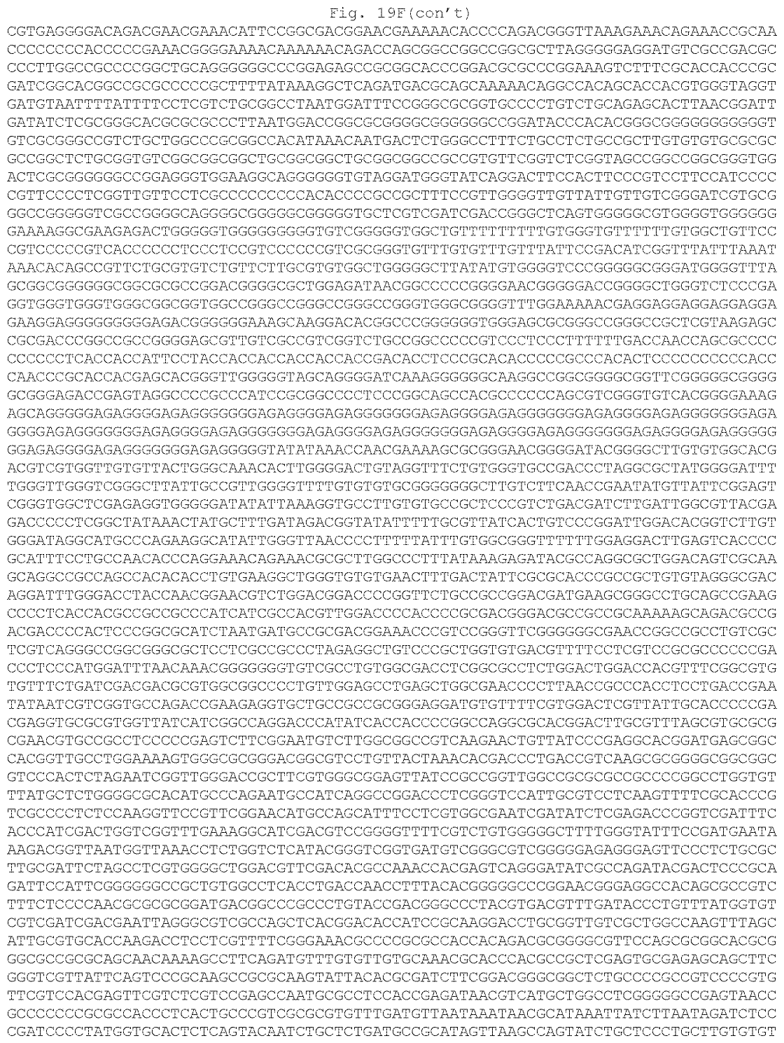

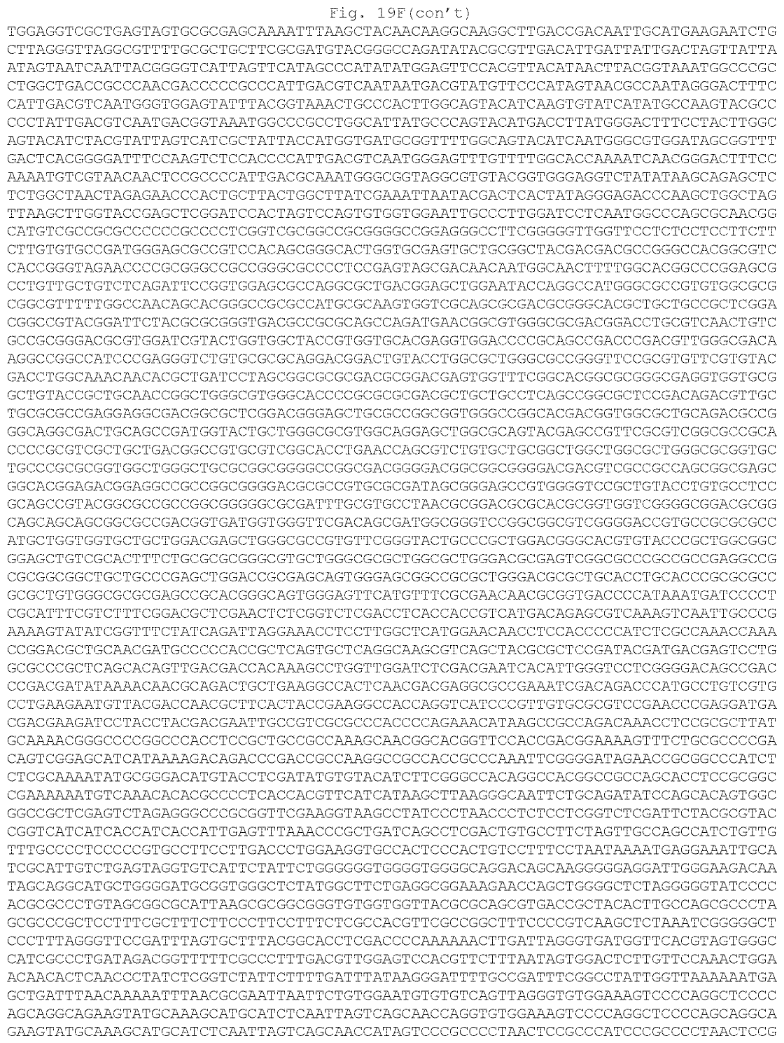

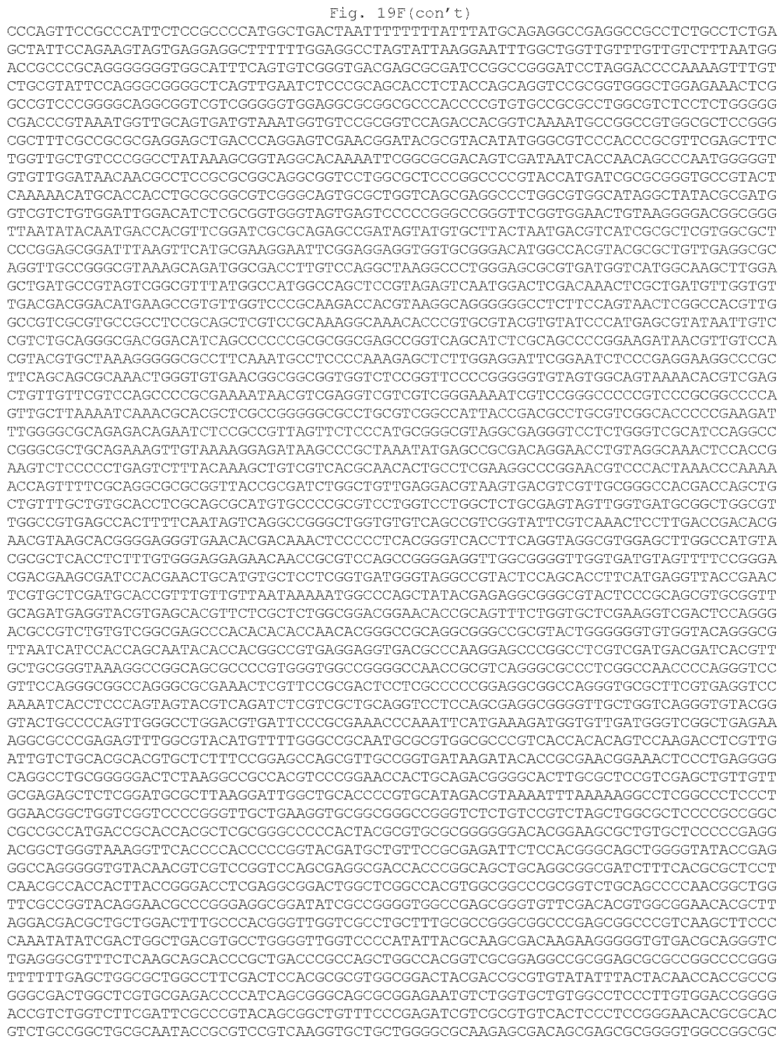

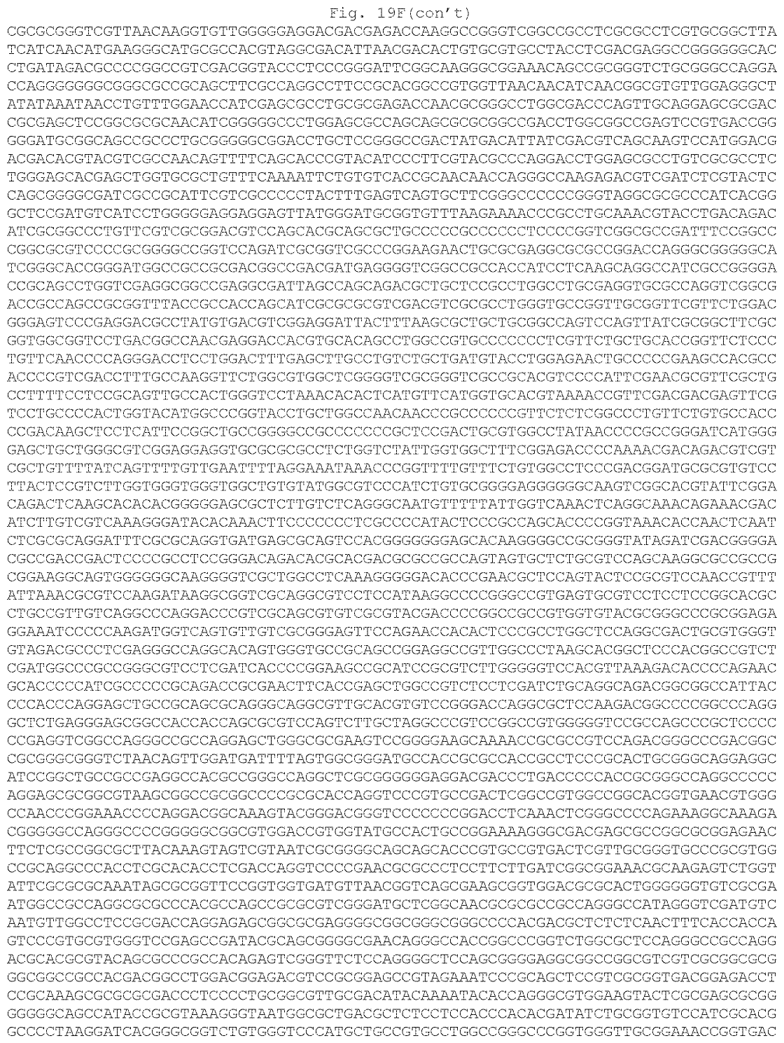

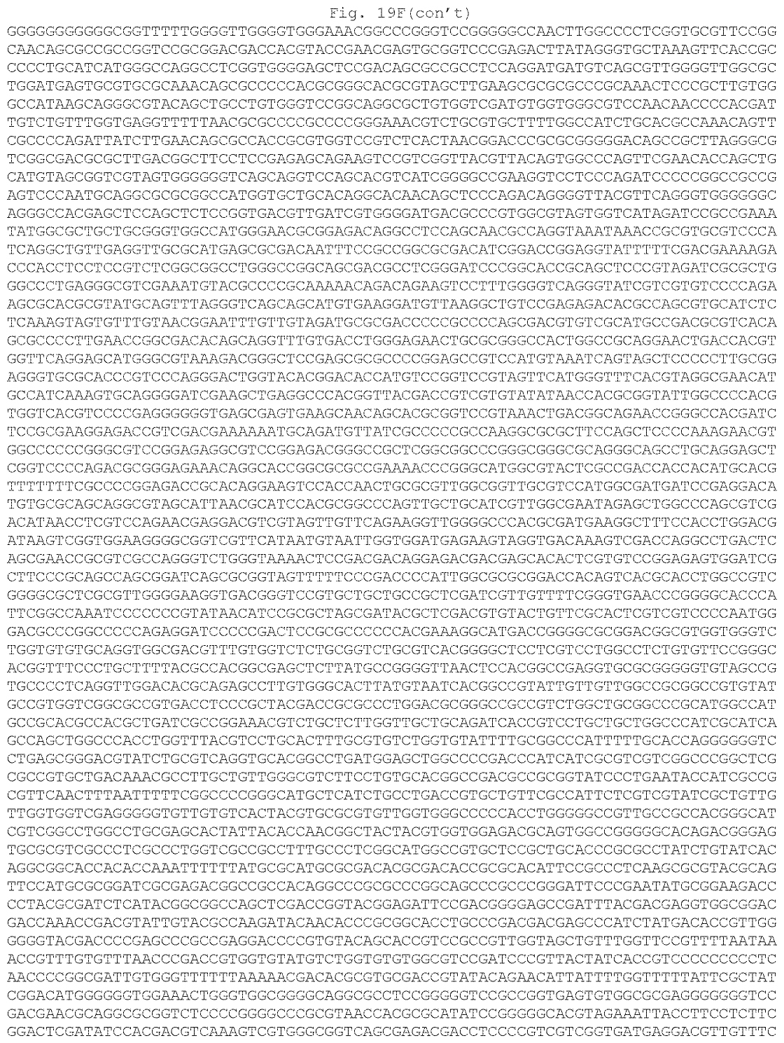

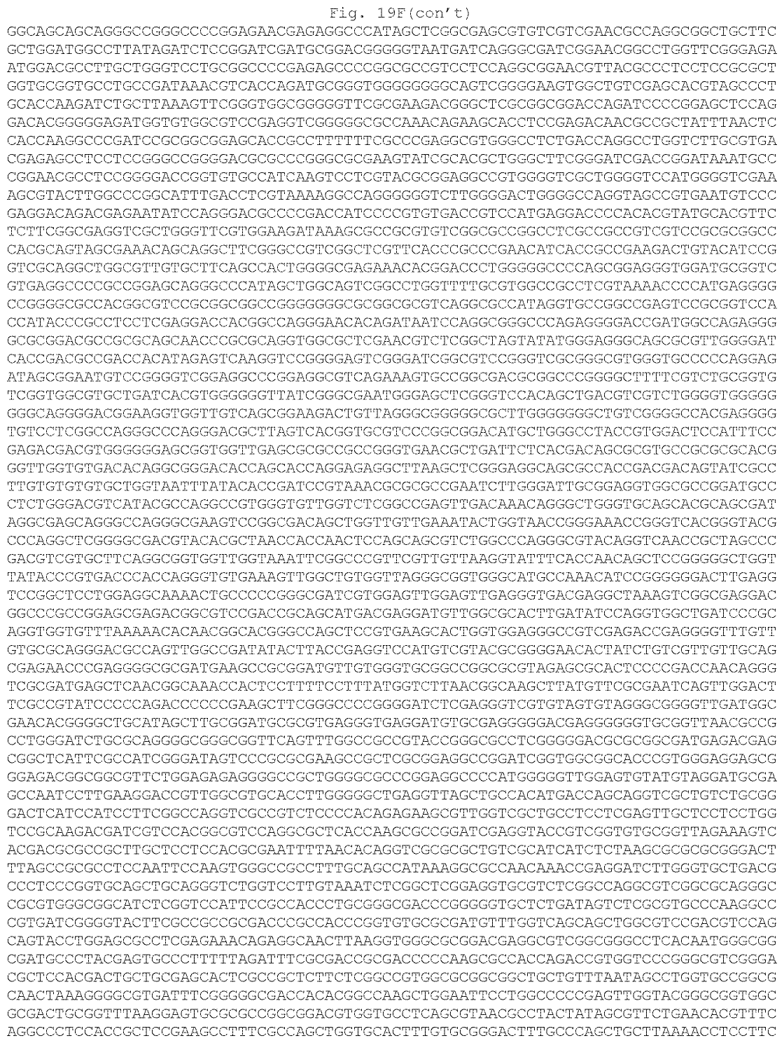

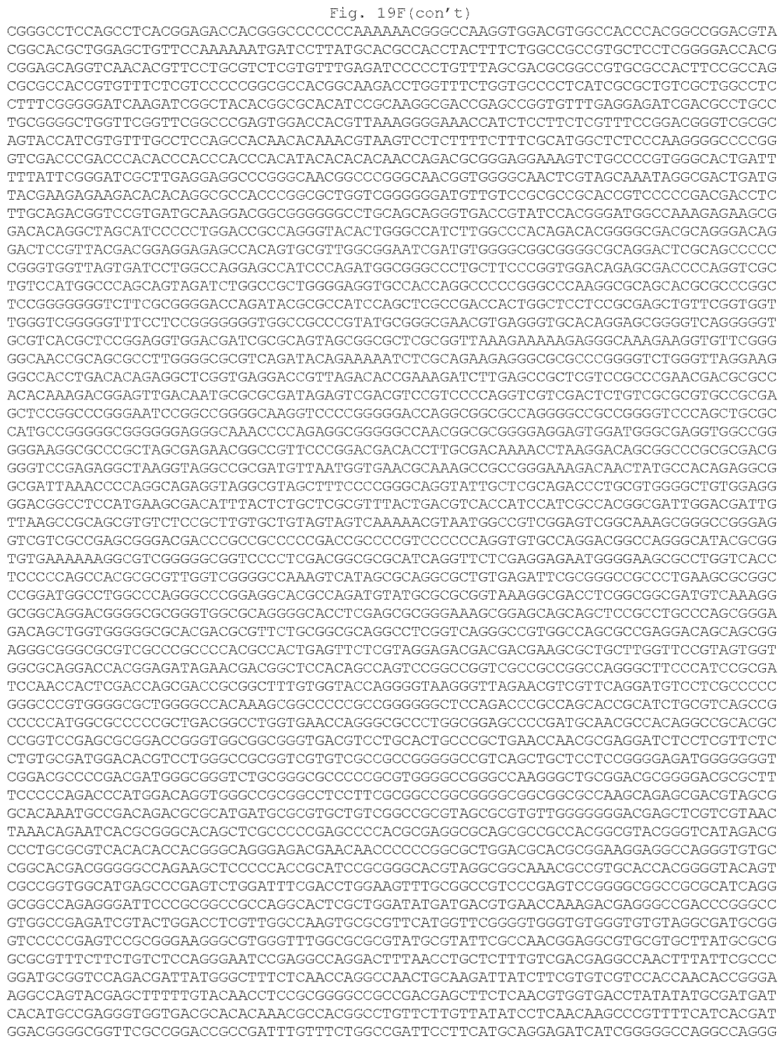

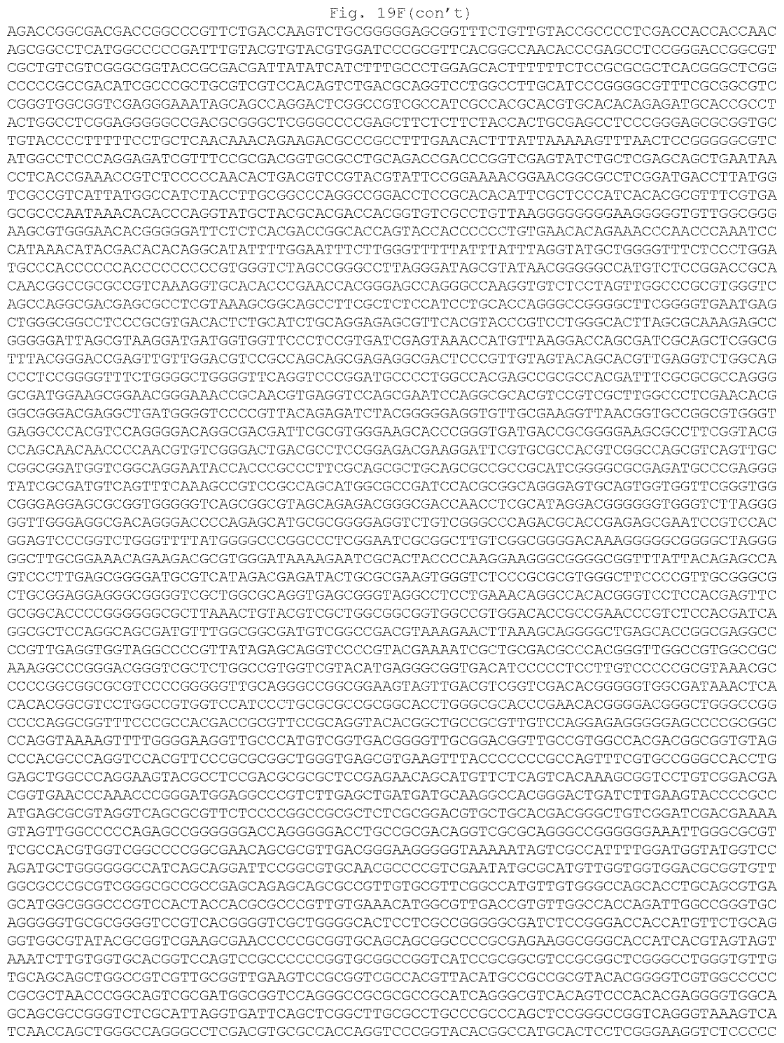

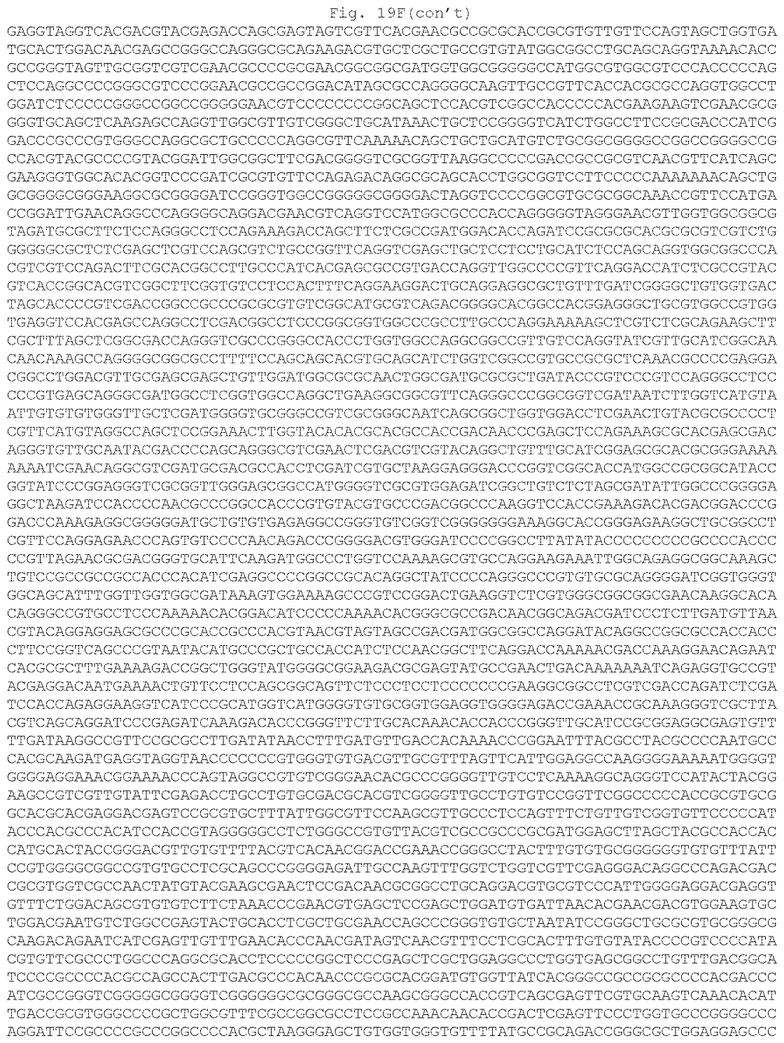

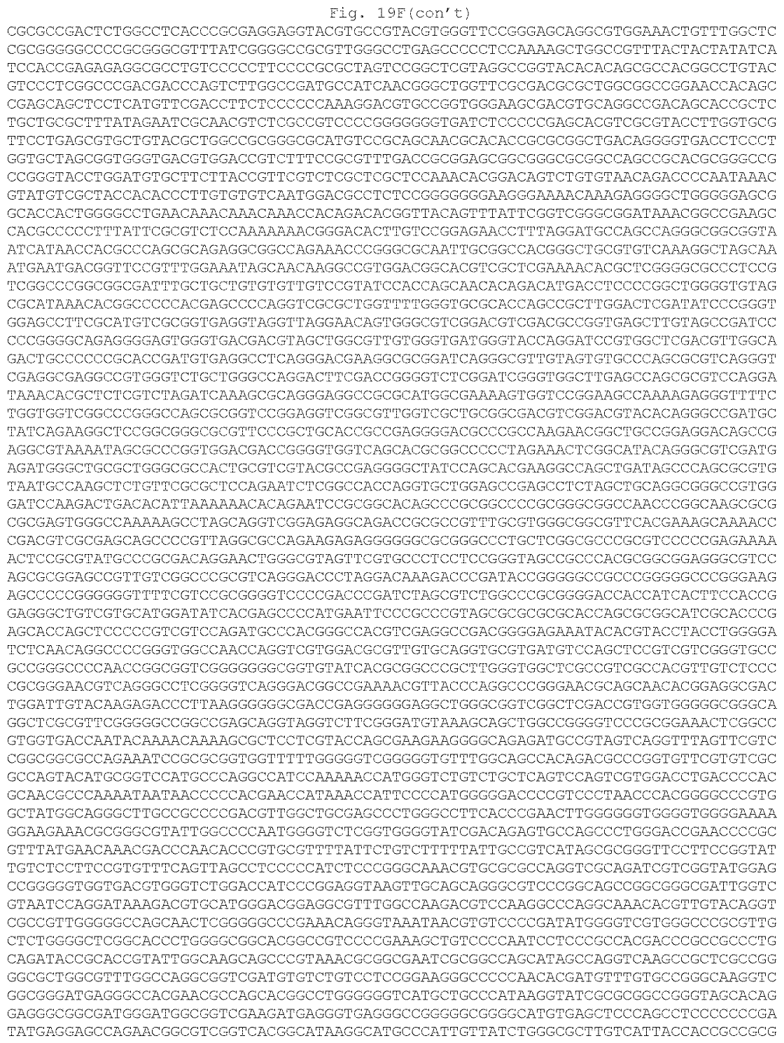

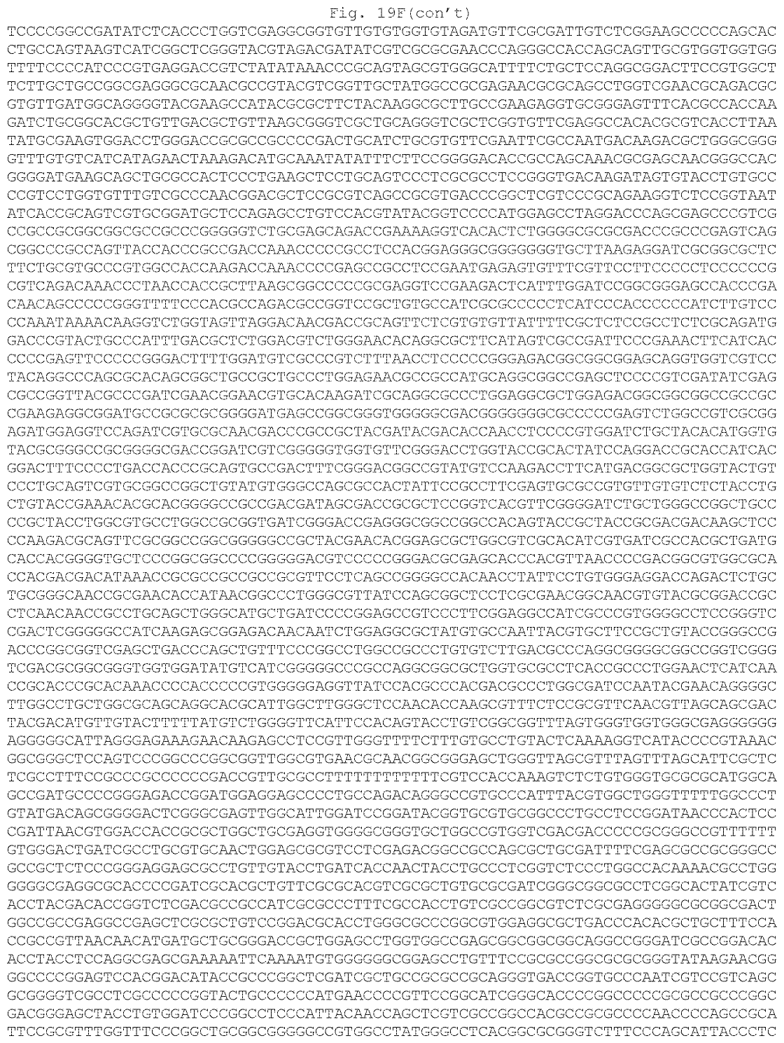

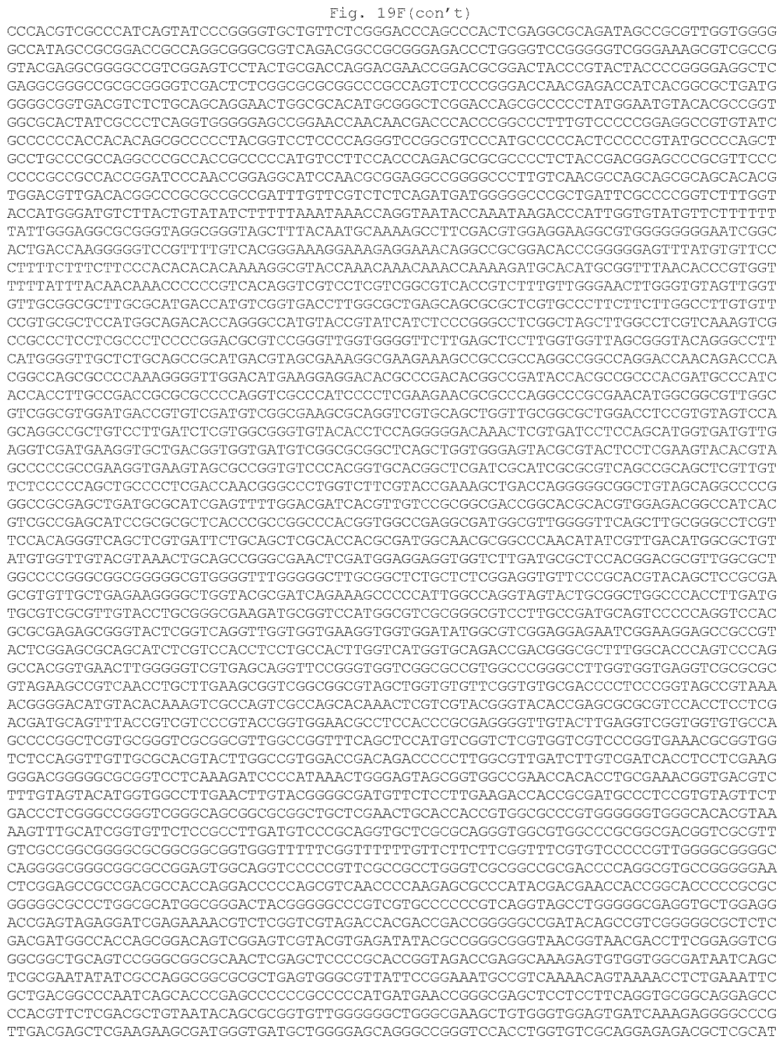

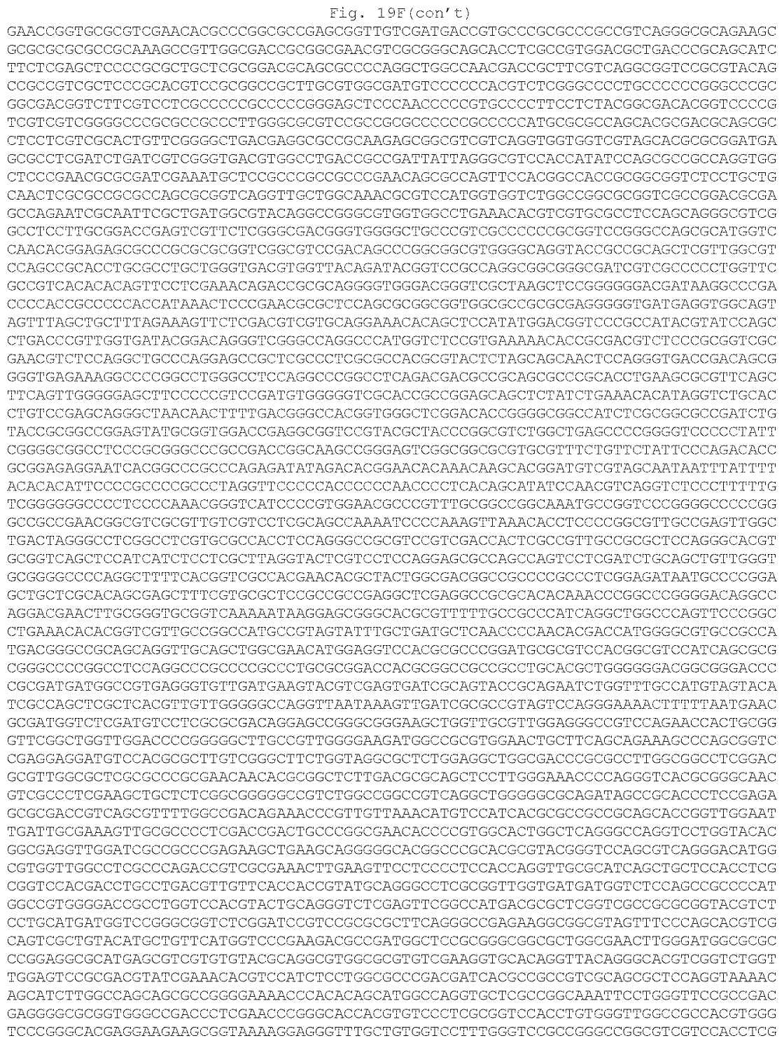

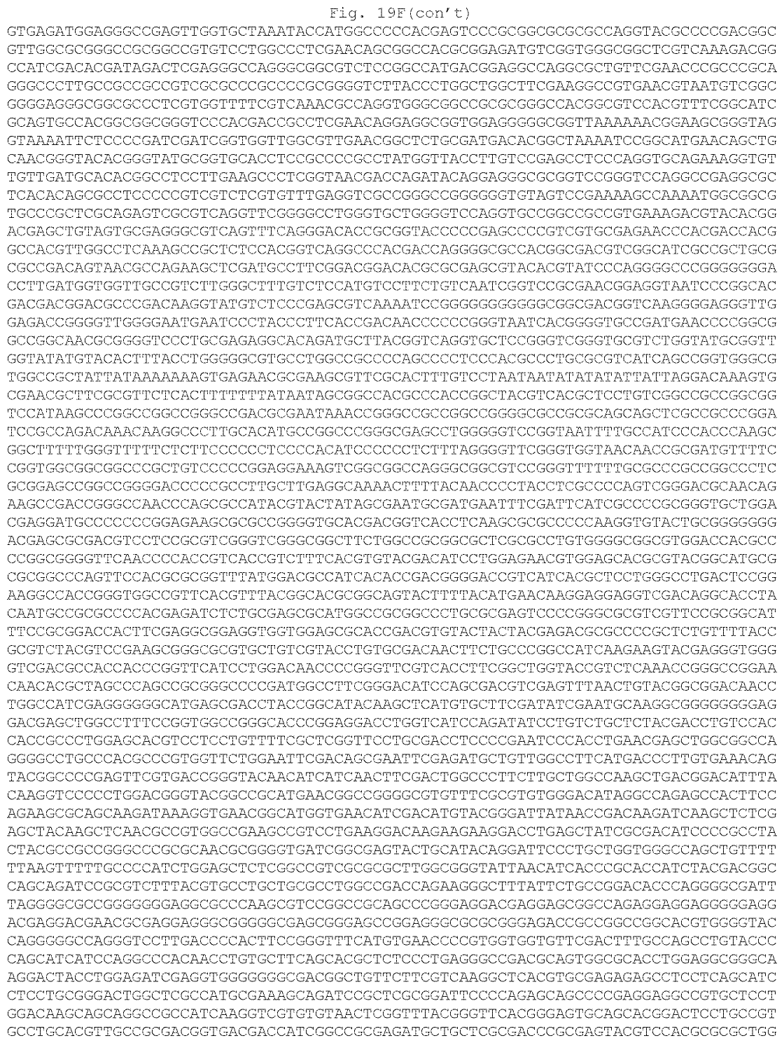

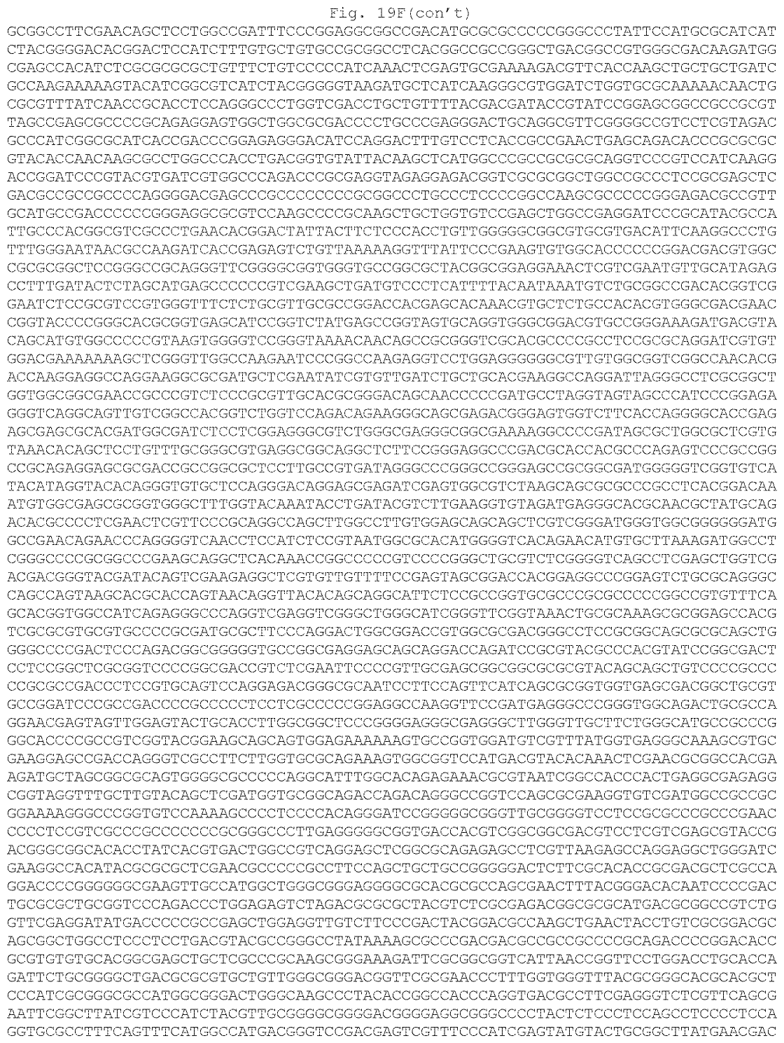

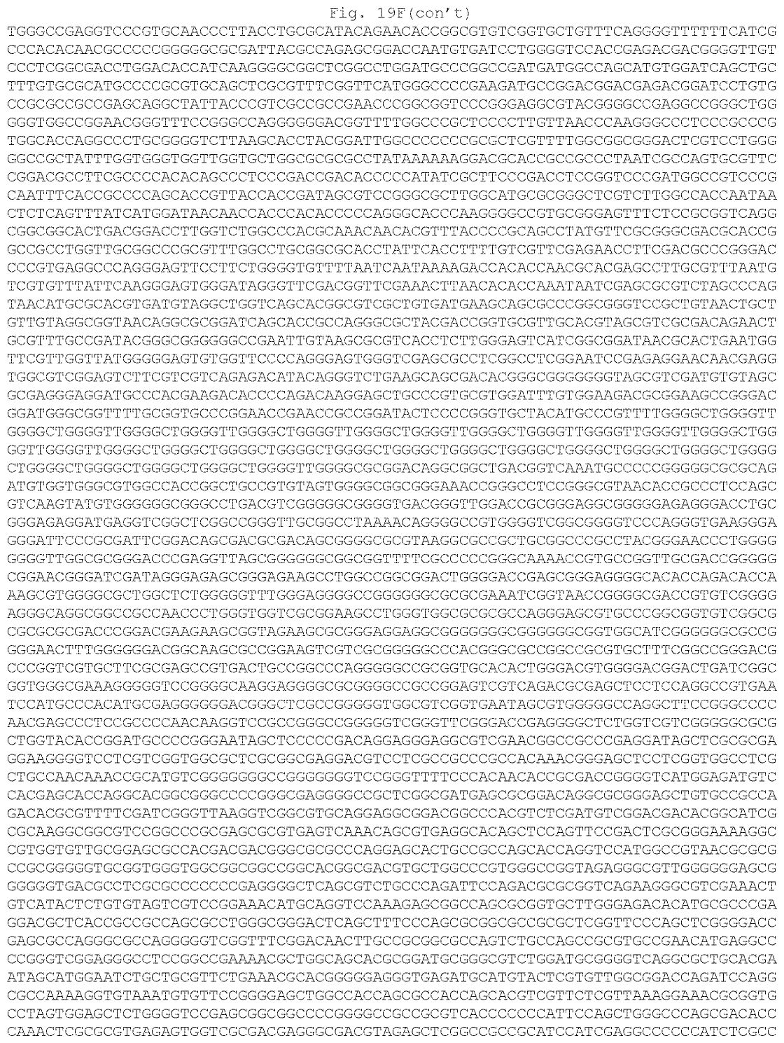

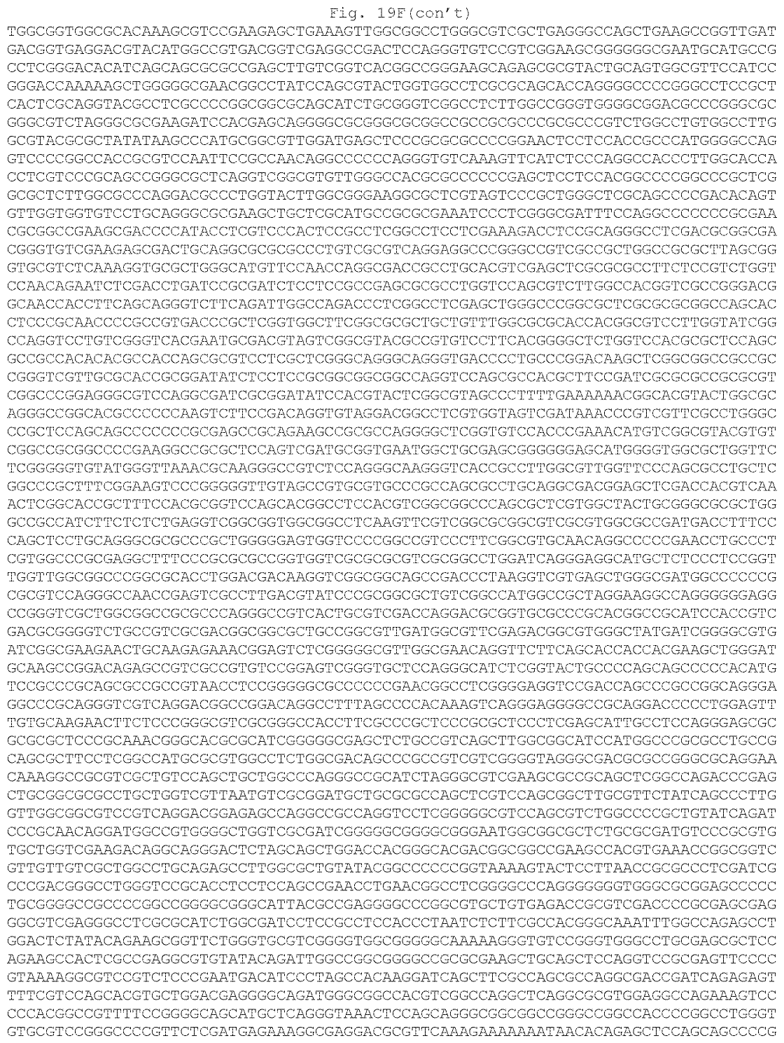

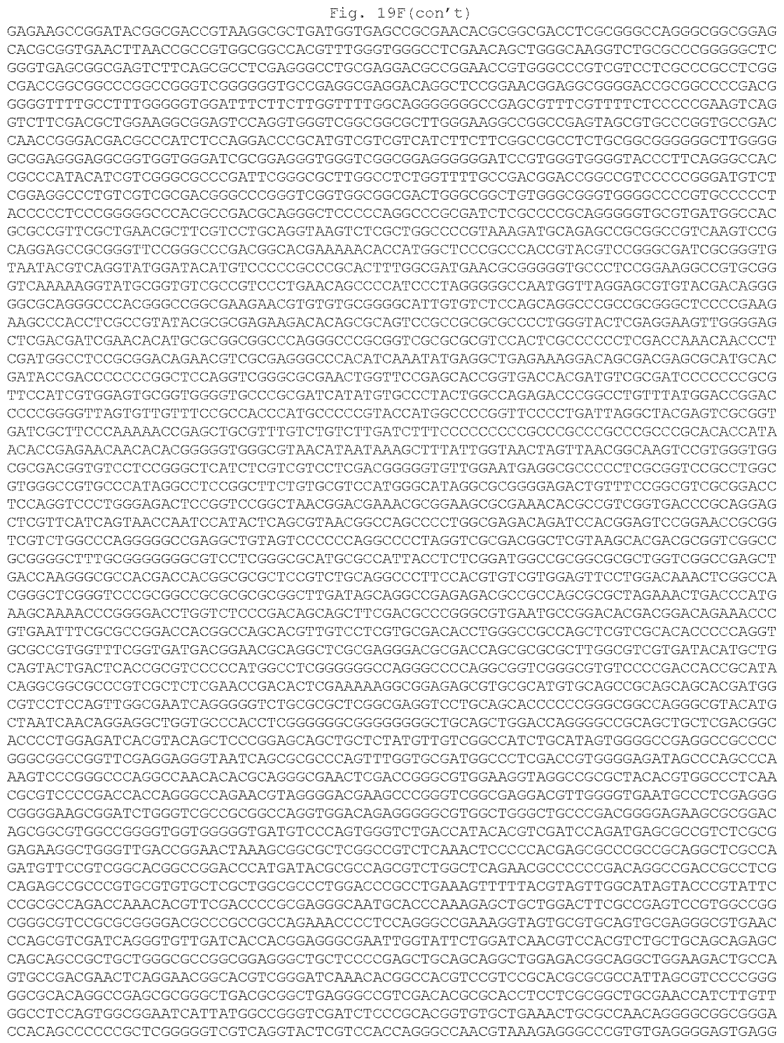

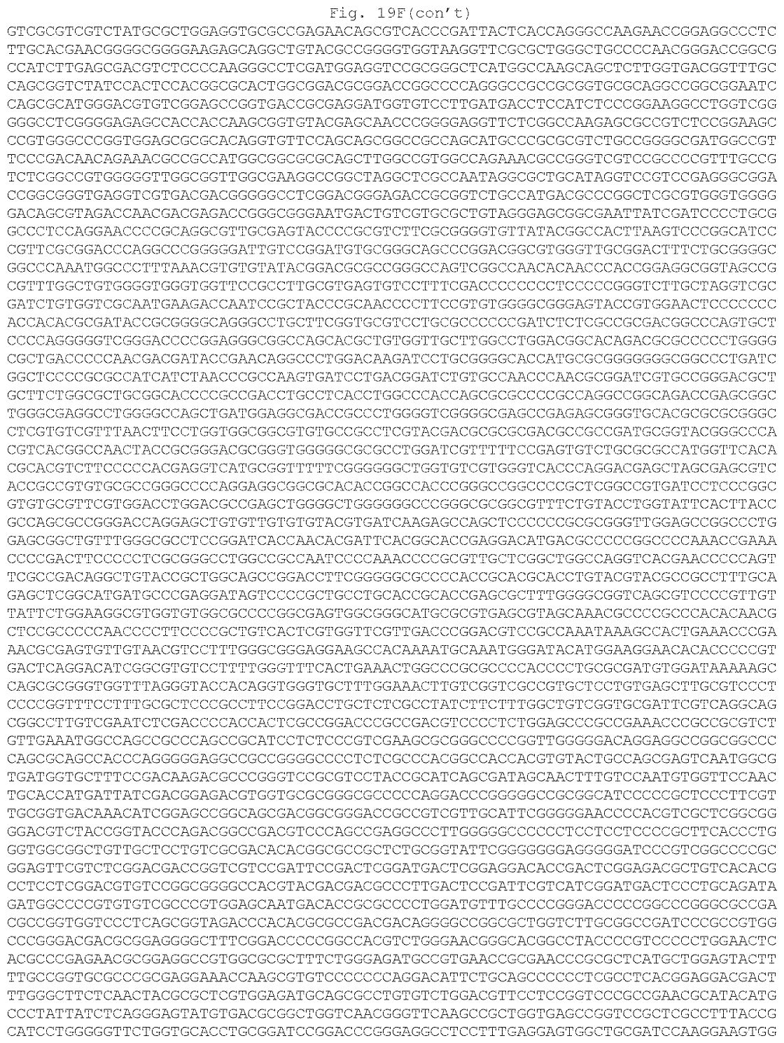

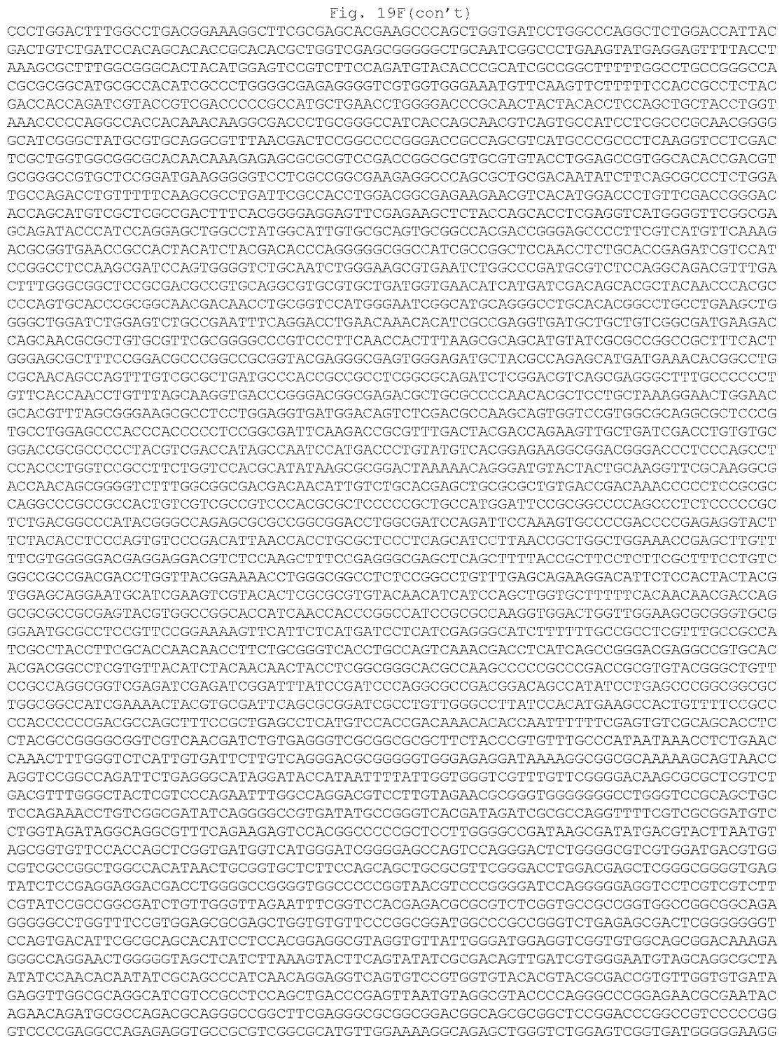

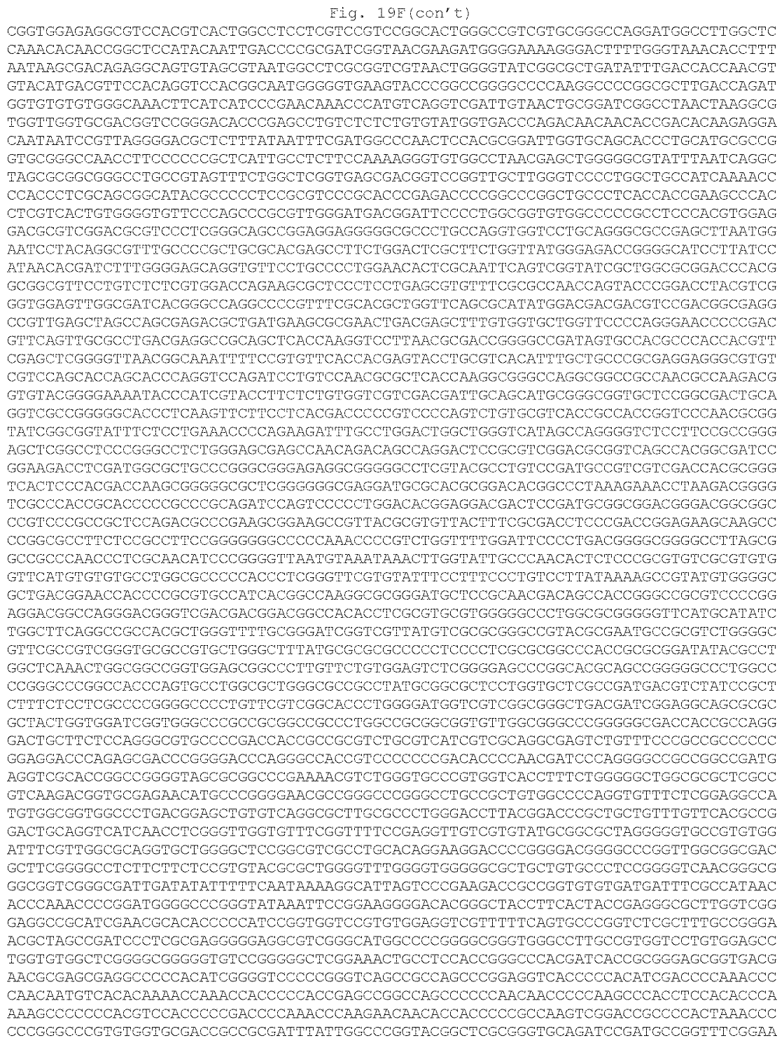

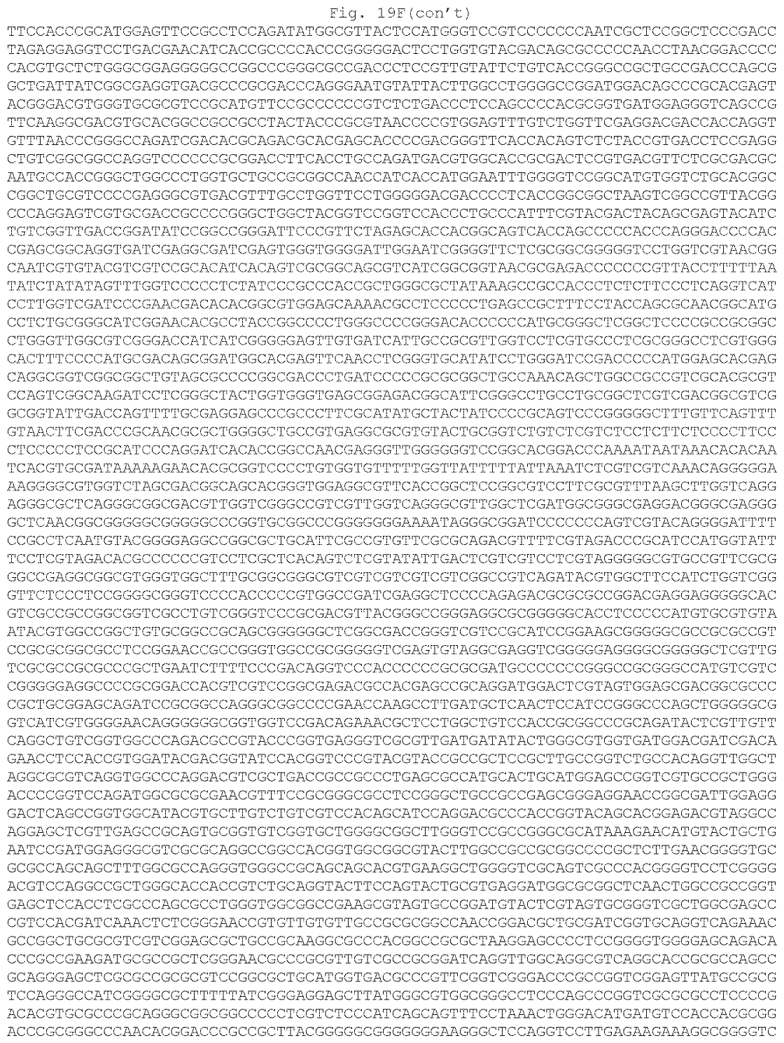

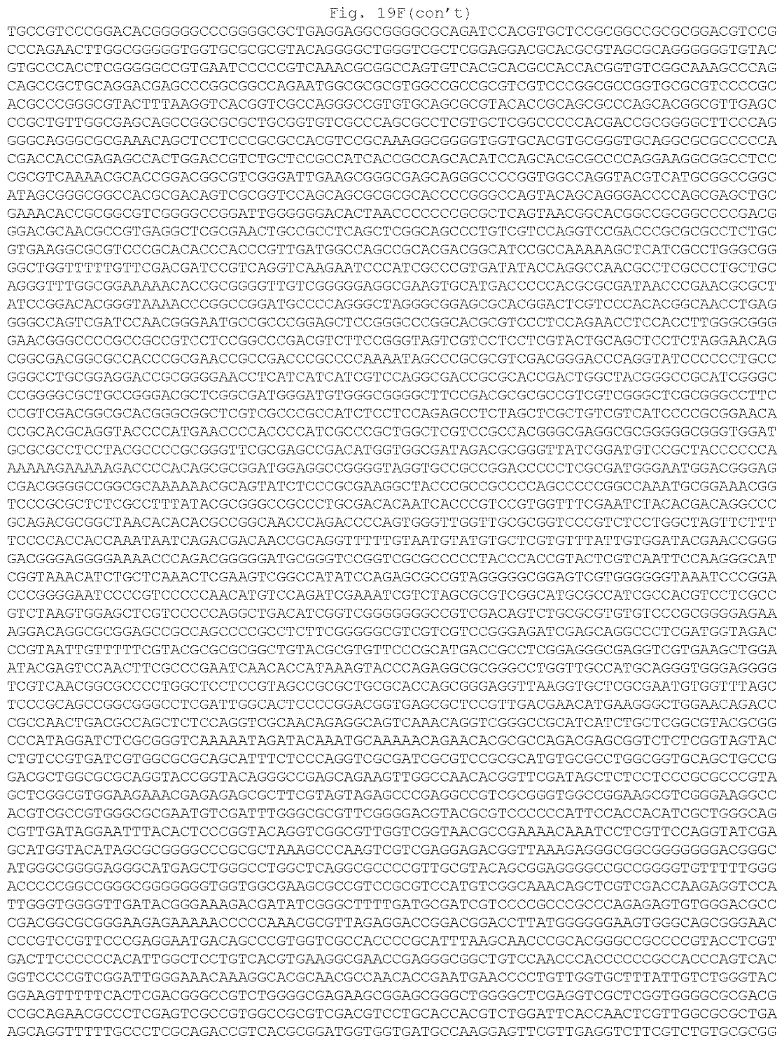

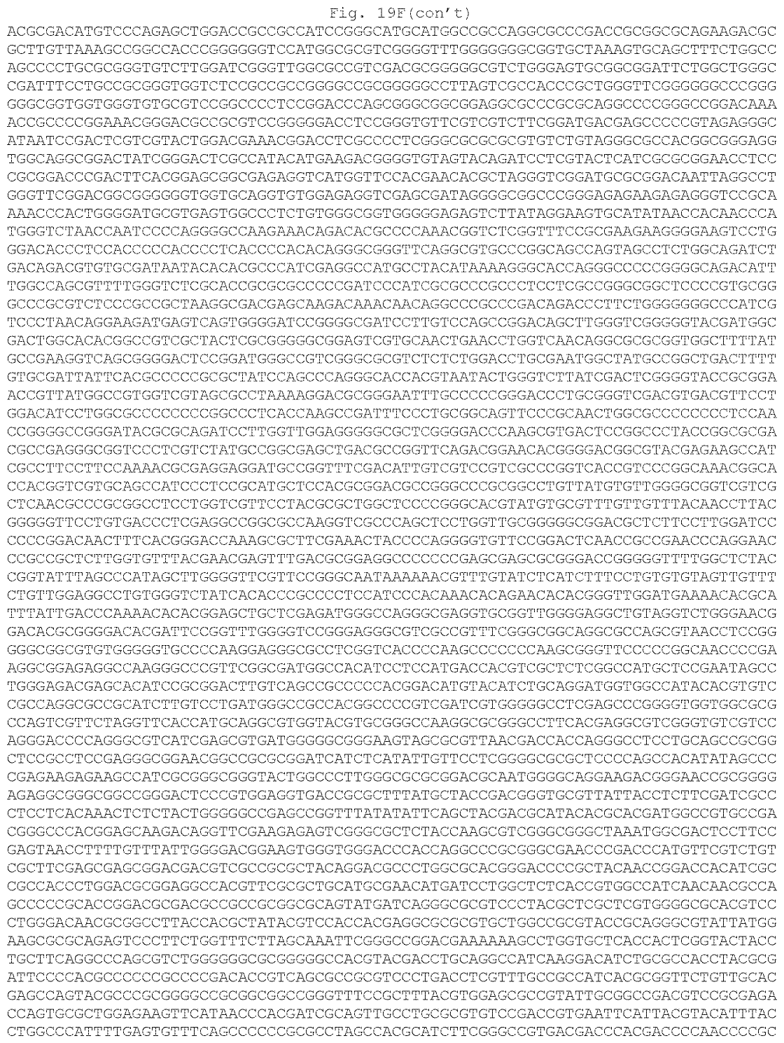

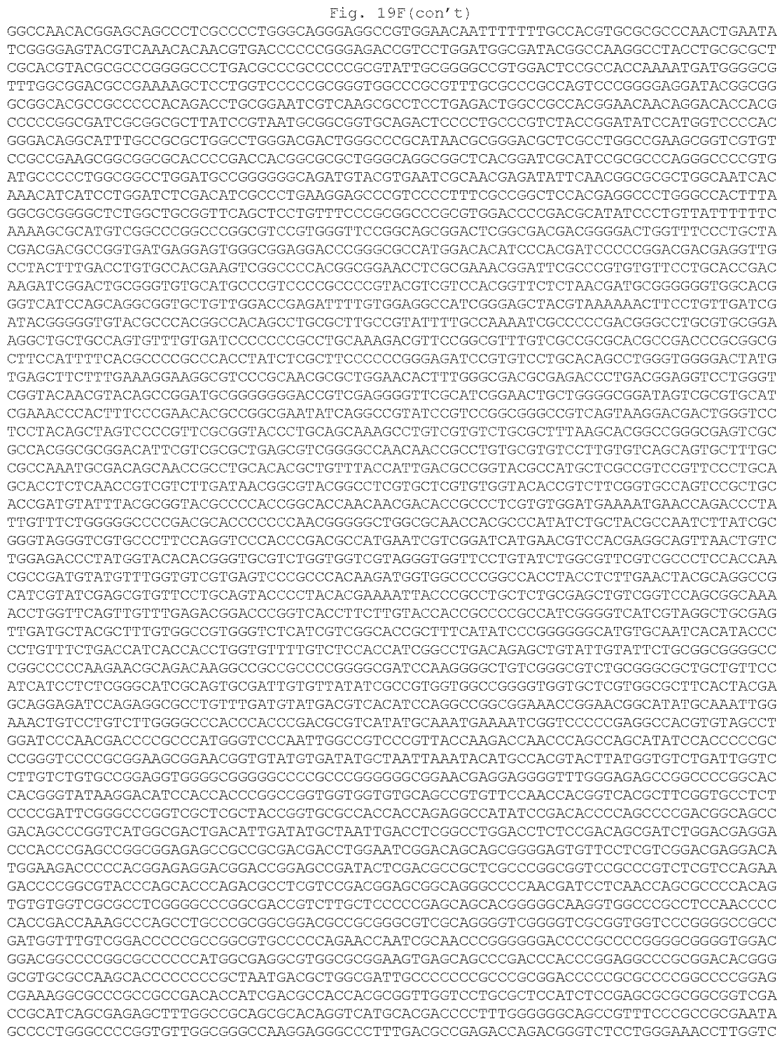

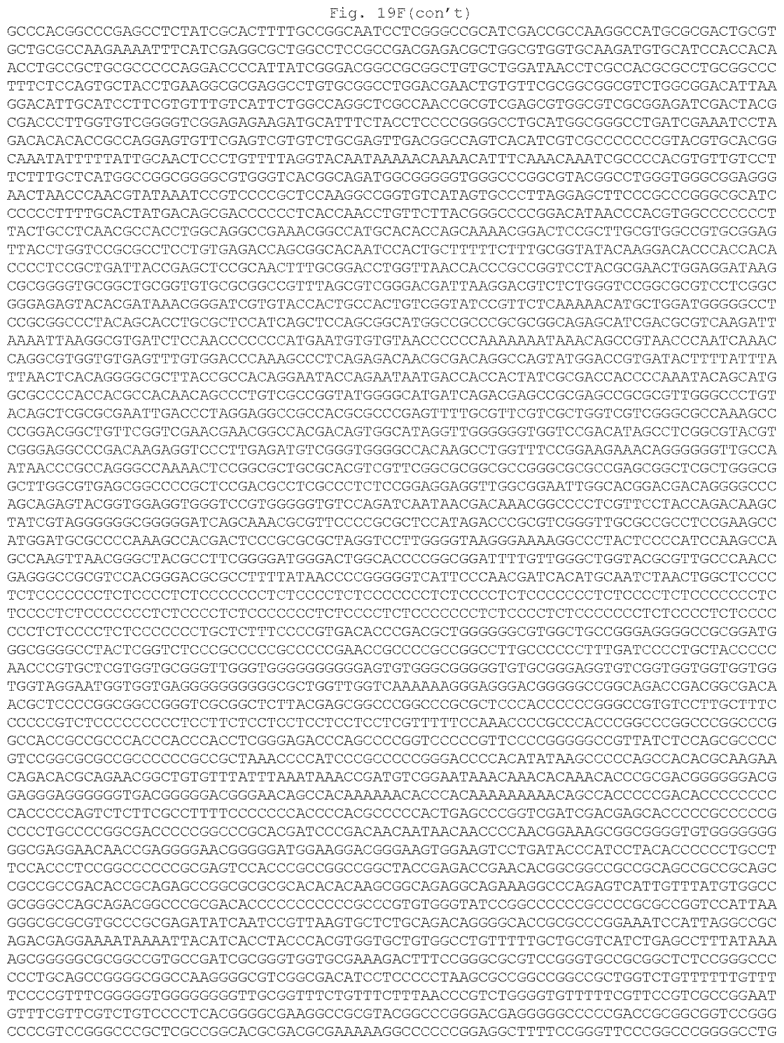

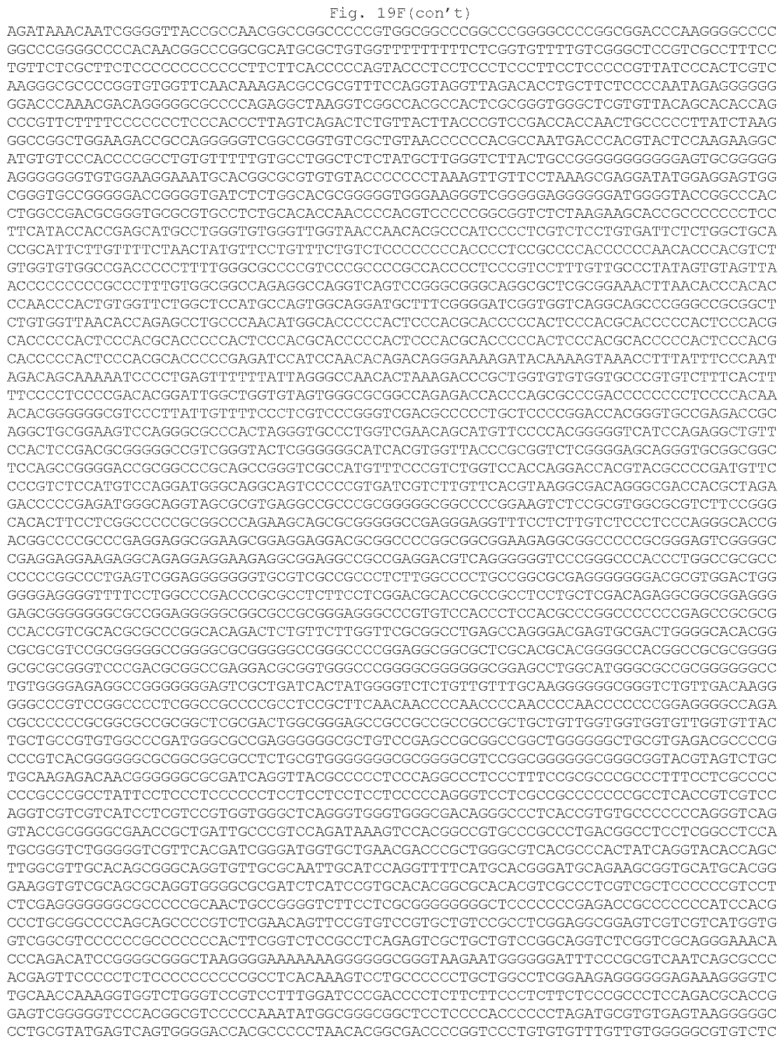

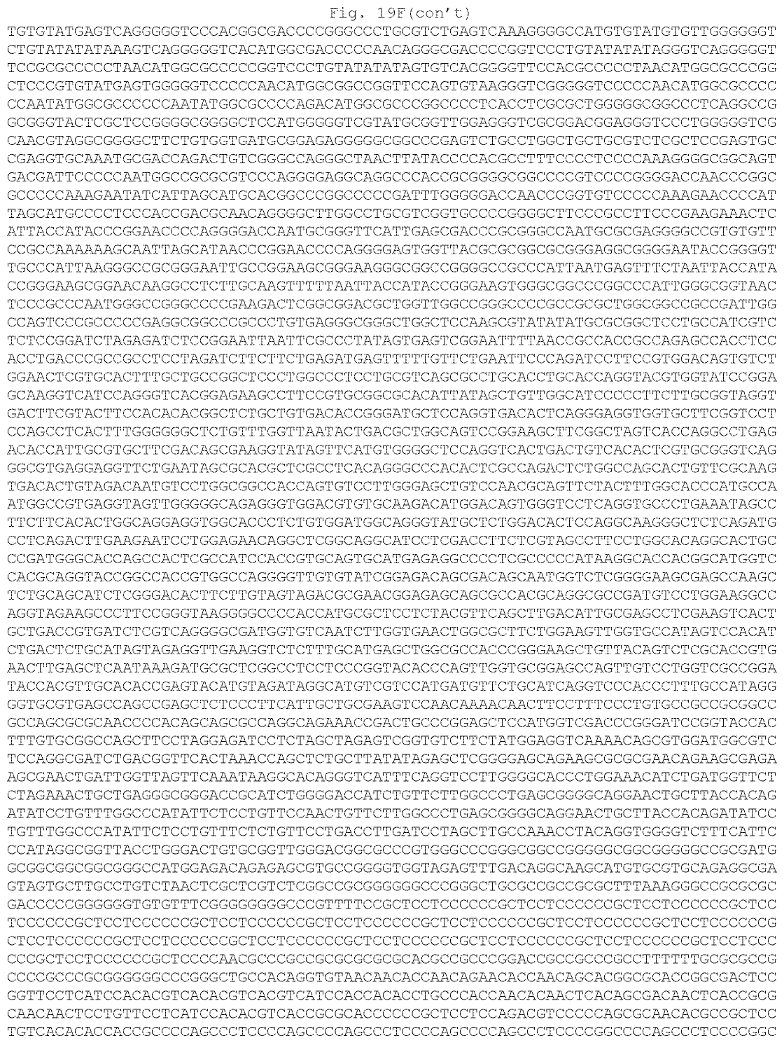

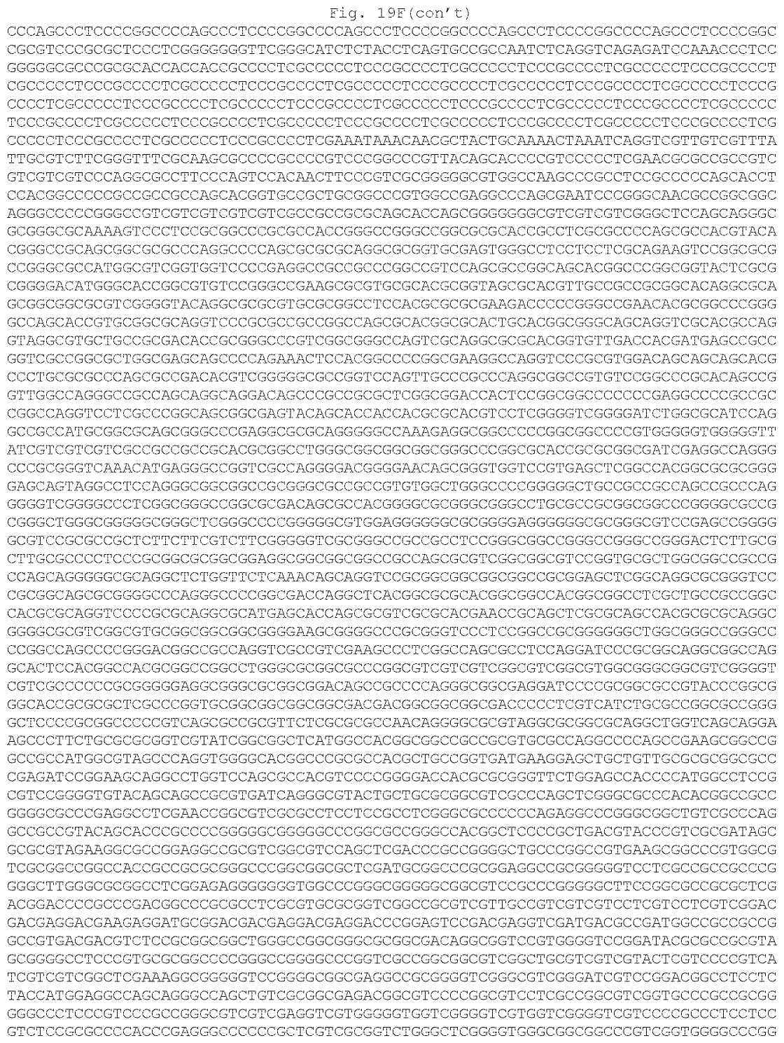

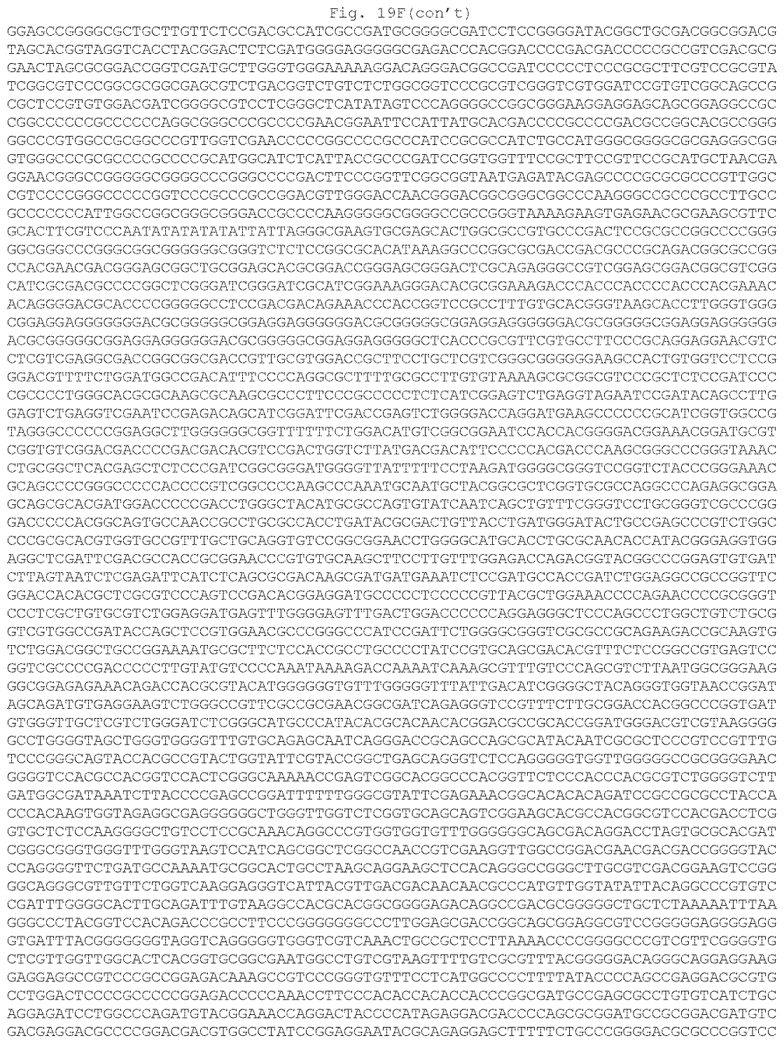

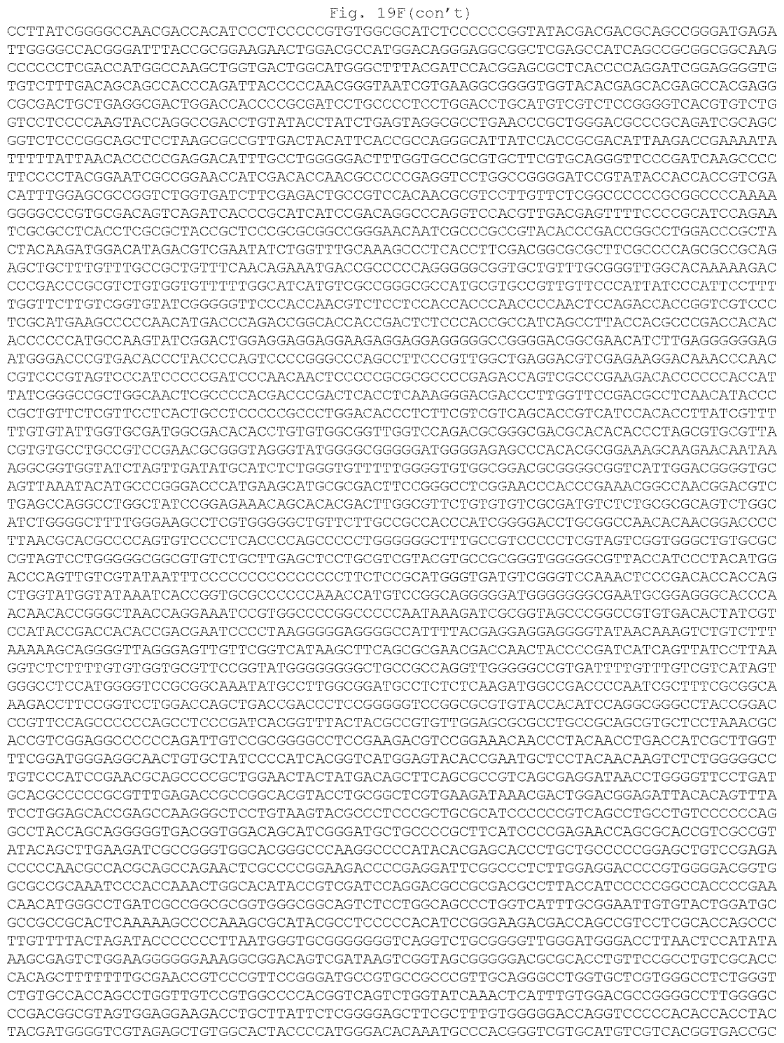

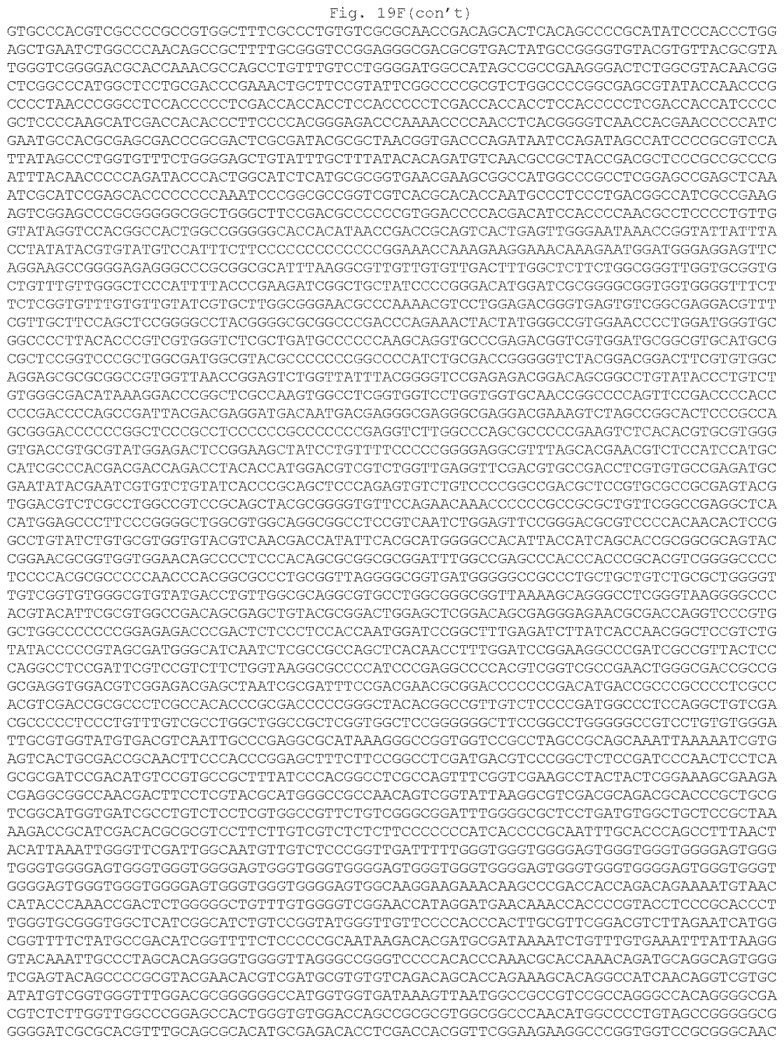

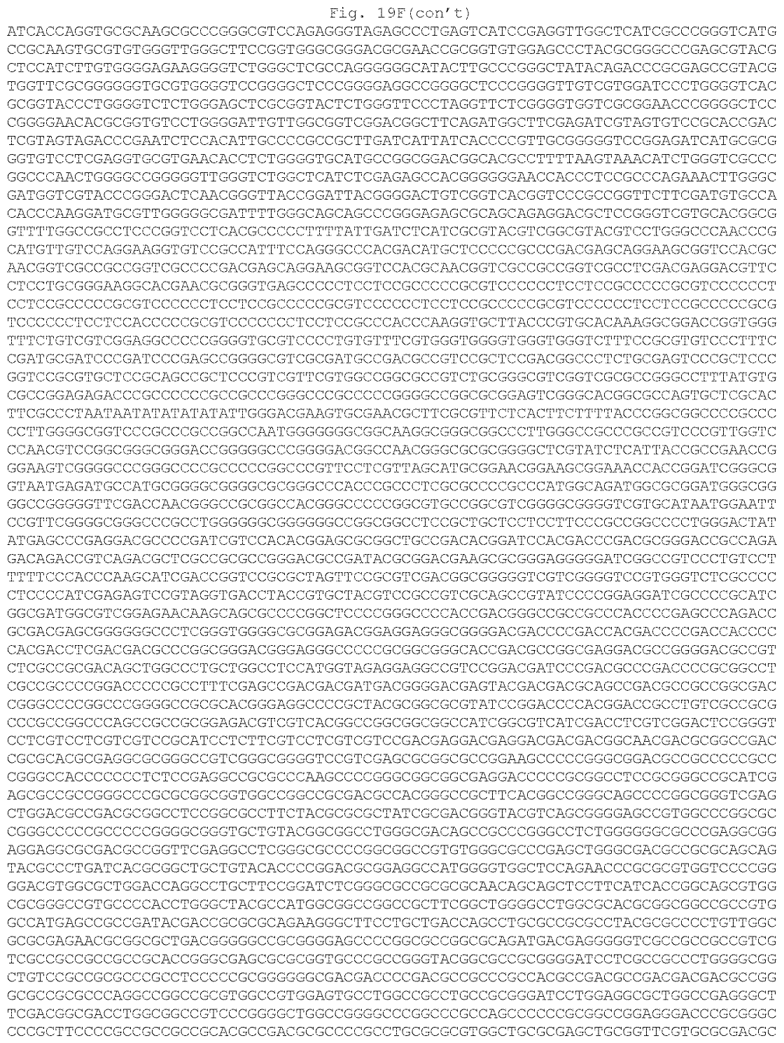

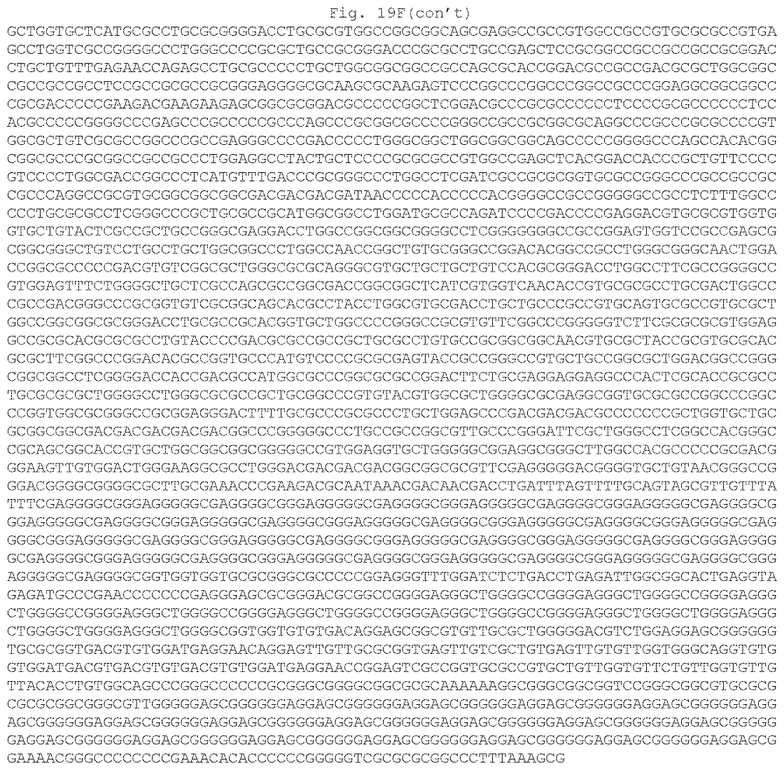

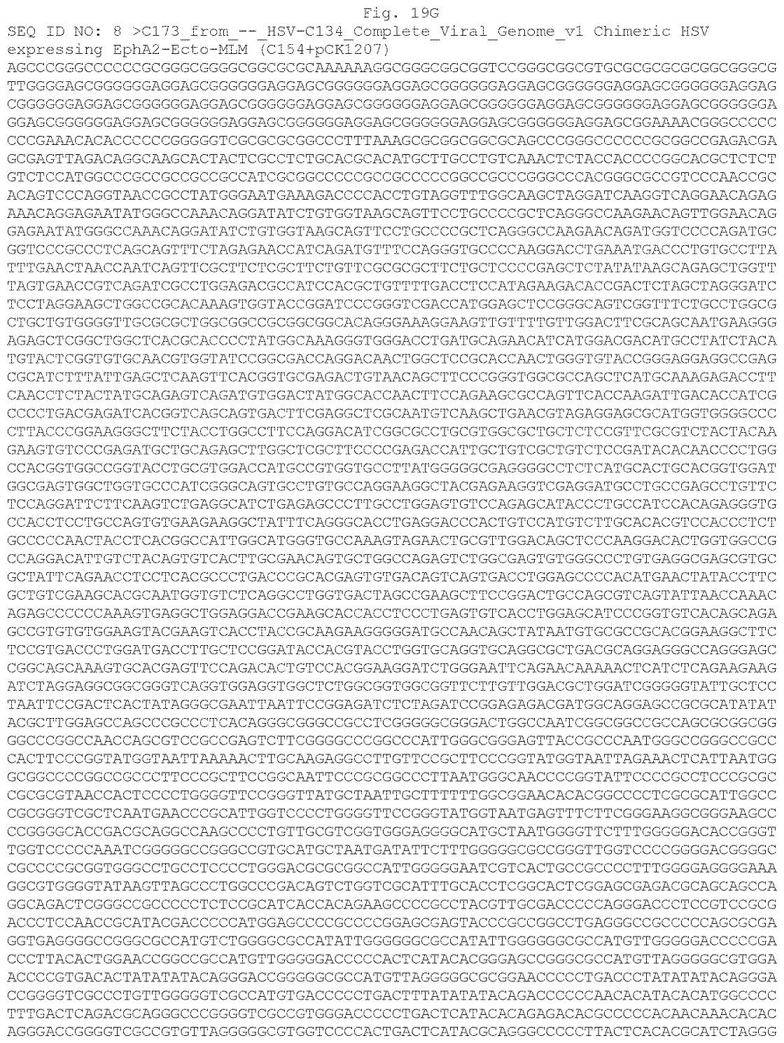

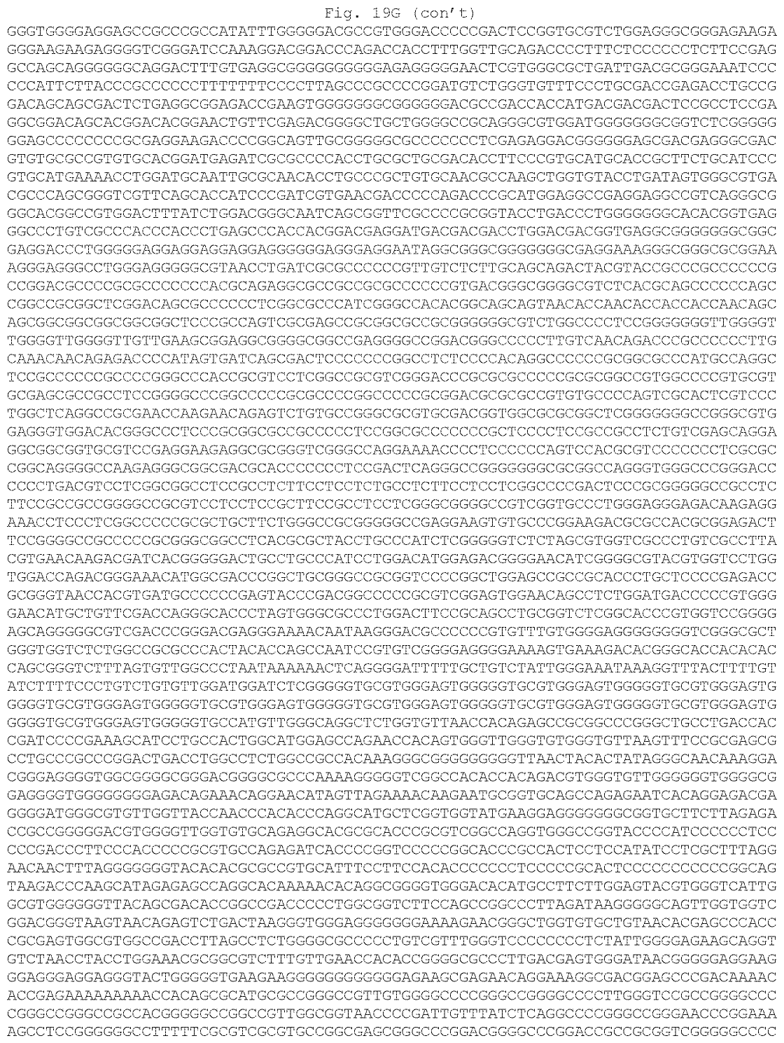

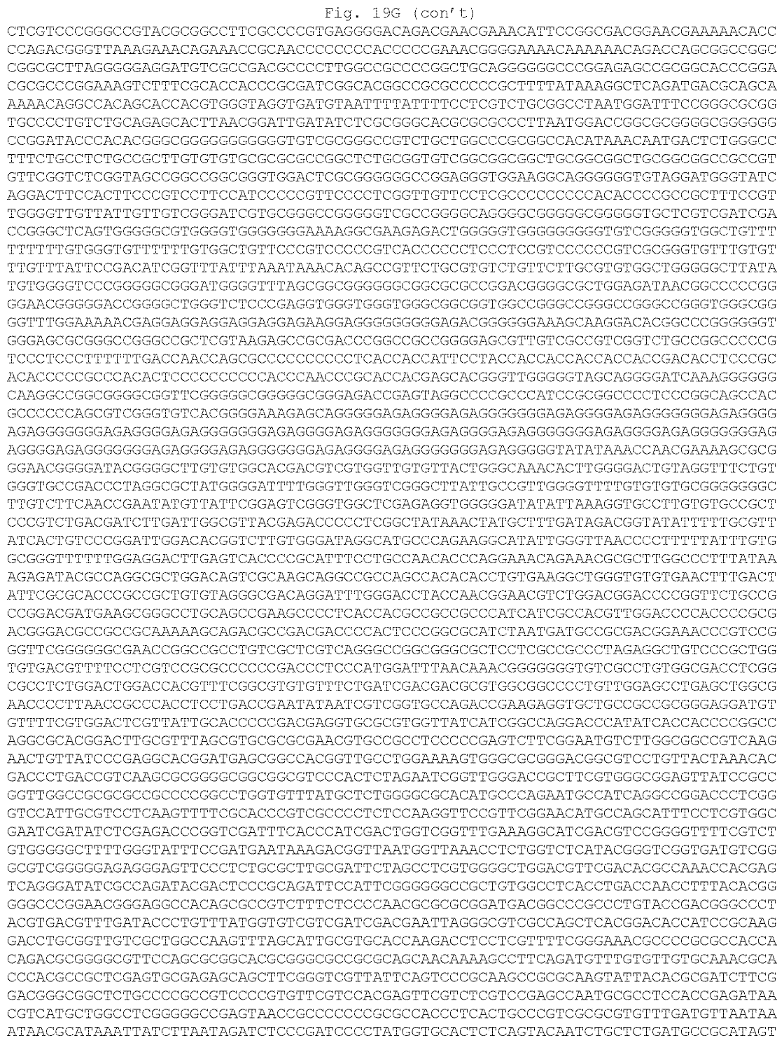

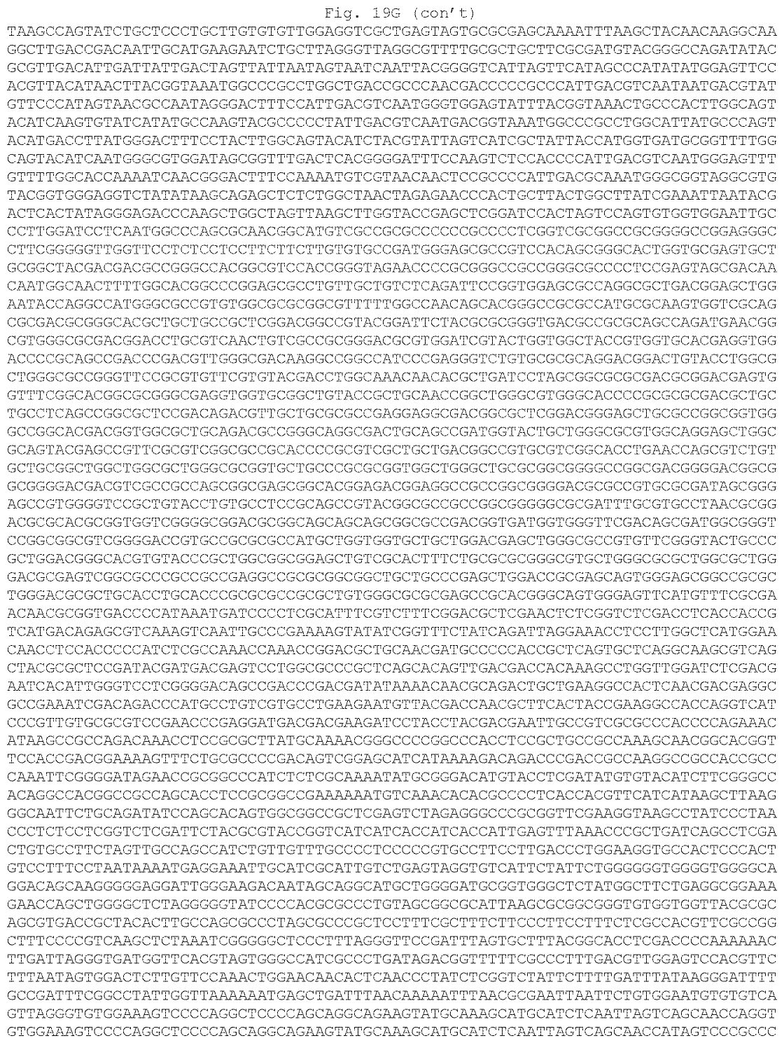

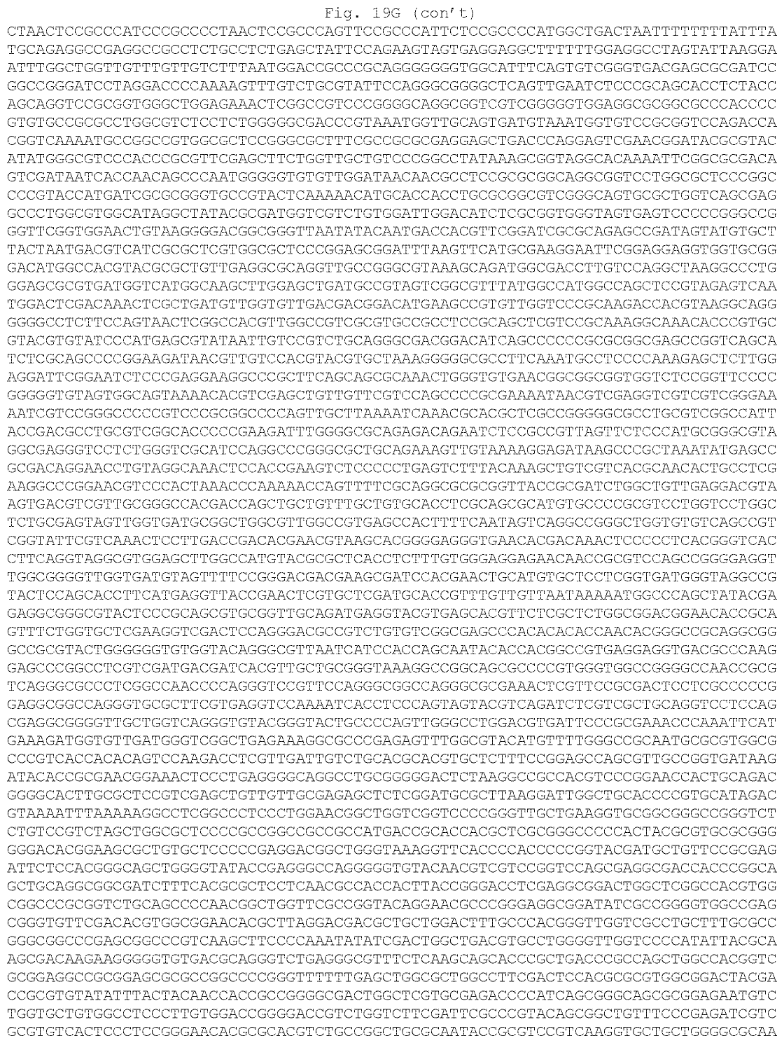

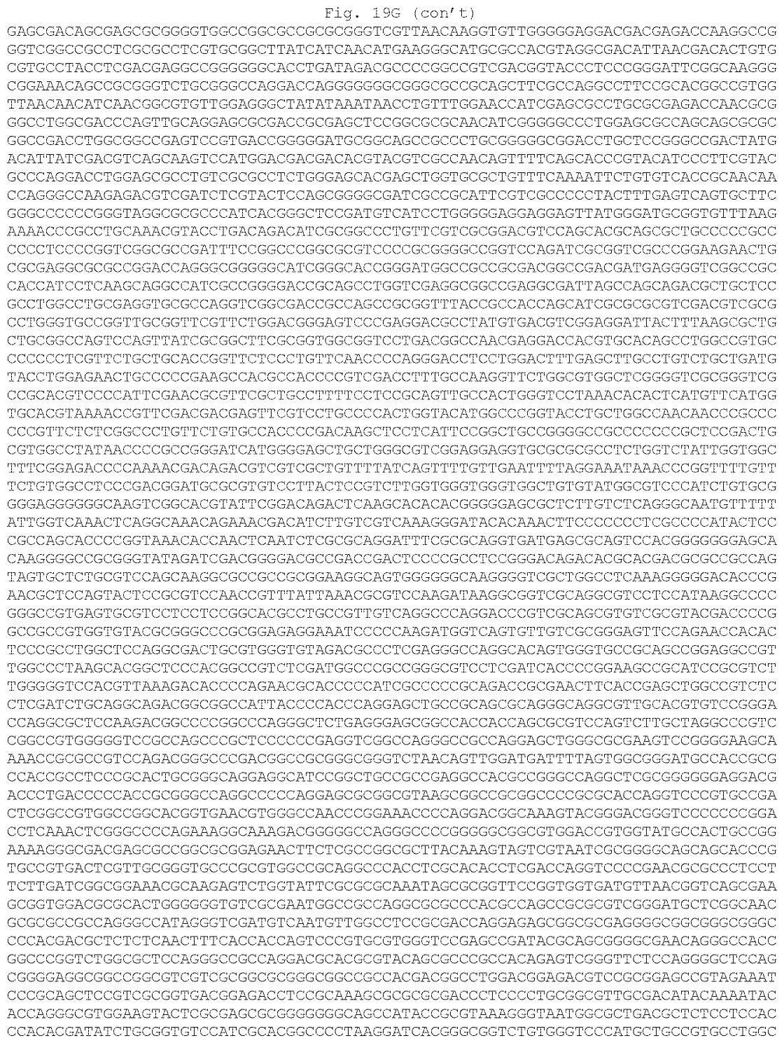

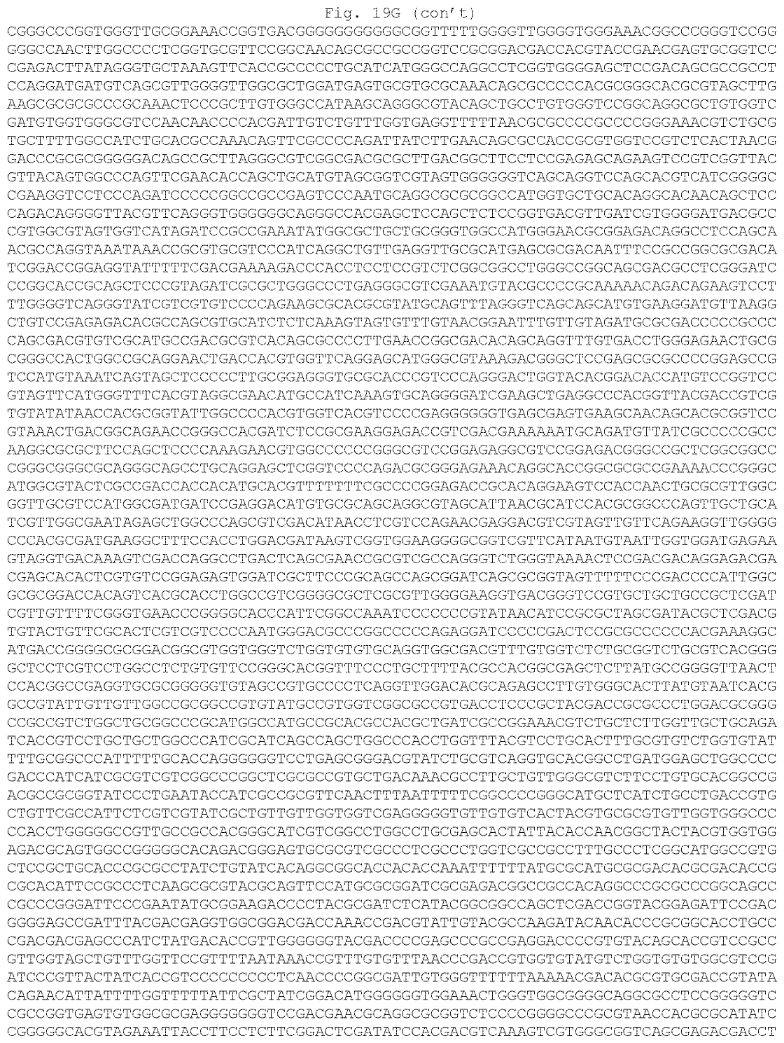

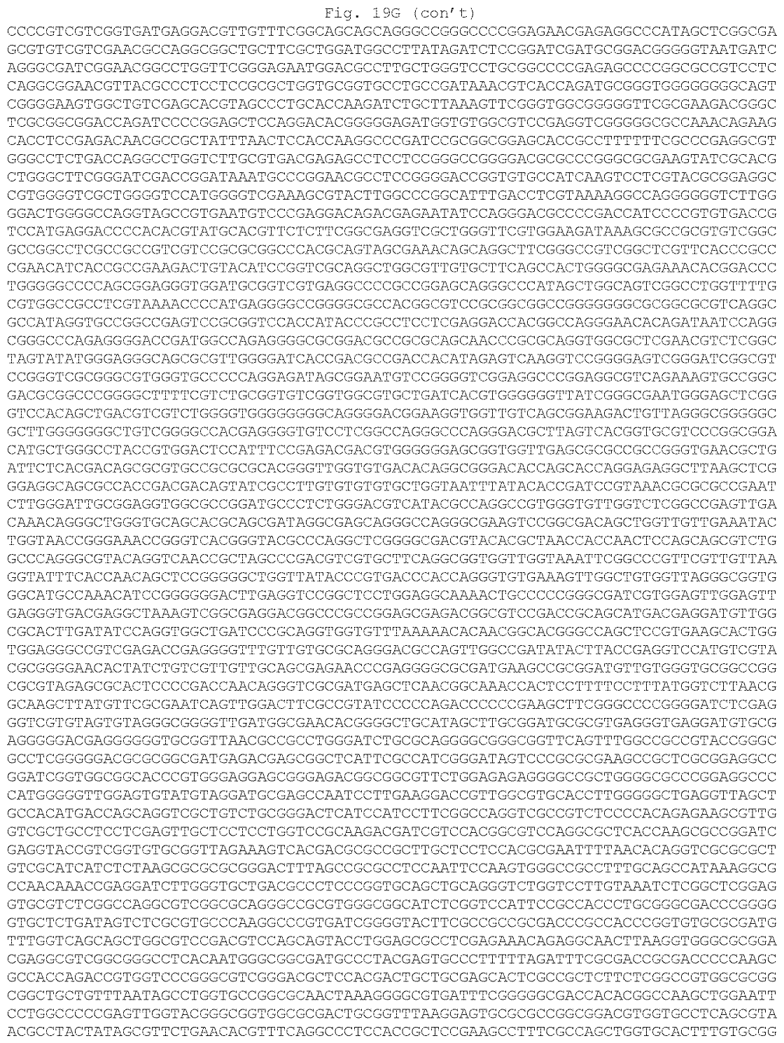

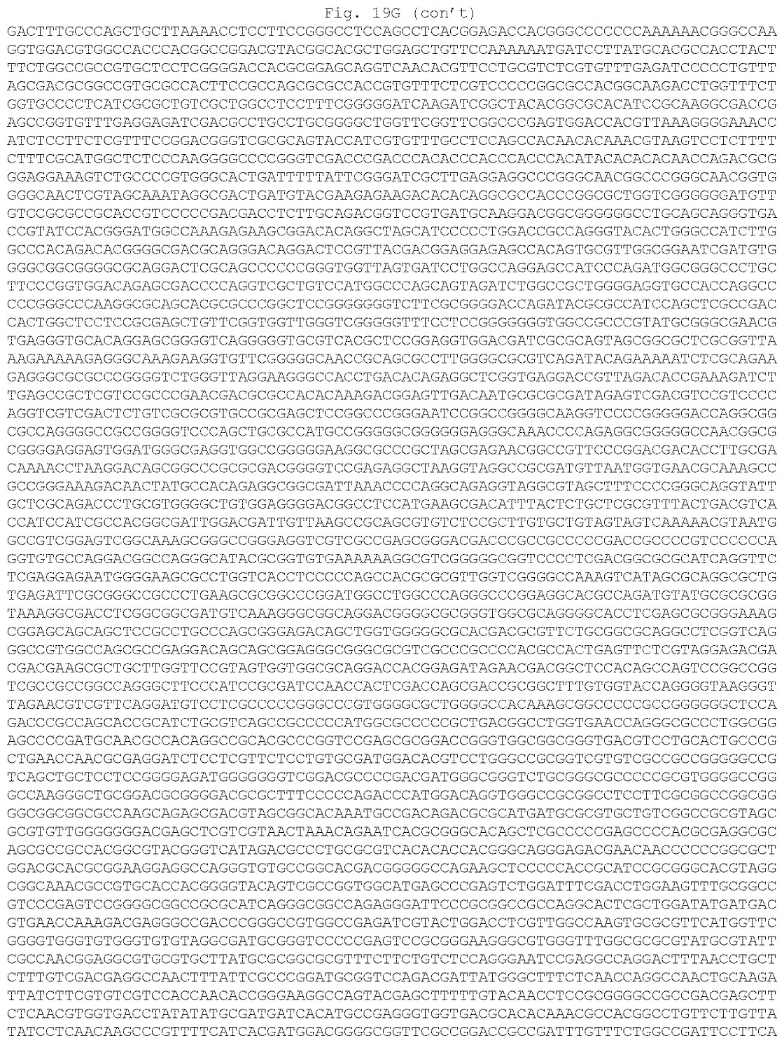

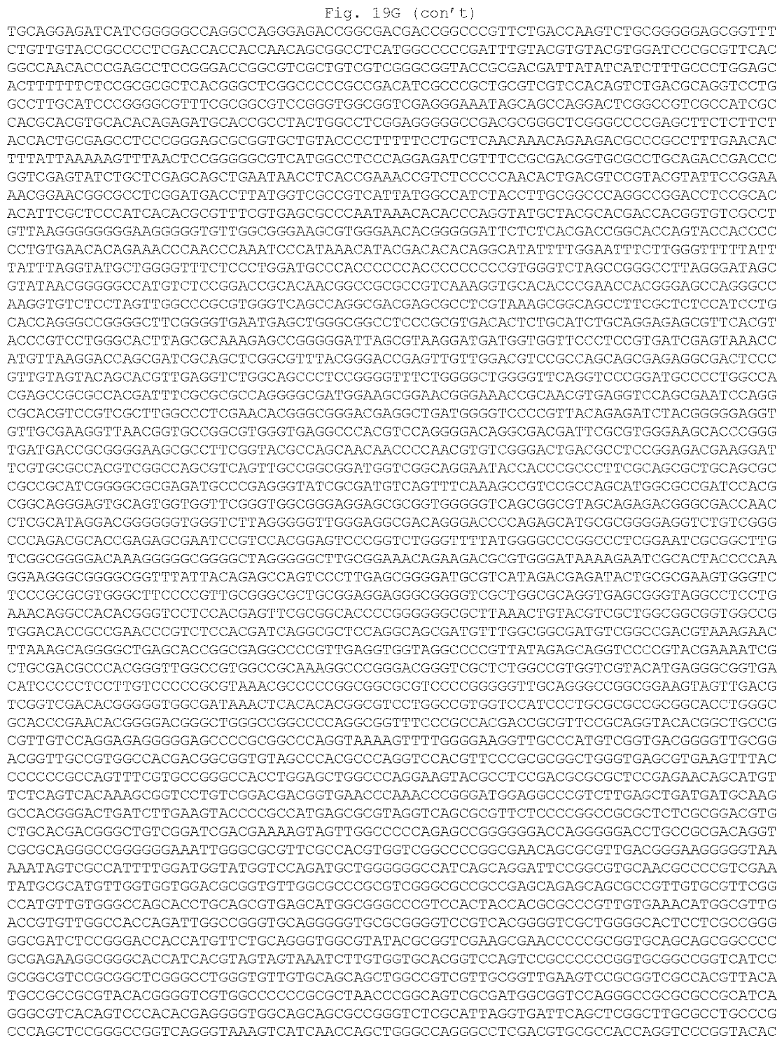

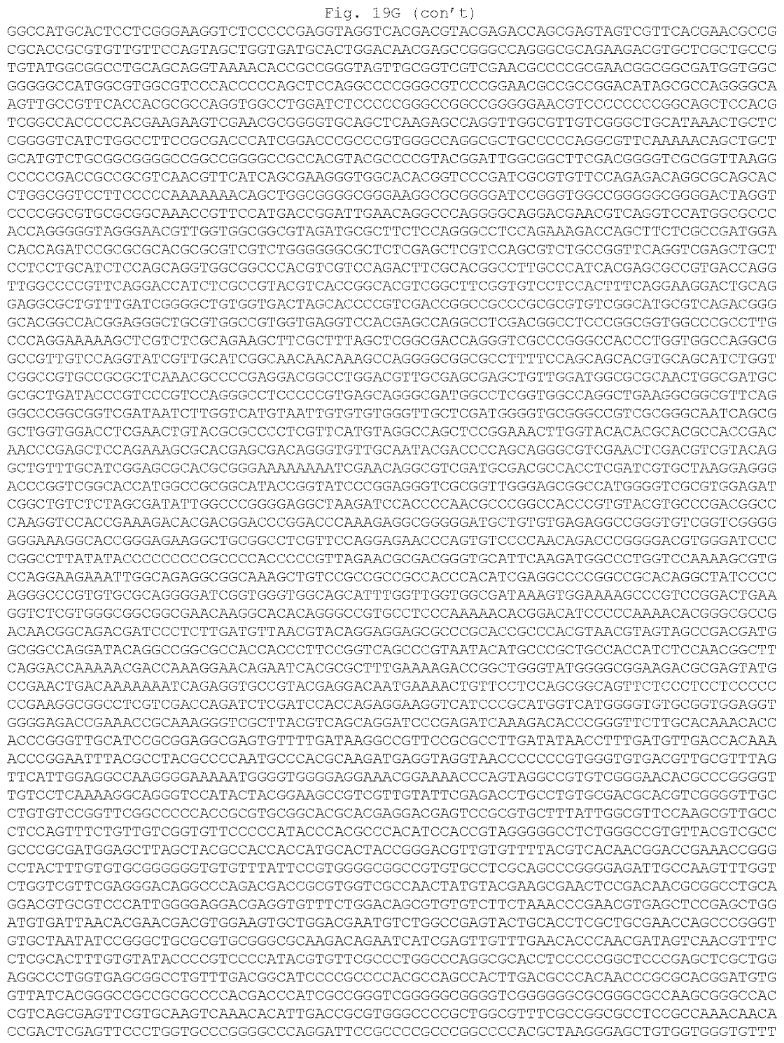

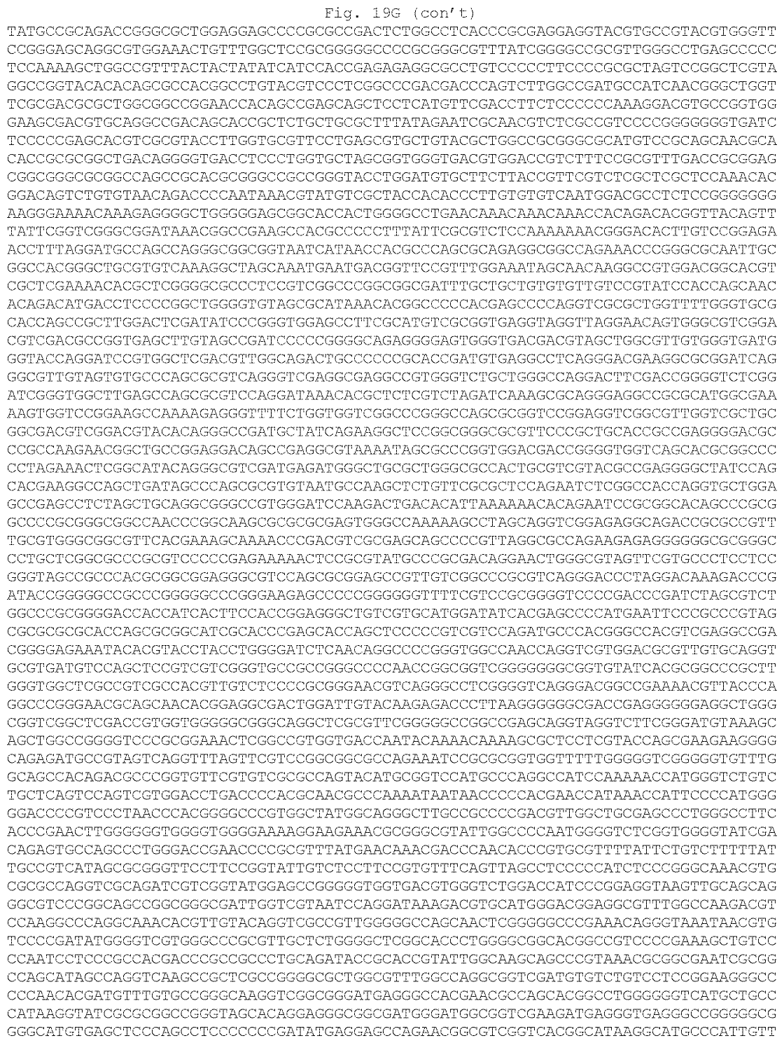

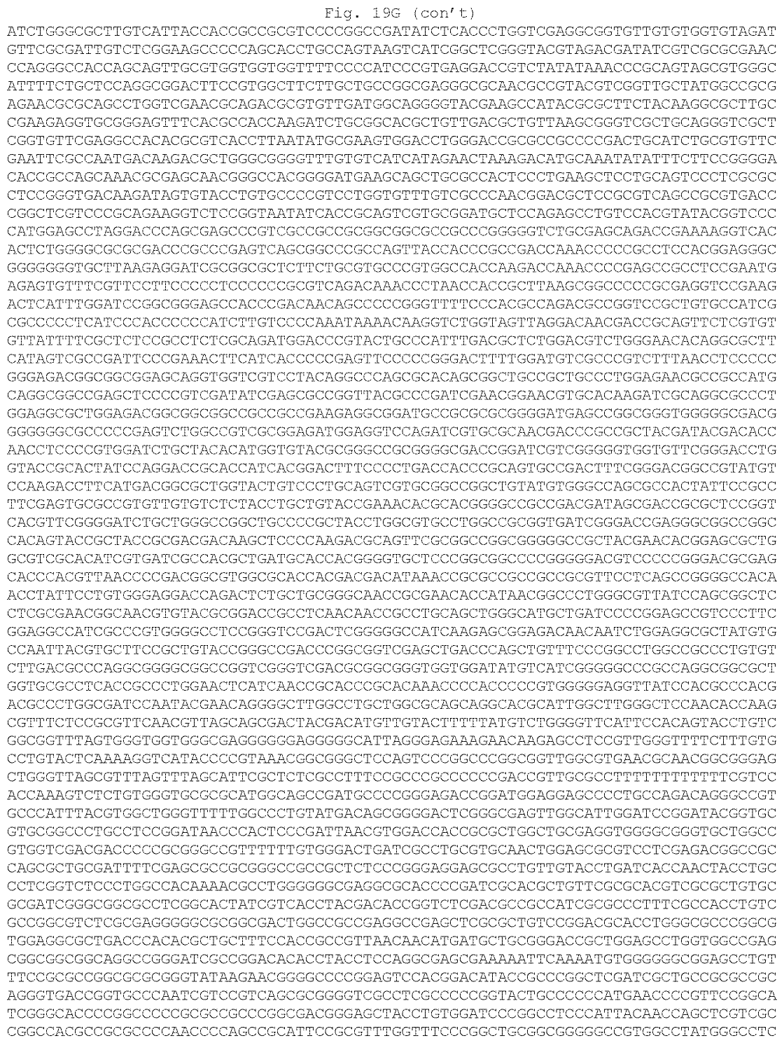

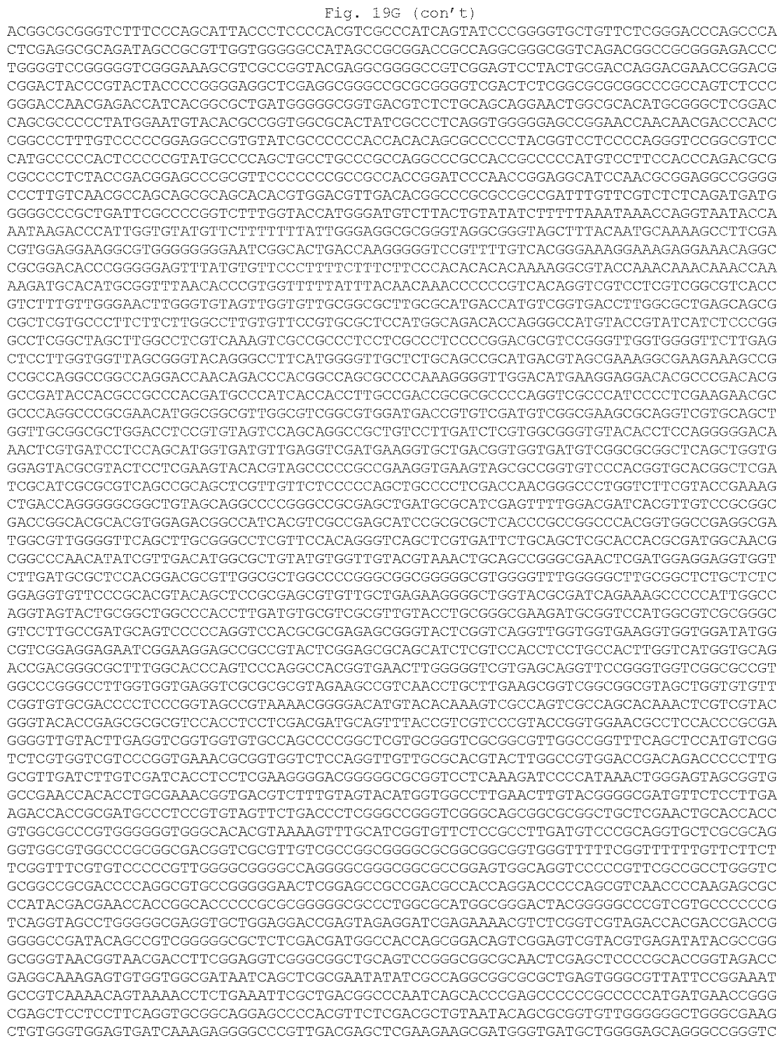

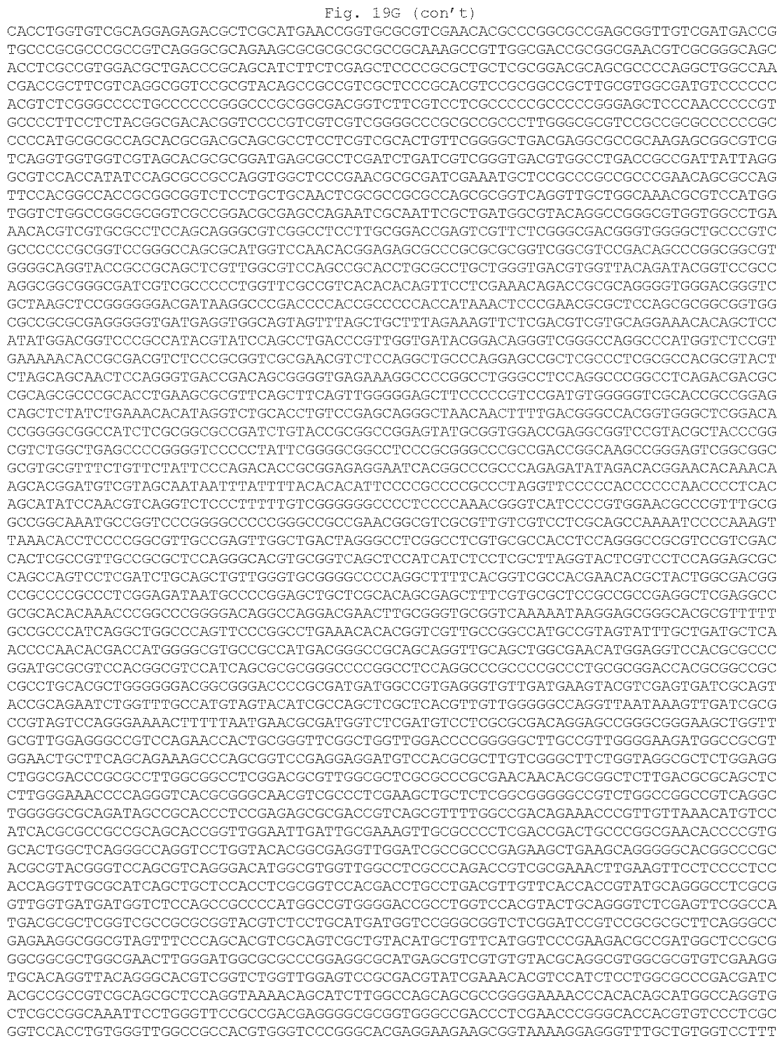

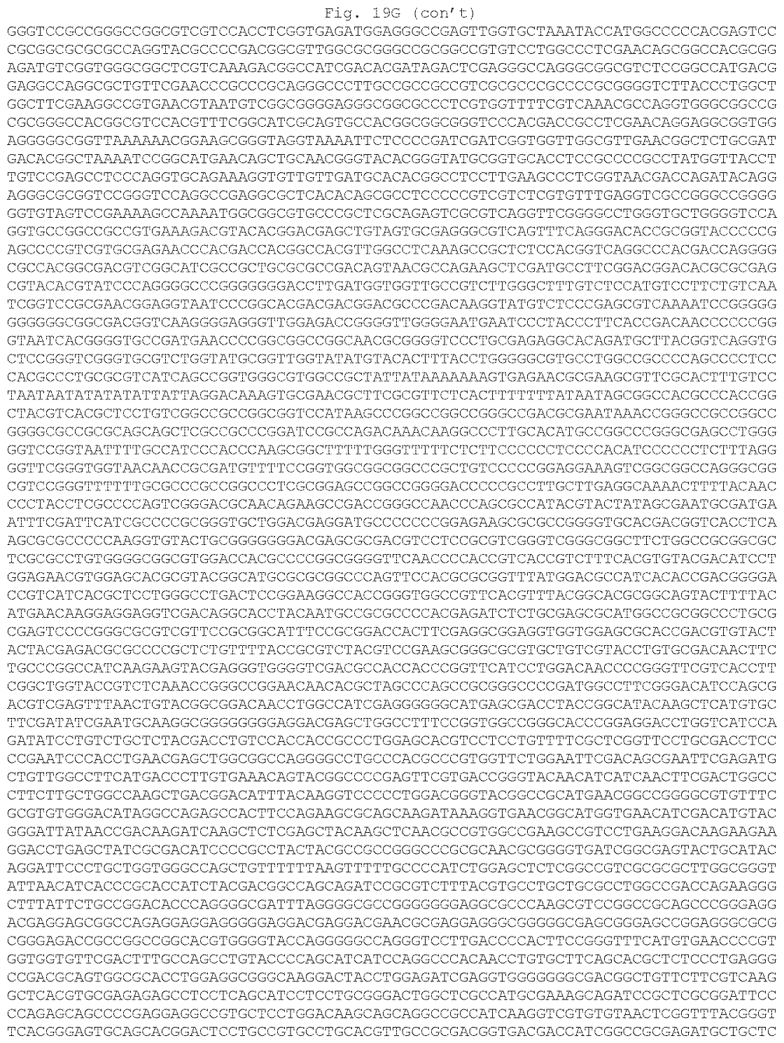

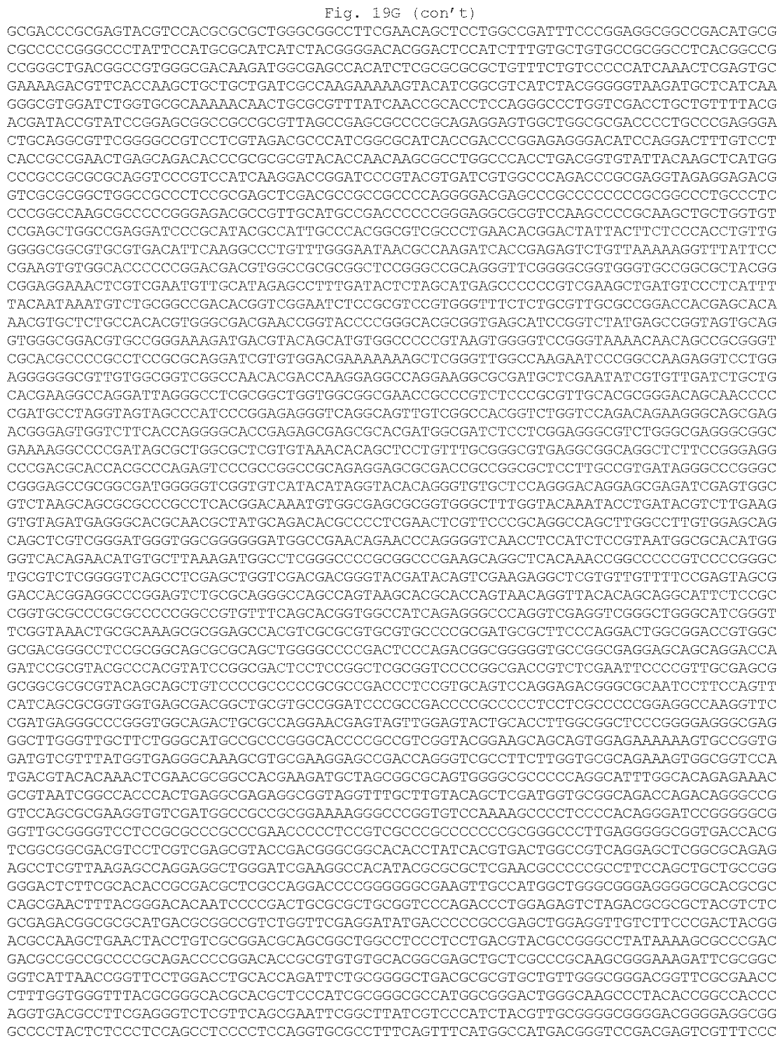

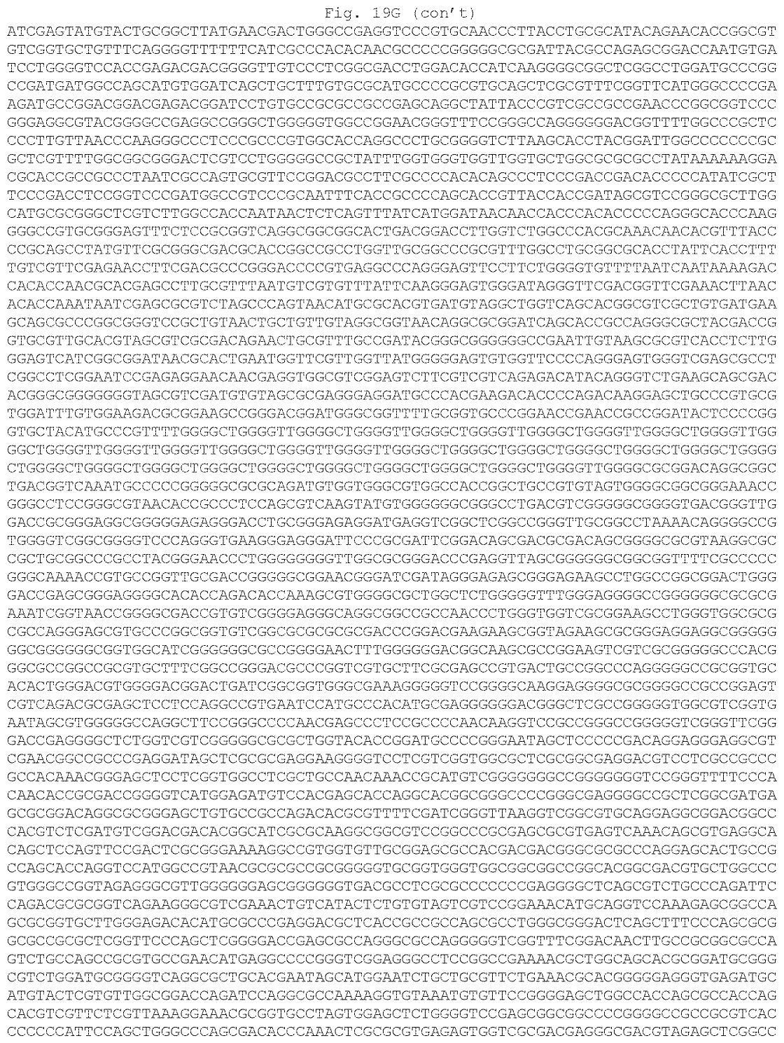

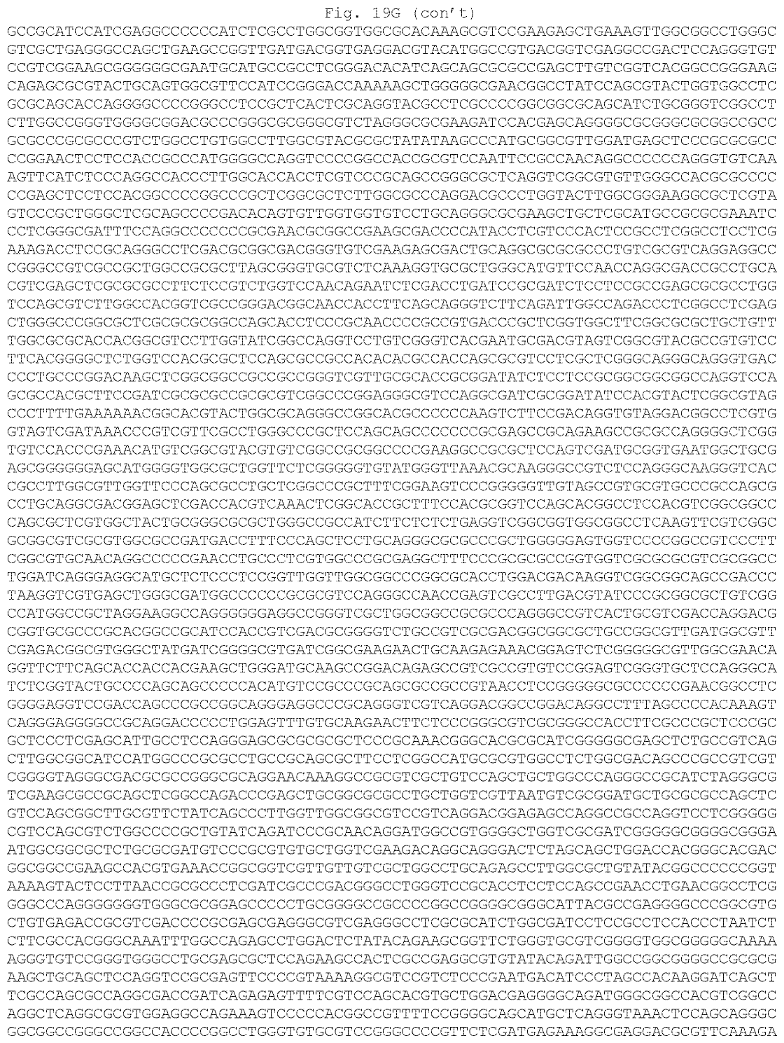

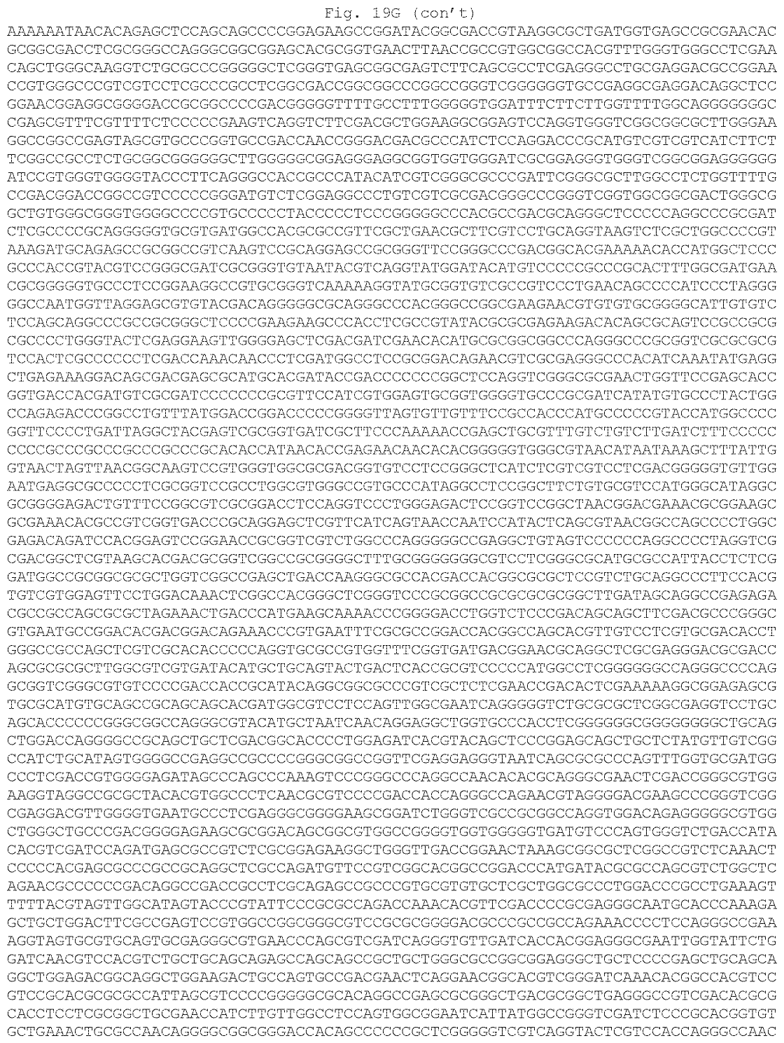

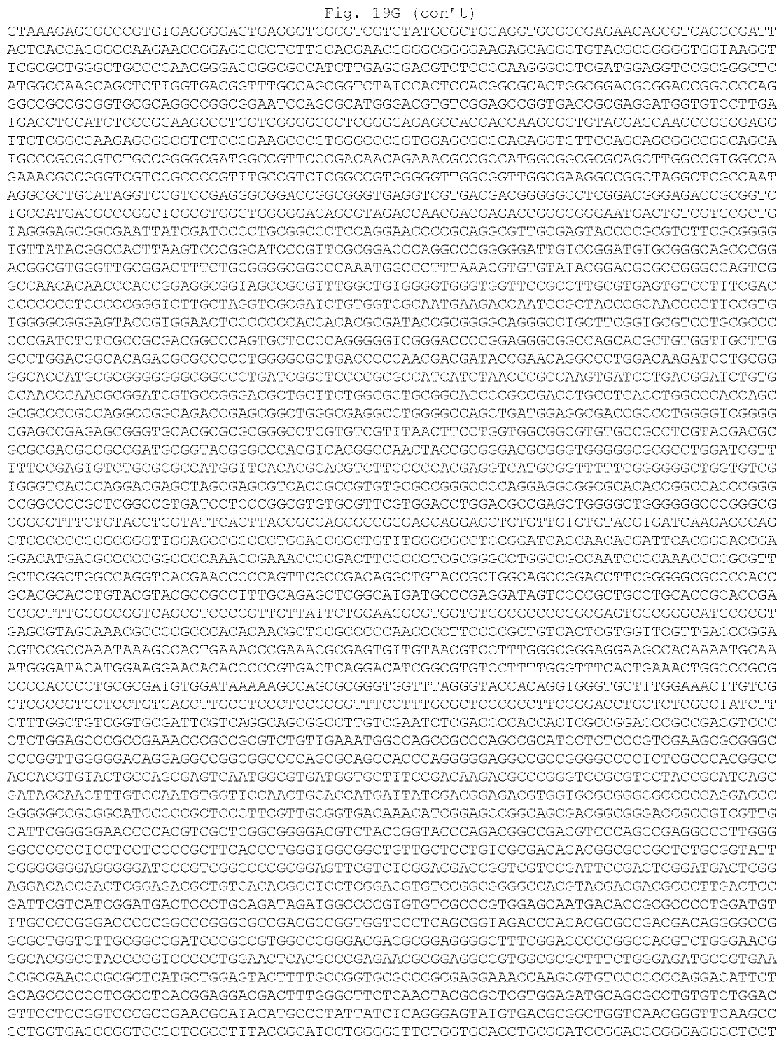

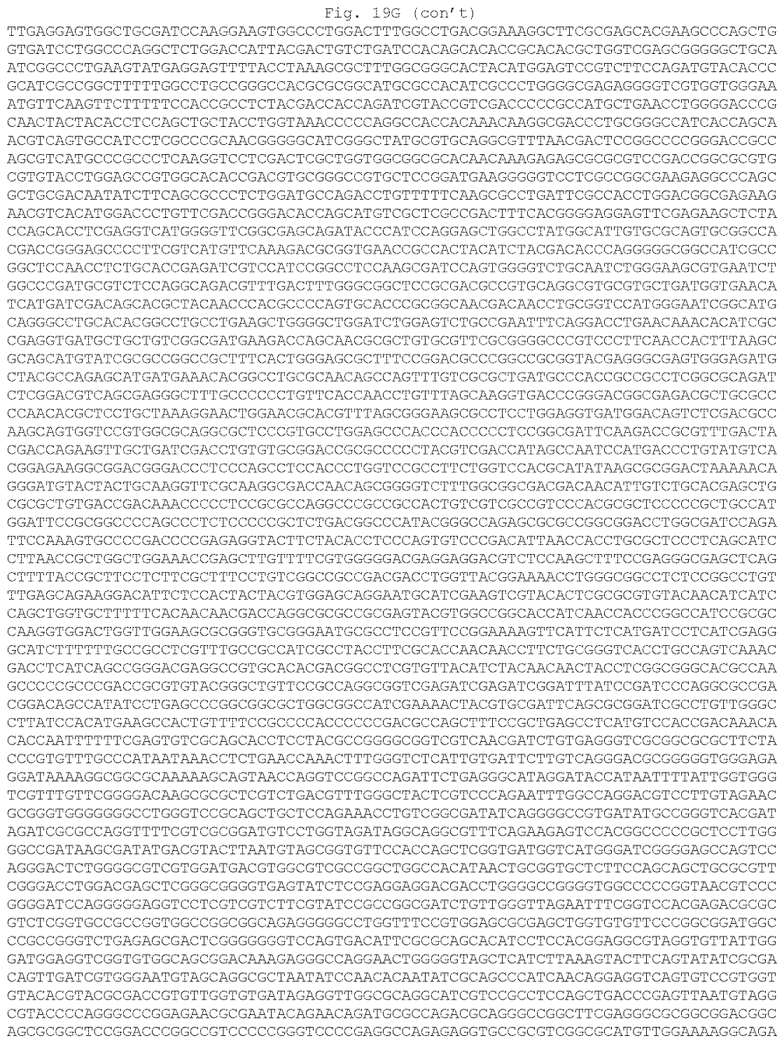

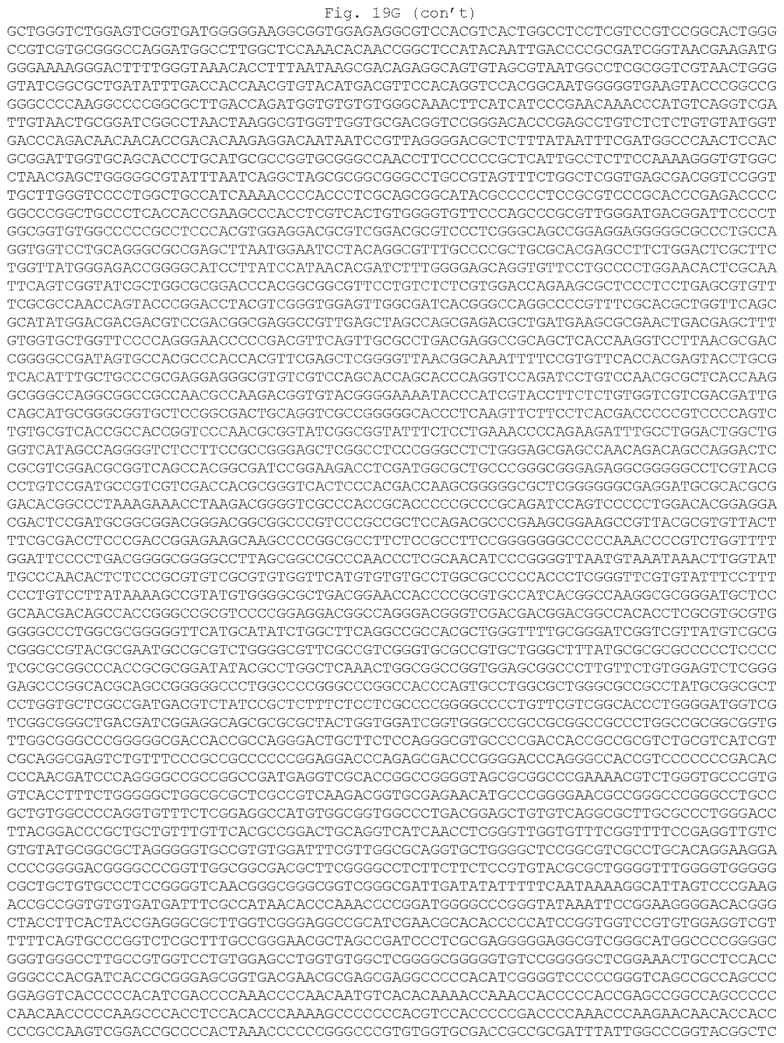

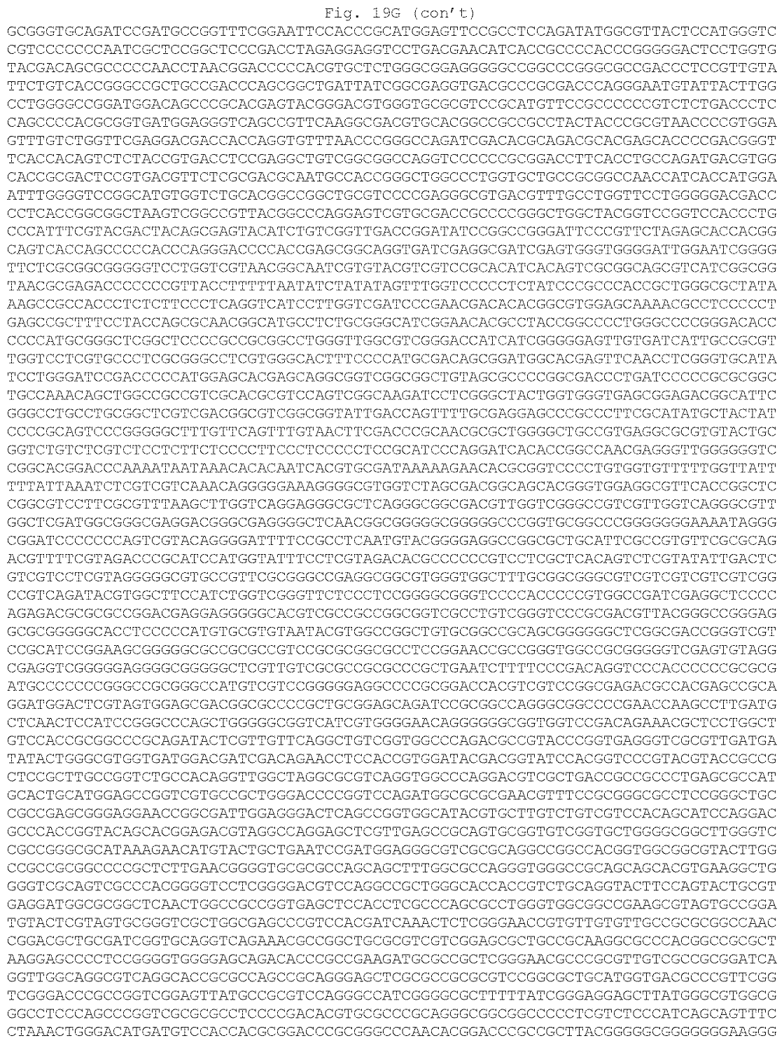

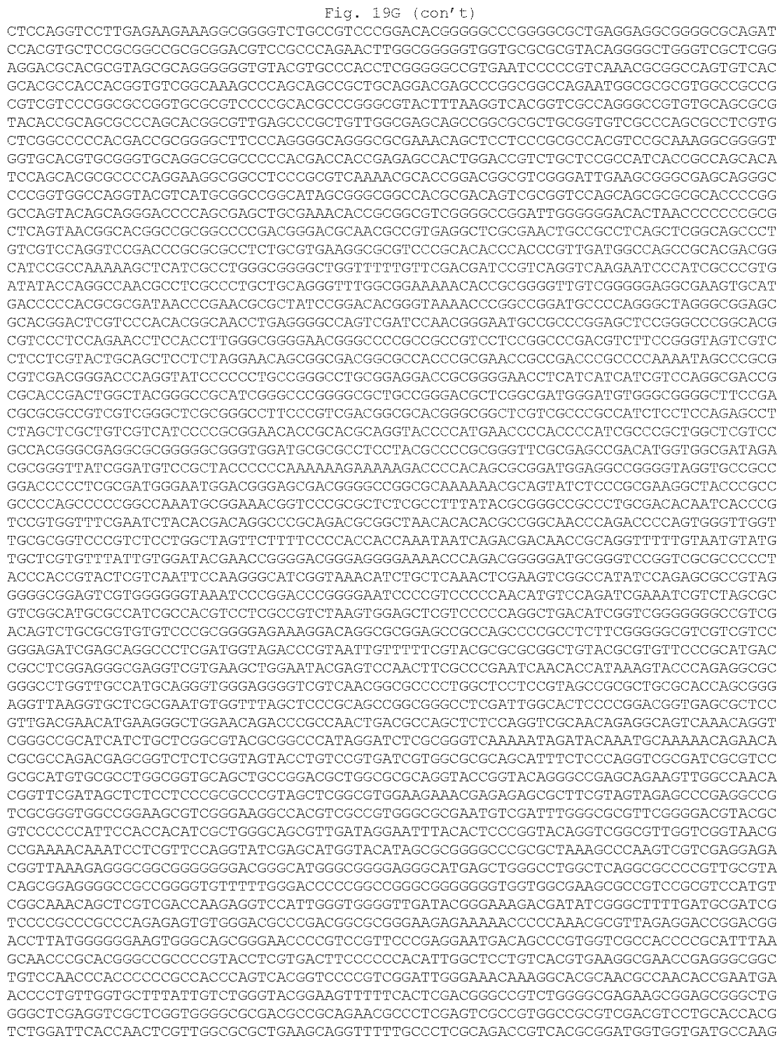

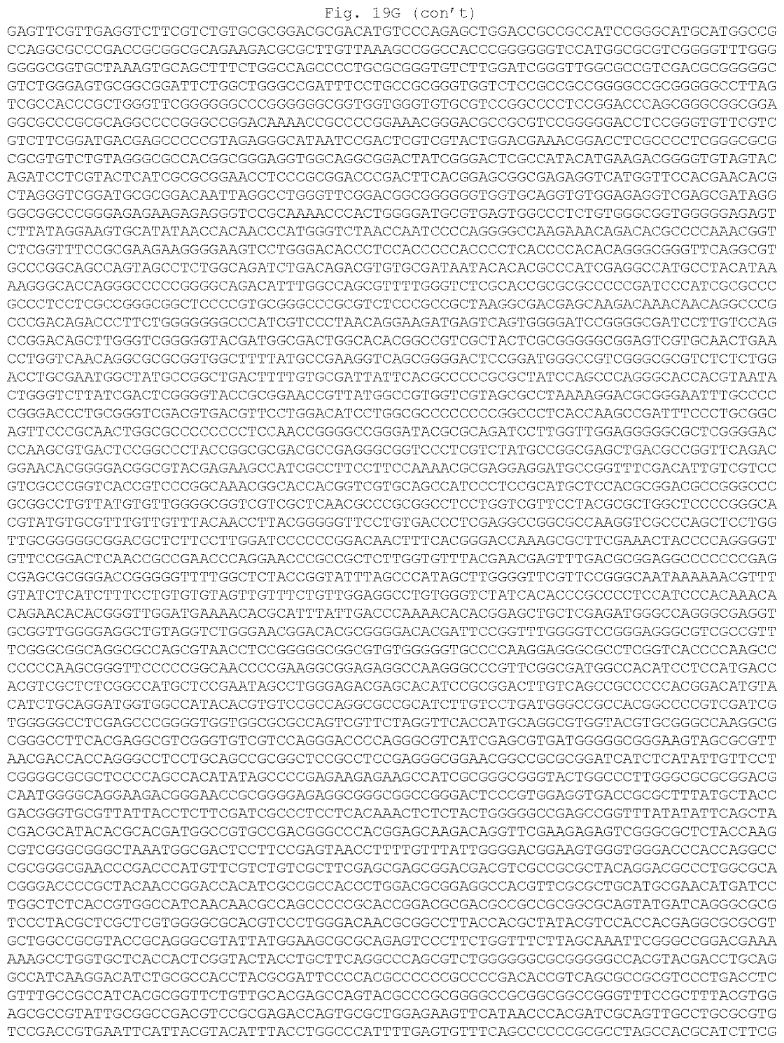

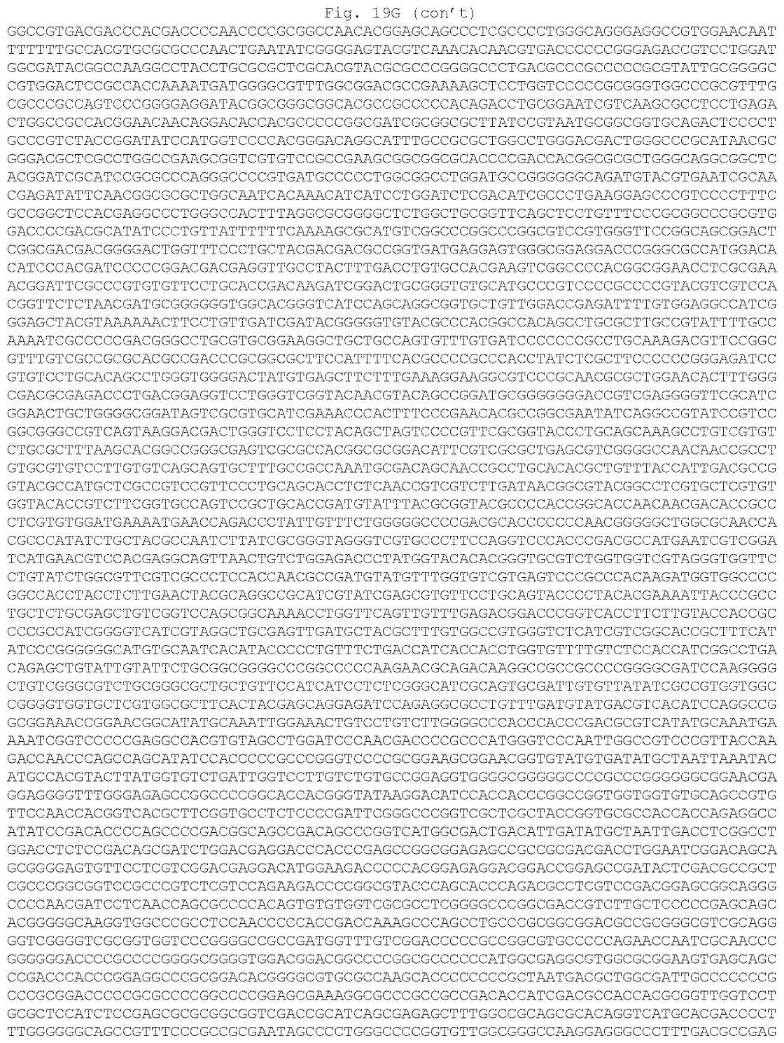

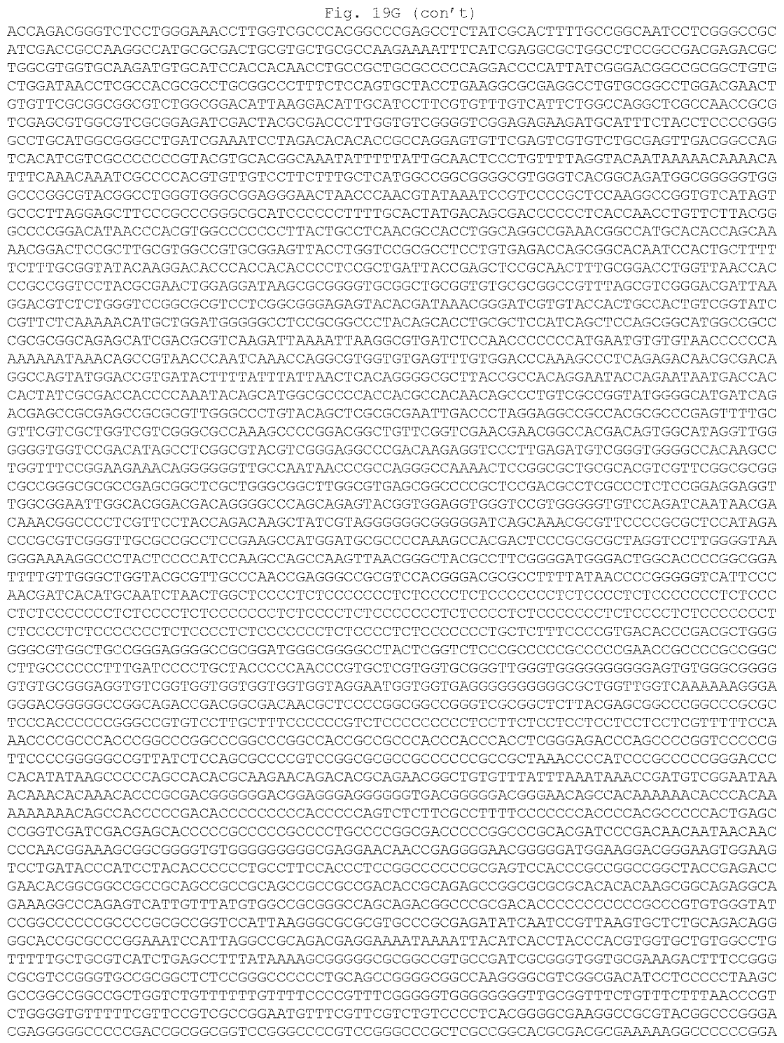

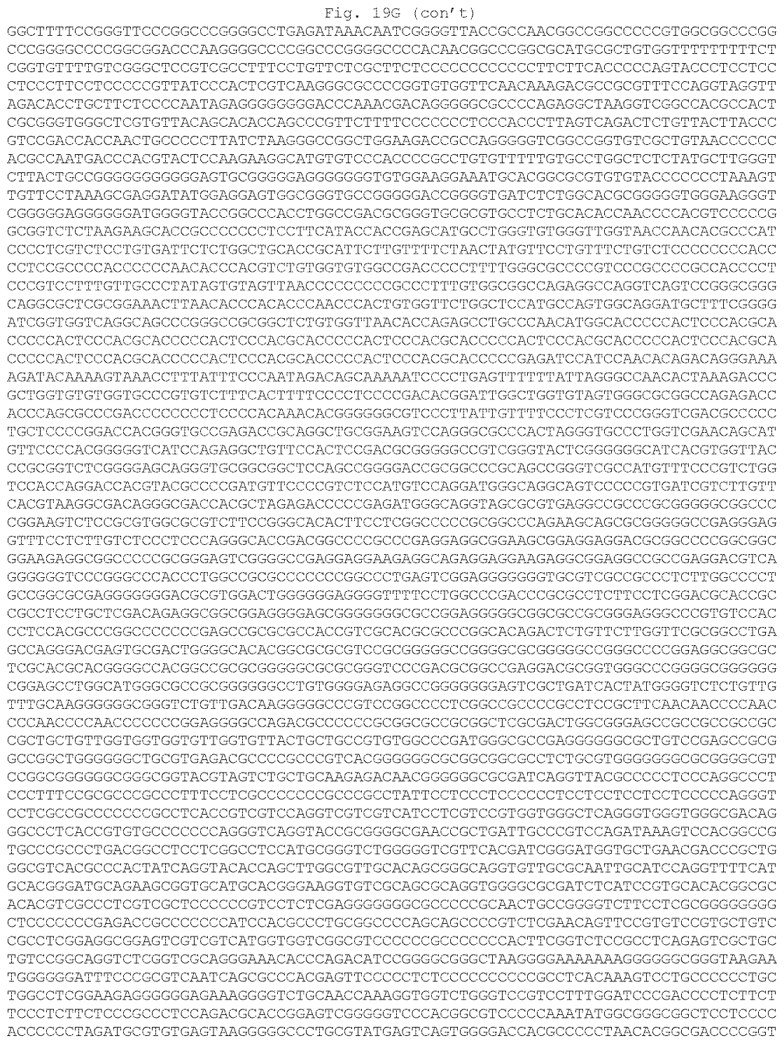

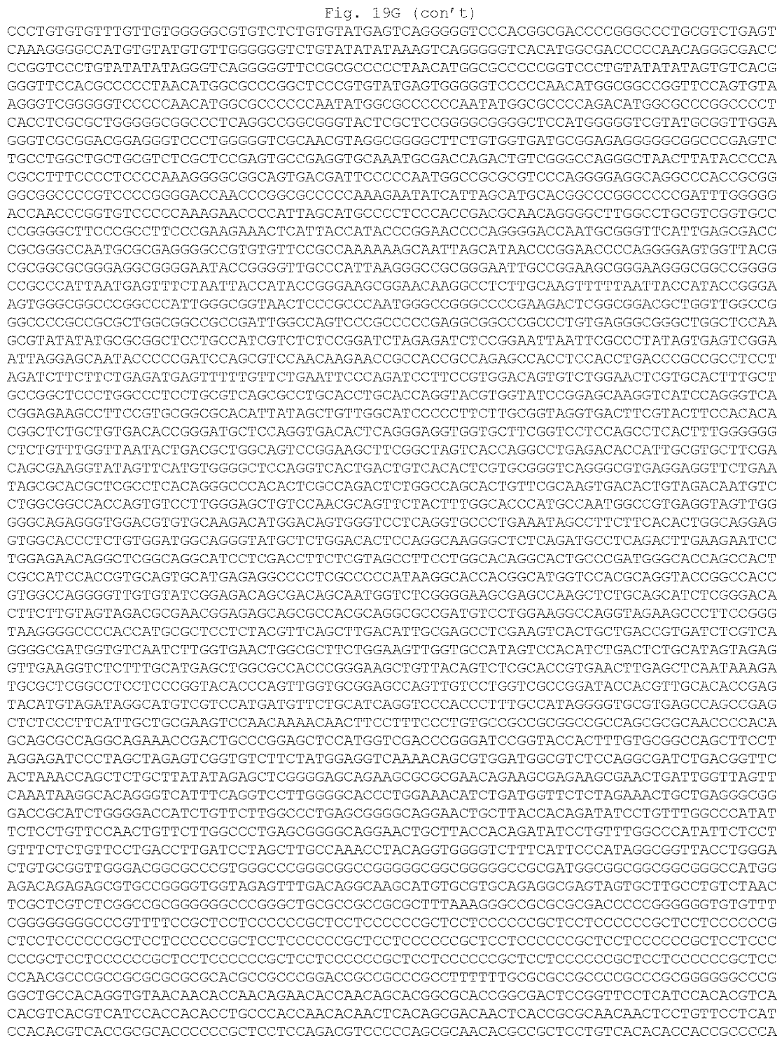

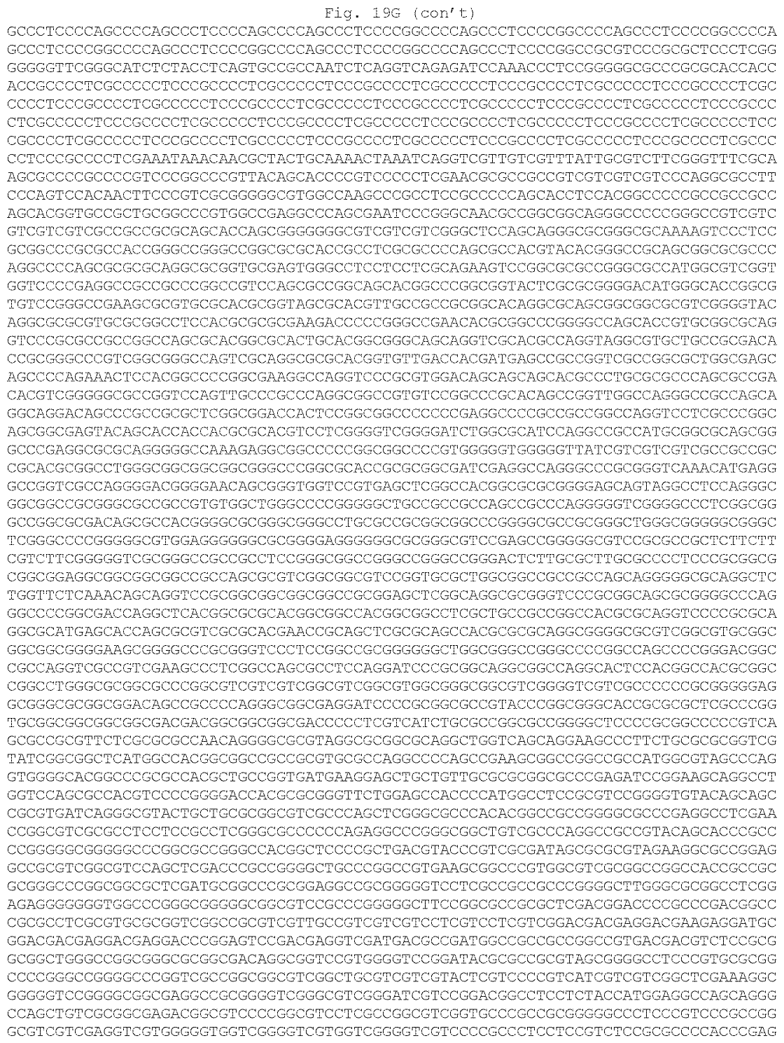

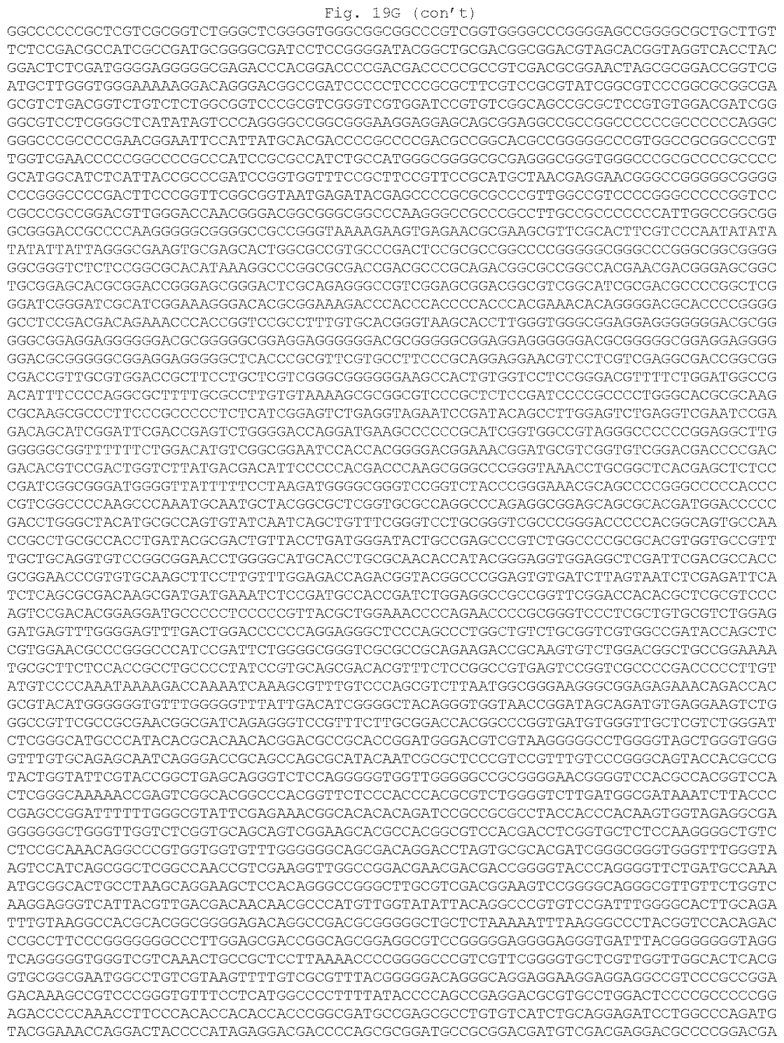

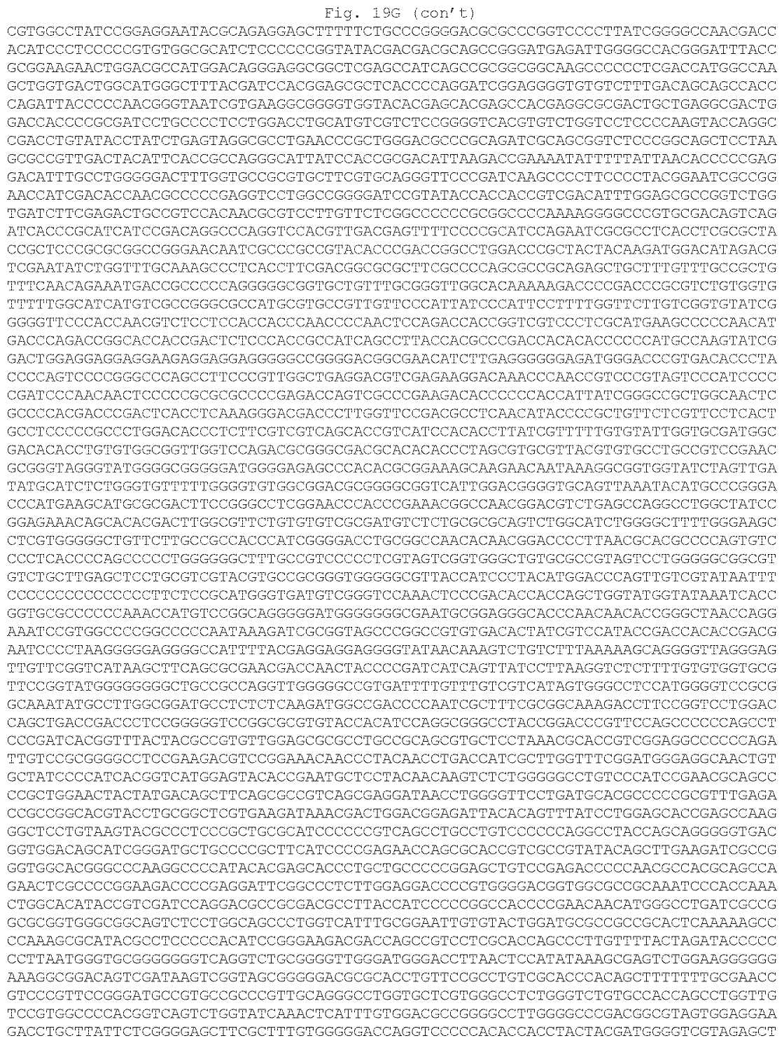

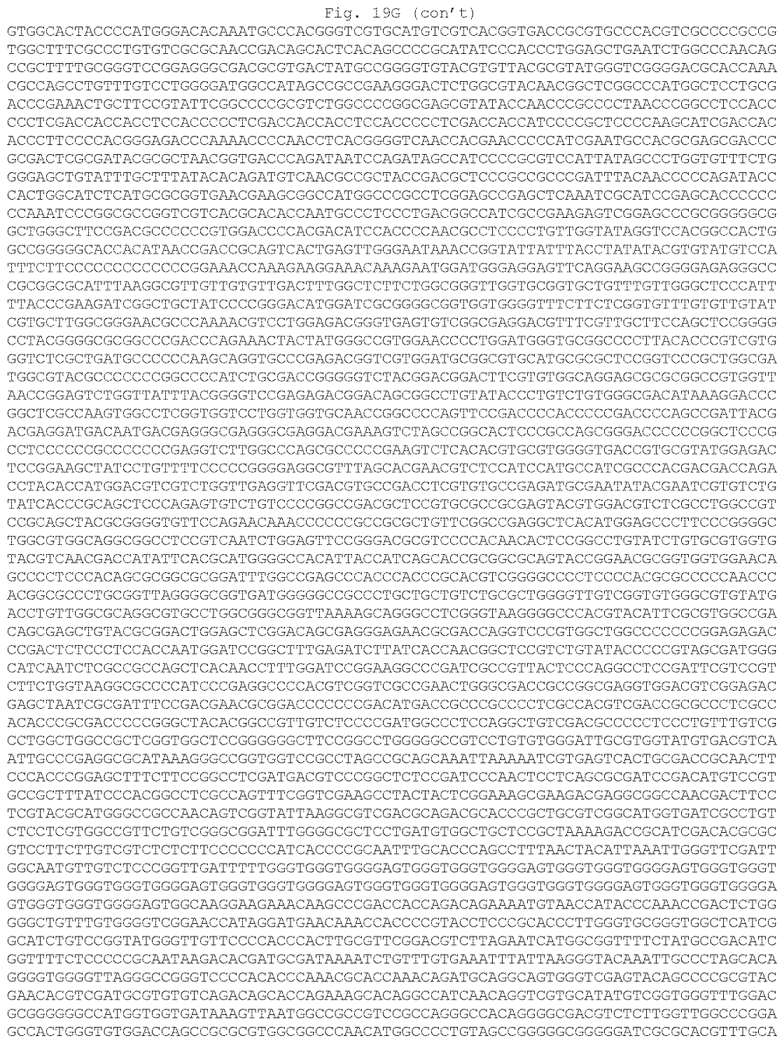

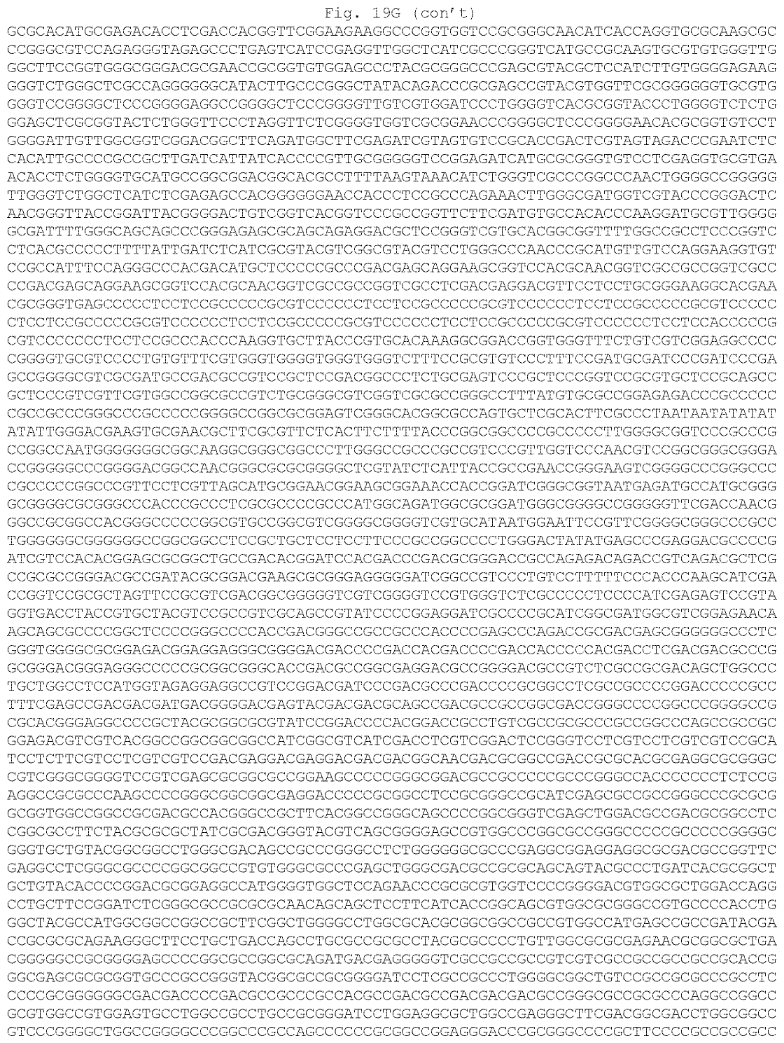

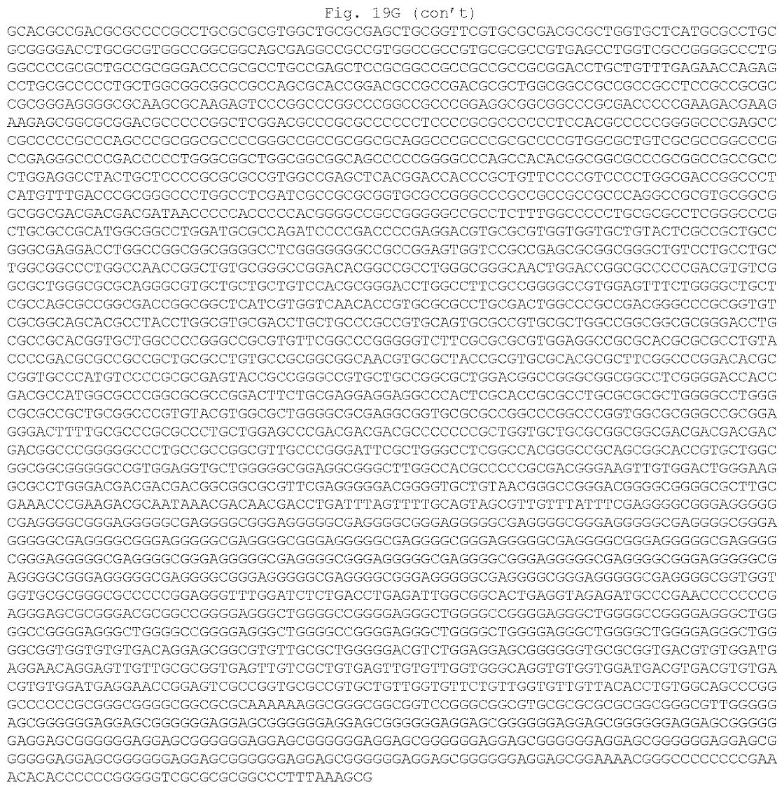

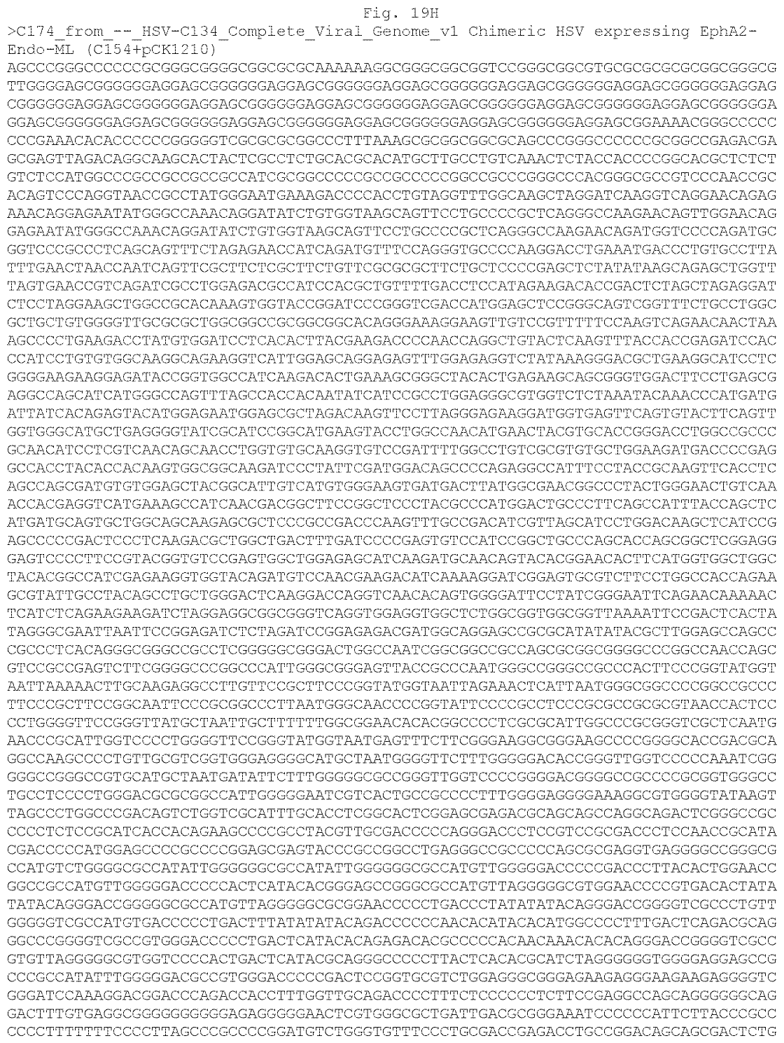

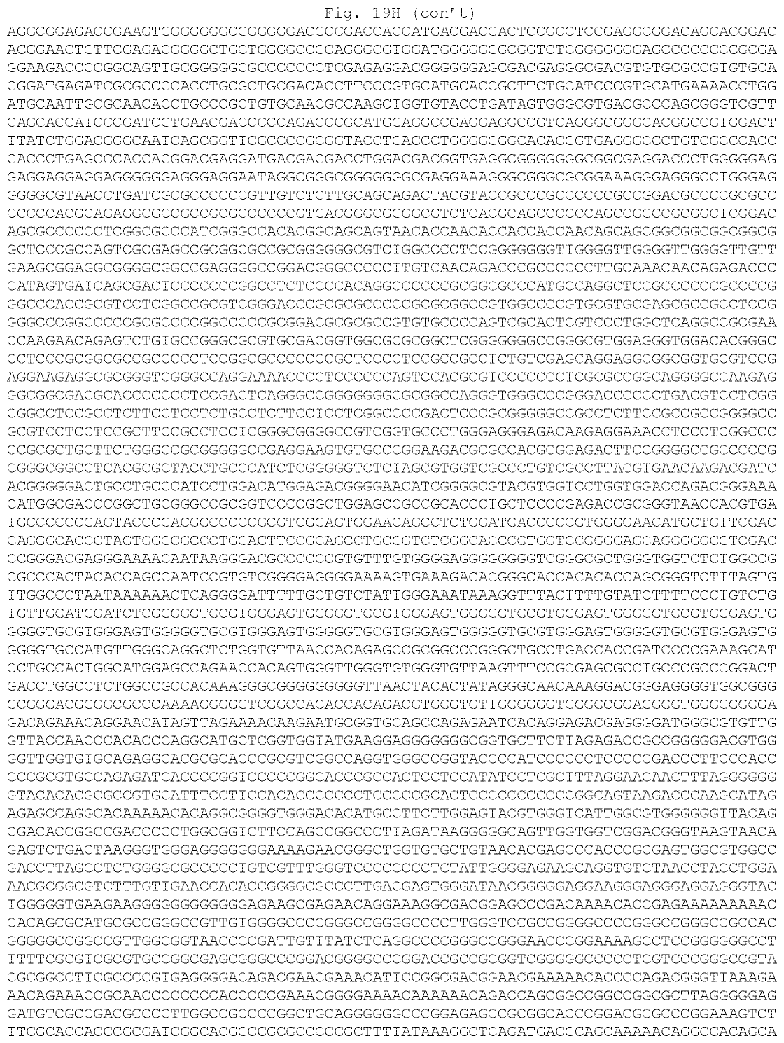

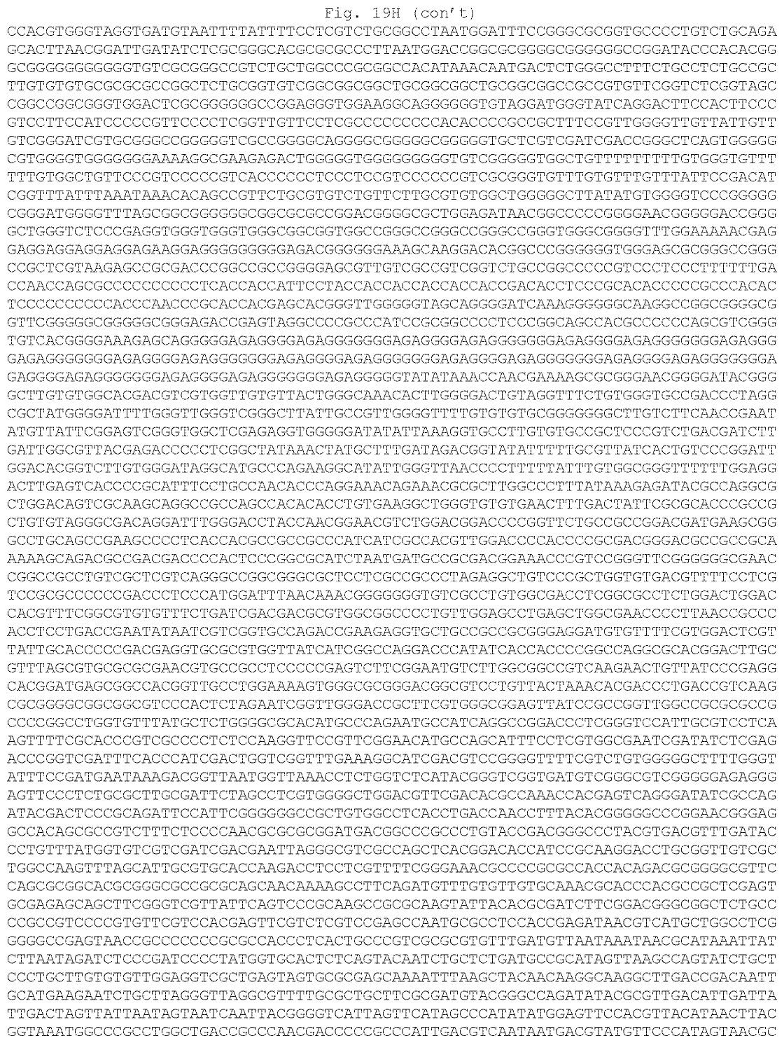

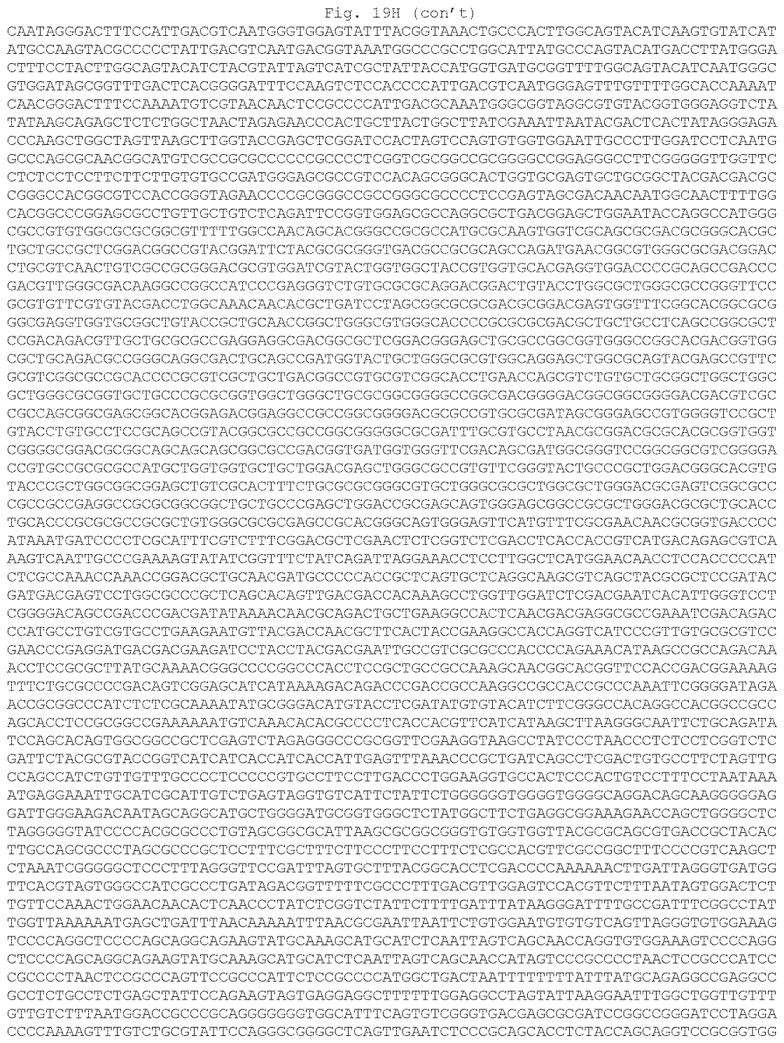

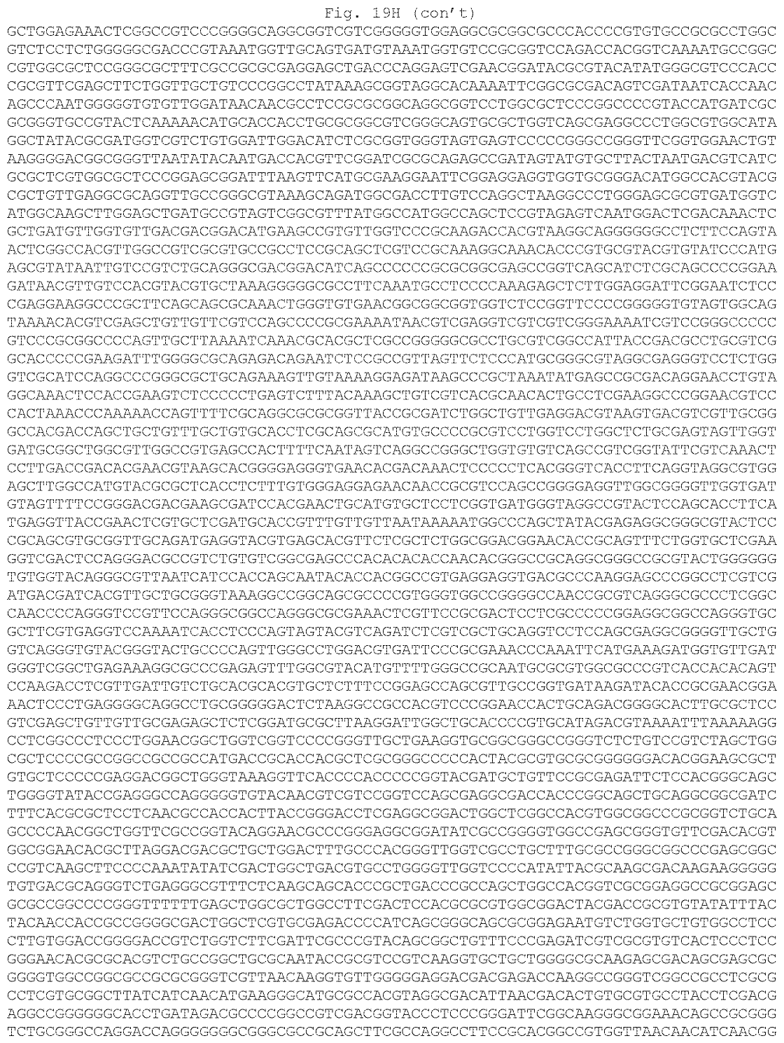

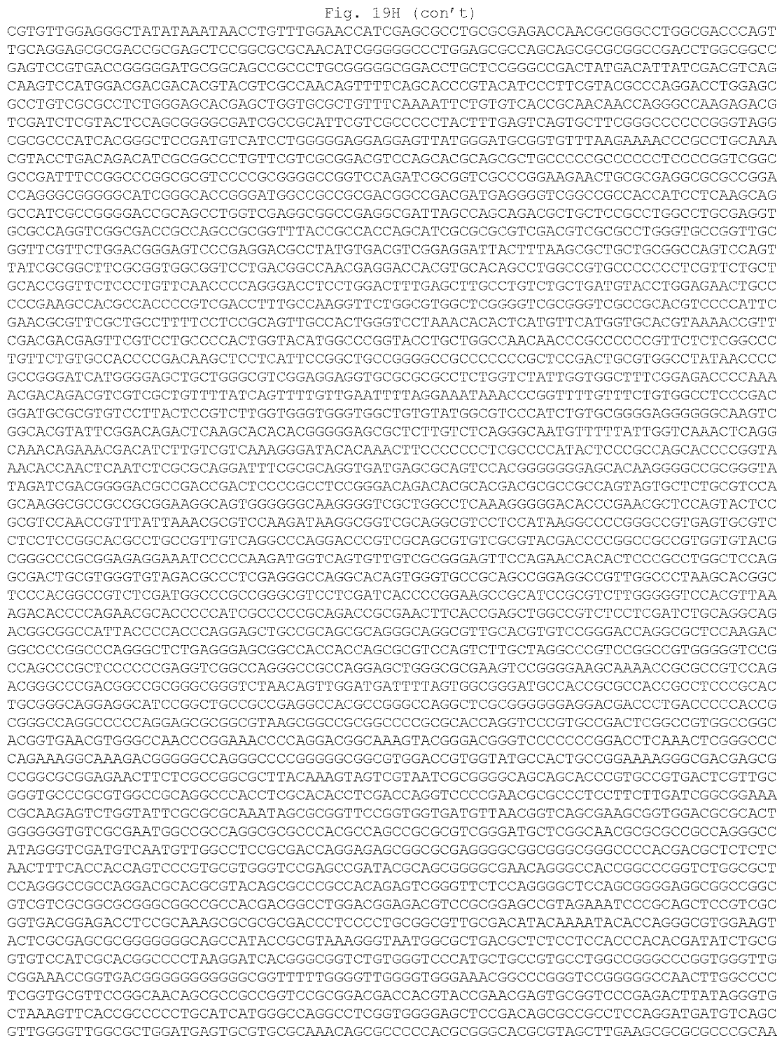

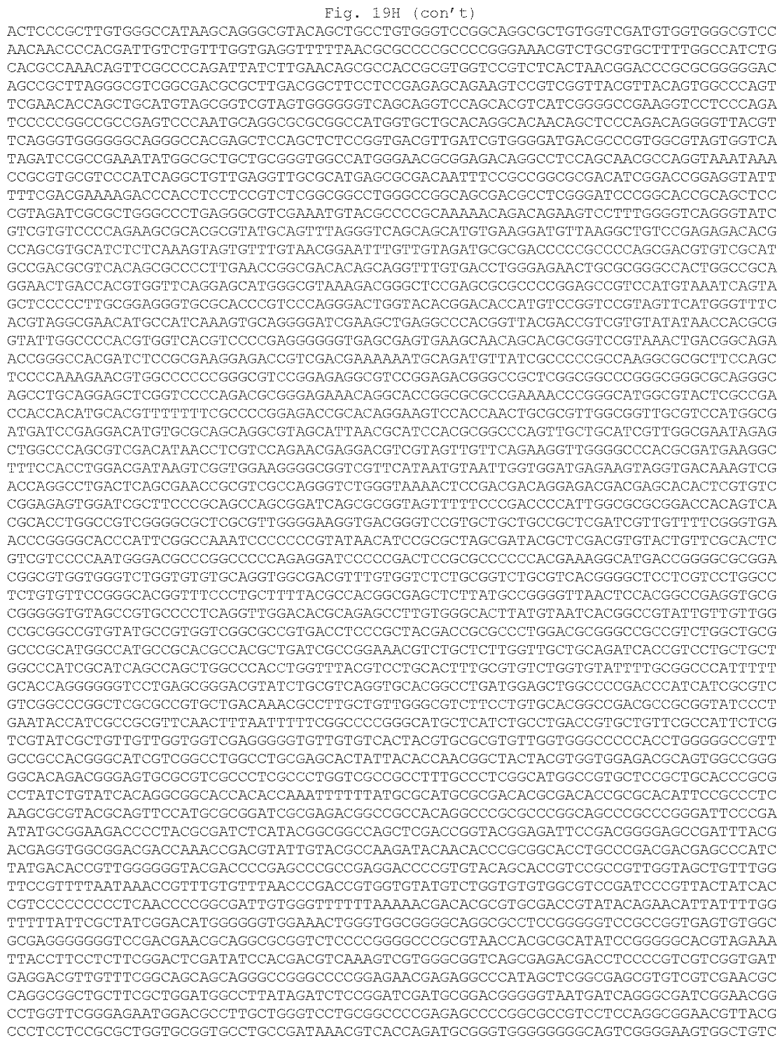

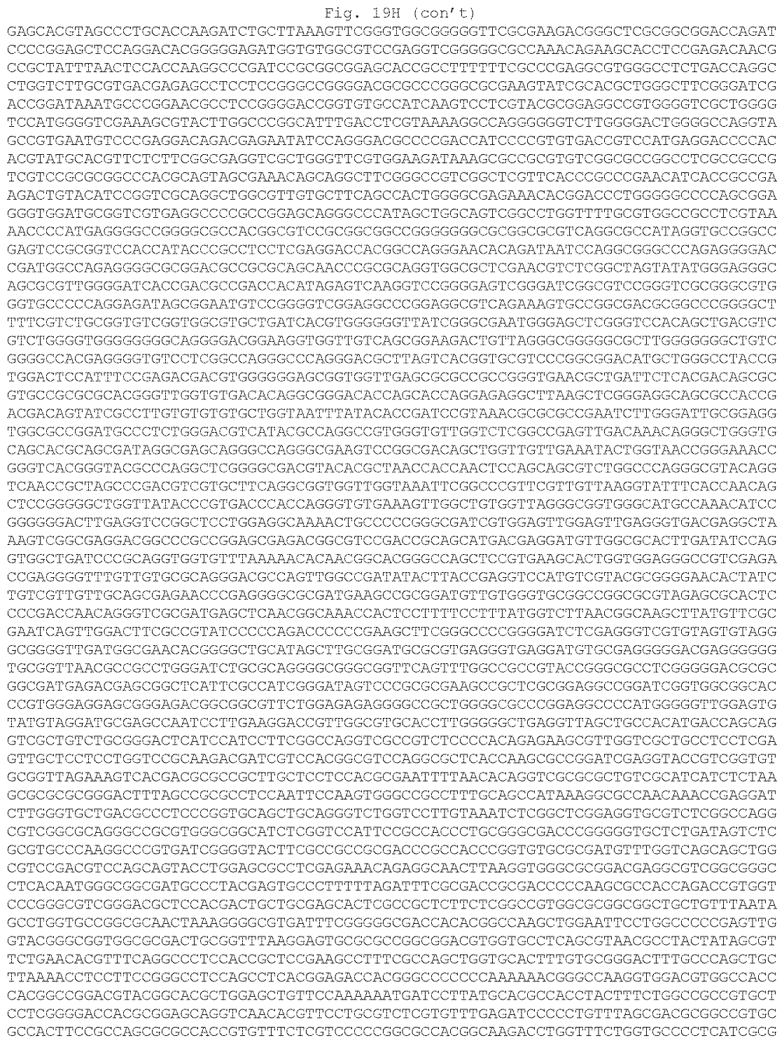

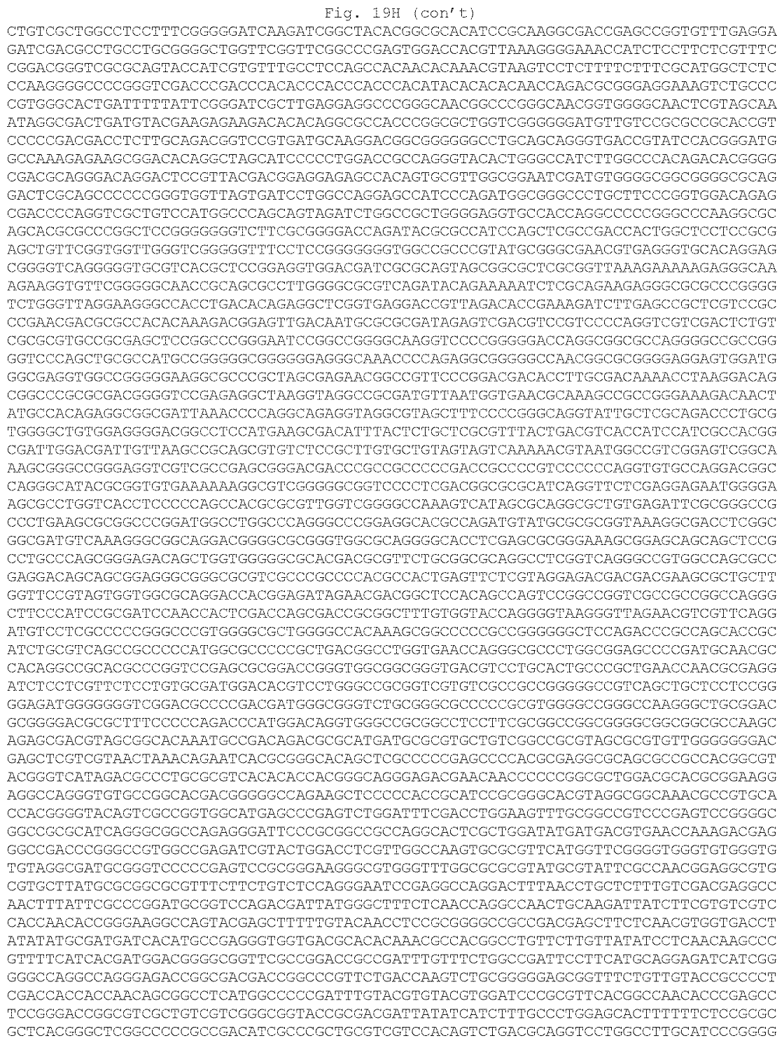

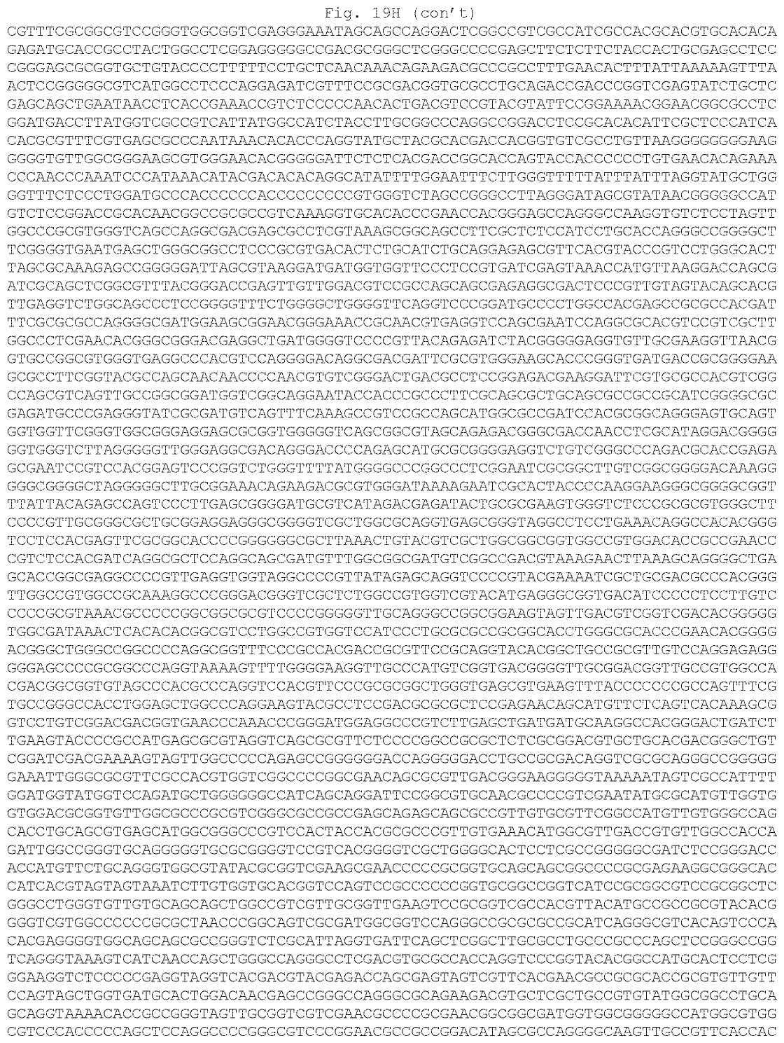

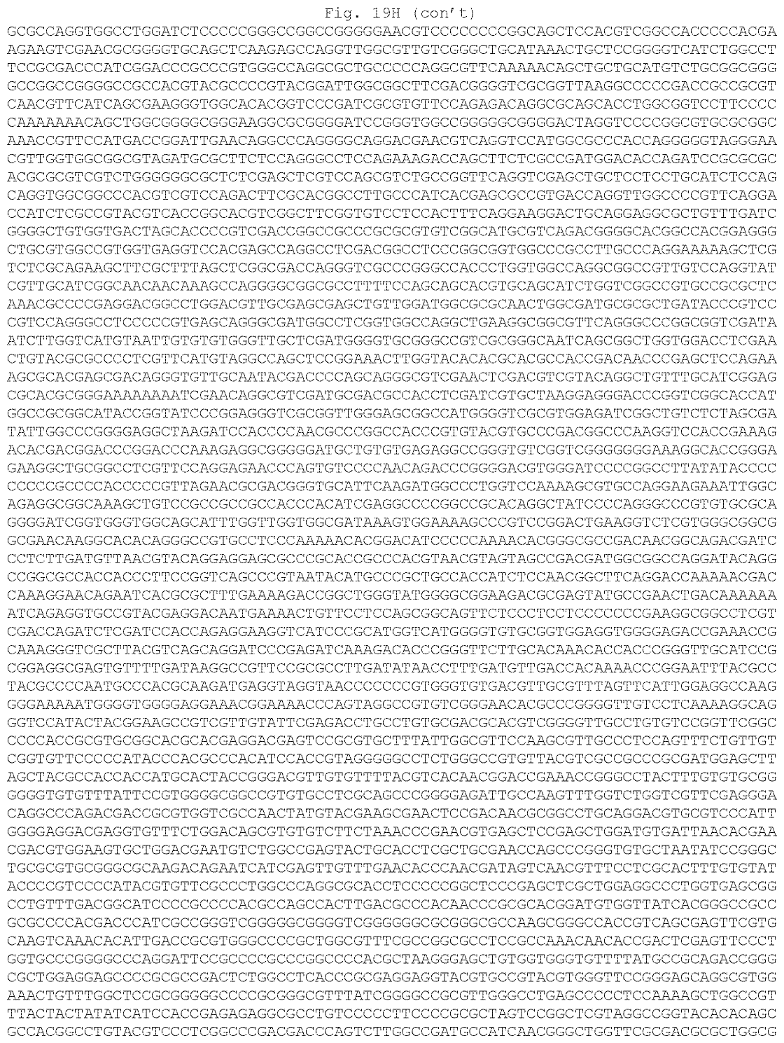

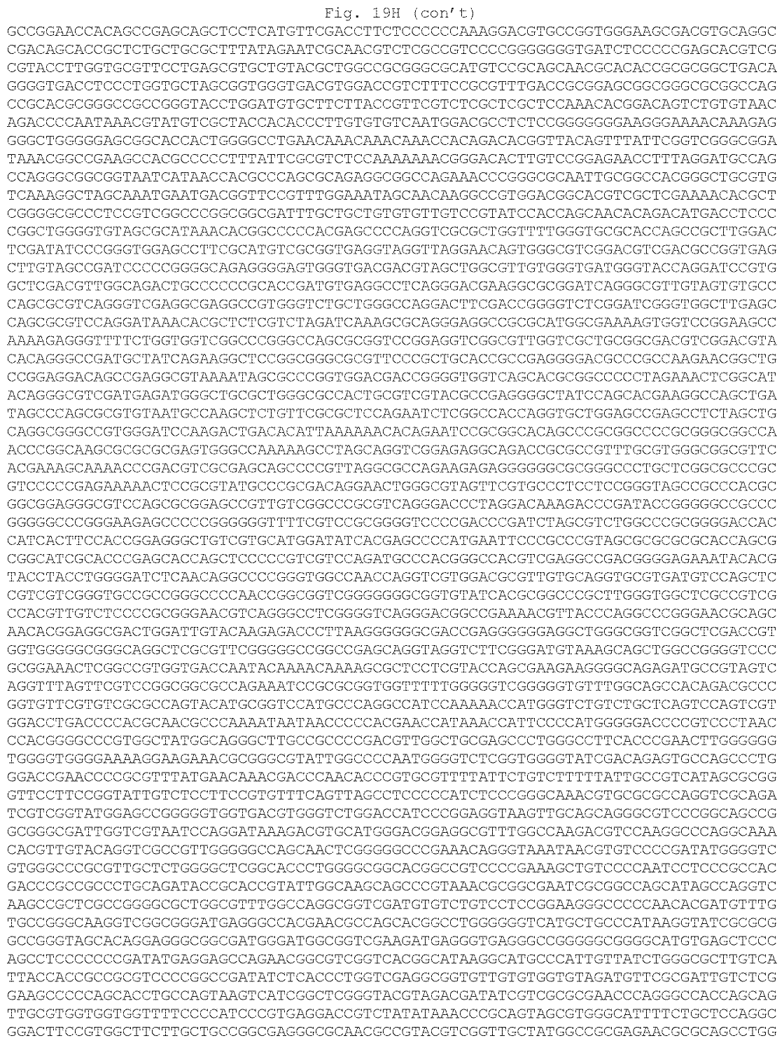

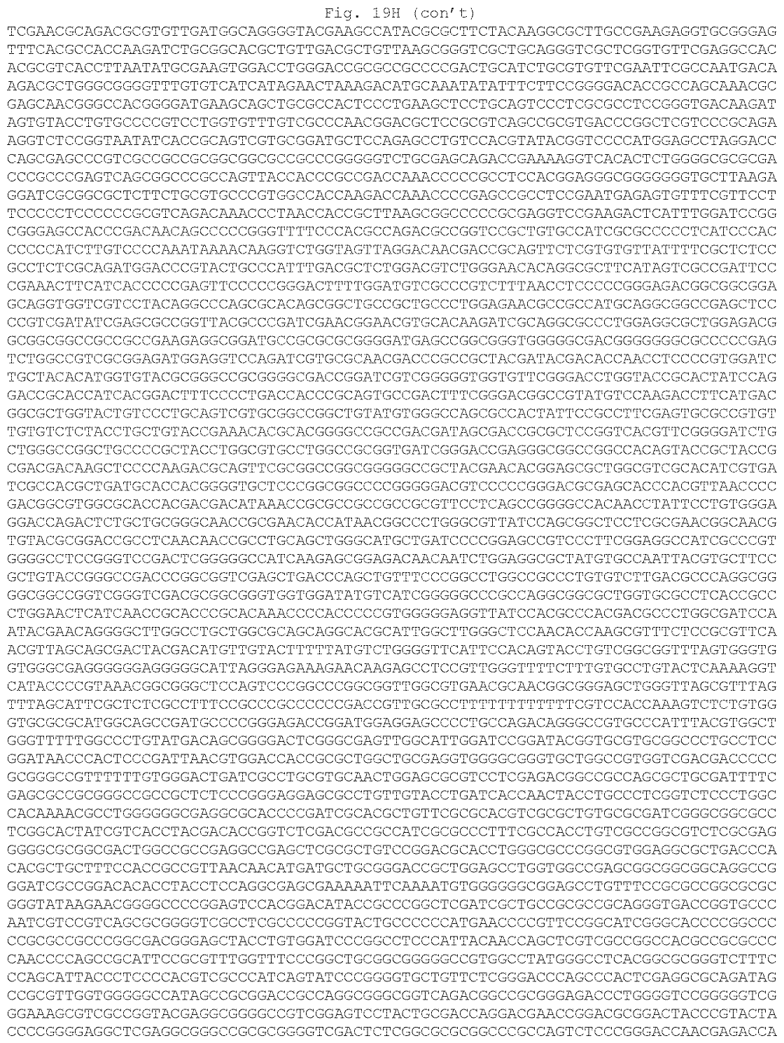

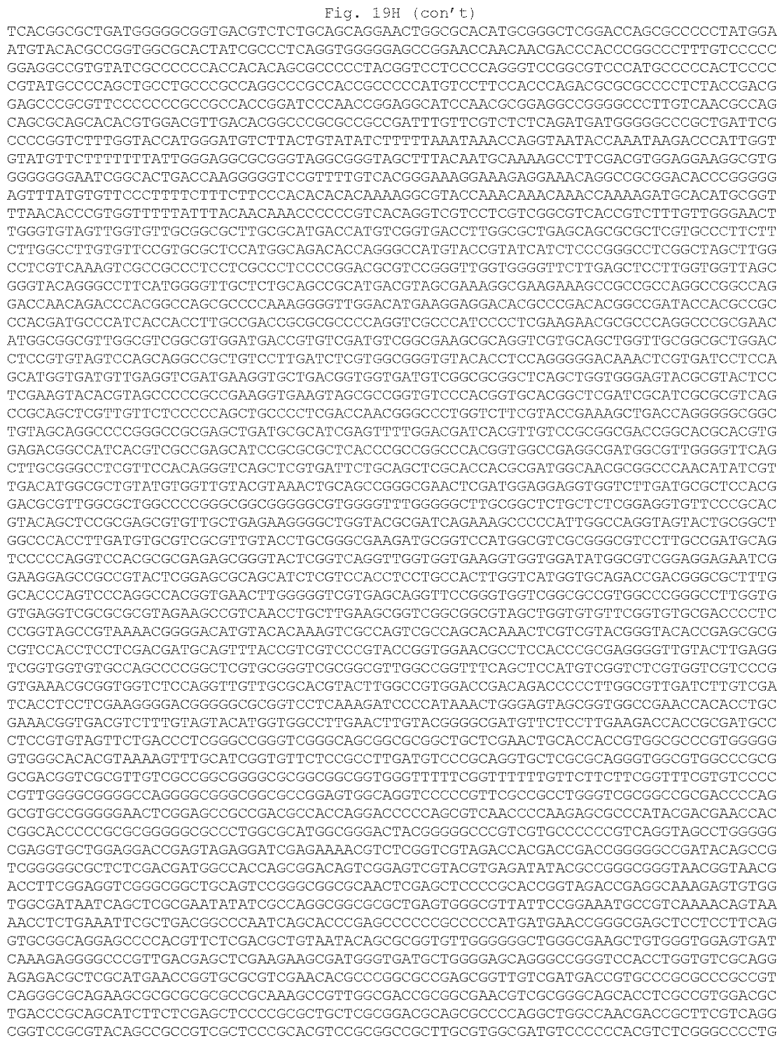

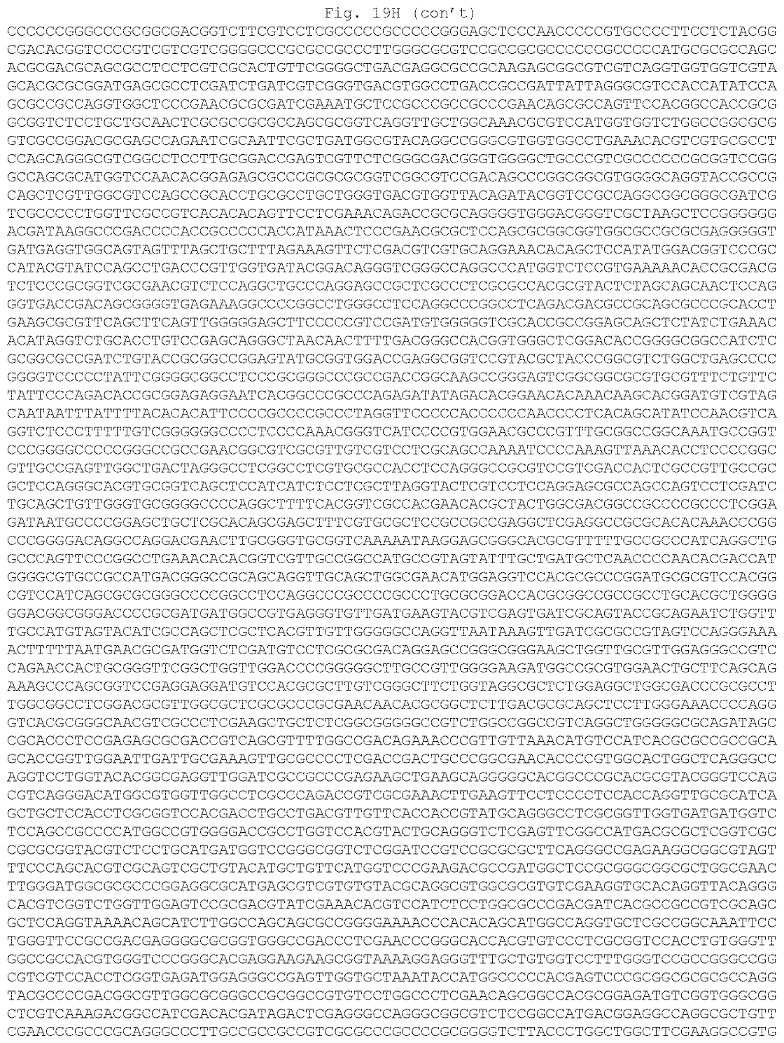

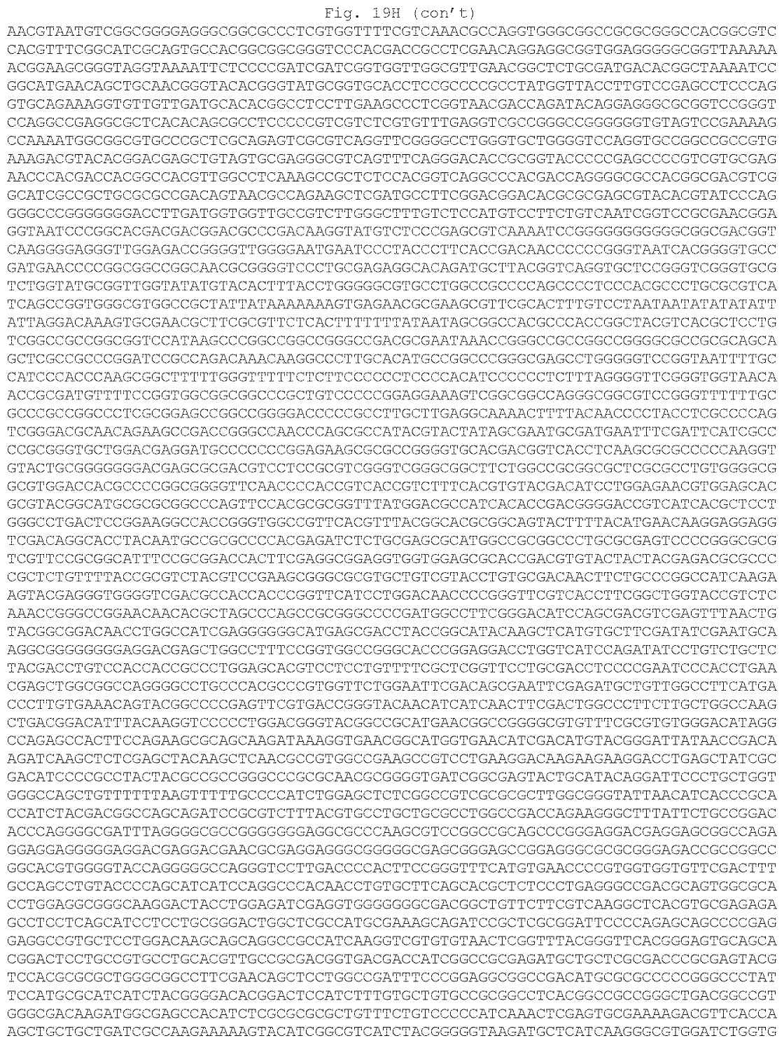

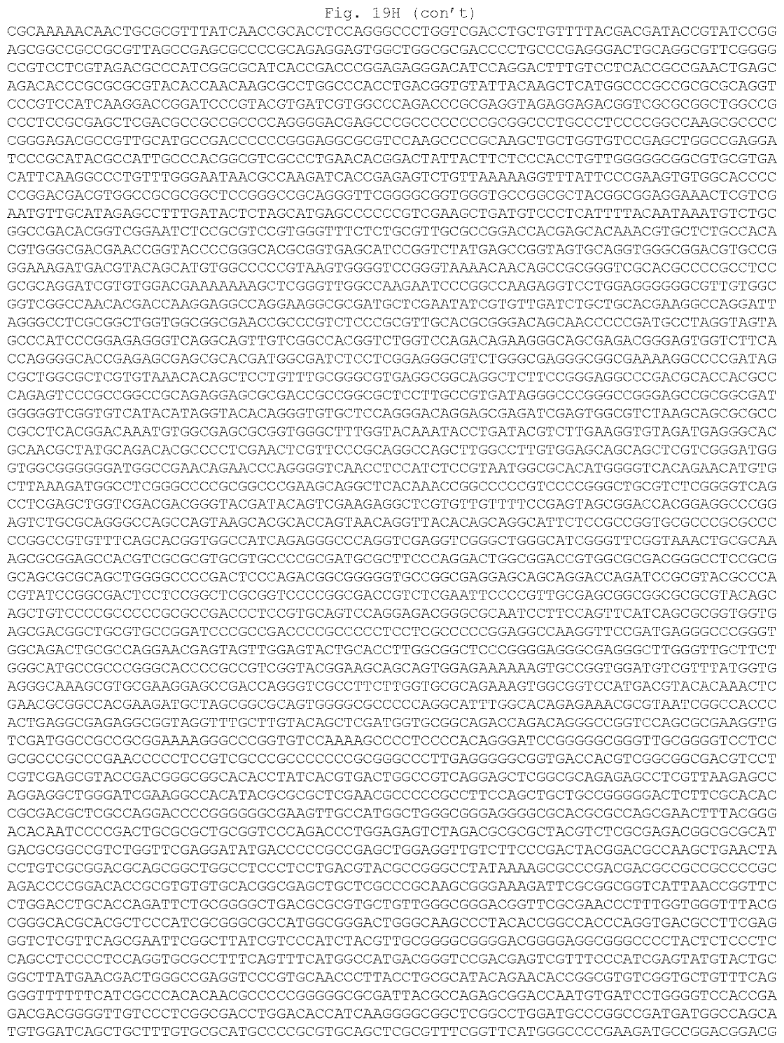

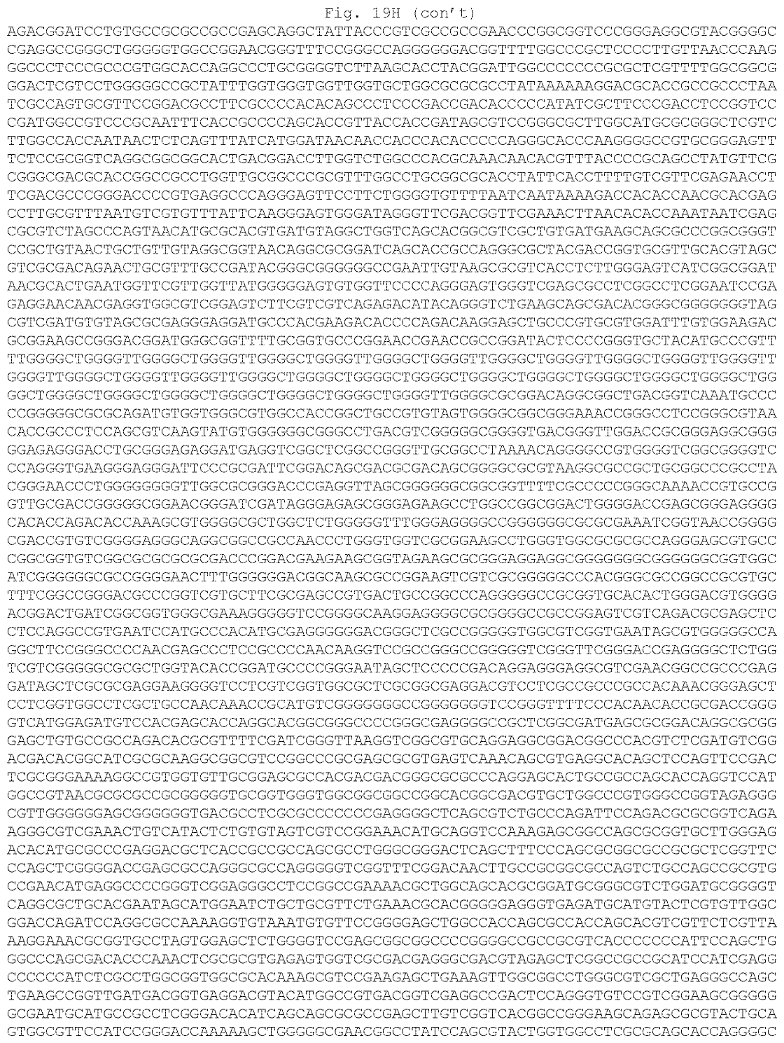

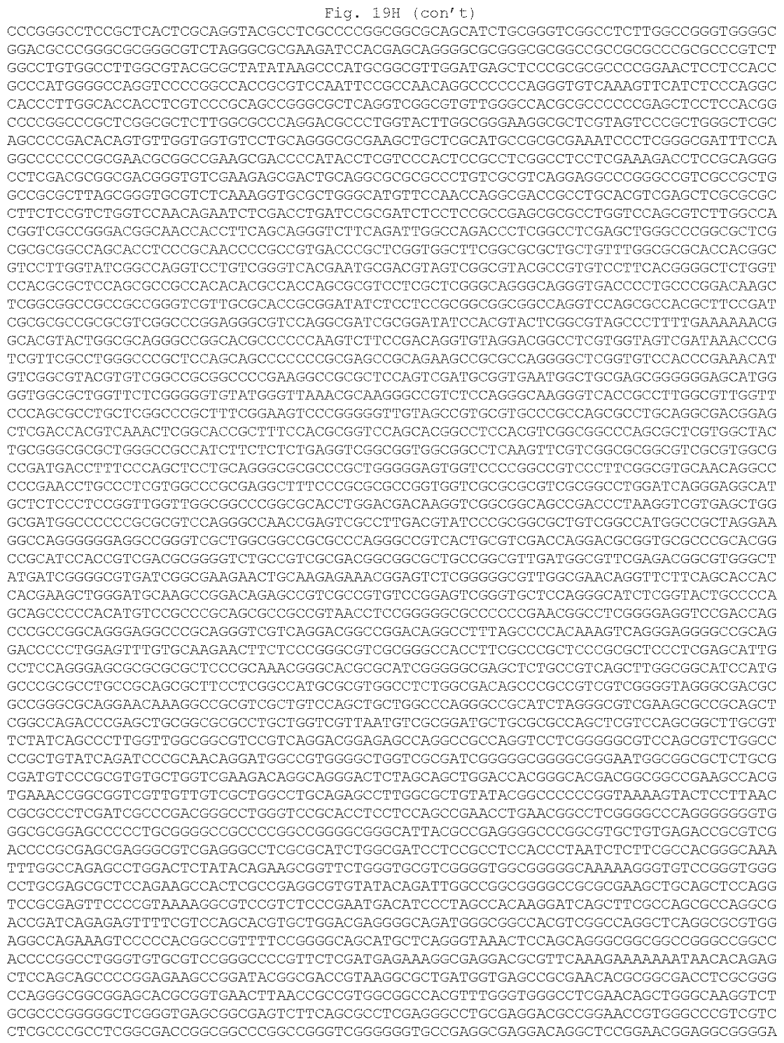

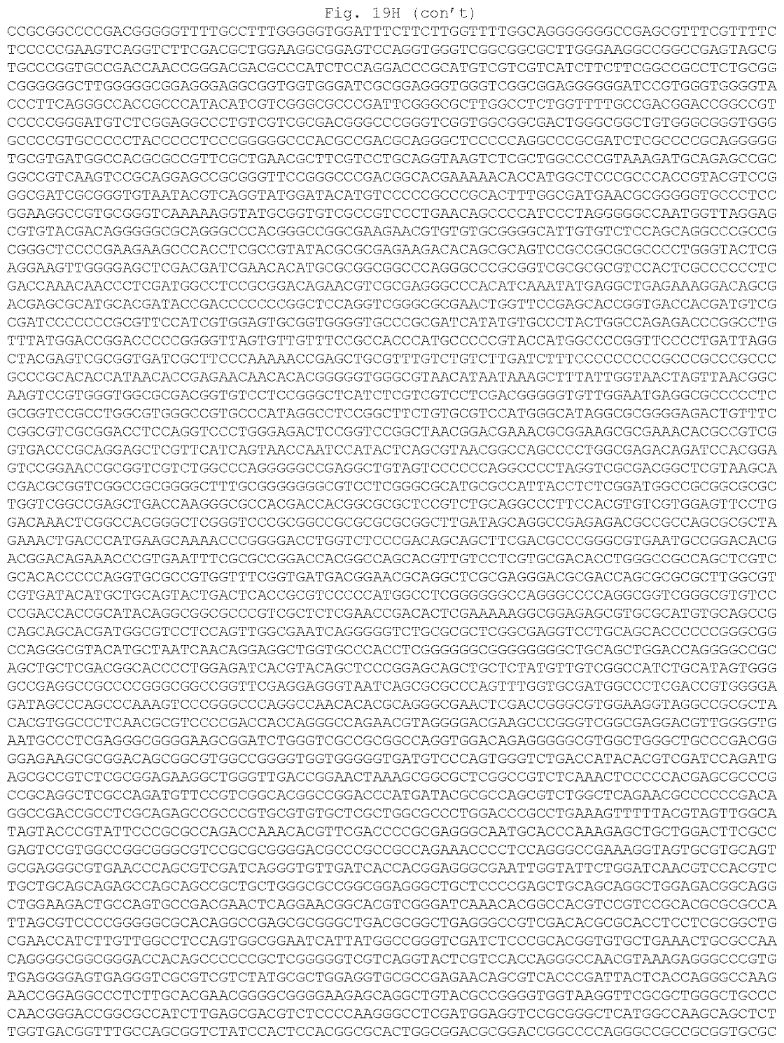

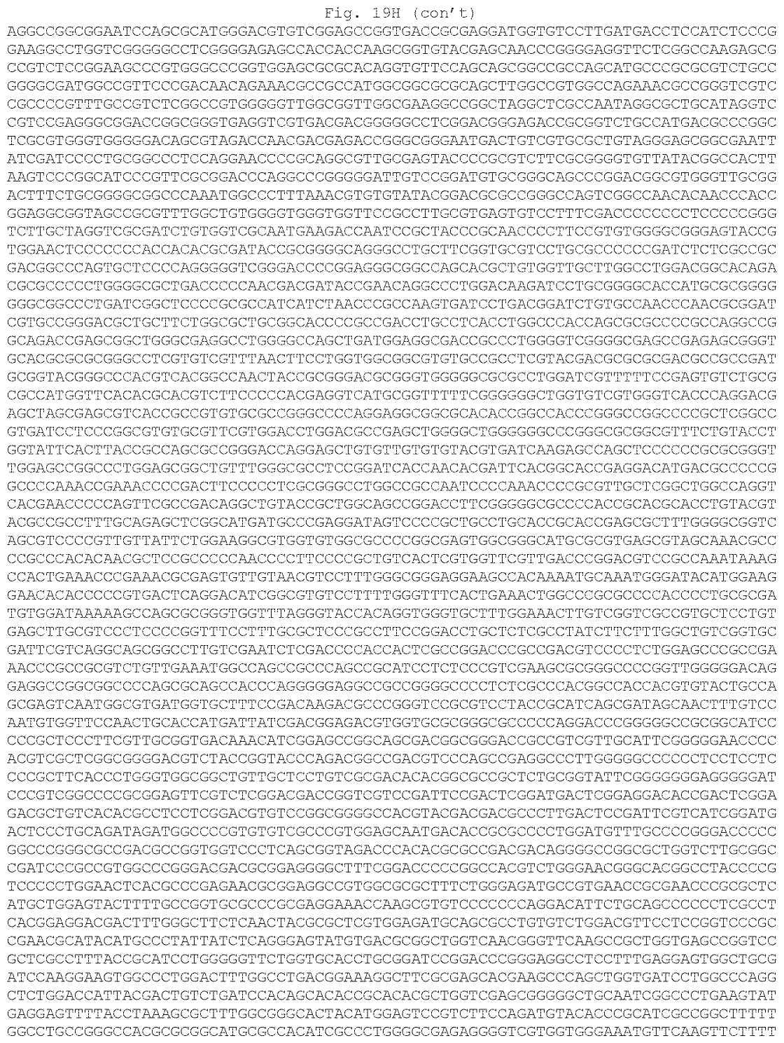

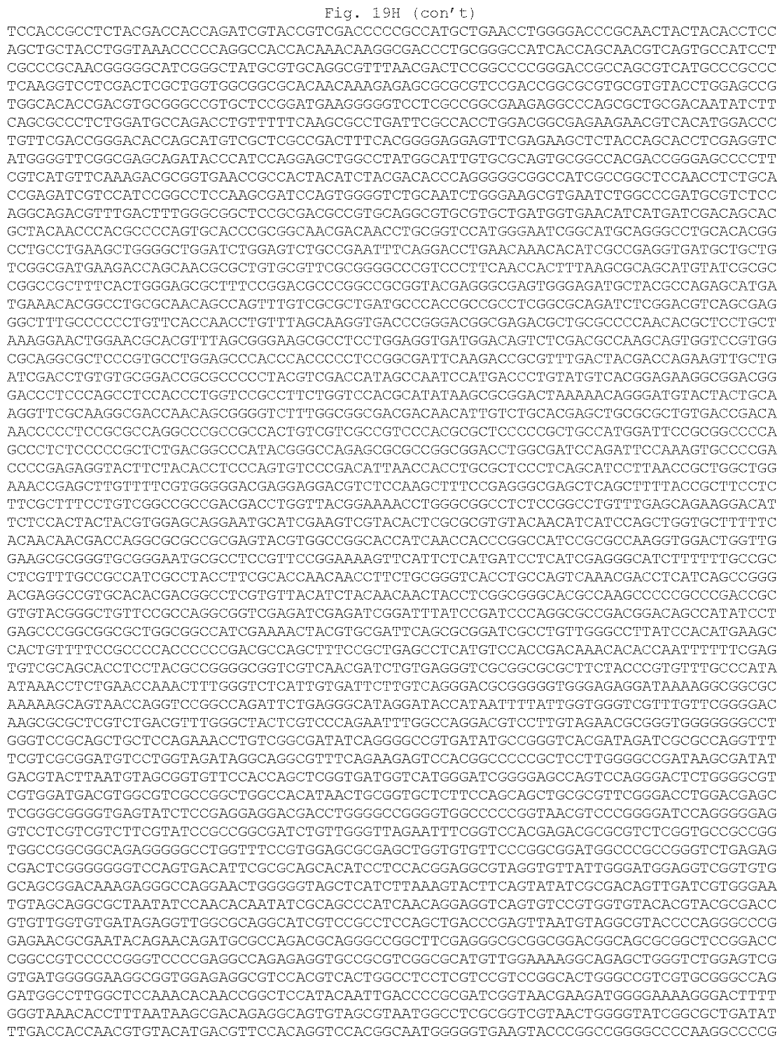

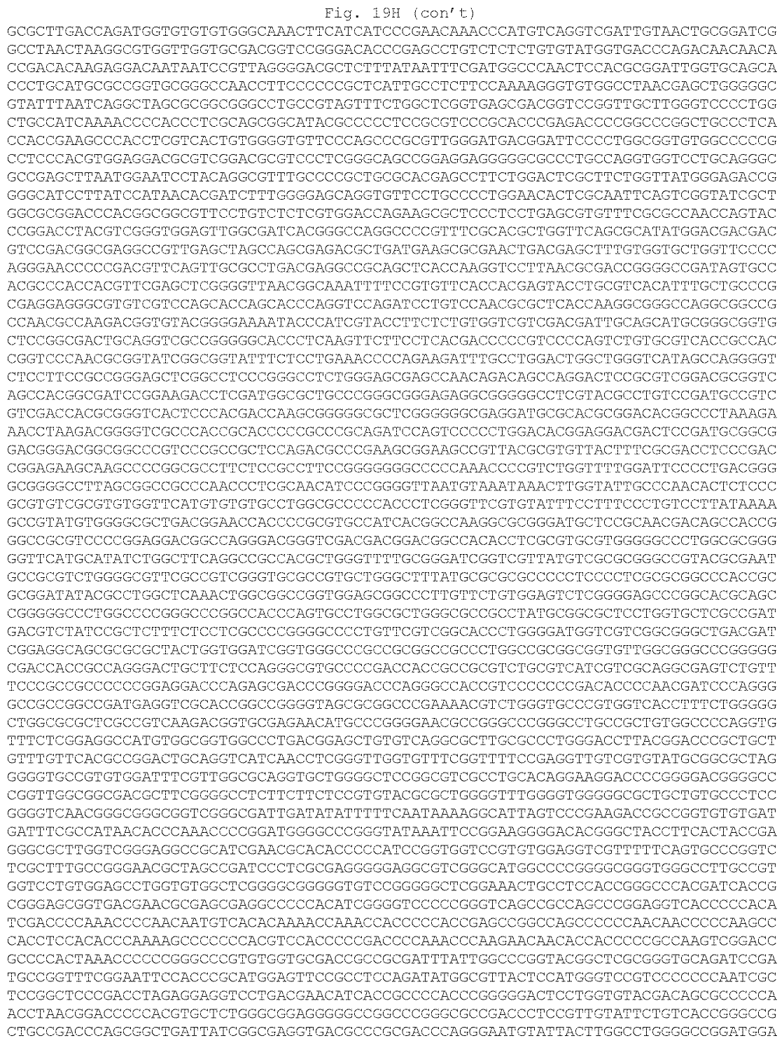

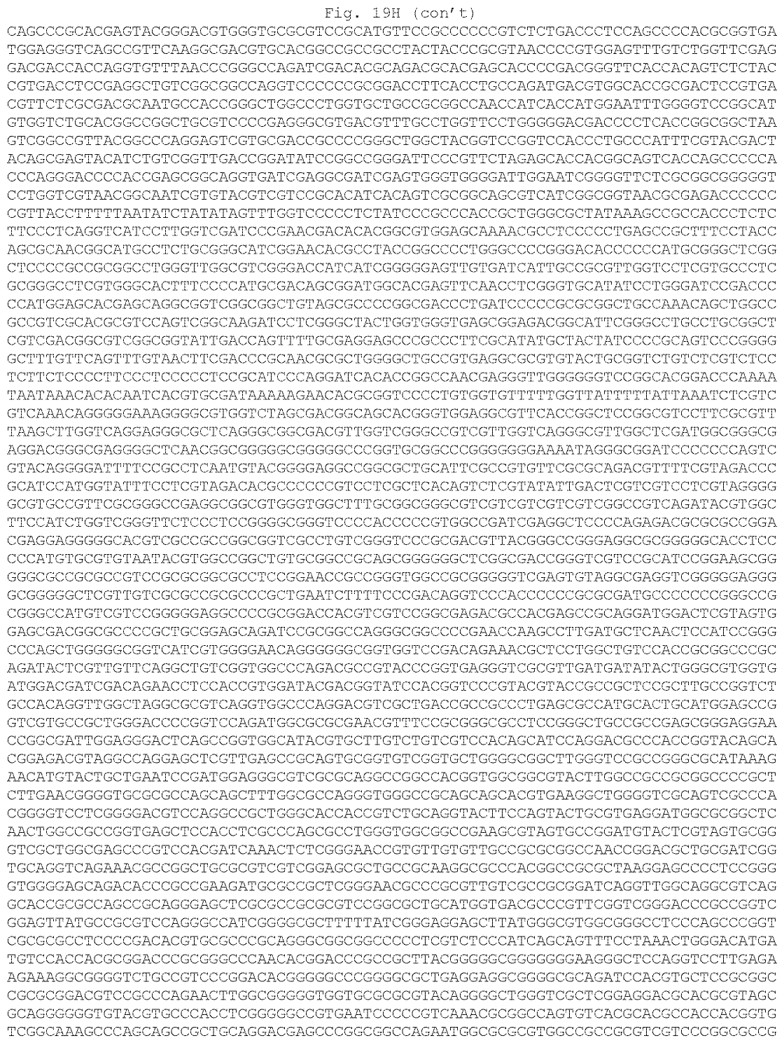

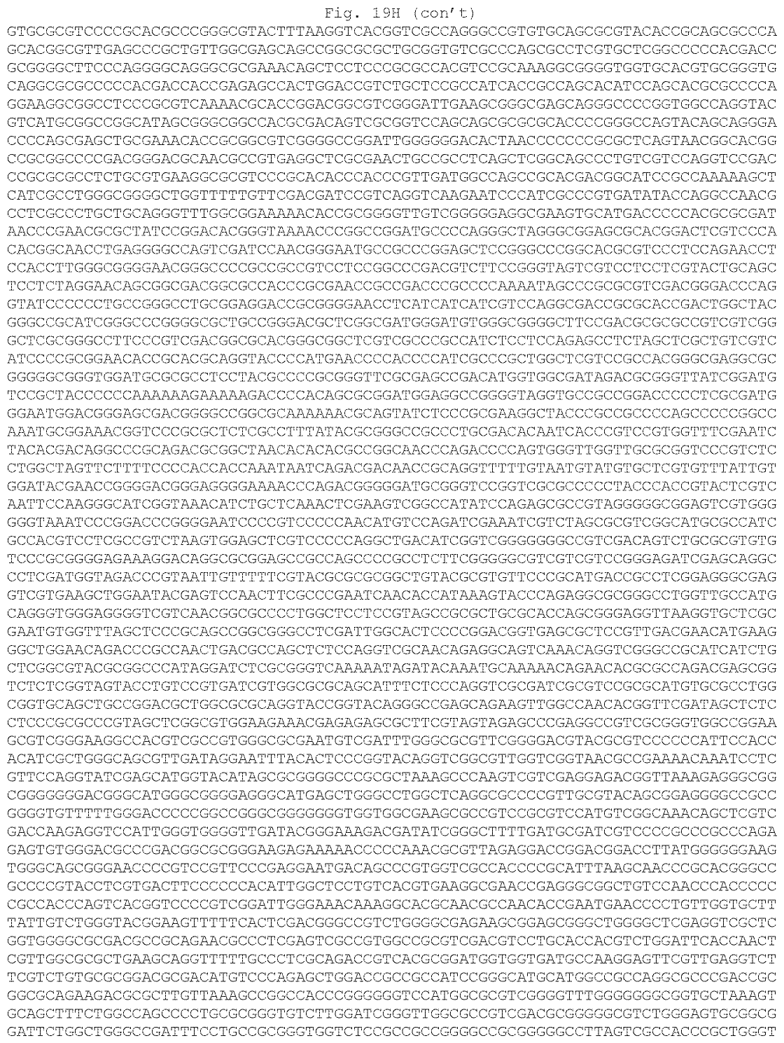

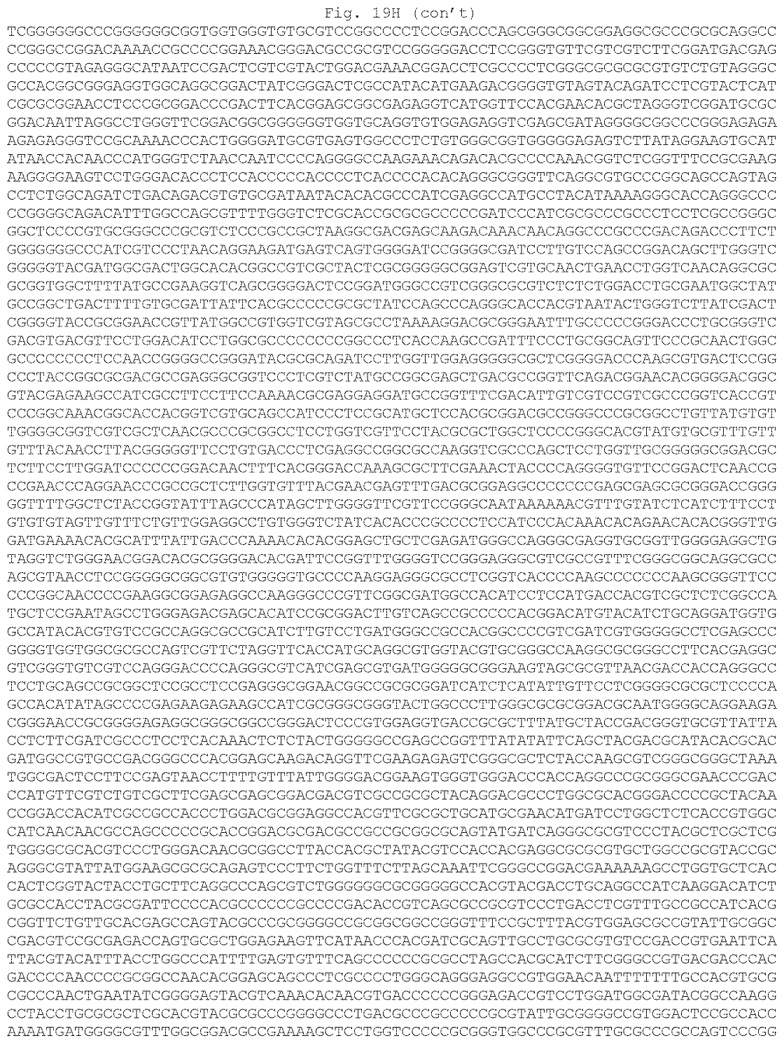

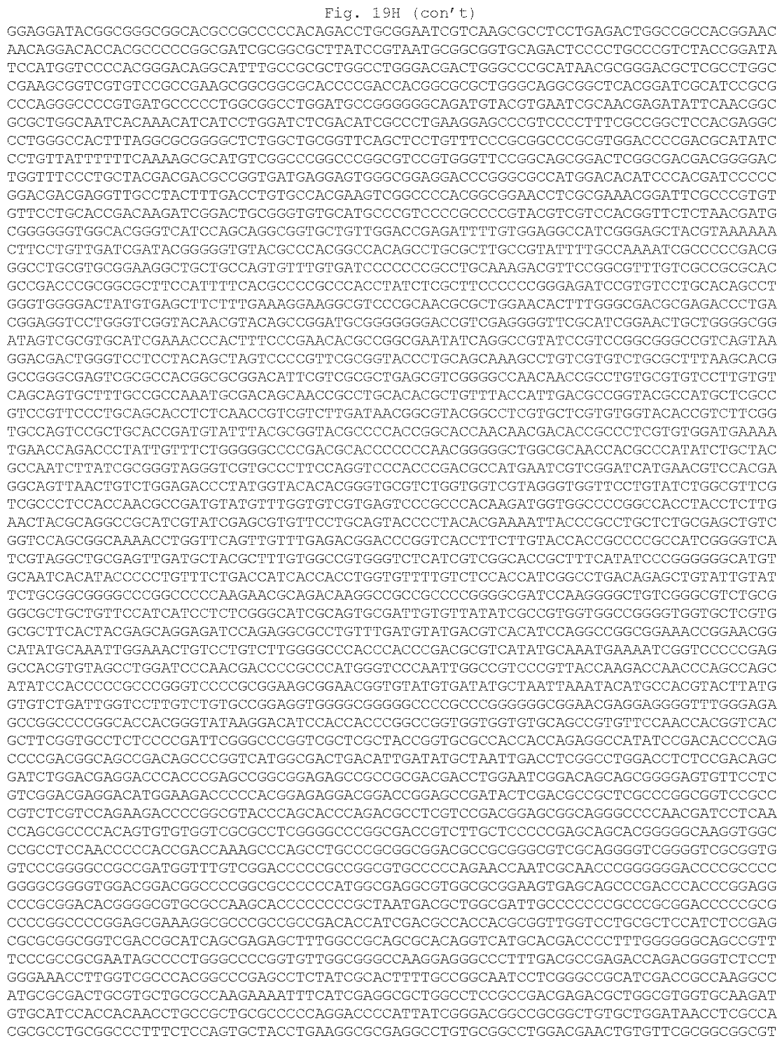

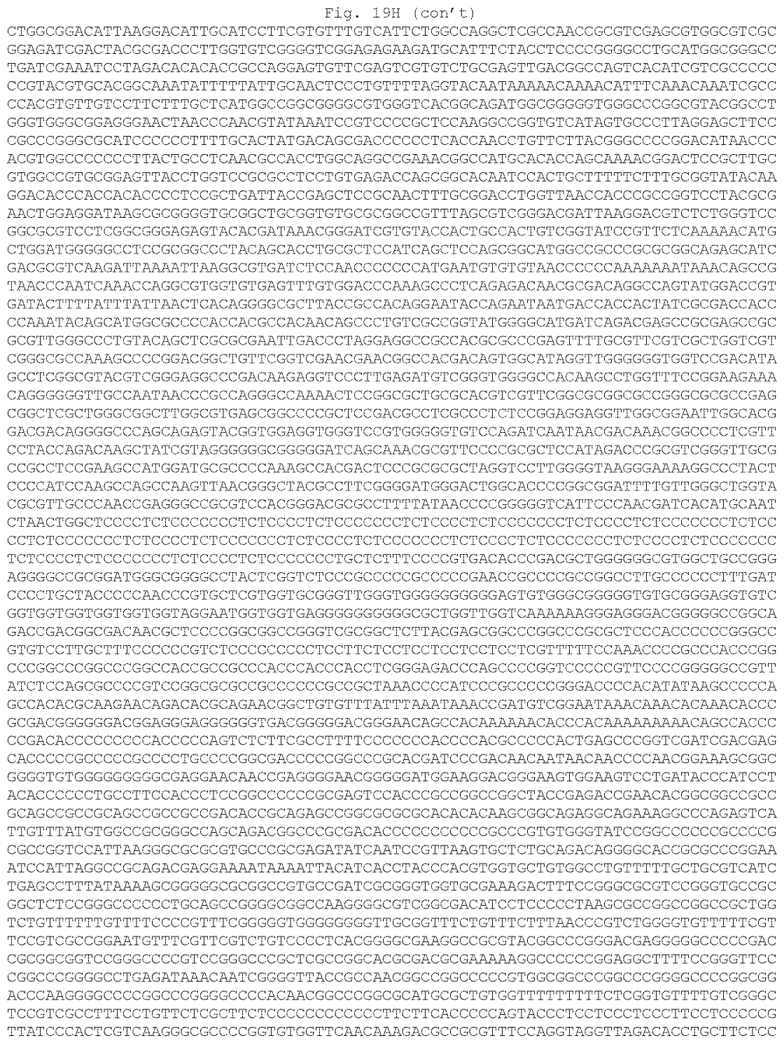

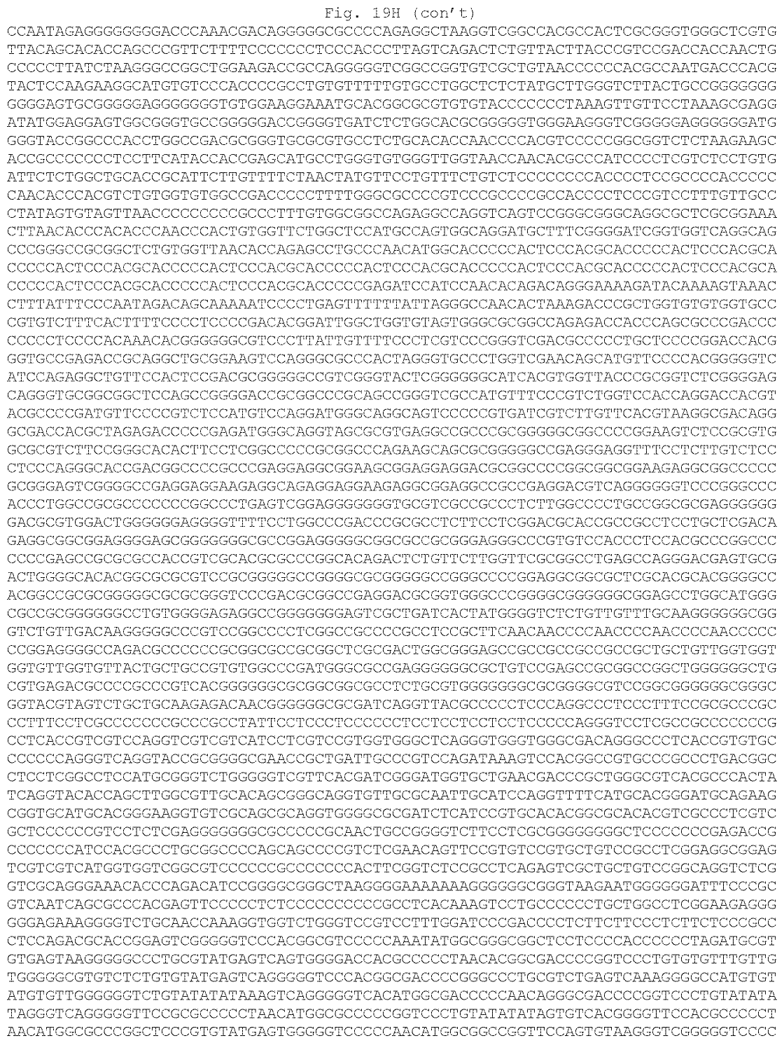

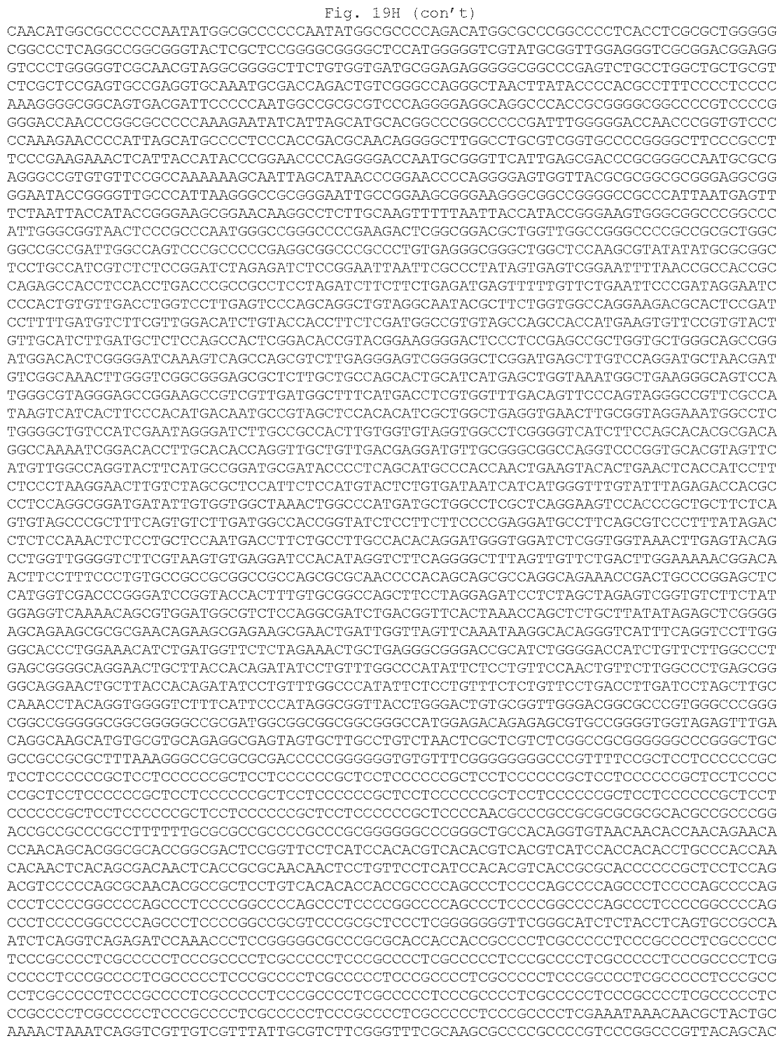

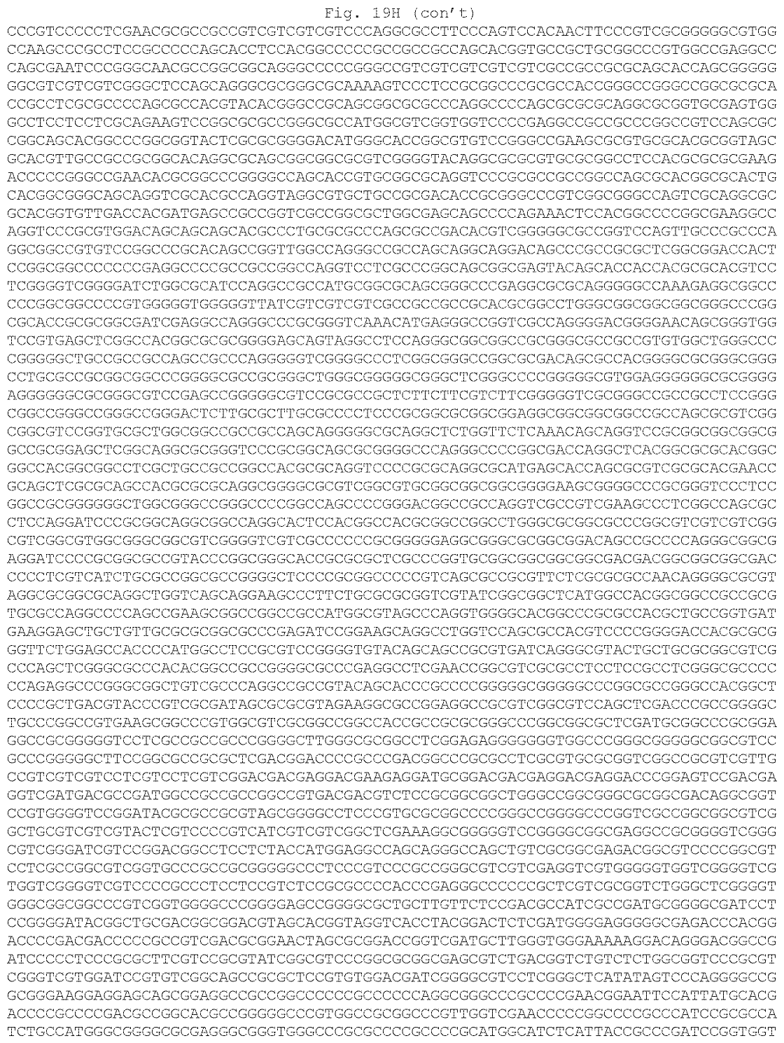

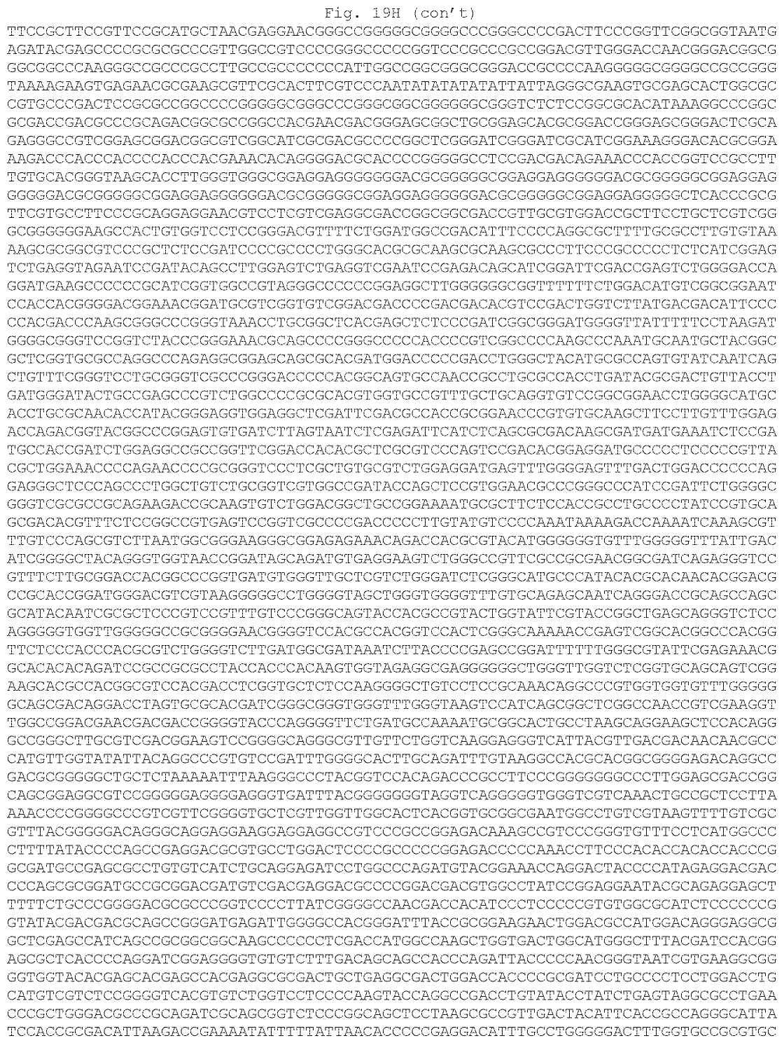

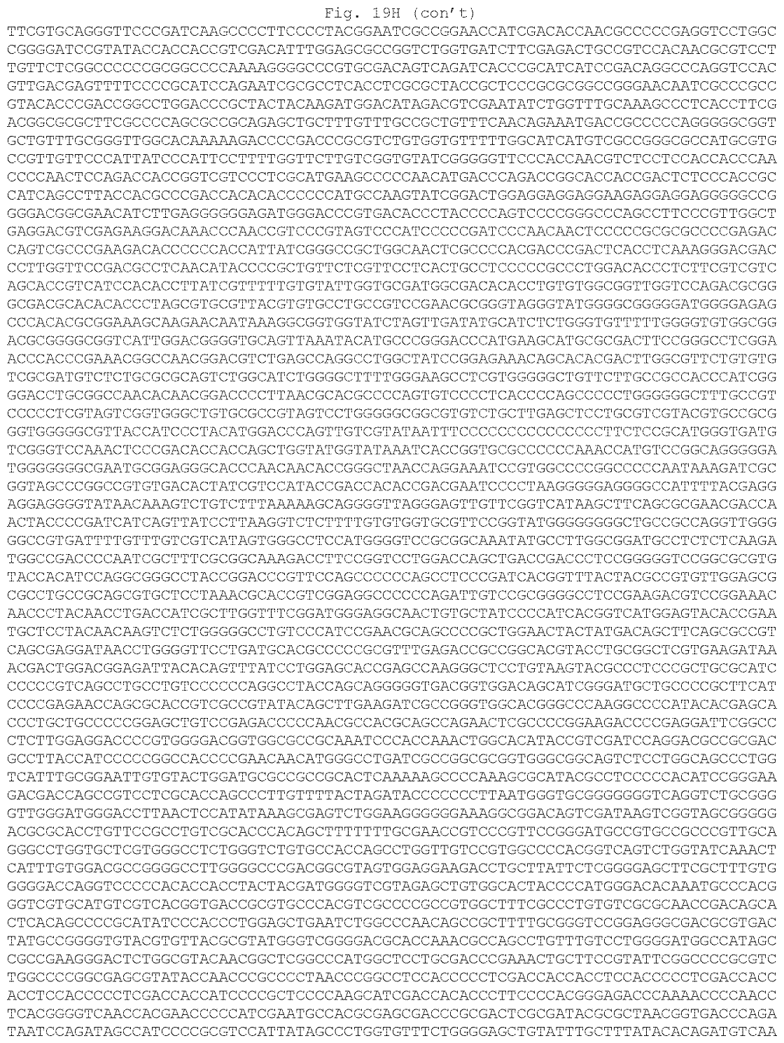

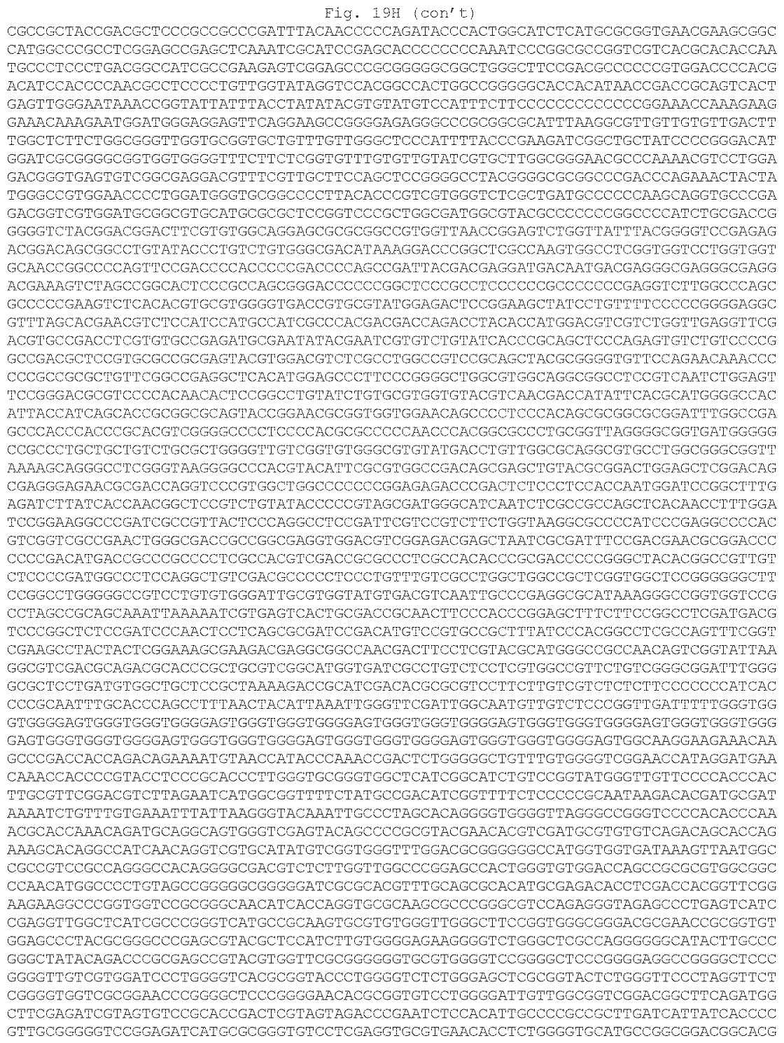

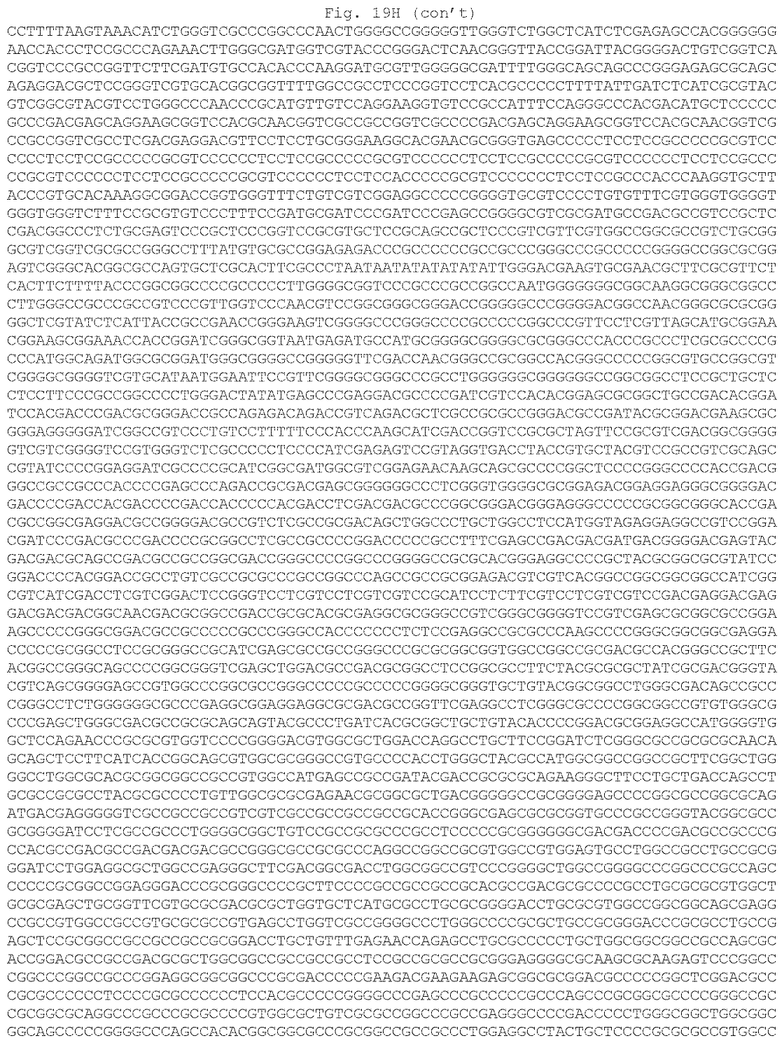

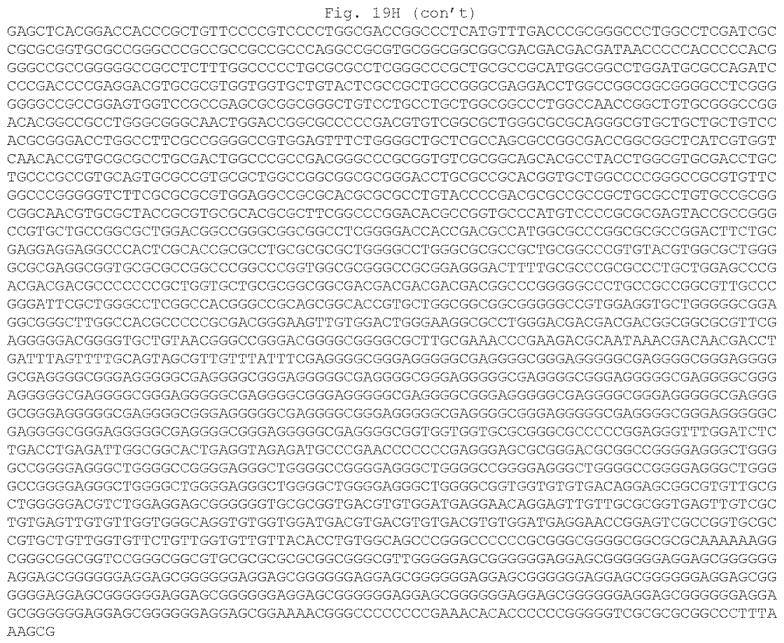

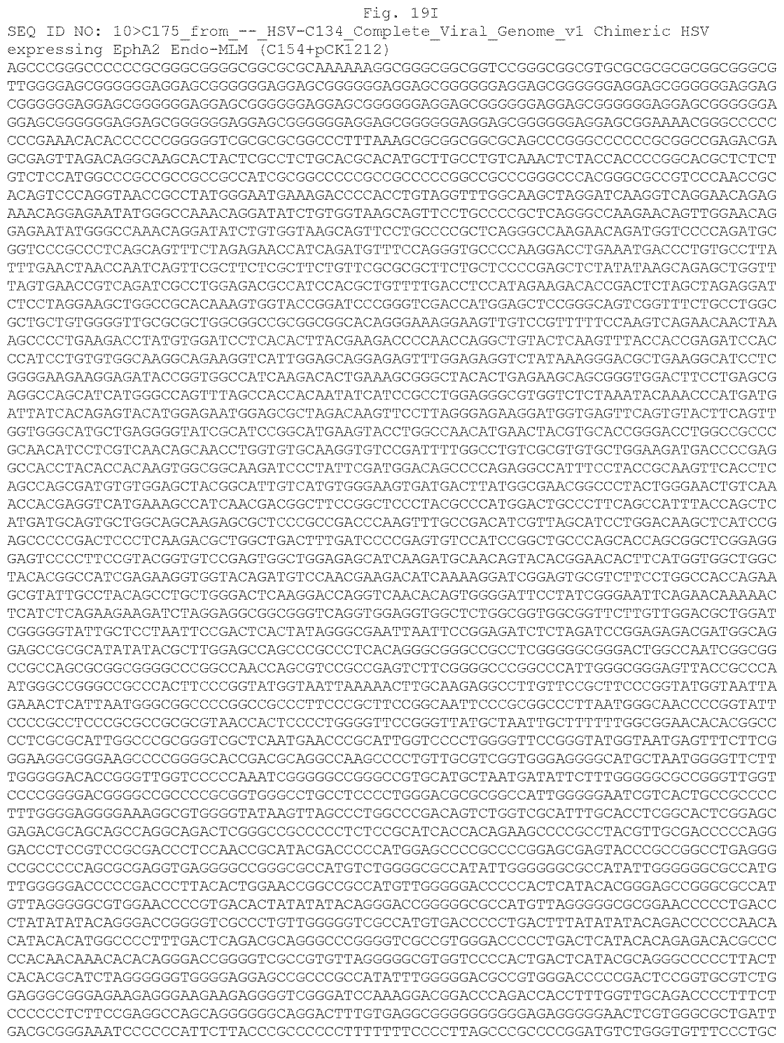

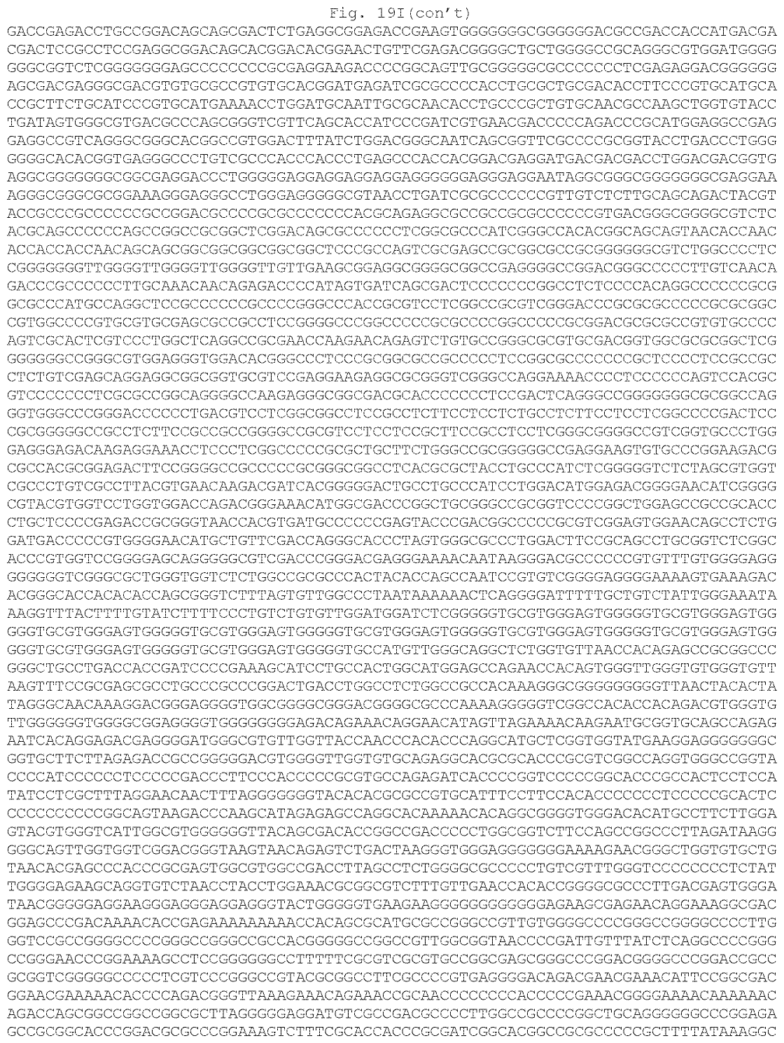

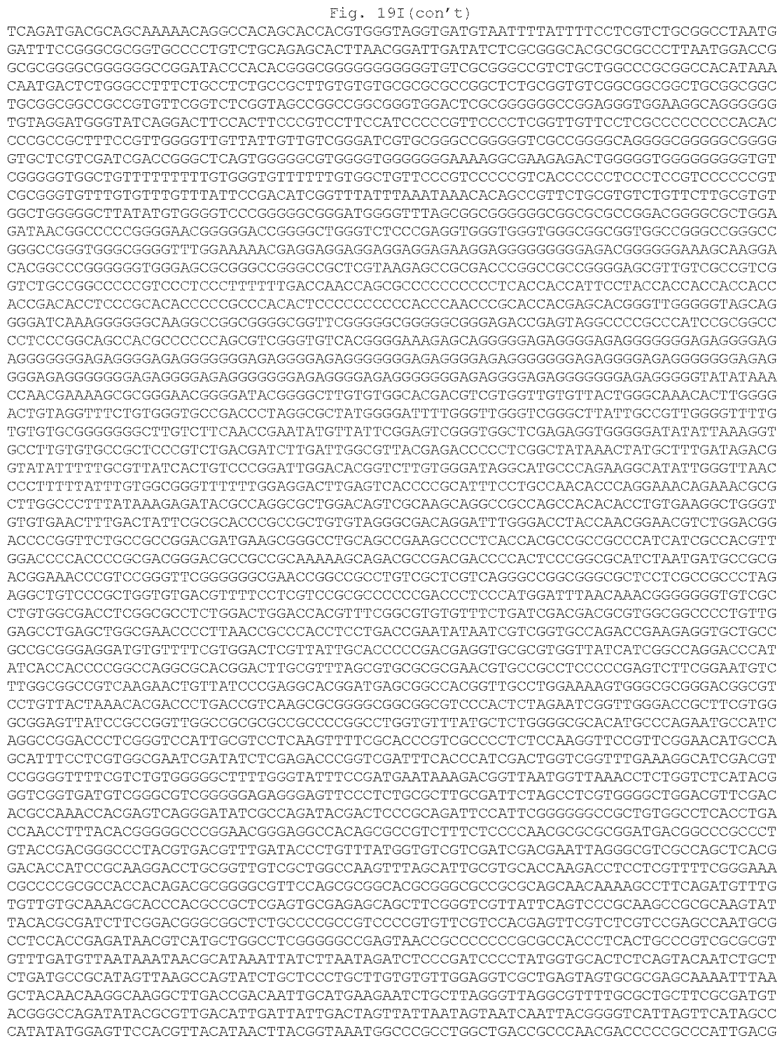

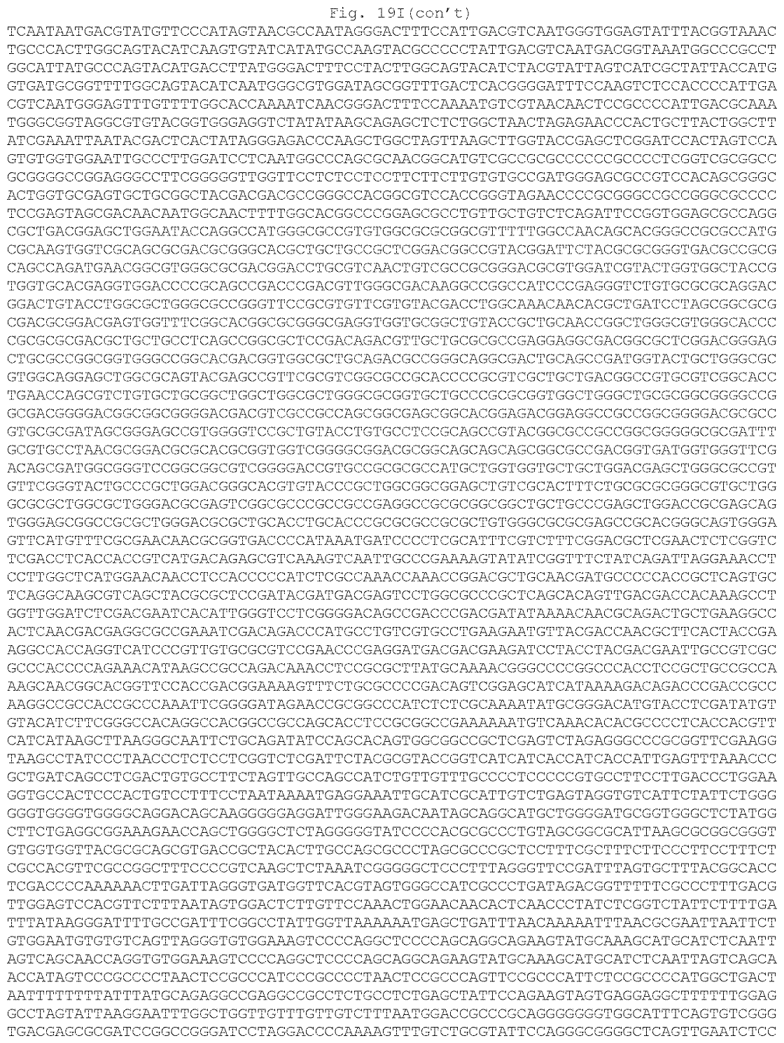

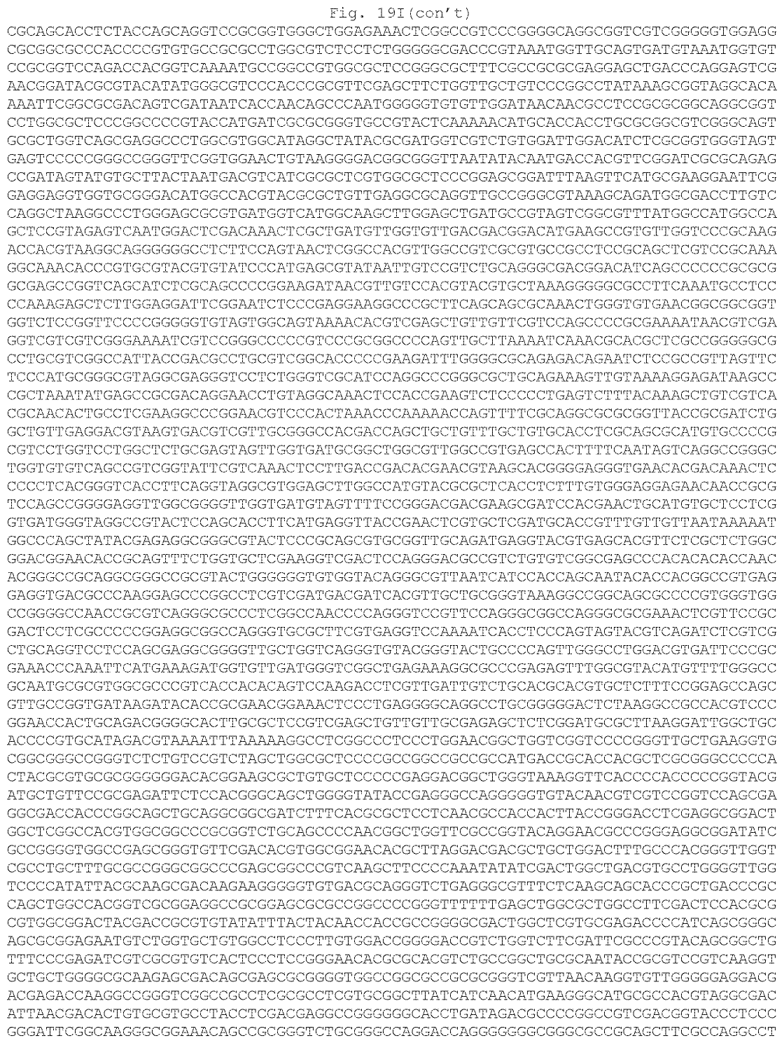

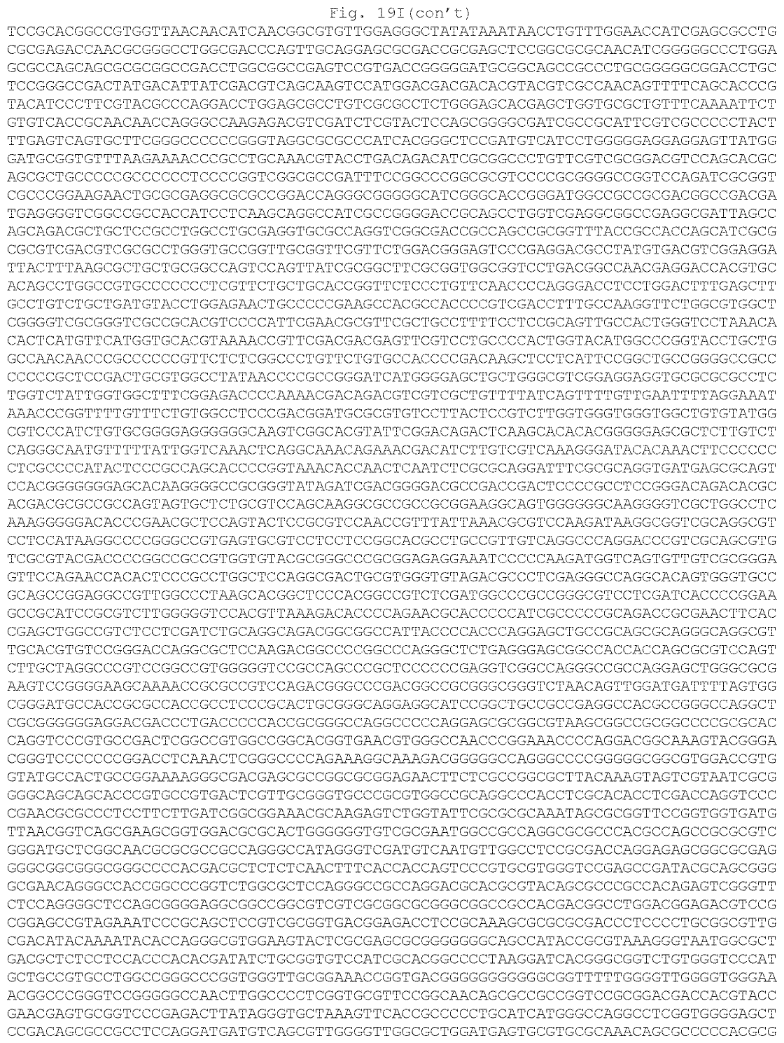

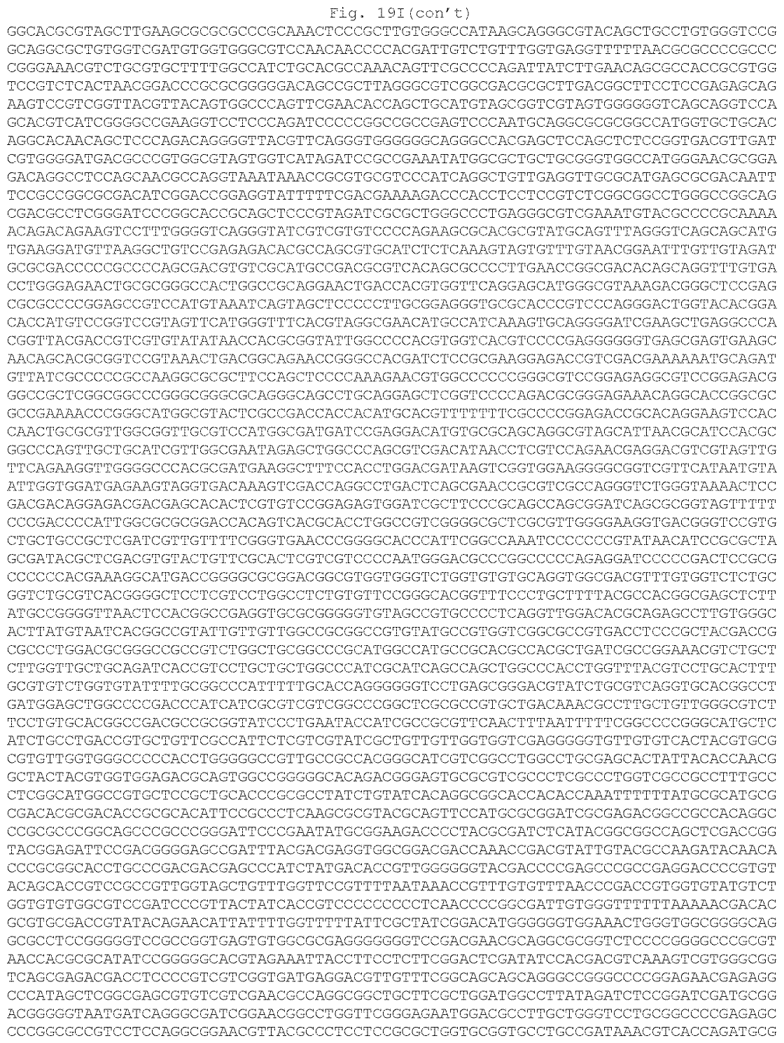

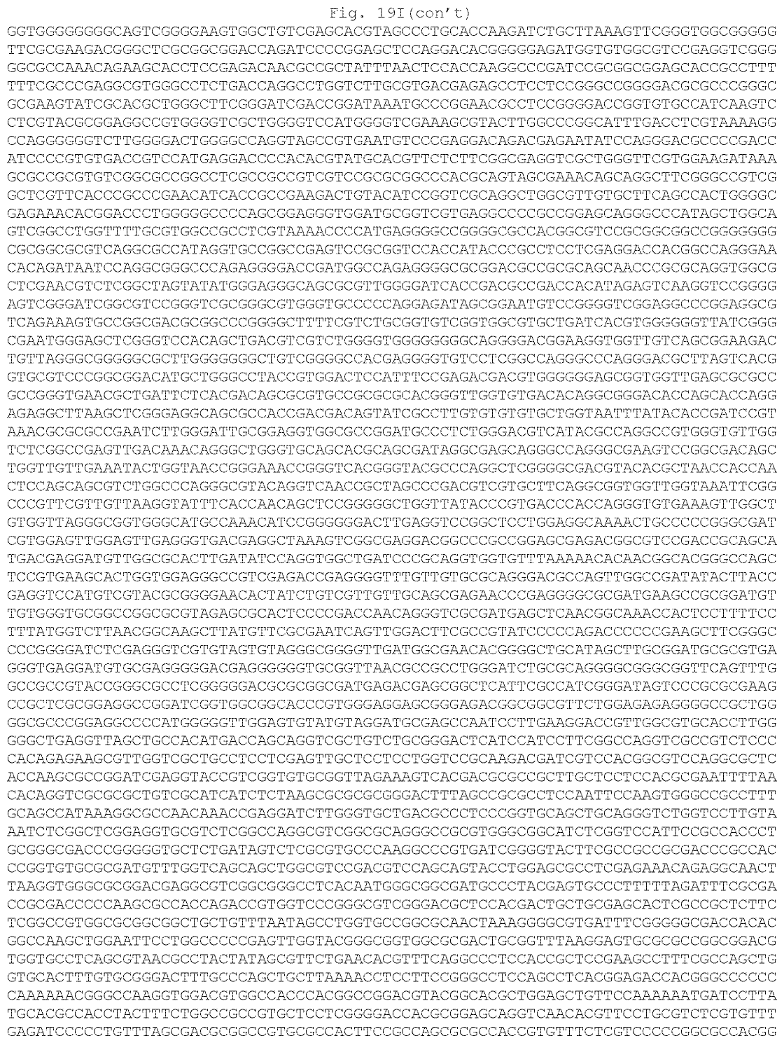

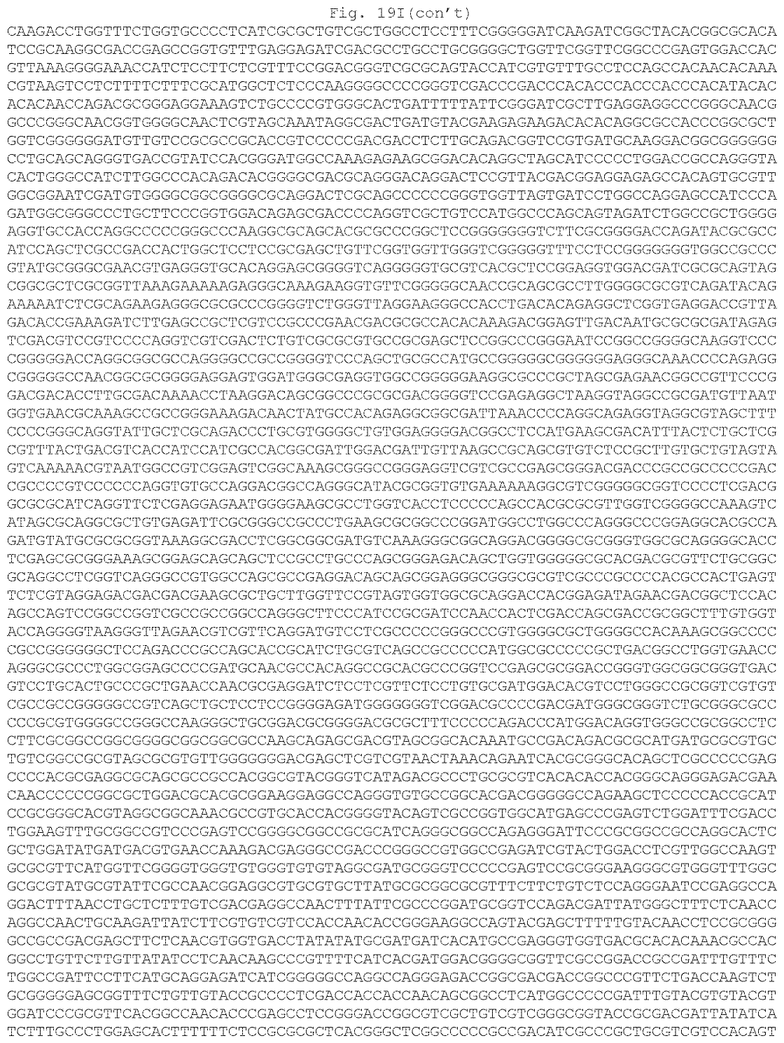

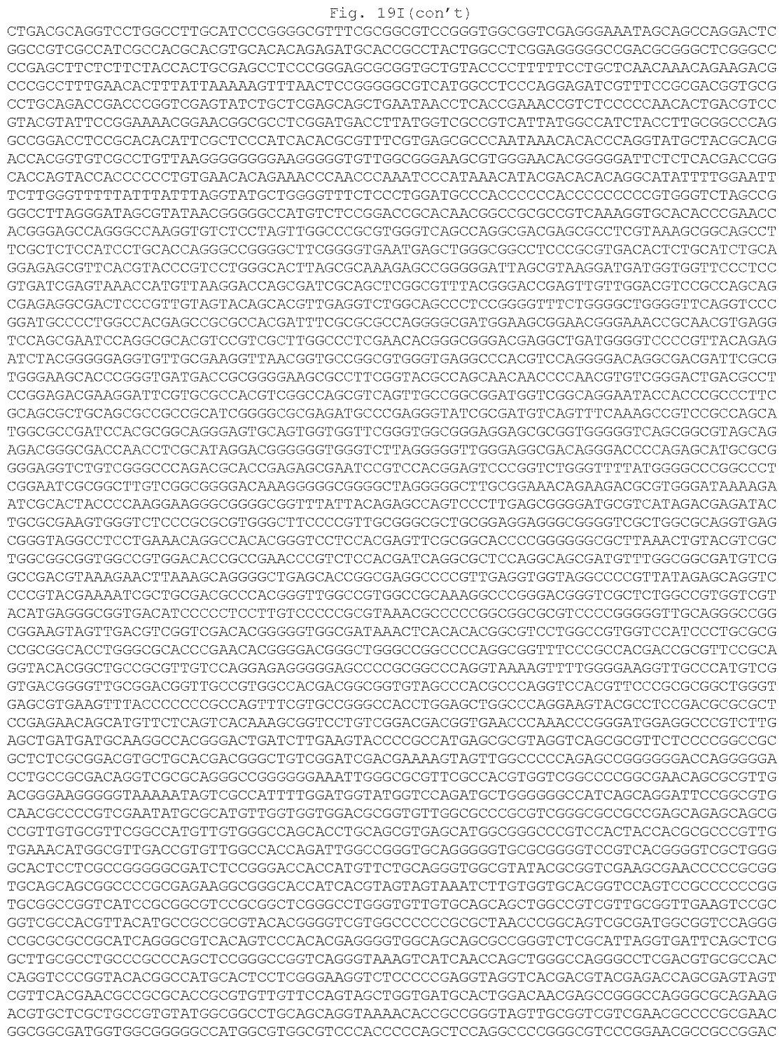

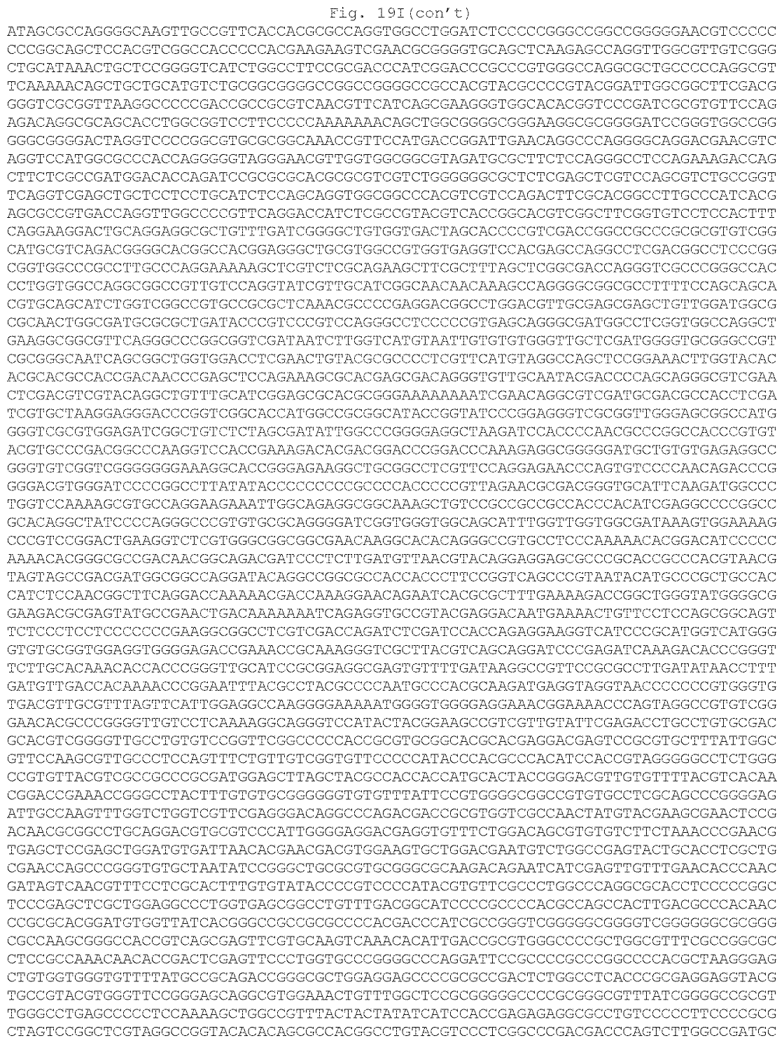

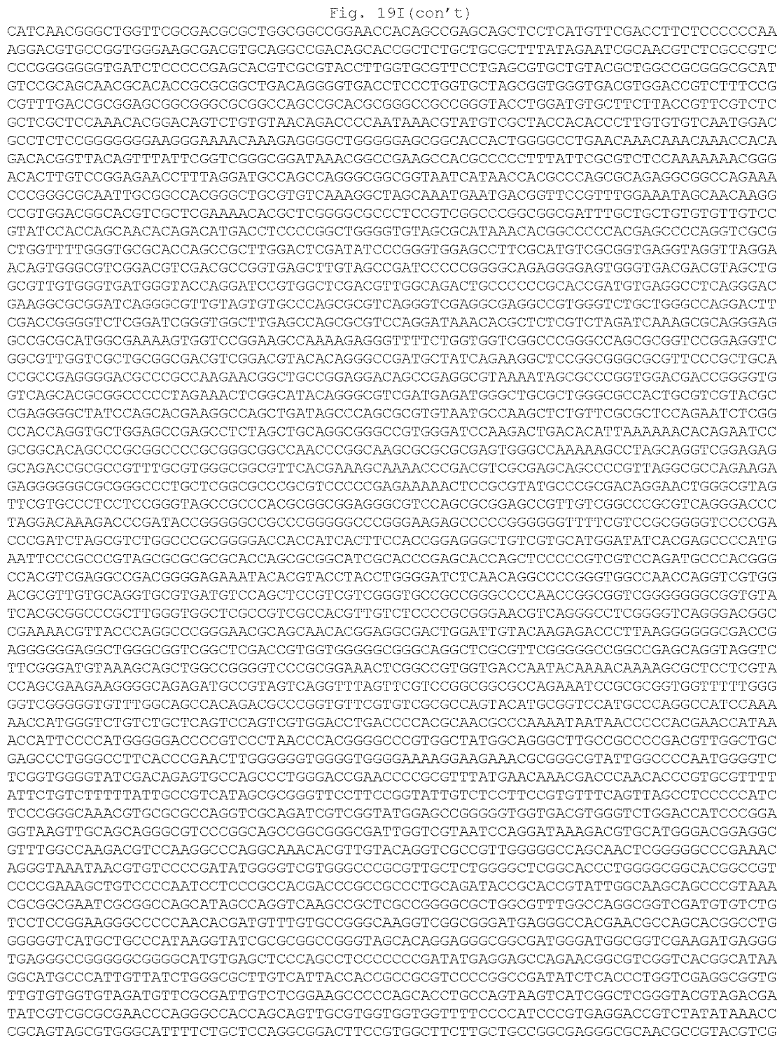

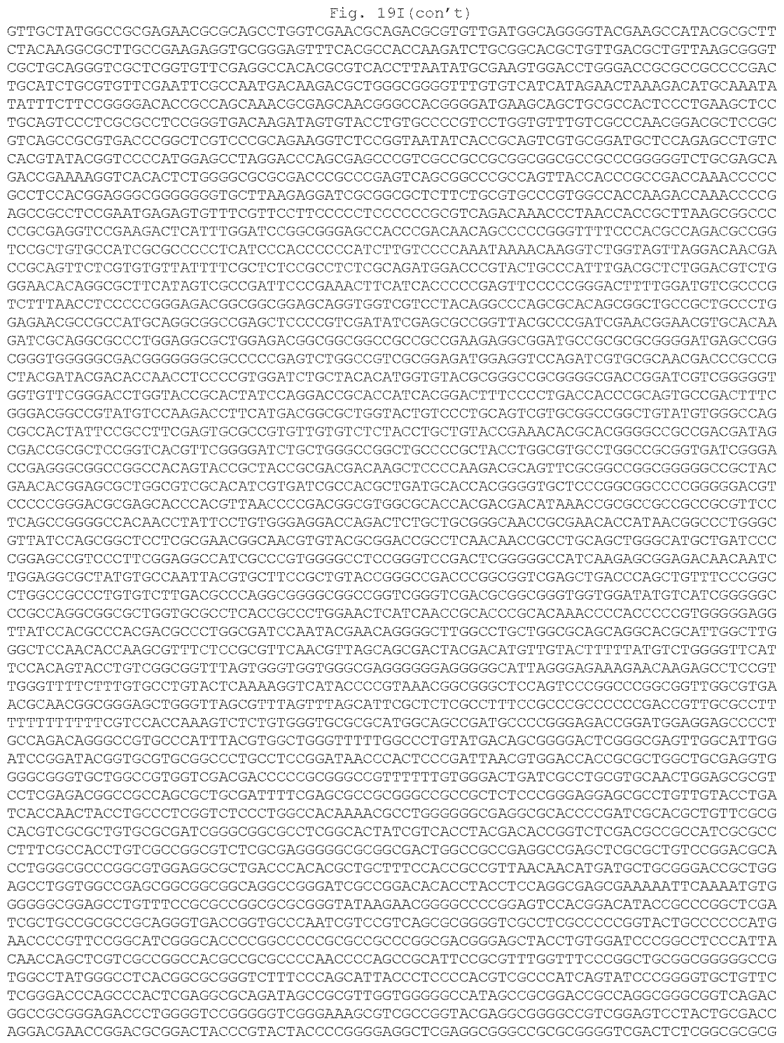

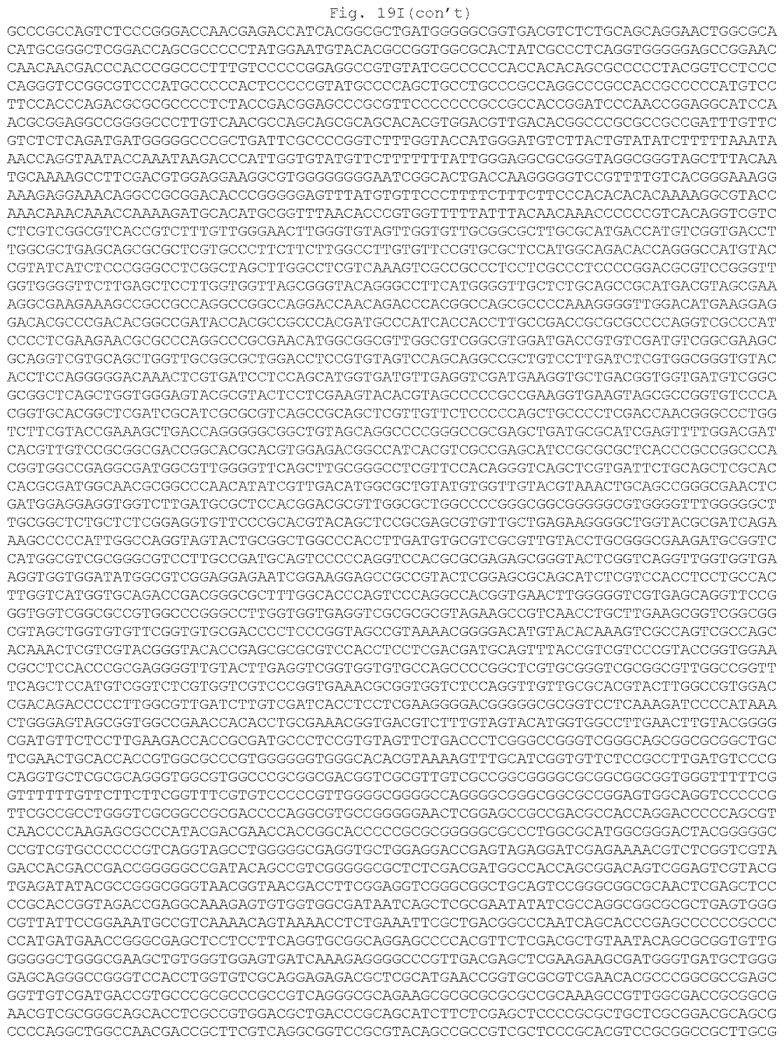

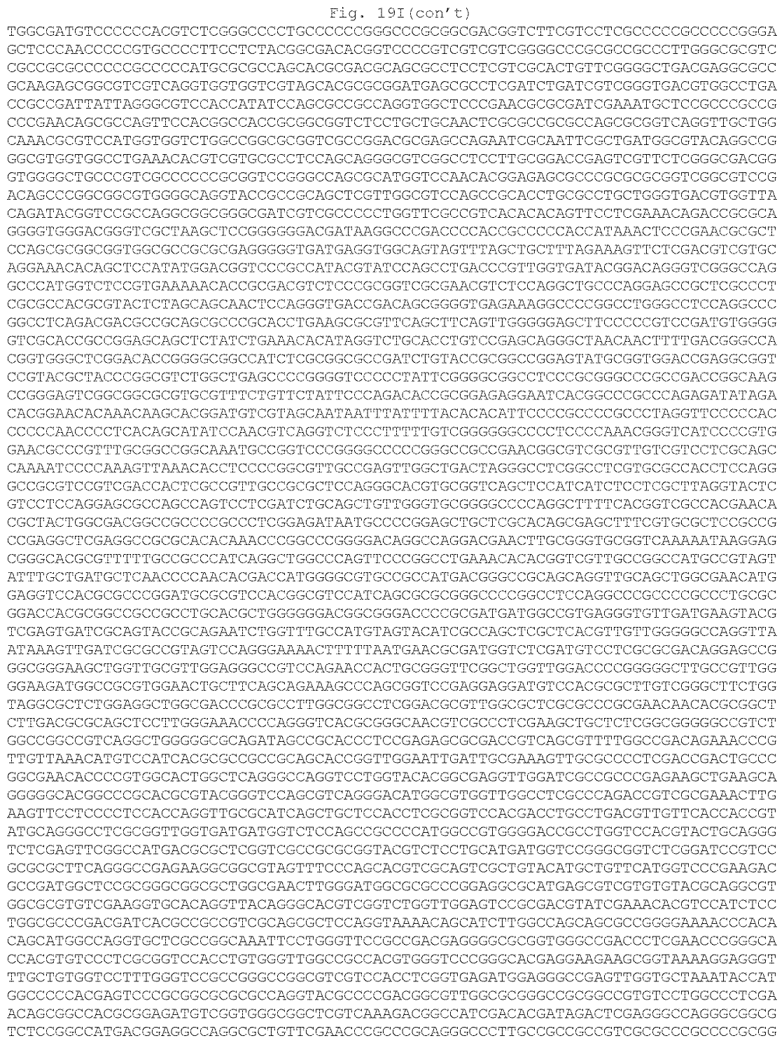

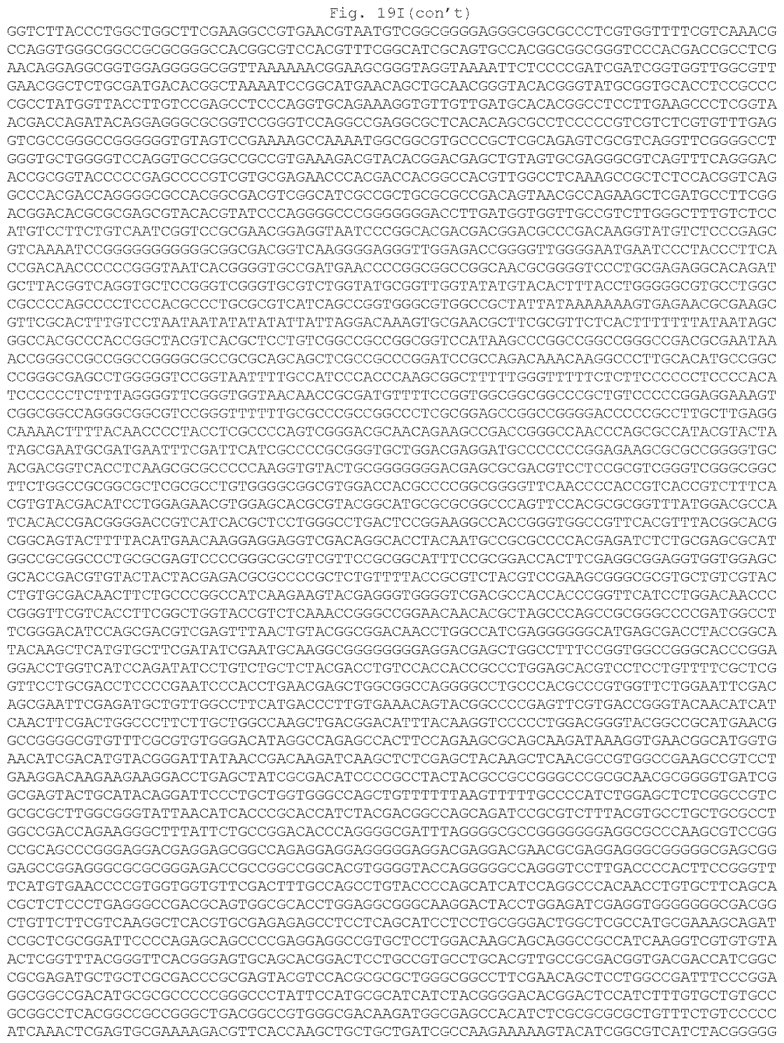

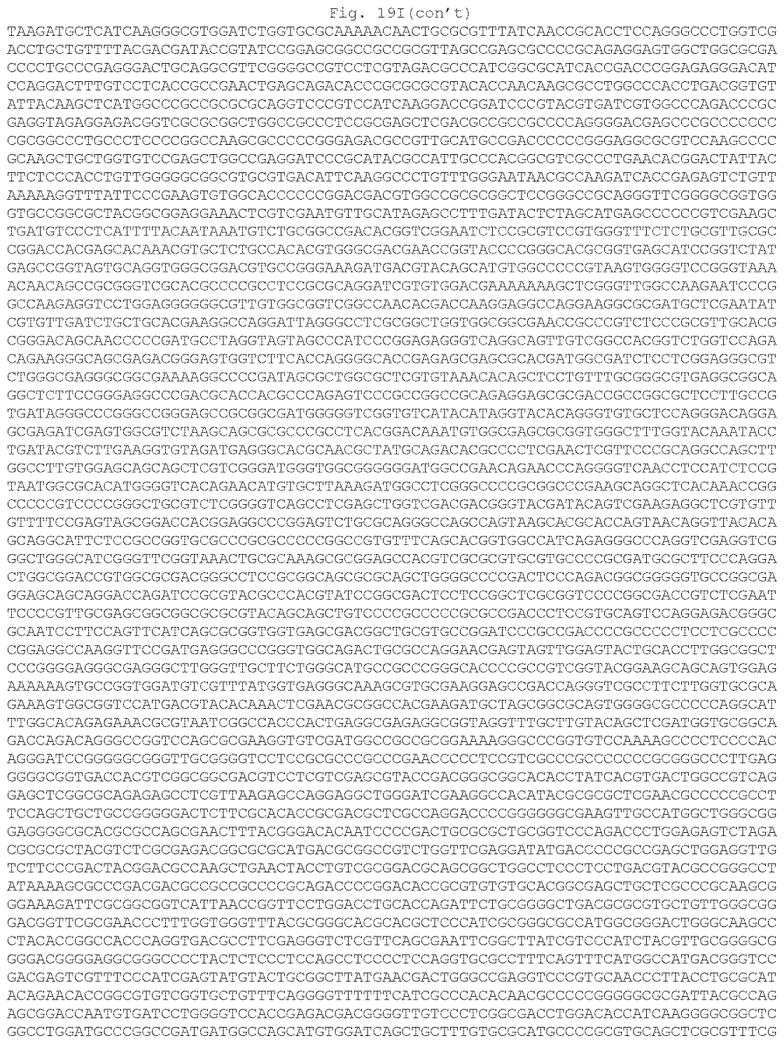

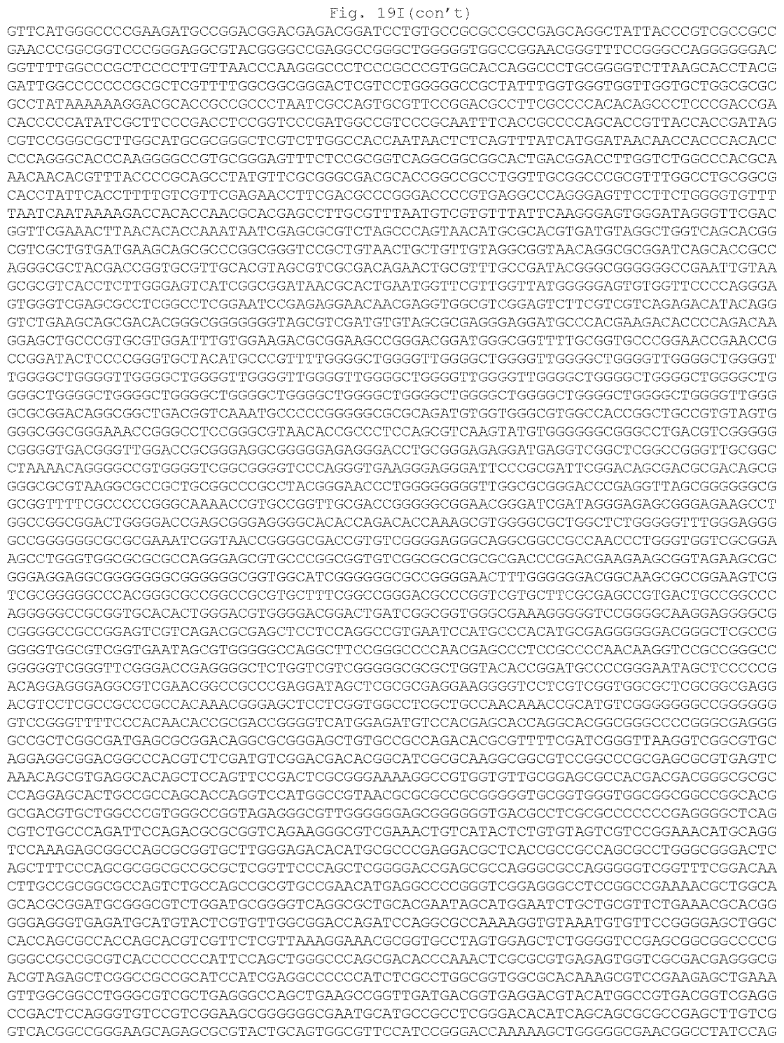

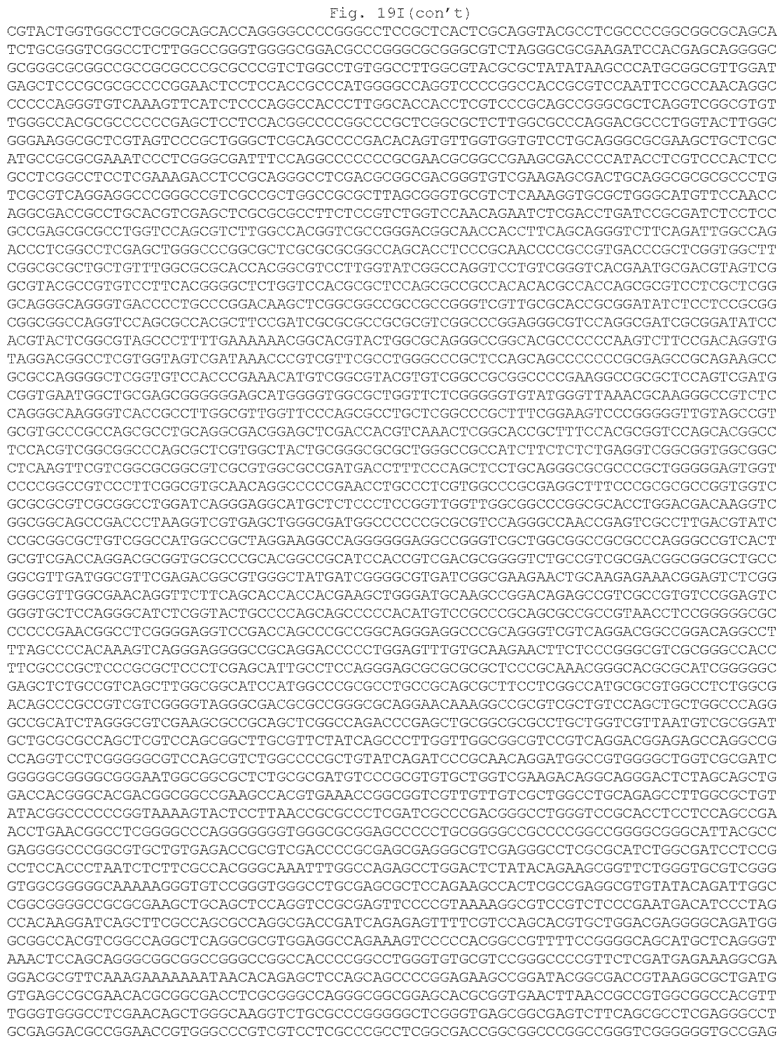

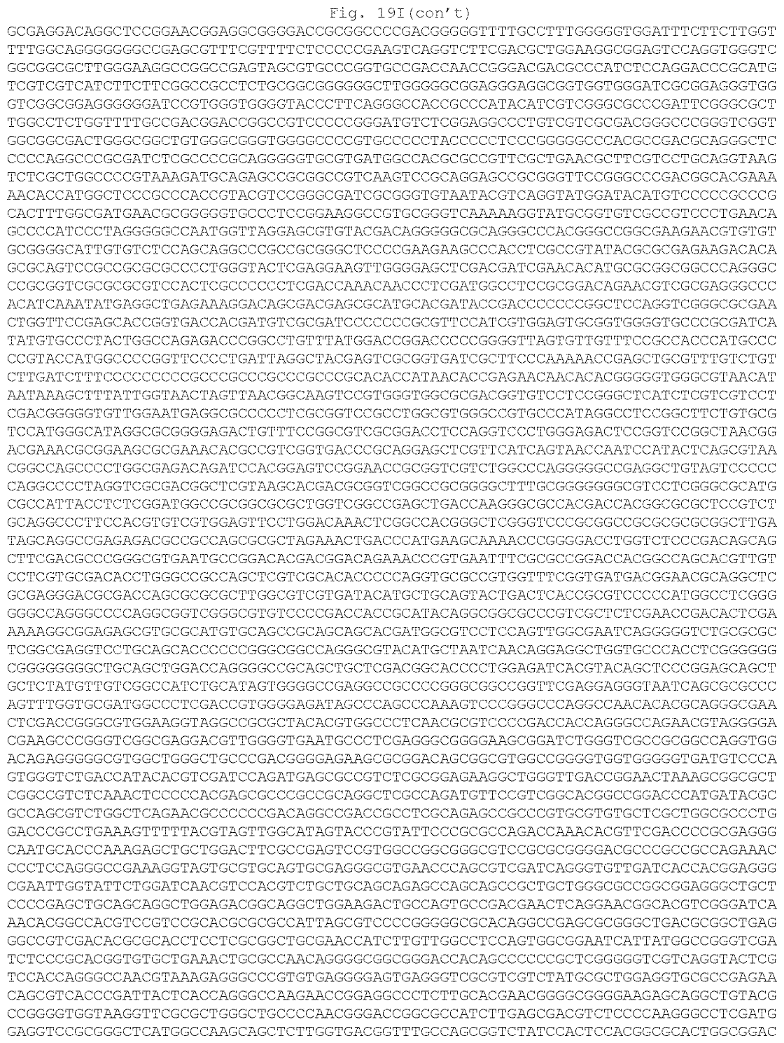

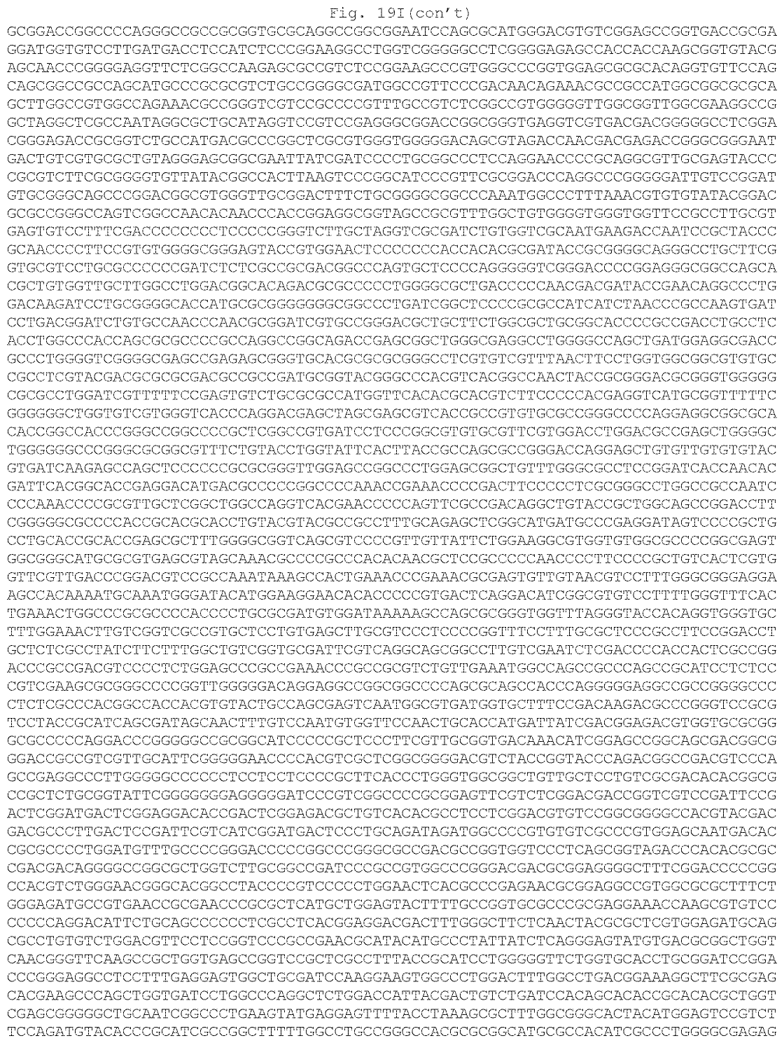

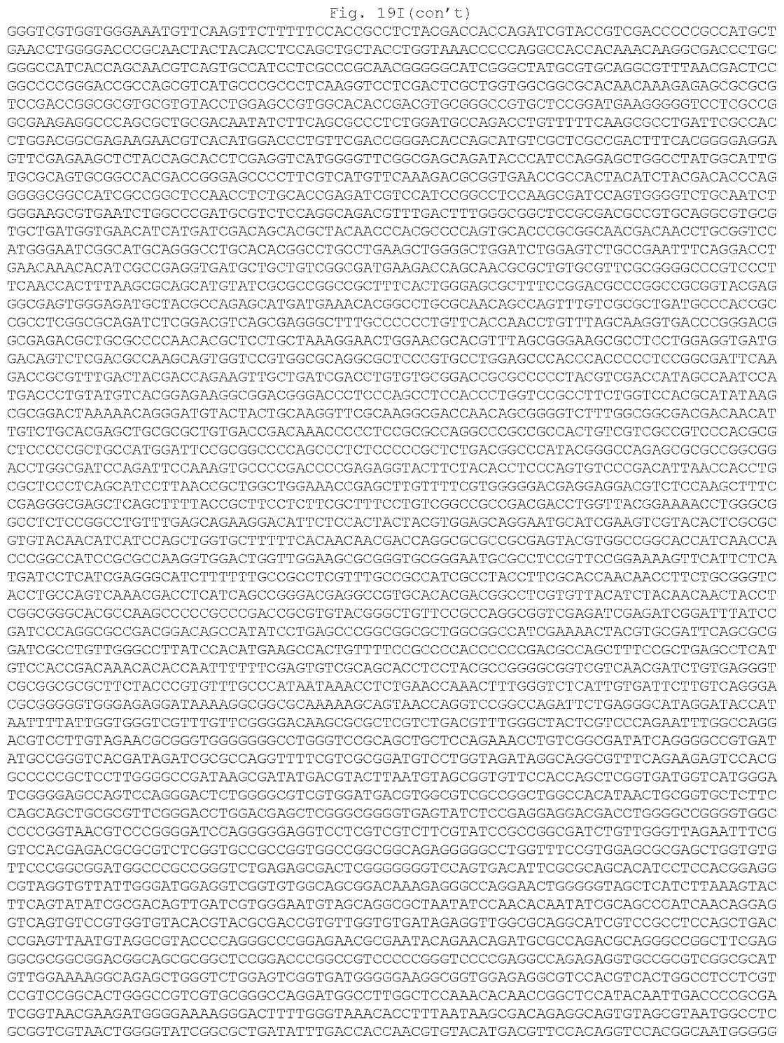

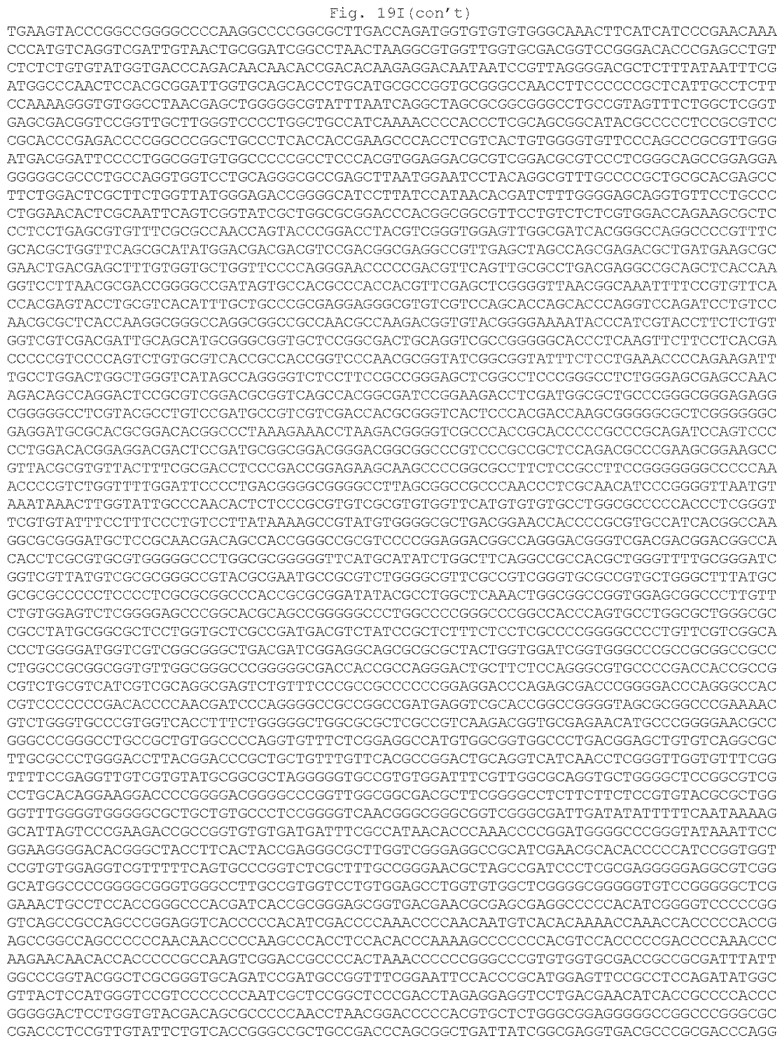

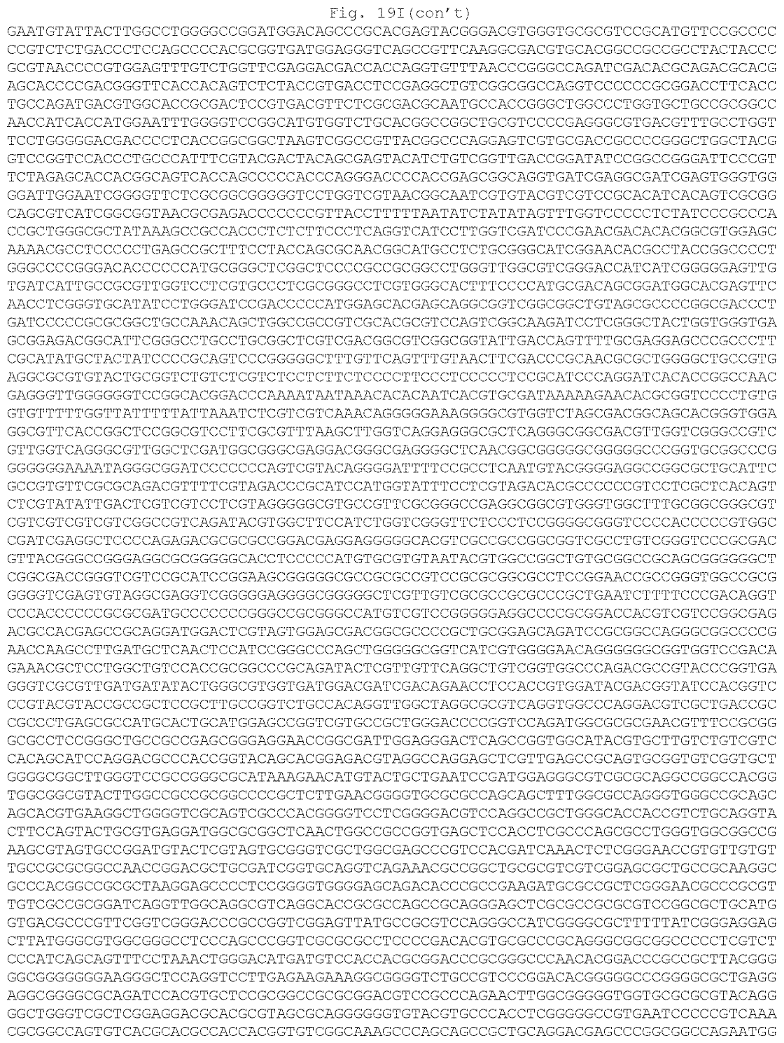

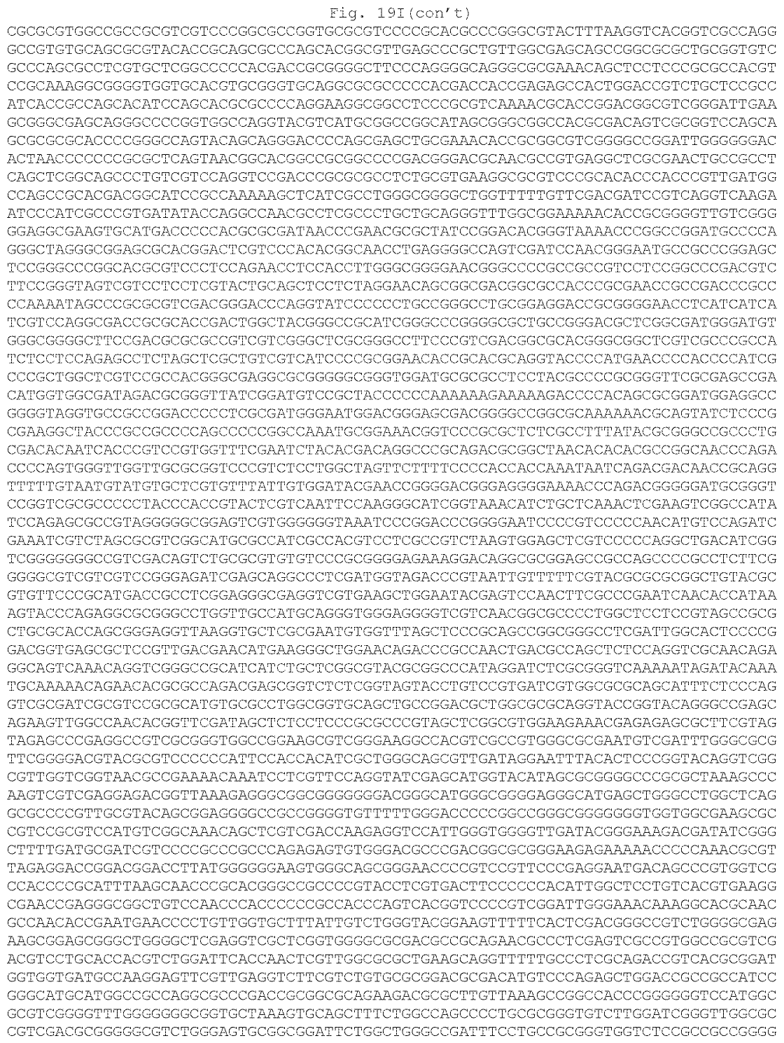

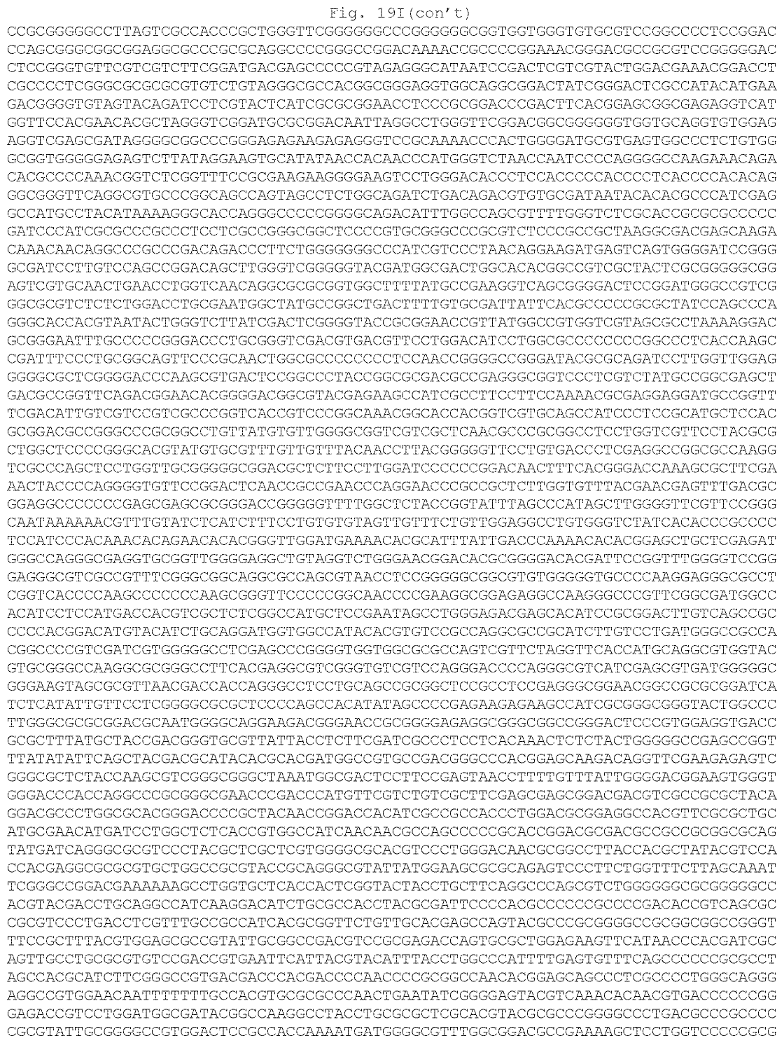

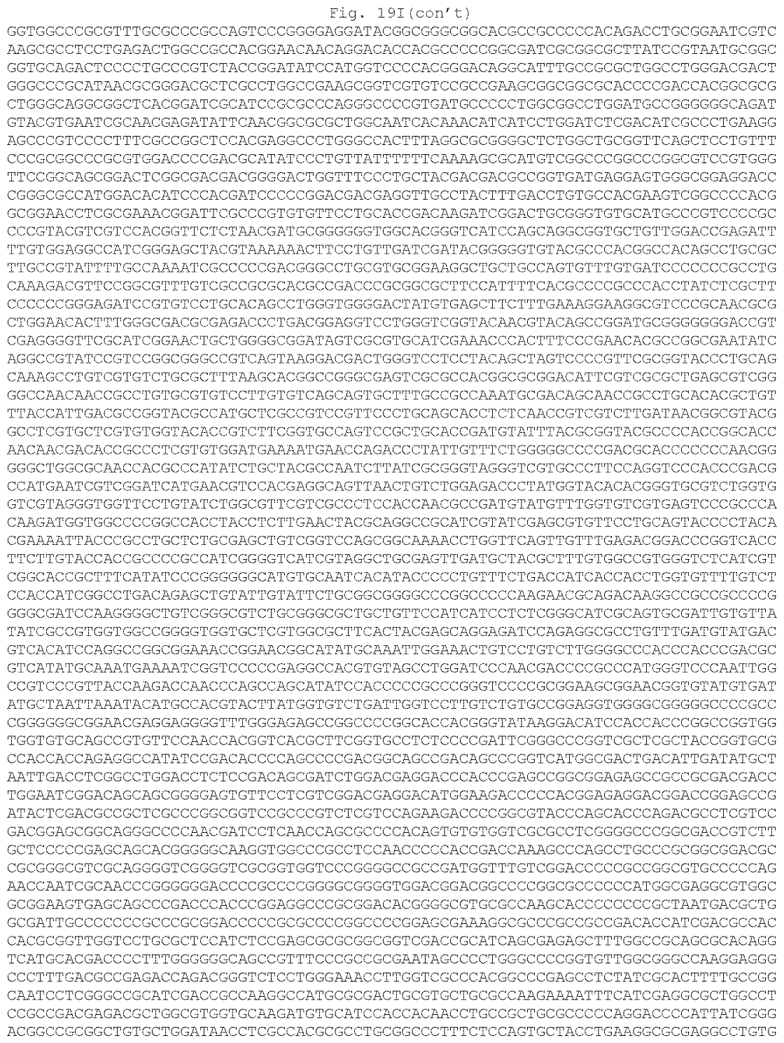

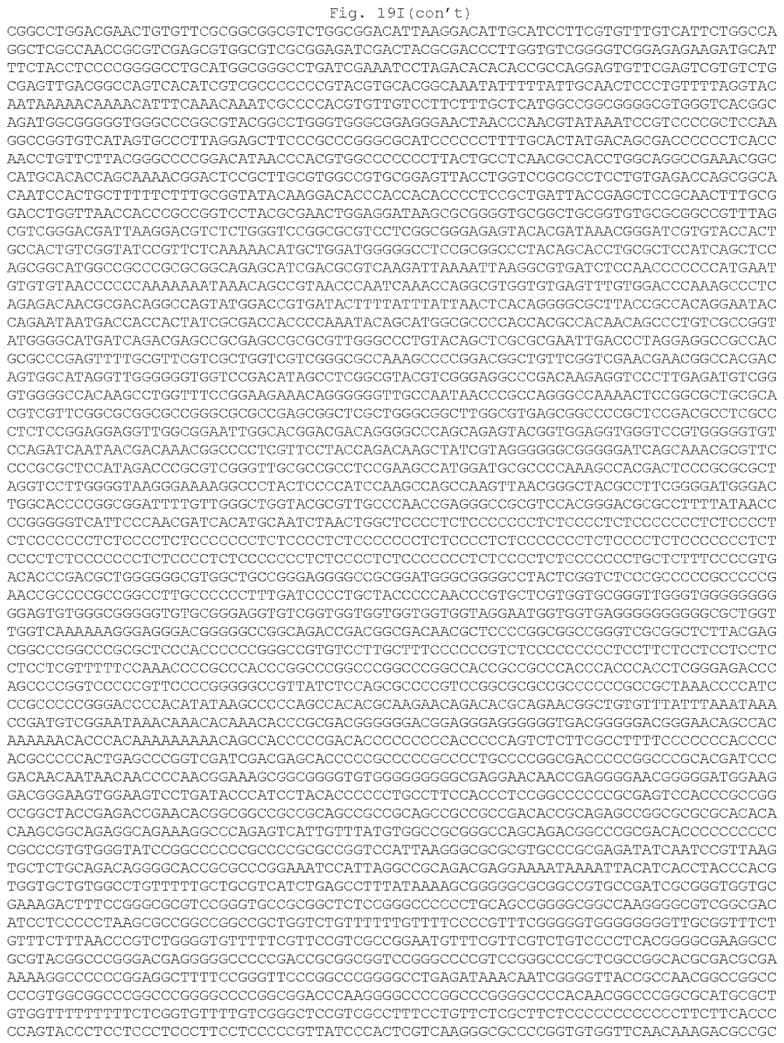

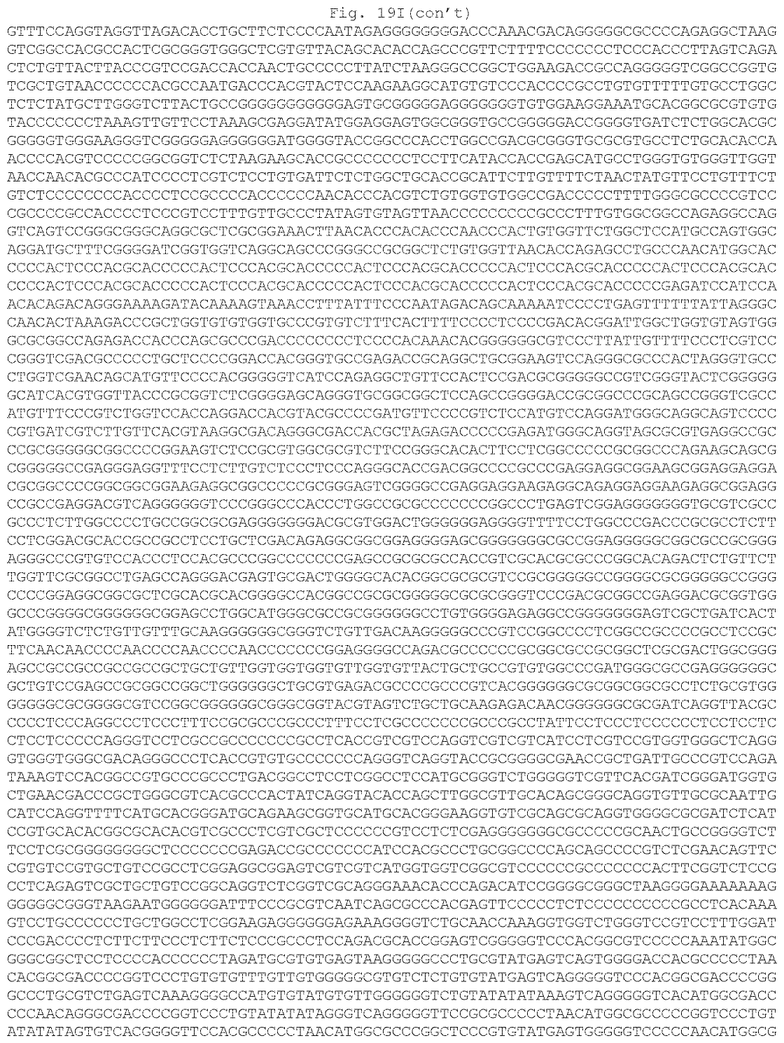

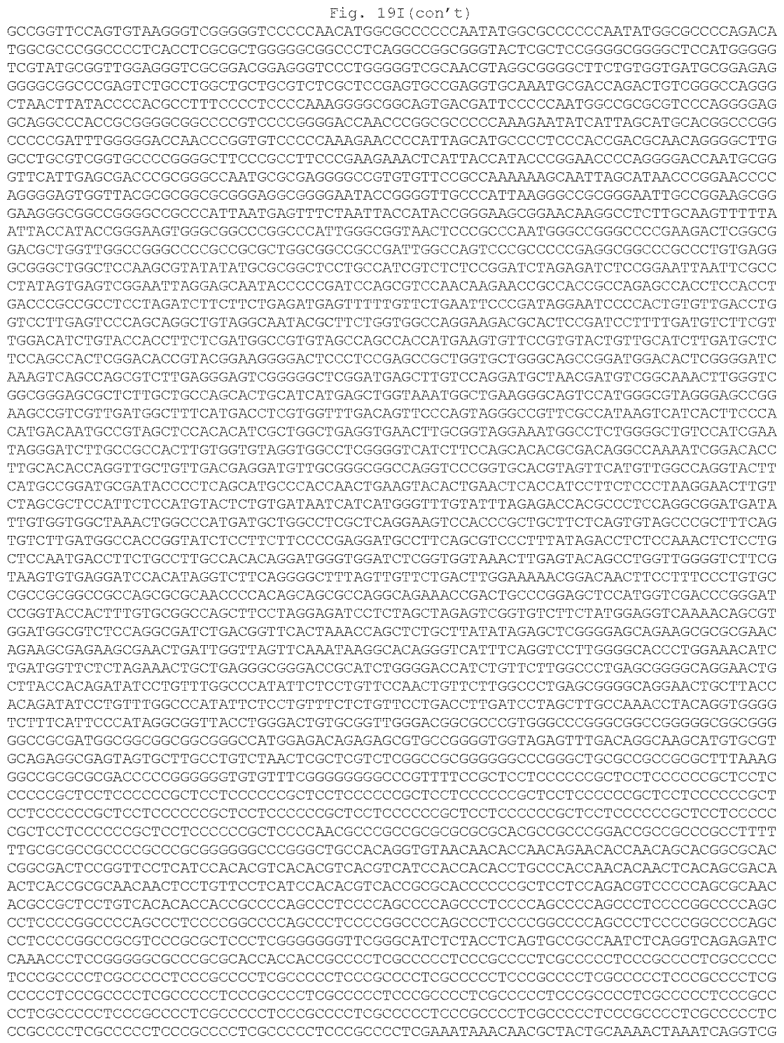

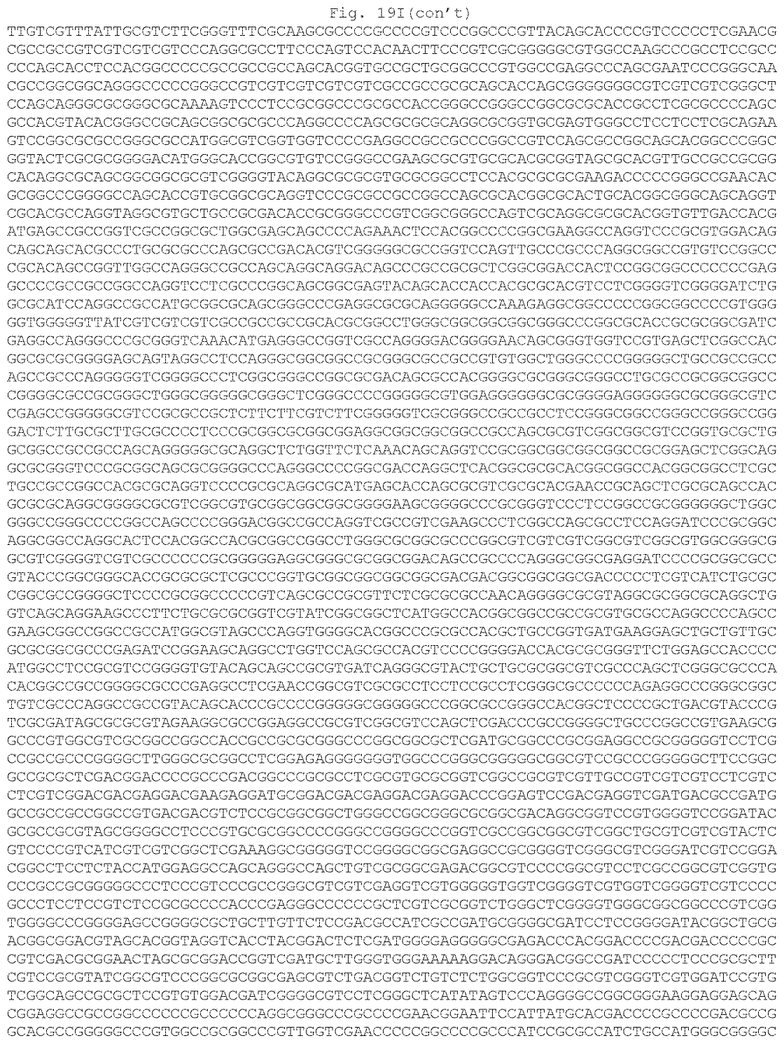

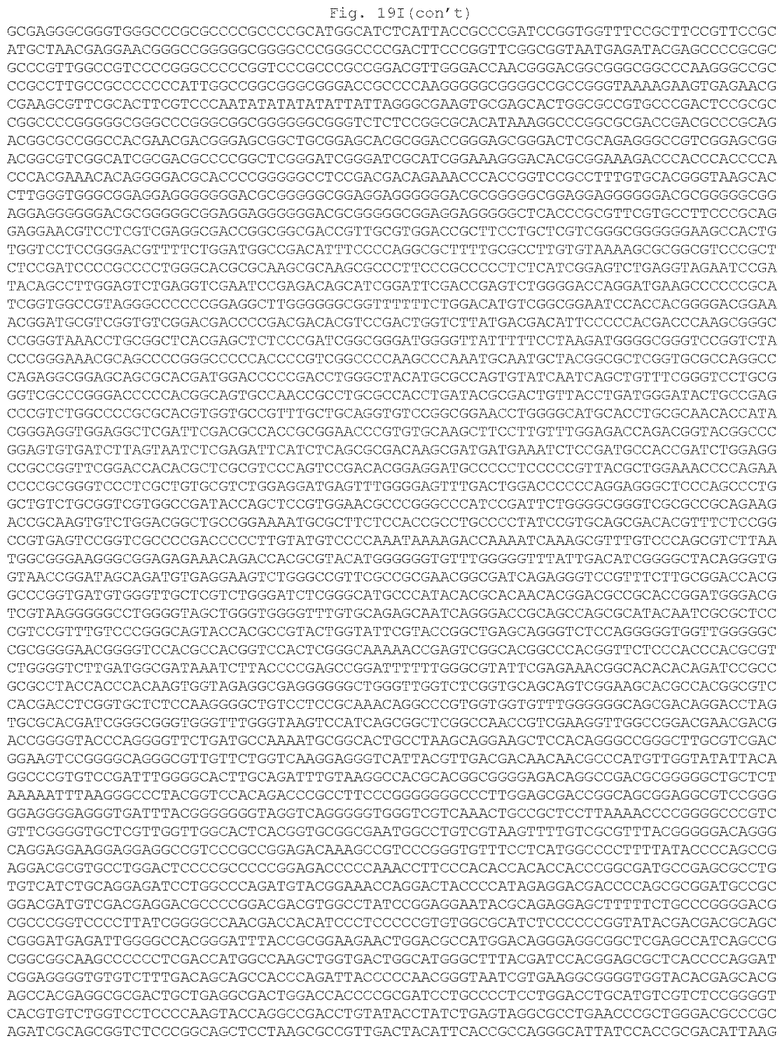

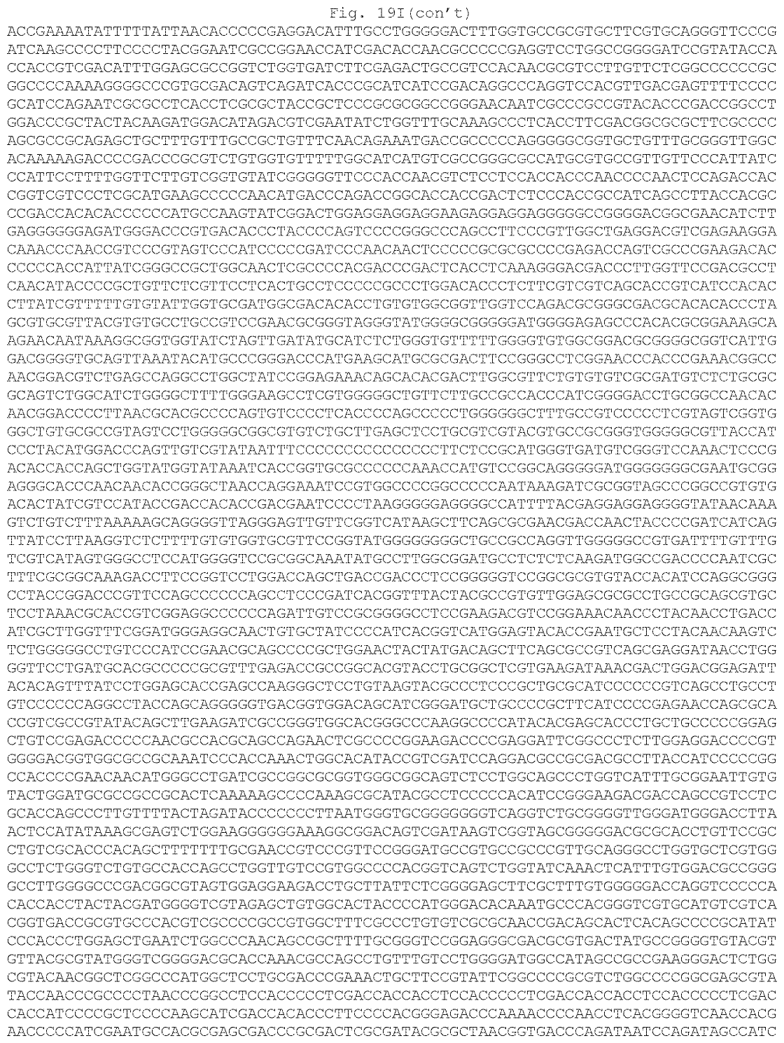

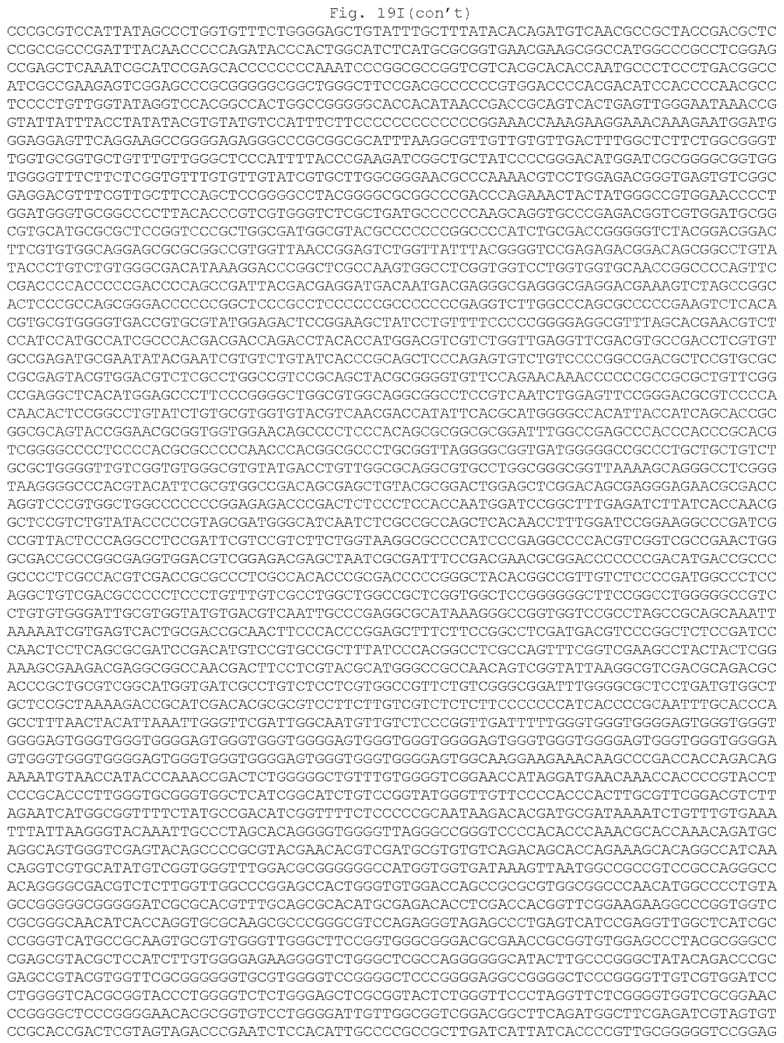

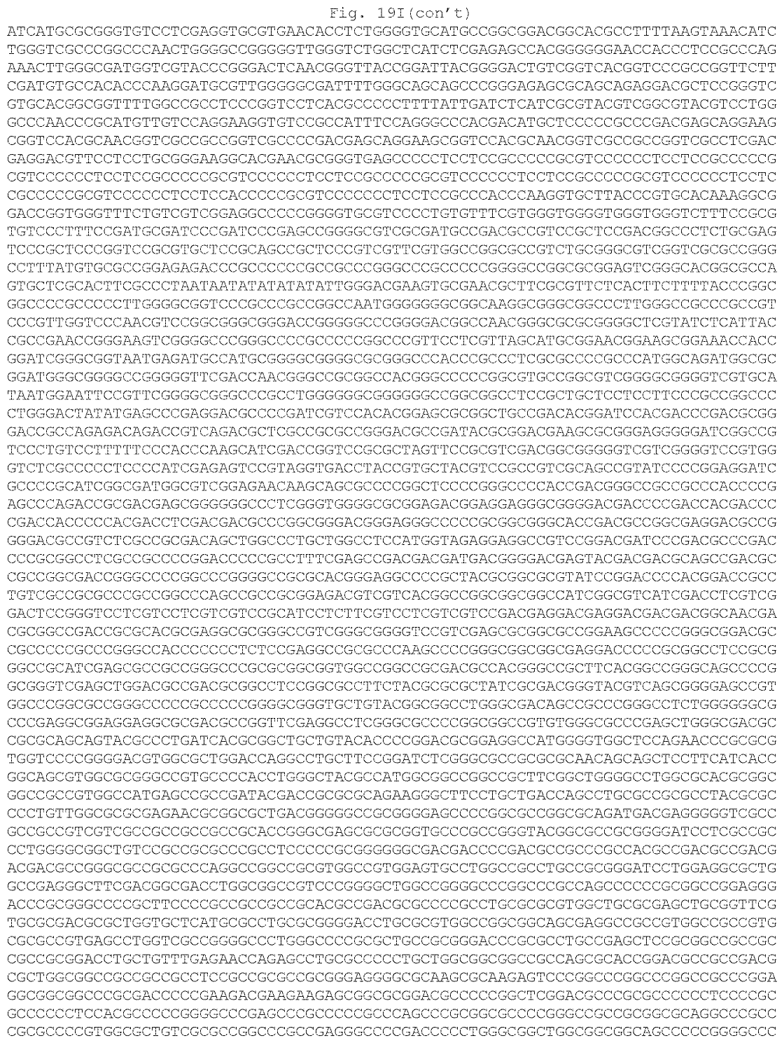

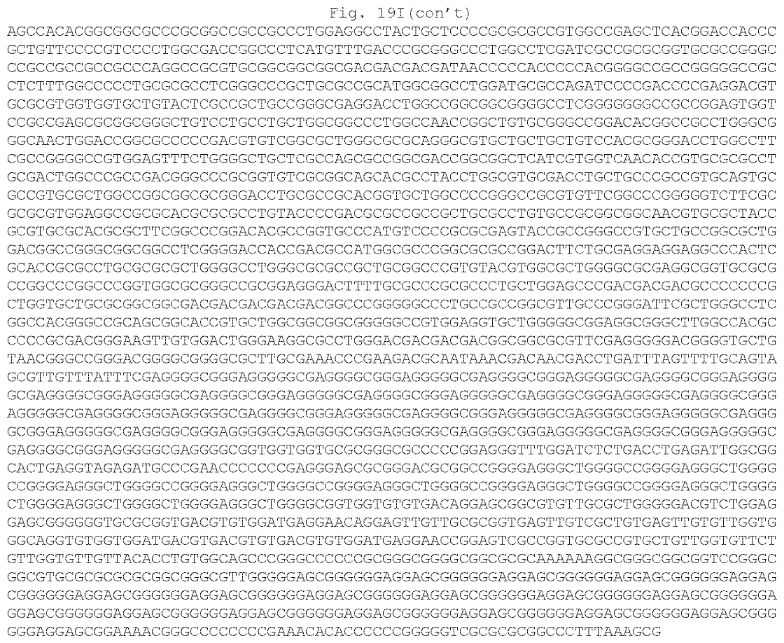

FIG. 19A-19C are a schematic representation of the viruses described herein. In these viruses, a mouse EpHA2 gene for use in some cell lines and examples. Of course the virus could be modified to encode a human EphA2 gene. Variations or isoforms of the exemplary sequences of also encompassed by the disclosure. FIGS. 19D-I disclose the complete genomic viral sequences of several chimeric viruses described herein.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A chimeric oncolytic virus, comprising:
a herpesvirus having a modified nucleic acid sequence, comprising:
a modification of the herpesvirus gamma (1)34.5 gene (γ₁34.5) or a nucleic acid with at least about 70% homology to the γ₁34.5 gene that reduces its expression;
a second viral nucleic acid sequence encoding a PKR evasion protein that does not cause virulence; and
a third nucleic acid sequence encoding a tumor-associated antigen.

2. The chimeric oncolytic virus of claim 1, wherein the herpesvirus is an α herpesvirus.

3. The chimeric oncolytic virus of claim 2, wherein the herpesvirus is an HSV-1 herpesvirus.

4. The chimeric oncolytic virus of claim 1, wherein the modification of the herpesvirus γ₁34.5 gene comprises a deletion or mutation of the γ₁34.5 gene.

5. The chimeric oncolytic virus of claim 1, wherein the second viral nucleic acid sequence is a cytomegalovirus (CMV) nucleic acid.

6. The chimeric oncolytic virus of claim 5, wherein the CMV nucleic acid comprises an IRS-1 gene or a nucleic acid having at least 70% homology to the IRS-1 gene.

7. The chimeric oncolytic virus of claim 1, wherein the tumor-associated antigen includes a dendritic cell-binding peptide.

8. The chimeric oncolytic virus of claim 1, wherein the tumor-associated antigen is a secreted protein.

9. The chimeric oncolytic virus of claim 1, wherein the tumor-associated antigen is a glioblastoma-associated antigen.

10. The chimeric oncolytic virus of claim 1, wherein the tumor-associated antigen is EphA2.

11. The chimeric oncolytic virus of claim 1, wherein the third nucleic acid sequence is inserted into the chimeric oncolytic virus at the γ₁34.5 locus.

12. The chimeric oncolytic virus of claim 1, wherein the herpesvirus includes a fourth nucleic acid sequence encoding a different tumor-associated antigen from that encoded by the third nucleic acid sequence.

13. A method of treating cancer by in a subject by contacting a cancer cell of the subject with a chimeric oncolytic virus, comprising:
a herpesvirus having a modified nucleic acid sequence, comprising:
a modification of the herpesvirus gamma (1)34.5 gene (γ₁34.5) or a nucleic acid with at least about 70% homology to the γ₁34.5 gene that reduces its expression;
a second viral nucleic acid sequence encoding a PKR evasion protein that does not cause virulence; and
a third nucleic acid sequence encoding a tumor-associated antigen.

14. The method of claim 13, wherein the herpesvirus is an HSV-1 herpesvirus.

15. The method of claim 13, wherein the modification of the herpesvirus γ₁34.5 gene comprises a deletion or mutation of the γ₁34.5 gene.

16. The method of claim 13, wherein the cancer is selected from the group consisting of adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer cell.

17. The method of claim 13, wherein the cancer is glioblastoma.

18. The method of claim 13, wherein the cancer cell is contacted ex vivo.

19. The method of claim 13, wherein the cancer cell is contacted in vivo.

20. The method of claim 19, wherein the chimeric oncolytic virus is administered in a pharmaceutically acceptable carrier.

21. The method of claim 16, further comprising administering chemotherapy or radiation therapy to the subject.

22. The method of claim 16, wherein the tumor-associated antigen is one found on the cancer being treated.

23. The method of claim 16, wherein tumor-associated antigen is EphA2.

24. The method of claim 16, wherein the herpesvirus is an HSV-1 herpesvirus.

25. The method of claim 16, wherein the second viral nucleic acid sequence is a cytomegalovirus (CMV) nucleic acid.

26. The method of claim 16, wherein the herpesvirus includes a fourth nucleic acid sequence encoding a different tumor-associated antigen from that encoded by the third nucleic acid sequence.

27. The method of claim 16, wherein the chimeric oncolytic virus provides a persistent antitumor effect.

28. A method of immunizing a subject against cancer, comprising administering to the subject a chimeric oncolytic virus, comprising:
a herpesvirus having a modified nucleic acid sequence, comprising:
a modification of the herpesvirus gamma (1)34.5 gene (γ₁34.5) or a nucleic acid with at least about 70% homology to the γ₁34.5 gene that reduces its expression;
a second viral nucleic acid sequence encoding a PKR evasion protein that does not cause virulence; and
a third nucleic acid sequence encoding a tumor-associated antigen wherein the chimeric oncolytic virus is administered under conditions effective to immunize the subject against cancer.

29. The method of claim 28, wherein the herpesvirus is an HSV-1 herpesvirus.

30. The method of claim 28, wherein the modification of the herpesvirus γ₁34.5 gene comprises a deletion or mutation of the γ₁34.5 gene.

31. The method of claim 28, wherein the cancer is selected from the group consisting of adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer cell.

32. The method of claim 28, wherein the cancer is glioblastoma.
